SEQUENCE LISTING (179 sequences)

SEQ ID NO: 1 - DNA, 30 bp, synthetic construct
  source 1..30: mol_type = other DNA; note = junction sequence
tctaatagtcttgtagccagtcagcatcat

SEQ ID NO: 2 - DNA, 30 bp, synthetic construct
  source 1..30: mol_type = other DNA; note = junction sequence
atcatactcattgctaattgtcttctatca

SEQ ID NO: 3 - DNA, 60 bp, synthetic construct
  source 1..60: mol_type = other DNA; note = junction sequence
tccatcgtcccttagtctaatagtcttgtagccagtcagcatcatcacaccaaaagttag

SEQ ID NO: 4 - DNA, 60 bp, synthetic construct
  source 1..60: mol_type = other DNA; note = junction sequence
ttctccatattgaccatcatactcattgctaattgtcttctatcattctatgtgctaccc

SEQ ID NO: 5 - DNA, 100 bp, synthetic construct
  source 1..100: mol_type = other DNA; note = junction sequence
cagagtgttatttgctttaatccatcgtcccttagtctaatagtcttgtagccagtcagcatcatcacaccaaaagttaggcccgaatagtttgaaatta

SEQ ID NO: 6 - DNA, 100 bp, synthetic construct
  source 1..100: mol_type = other DNA; note = junction sequence
ttggtaattactctttctttttctccatattgaccatcatactcattgctaattgtcttctatcattctatgtgctacccgaaaagaaaaaaaaacagac

SEQ ID NO: 7 - DNA, 1050 bp, synthetic construct
  source 1..1050: mol_type = other DNA; note = 5' genomic flank +5' end of insert
aattaaggtgaattgtgactgaacggaatcttcgacatcctaacccggtatcggggaaagtacctgtaaaaatacttcgatgatcaagttagttaagtttaaaatacttatgtatcagtatatgaaatgattgaaacgtatcttgctgtgttttctttggatgtgtgtgtgtgtcctactttattagagatccattgaaaagtgagaacttcaagctattaagacggattttttcagtgtgacacttaataatttgtaaatttgttctaagatttgtattaaattttaattcttattgtattggattttttttggaaattagcagatatggtattaagctttttgaaatttcacttattatttattttttattattttctctttatttttttagtctttttatctctcttactttcacctaggttattttatacaacctgaaaaaaatcaatgaaataaaataatatttttataaaattaactttattatcattatttttttatgattttgccacccatatcattaaattataaaggatataagtgaaaaaaattaatacttcattaaaaaattaaaataataattattttagtataatttttttttcttactaacgattattattaaatgaagggaatagtagtatcatggccatttactttcttaggagagacaaagttagttgagaatcgacacaattaaaatgttagttagaggtgatctgttagttaaataaataaagagggtaggagaaaggtagattcattccatcttgtatcattctatttataagcatttactctgtgaaaggaaaacctttgtgaaggatattcattcaaaacttttgcatctatcaatttcactcctctctttccctagttcgtaaataaaaattaaaaaaaaaaatcaacacggtgactggaagccacacctacagtctacagatatgcagagtgttatttgctttaatccatcgtcccttagtctaatagtcttgtagccagtcagcatcatcacaccaaaagttaggcccgaatagtttgaaatta

SEQ ID NO: 8 - DNA, 1050 bp, synthetic construct
  source 1..1050: mol_type = other DNA; note = 3' end of insert +3' genomic flank
ttggtaattactctttctttttctccatattgaccatcatactcattgctaattgtcttctatcattctatgtgctacccgaaaagaaaaaaaaacagacatctgttaaagagtagctgcagctttattctcttatgcatctgtcaaaccaaggagaaaaaaaatgtcaaactgaaaatttatacgtttttcctttatttcctttttaaacaattcttacattcgctattttaaacgaacatacaaatgtcttactgttttattatttcaattatcaccgttgaattgtgtcaaatttctagcaattctaggtctttaatattgactaagtaactttacccccctaaaaaaacaatagattctctctcatttccttgtgcttactaaaatcgaccctttgcaaaattatagttagaaacaccatacttgcagttgttggagtattacagtagaggctataaaaaaaaattacacagtaagaatggaagatactcgactcaaggtcaatttgacaagacaagaatacccgacctttccattaagtaacctaccaagcgtcacgactacagcaataggcataggtgaccgttgacaaaaaaaaaagagaaaaacattggtgtcctttcccatttctaattttaactattgtcttataaatagtgaaatatccctacacttcatagttttagctaatatacacagaataccacgatctcggatttggctgaactacaccgggcacactaaactcagttatcaaaagcagtatctatcattacgagaacttcctgattgcattgcaatacaacacaagaaaatactaacaacgttagaccaagtttgcataaaaattgatactttatacccaaatgtaagatctaaattgacagcaaattaatgaccctatgcacaaaacaaatgtaaacttcgtgcagcataaatagagctgttgcttcttgcacctcaaagttacttccagcataaacaaagggatgtaggaaacaacccataaatagttgtcaggaaaagtaacggggtattaccttagaa

SEQ ID NO: 9 - DNA, 10196 bp, synthetic construct
  source 1..10196: mol_type = other DNA; note = T-DNA insert
gccagtcagcatcatcacaccaaaagttaggcccgaatagtttgaaattagaaagctcgcaattgaggtctgtcgaccctgcactaactataacggtcctaaggtagcgatttattagagtagattagaatcttttatgccaagtattgataaattaaatcaagaagataaactatcataatcaacatgaaattaaaagaaaaatctcatatatagtattagtattctctatatatattatgattgcttattcttaatgggttgggttaaccaagacatagtcttaatggaaagaatcttttttgaactttttccttattgattaaattcttctatagaaaagaaagaaattatttgaggaaaagtatatacaaaaagaaaaatagaaaaatgtcagtgaagcagatgtaatggatgacctaatccaaccaccaccataggatgtttctacttgagtcggtcttttaaaaacgcacggtggaaaatatgacacgtatcatatgattccttcctttagtttcgtgataataatcctcaactgatatcttcctttttttgttttggctaaagatattttattctcattaatagaaaagacggttttgggcttttggtttgcgatataaagaagaccttcgtgtggaagataataattcatcctttcgtctttttctgactcttcaatctctcccaaagcctaaagcgatctctgcaaatctctcgcgactctctctttcaaggtatattttctgattctttttgtttttgattcgtatctgatctccaatttttgttatgtggattattgaatcttttgtataaattgcttttgacaatattgttcgtttcgtcaatccagcttctaaattttgtcctgattactaagatatcgattcgtagtgtttacatctgtgtaatttcttgcttgattgtgaaattaggattttcaaggacgatctattcaatttttgtgttttctttgttcgattctctctgttttaggtttcttatgtttagatccgtttctctttggtgttgttttgatttctcttacggcttttgatttggtatatgttcgctgattggtttctacttgttctattgttttatttcagggtatggcgacggctacgacgactgctacggcggcgtttagtggtgtagtcagtgtaggaacggagactcgaaggatttattcgttttctcatcttcaaccttctgcggcttttccggcgaagcctagttccttcaaatctctcaaattaaagcagagcgcgaggctcacacggcggcttgatcatcggccgttcgttgtccgatgtatgctcactttcgttagaaacgcttggtacgttgctgcacttcctgaggagttgagcgagaagcctctaggaagaactatcctcgatactccactagctctctatcgtcaacctgacggagttgtcgctgccctgcttgatatttgtccgcatcgcttcgctccgttgagtgacggtattctagtcaacggacatctccagtgtccatatcacggtctggaatttgacggaggtggccagtgtgtccacaacccgcacggcaacggagcccgccctgcttctctgaacgtgcgatcattccctgtcgtggaaagagacgcattgatctggatctggcctggagatccagcactcgcagatcccggtgctatccctgactttgggtgtcgtgttgatccagcttaccgtactgtcggaggttacggtcacgtggactgcaactacaagctccttgtggataacctcatggatcttggacacgctcagtacgtgcaccgcgctaacgcccaaacagacgccttcgatagacttgagcgtgaggtgatcgttggcgacggcgagatccaggcgctcatgaagatccctggtggcacaccctcagttctcatggctaagttcttgcgtggtgctaacacaccagttgacgcctggaacgacatccggtggaataaggtgtcggctatgctgaacttcatcgcggtcgcgccggaagggacgccgaaggagcagtcaatccactcccgaggaacccatatccttactcctgagaccgaggcaagctgccattacttcttcggtagttcccgcaacttcggtatagacgatccagagatggacggtgttctcaggagctggcaagctcaagccctggtgaaggaggacaaagtggtcgttgaagctatcgaaaggcggagggcttacgtcgaagcgaacgggatcagacccgccatgttgtcctgcgacgaggcagccgtcagggtatccagggagattgagaagctcgaacaactagaagcggcgtgaggcctagcaggccggtaccgagtactctccaacatggacacaccatgggattgtgtaacataaataatgtgtcgtttgtaatgaatgctcgcaacttttctagctaattaagctagagttgaacttgagctacttttatgtaccctaaagaggcacaatctttgctgttgatgtactatgatcatgttataatatgatgaaaatggagtgtgcctcattttataatttttattttcctgagtatatgtttttagggctaaacaccttataaaaaaaggtcacttagaatatgaaacatgaacttttgtaaaaaagtagagattaaaattgaaatcaaaaatttttataggatcaatattcgaagaatttttttagagggattaaaattaaatatagtttcggactgacccaaggcacaatccggctccgctcgggttcgacctgagtccaccatgcatctgtcaccttaccattgacacgccctaaaatacattagatcgcagtacaaattgagagttatgacggccacgagcgaactcctgcaggtgtttaaactagggataatagctgtttgccaatcgttcttcttggcgcgcccatagagcagtcttgtctcaaaaaaatcgaattaagcagttgaaagtgttcgtcttatcaagatgaaaatctgacaaaaattttgttaaaatttggtagtatttgataagatatgtaccttaaaattgatgttaataatcttcttatccaaaaaaagaaccaatccattgcagtcttaaatgaaatatttcaagaaaggattgagccaaaaaccgtgtttataaaattttcaaatccacaatttccacattcacttggatattacaagtgtggcatctcataaaaaaaaagaaaaagaaaaaccacgtggactattatatccagccacgtggctttaaaatcttatccaaaatcacctctcatccaacggataaggtaaccacacagccttatccaaccacatcacacgaatccactctatatatactccatataaccatcactcacacgtctttcatcacacaagtaaaacagcatcactaaaagaaaaaaaacaagaatttcaggtttagctttctctccgatctcggctttcgatctgatctcgtctgatctccgatttcgatccccgtccgatctcgatctgatcccatccgatctgtgattgattcgttgagattcagatctgatcttaggatctctctgatagatcatagattcaatctctcgagatagagatgattgatgttgttcagatcggttttgatctctgatctgatcagctcgattgattcgatttgattctcgattcgatttgatcgacaattgatctgattctctaattgatgttctgtttttgttacaggttttggtaccatgtctcccgagcgacggccagtagagattagacccgcaacagccgccgacatggcggcagtatgtgacatcgtaaatcactacatcgagacatccacagtaaactttcgtaccgaaccacaaactcctcaggaatggattgatgacttggagagactacaagaccgctatccgtggctcgtggcagaggtcgaaggcgtcgtcgcgggaatcgcctacgctggaccgtggaaggctcgaaatgcttacgactggactgtggaatccactgtttacgtctcgcatcgccatcagaggctgggtctgggcagcaccctgtacacccacttactcaaatctatggaggctcagggctttaagtcagtggtggccgtcatcggactacctaacgatccttctgtgcgcctacatgaagcactaggctacacggcacgcggaacattgagggccgccgggtacaaacacggaggatggcacgacgttggattctggcaacgggatttcgaacttccagcacctccaagacccgttagaccagttacccagatttgattaattaaacacatattcaagctctattactctgtttttaggttgagaatattgtgaagtgtgttgtgtgtgcgttctgttttatgtaaaagtaaattctgtgaattatattttgtaaggaataaattatgtaagaatggtttaattatctattgtctcacatcgttttttatgtctattttaagtattggtattgtattctatttatacacaagagttaataatagcataacataaattccacaaggatcaagtaaaataatatccacatgaaacatctaagaaaggaaaacaatatggctcaatttcctatctttctttatttatgttttagtcatctttctttccctatttcttgttgaaagctaataggagtatggtactttctacgccatttttttttaagtactctcttttacacaatattttatcaaattgtagtgacattttattttacgatgcagttaatgttcgttgtgttactgtcaatgattgtccaaaggtgaaaggccgtgacggccacgagcgaactcctgcaggtgtttaaactagggataatagctgtttgccaatcgttcttcttggcgcgccgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacaggtggtaccatgcacgcggctttgacacctttgaccaacaagtatcggttcatcgacgttcaaccactcacaggcgtgctcggcgcagagattaccggagtggacctgagggagcccttagacgactccacttggaacgagatcctcgacgcctttcacacctaccaagttatctactttcctggacaggcgatcaccaacgagcagcacattgccttctcaaggaggttcggaccggtagatccagttccaattctcaaatccattgagggttatcccgaggtgcagatgattagacgagaagccaacgagtcctcacggtacataggcgacgattggcacgcagacagcaccttccttgacgctcctccggctgccgtggttatgcgcgcaatagaggtgccggagtacggcggcgataccggtttcctatcaatgtactctgcatgggagacgctctcaccaacgatgcaagccaccattgaaggtctaaacgtggttcactcagctactaaggtcttcggaagtctttaccaggcgacgaattggaggttcagtaacaccagtgtgaaggtgatggatgtggacgctggagacagggagacggtgcatccactcgtagttacacaccctgtaactggacgcagagccctttactgcaaccaggtttactgccagaagatccagggaatgactgatgcggagtctaagtccctgcttcaattcctctacgaacacgccacccaattcgacttcacttgtcgtgttcggtggaagaaggaccaagtgctcgtgtgggataacctttgcaccatgcaccgagcagtaccagactacgccgggaaattccgctatctcacccgcactacagtggccggagacaagcctagccgctgaggccggccgcatgaataatcaagctcataaatttcatggctttgaatttgtactattttggttactagaaagtgtatttgtgtgtttatgcagtaataaatctctaagagatatatgtttgttattttttataattatccaaaaaatcgttaatgttgaaaattgattcaaaattgatattgaagttctgaaaaaatcgtggcgtgattaaaaatccaaacttttttataaaataatattgtgtctatatcttttataaatgacgataaatgggataaagtaaatgaaacaaaaccgttaatgcaatgttcatctgcacaatatatataattaaaaaacattataaaaccttgtttcttcactcatttacaatcttgaaattttagtctttaccatttgaaagtacaatcttttcatgaaagtttatagtacaaatcaagagtttggataagctgctctgctttttataatcactgggaaatgatttatgacttggaaaaacaacttgggccgtgacggccacgagcgaactcctgcaggtgtttaaactagggataacagggtaataggtctcacgcggcaaatcctaccacctcatttaaactagggataatagctgtttgccaatcgttcttcttggcgcgccaattaaattcaacacgtttgttatatattttttattgaaattattcttcattcgtcttttaatggataaaaaggtataatcaagtatattttatacacatctttctatttgtgtgtaccaaatgttaaaatggccaattttgaccaaaaaaccgcataattttcttaatttcttaaatatgattaattcatcaataacttggaatttcacaatacacaaaagtgggtgtagttaccgttattatatttatacacaacaactcatctcctcatagaaagaaaagaaaaataaaataagaaatcaaaaaacgacaagataaccaatctccacatcatccacgtggcgtaaggataaggtcacaaccaccactcagccacgtggcagaatcttatccaatcactctcaccacacaaaccttatccacttctatatataatctcttcttctcattatcactcaccacacatccttgcaaaagtaaagagaaaaaacaaacaagacaggtaaacccagatctctttcttctcttctcttcatctcgatctctccattttcataaacccaattttttctctgatttgtttgatttggtttggatctttctgtgtttccatggttttaggaattttaggatagatttttgtttgttcatgttattcatcggatatatagatttcaaattcttttgcaatttttctctctctttagttttgctcaattttggttgttgttgtgatgagtgttctctttatgggtttatctgagcttggtgagagttttttgatattgattttgcaggtggtaccctcagcgctgtgcctgttgcgatcgcaccatggctgacgagtcatggagggcgccggcgatagtgcaagagctggcggcagccggcgtcgaggagccgccgagccgatacctgctacgggagaaagaccgttctgacgtcaagctggtcgccgccgagctgccggagcctctcccagtcgttgatctcagccggctagatggtgccgaggaggccaccaagctcagggtggctctgcagaattggggcttcttcctgcttaccaaccatggagtagaagcctctctgatggacagcgtgatgaacttgtcgagagagttcttcaaccagccaatcgaacggaagcagaaattcagcaacttgatcgatggcaagaacttccagattcaagggtatggaactgaccgggtggttacccaagatcagatcctggactggtctgatcggttgcatctcagagttgaacccaaggaggagcaagatcttgccttctggcctgaccatcctgaatctttcagggatgttctgaacaagtatgcatcaggaactaaaagaattagagacgatatcattcaggctatggccaagcttcttgagcttgatgaggattacttcttggaccgactcaacgaagctcctgcattcgcaagattcaactactaccctccctgtccaaggcctgaccttgtgttcggcatcaggcctcactccgacggcaccctcttgacgattcttctcgtcgacaaagatgtcagtggcctgcaagttcagagggatggcaagtggtccaacgttgaggcaactcctcacacattgctgatcaacttaggtgacaccatggaggtaatgtgcaatggcatcttcaggagcccggtgcacagggtggtgacaaacgccgagaaggagaggatctccctggccatgttatacagcgtgaacgatgagaaagacattgagccggcggctggtttgctggatgagaatcggcctgcaagatacaggaaagtgagcgtcgaagagttcagggccgggatctttggaaaattctctcgaggagagaggtacatcgactccctgaggatctgagcgatcgcttaattaattaattaagagctcatgtctgctgcggcggccttcacagtttgtttatttcctactgtttgctgcggcgattgttgttgttttctgttttataaataataaaggaggaggagatttgttttggtttgtgtttgtttccatccttgctgctccatcacactatctgtaatttgtaaacagcgacaataaataaattaataaatttggtttctcatacctatatgtgtctgtttggaggcttgtttgtttgagacatctgtctggttgtttttttgctgccagccggtagtataaattttgtttttggacgacgaaaaatagagtgaggttgatttgcggccgcacaacaaacgccggcgctctcctctcttaaggtagccggtgacacccttgggccgcatcgatcgtgaagtttctcatctaagcccccatttggacgtgaatgtagacacgtcgaaataaagatttccgaattagaataatttgtttattgctttcgcctataaatacgacggatcgtaatttgtcgttttatcaaaatgtactttcattttataataacgctgcggacatctacatttttgaattgaaaaaaaattggtaattactctttctttttctccatattgaccatcatactcattgct

SEQ ID NO: 10 - DNA, 12196 bp, synthetic construct
  source 1..12196: mol_type = other DNA; note = 5' genomic flank +T-DNA insert +3' genomic flank
aattaaggtgaattgtgactgaacggaatcttcgacatcctaacccggtatcggggaaagtacctgtaaaaatacttcgatgatcaagttagttaagtttaaaatacttatgtatcagtatatgaaatgattgaaacgtatcttgctgtgttttctttggatgtgtgtgtgtgtcctactttattagagatccattgaaaagtgagaacttcaagctattaagacggattttttcagtgtgacacttaataatttgtaaatttgttctaagatttgtattaaattttaattcttattgtattggattttttttggaaattagcagatatggtattaagctttttgaaatttcacttattatttattttttattattttctctttatttttttagtctttttatctctcttactttcacctaggttattttatacaacctgaaaaaaatcaatgaaataaaataatatttttataaaattaactttattatcattatttttttatgattttgccacccatatcattaaattataaaggatataagtgaaaaaaattaatacttcattaaaaaattaaaataataattattttagtataatttttttttcttactaacgattattattaaatgaagggaatagtagtatcatggccatttactttcttaggagagacaaagttagttgagaatcgacacaattaaaatgttagttagaggtgatctgttagttaaataaataaagagggtaggagaaaggtagattcattccatcttgtatcattctatttataagcatttactctgtgaaaggaaaacctttgtgaaggatattcattcaaaacttttgcatctatcaatttcactcctctctttccctagttcgtaaataaaaattaaaaaaaaaaatcaacacggtgactggaagccacacctacagtctacagatatgcagagtgttatttgctttaatccatcgtcccttagtctaatagtcttgtagccagtcagcatcatcacaccaaaagttaggcccgaatagtttgaaattagaaagctcgcaattgaggtctgtcgaccctgcactaactataacggtcctaaggtagcgatttattagagtagattagaatcttttatgccaagtattgataaattaaatcaagaagataaactatcataatcaacatgaaattaaaagaaaaatctcatatatagtattagtattctctatatatattatgattgcttattcttaatgggttgggttaaccaagacatagtcttaatggaaagaatcttttttgaactttttccttattgattaaattcttctatagaaaagaaagaaattatttgaggaaaagtatatacaaaaagaaaaatagaaaaatgtcagtgaagcagatgtaatggatgacctaatccaaccaccaccataggatgtttctacttgagtcggtcttttaaaaacgcacggtggaaaatatgacacgtatcatatgattccttcctttagtttcgtgataataatcctcaactgatatcttcctttttttgttttggctaaagatattttattctcattaatagaaaagacggttttgggcttttggtttgcgatataaagaagaccttcgtgtggaagataataattcatcctttcgtctttttctgactcttcaatctctcccaaagcctaaagcgatctctgcaaatctctcgcgactctctctttcaaggtatattttctgattctttttgtttttgattcgtatctgatctccaatttttgttatgtggattattgaatcttttgtataaattgcttttgacaatattgttcgtttcgtcaatccagcttctaaattttgtcctgattactaagatatcgattcgtagtgtttacatctgtgtaatttcttgcttgattgtgaaattaggattttcaaggacgatctattcaatttttgtgttttctttgttcgattctctctgttttaggtttcttatgtttagatccgtttctctttggtgttgttttgatttctcttacggcttttgatttggtatatgttcgctgattggtttctacttgttctattgttttatttcagggtatggcgacggctacgacgactgctacggcggcgtttagtggtgtagtcagtgtaggaacggagactcgaaggatttattcgttttctcatcttcaaccttctgcggcttttccggcgaagcctagttccttcaaatctctcaaattaaagcagagcgcgaggctcacacggcggcttgatcatcggccgttcgttgtccgatgtatgctcactttcgttagaaacgcttggtacgttgctgcacttcctgaggagttgagcgagaagcctctaggaagaactatcctcgatactccactagctctctatcgtcaacctgacggagttgtcgctgccctgcttgatatttgtccgcatcgcttcgctccgttgagtgacggtattctagtcaacggacatctccagtgtccatatcacggtctggaatttgacggaggtggccagtgtgtccacaacccgcacggcaacggagcccgccctgcttctctgaacgtgcgatcattccctgtcgtggaaagagacgcattgatctggatctggcctggagatccagcactcgcagatcccggtgctatccctgactttgggtgtcgtgttgatccagcttaccgtactgtcggaggttacggtcacgtggactgcaactacaagctccttgtggataacctcatggatcttggacacgctcagtacgtgcaccgcgctaacgcccaaacagacgccttcgatagacttgagcgtgaggtgatcgttggcgacggcgagatccaggcgctcatgaagatccctggtggcacaccctcagttctcatggctaagttcttgcgtggtgctaacacaccagttgacgcctggaacgacatccggtggaataaggtgtcggctatgctgaacttcatcgcggtcgcgccggaagggacgccgaaggagcagtcaatccactcccgaggaacccatatccttactcctgagaccgaggcaagctgccattacttcttcggtagttcccgcaacttcggtatagacgatccagagatggacggtgttctcaggagctggcaagctcaagccctggtgaaggaggacaaagtggtcgttgaagctatcgaaaggcggagggcttacgtcgaagcgaacgggatcagacccgccatgttgtcctgcgacgaggcagccgtcagggtatccagggagattgagaagctcgaacaactagaagcggcgtgaggcctagcaggccggtaccgagtactctccaacatggacacaccatgggattgtgtaacataaataatgtgtcgtttgtaatgaatgctcgcaacttttctagctaattaagctagagttgaacttgagctacttttatgtaccctaaagaggcacaatctttgctgttgatgtactatgatcatgttataatatgatgaaaatggagtgtgcctcattttataatttttattttcctgagtatatgtttttagggctaaacaccttataaaaaaaggtcacttagaatatgaaacatgaacttttgtaaaaaagtagagattaaaattgaaatcaaaaatttttataggatcaatattcgaagaatttttttagagggattaaaattaaatatagtttcggactgacccaaggcacaatccggctccgctcgggttcgacctgagtccaccatgcatctgtcaccttaccattgacacgccctaaaatacattagatcgcagtacaaattgagagttatgacggccacgagcgaactcctgcaggtgtttaaactagggataatagctgtttgccaatcgttcttcttggcgcgcccatagagcagtcttgtctcaaaaaaatcgaattaagcagttgaaagtgttcgtcttatcaagatgaaaatctgacaaaaattttgttaaaatttggtagtatttgataagatatgtaccttaaaattgatgttaataatcttcttatccaaaaaaagaaccaatccattgcagtcttaaatgaaatatttcaagaaaggattgagccaaaaaccgtgtttataaaattttcaaatccacaatttccacattcacttggatattacaagtgtggcatctcataaaaaaaaagaaaaagaaaaaccacgtggactattatatccagccacgtggctttaaaatcttatccaaaatcacctctcatccaacggataaggtaaccacacagccttatccaaccacatcacacgaatccactctatatatactccatataaccatcactcacacgtctttcatcacacaagtaaaacagcatcactaaaagaaaaaaaacaagaatttcaggtttagctttctctccgatctcggctttcgatctgatctcgtctgatctccgatttcgatccccgtccgatctcgatctgatcccatccgatctgtgattgattcgttgagattcagatctgatcttaggatctctctgatagatcatagattcaatctctcgagatagagatgattgatgttgttcagatcggttttgatctctgatctgatcagctcgattgattcgatttgattctcgattcgatttgatcgacaattgatctgattctctaattgatgttctgtttttgttacaggttttggtaccatgtctcccgagcgacggccagtagagattagacccgcaacagccgccgacatggcggcagtatgtgacatcgtaaatcactacatcgagacatccacagtaaactttcgtaccgaaccacaaactcctcaggaatggattgatgacttggagagactacaagaccgctatccgtggctcgtggcagaggtcgaaggcgtcgtcgcgggaatcgcctacgctggaccgtggaaggctcgaaatgcttacgactggactgtggaatccactgtttacgtctcgcatcgccatcagaggctgggtctgggcagcaccctgtacacccacttactcaaatctatggaggctcagggctttaagtcagtggtggccgtcatcggactacctaacgatccttctgtgcgcctacatgaagcactaggctacacggcacgcggaacattgagggccgccgggtacaaacacggaggatggcacgacgttggattctggcaacgggatttcgaacttccagcacctccaagacccgttagaccagttacccagatttgattaattaaacacatattcaagctctattactctgtttttaggttgagaatattgtgaagtgtgttgtgtgtgcgttctgttttatgtaaaagtaaattctgtgaattatattttgtaaggaataaattatgtaagaatggtttaattatctattgtctcacatcgttttttatgtctattttaagtattggtattgtattctatttatacacaagagttaataatagcataacataaattccacaaggatcaagtaaaataatatccacatgaaacatctaagaaaggaaaacaatatggctcaatttcctatctttctttatttatgttttagtcatctttctttccctatttcttgttgaaagctaataggagtatggtactttctacgccatttttttttaagtactctcttttacacaatattttatcaaattgtagtgacattttattttacgatgcagttaatgttcgttgtgttactgtcaatgattgtccaaaggtgaaaggccgtgacggccacgagcgaactcctgcaggtgtttaaactagggataatagctgtttgccaatcgttcttcttggcgcgccgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacaggtggtaccatgcacgcggctttgacacctttgaccaacaagtatcggttcatcgacgttcaaccactcacaggcgtgctcggcgcagagattaccggagtggacctgagggagcccttagacgactccacttggaacgagatcctcgacgcctttcacacctaccaagttatctactttcctggacaggcgatcaccaacgagcagcacattgccttctcaaggaggttcggaccggtagatccagttccaattctcaaatccattgagggttatcccgaggtgcagatgattagacgagaagccaacgagtcctcacggtacataggcgacgattggcacgcagacagcaccttccttgacgctcctccggctgccgtggttatgcgcgcaatagaggtgccggagtacggcggcgataccggtttcctatcaatgtactctgcatgggagacgctctcaccaacgatgcaagccaccattgaaggtctaaacgtggttcactcagctactaaggtcttcggaagtctttaccaggcgacgaattggaggttcagtaacaccagtgtgaaggtgatggatgtggacgctggagacagggagacggtgcatccactcgtagttacacaccctgtaactggacgcagagccctttactgcaaccaggtttactgccagaagatccagggaatgactgatgcggagtctaagtccctgcttcaattcctctacgaacacgccacccaattcgacttcacttgtcgtgttcggtggaagaaggaccaagtgctcgtgtgggataacctttgcaccatgcaccgagcagtaccagactacgccgggaaattccgctatctcacccgcactacagtggccggagacaagcctagccgctgaggccggccgcatgaataatcaagctcataaatttcatggctttgaatttgtactattttggttactagaaagtgtatttgtgtgtttatgcagtaataaatctctaagagatatatgtttgttattttttataattatccaaaaaatcgttaatgttgaaaattgattcaaaattgatattgaagttctgaaaaaatcgtggcgtgattaaaaatccaaacttttttataaaataatattgtgtctatatcttttataaatgacgataaatgggataaagtaaatgaaacaaaaccgttaatgcaatgttcatctgcacaatatatataattaaaaaacattataaaaccttgtttcttcactcatttacaatcttgaaattttagtctttaccatttgaaagtacaatcttttcatgaaagtttatagtacaaatcaagagtttggataagctgctctgctttttataatcactgggaaatgatttatgacttggaaaaacaacttgggccgtgacggccacgagcgaactcctgcaggtgtttaaactagggataacagggtaataggtctcacgcggcaaatcctaccacctcatttaaactagggataatagctgtttgccaatcgttcttcttggcgcgccaattaaattcaacacgtttgttatatattttttattgaaattattcttcattcgtcttttaatggataaaaaggtataatcaagtatattttatacacatctttctatttgtgtgtaccaaatgttaaaatggccaattttgaccaaaaaaccgcataattttcttaatttcttaaatatgattaattcatcaataacttggaatttcacaatacacaaaagtgggtgtagttaccgttattatatttatacacaacaactcatctcctcatagaaagaaaagaaaaataaaataagaaatcaaaaaacgacaagataaccaatctccacatcatccacgtggcgtaaggataaggtcacaaccaccactcagccacgtggcagaatcttatccaatcactctcaccacacaaaccttatccacttctatatataatctcttcttctcattatcactcaccacacatccttgcaaaagtaaagagaaaaaacaaacaagacaggtaaacccagatctctttcttctcttctcttcatctcgatctctccattttcataaacccaattttttctctgatttgtttgatttggtttggatctttctgtgtttccatggttttaggaattttaggatagatttttgtttgttcatgttattcatcggatatatagatttcaaattcttttgcaatttttctctctctttagttttgctcaattttggttgttgttgtgatgagtgttctctttatgggtttatctgagcttggtgagagttttttgatattgattttgcaggtggtaccctcagcgctgtgcctgttgcgatcgcaccatggctgacgagtcatggagggcgccggcgatagtgcaagagctggcggcagccggcgtcgaggagccgccgagccgatacctgctacgggagaaagaccgttctgacgtcaagctggtcgccgccgagctgccggagcctctcccagtcgttgatctcagccggctagatggtgccgaggaggccaccaagctcagggtggctctgcagaattggggcttcttcctgcttaccaaccatggagtagaagcctctctgatggacagcgtgatgaacttgtcgagagagttcttcaaccagccaatcgaacggaagcagaaattcagcaacttgatcgatggcaagaacttccagattcaagggtatggaactgaccgggtggttacccaagatcagatcctggactggtctgatcggttgcatctcagagttgaacccaaggaggagcaagatcttgccttctggcctgaccatcctgaatctttcagggatgttctgaacaagtatgcatcaggaactaaaagaattagagacgatatcattcaggctatggccaagcttcttgagcttgatgaggattacttcttggaccgactcaacgaagctcctgcattcgcaagattcaactactaccctccctgtccaaggcctgaccttgtgttcggcatcaggcctcactccgacggcaccctcttgacgattcttctcgtcgacaaagatgtcagtggcctgcaagttcagagggatggcaagtggtccaacgttgaggcaactcctcacacattgctgatcaacttaggtgacaccatggaggtaatgtgcaatggcatcttcaggagcccggtgcacagggtggtgacaaacgccgagaaggagaggatctccctggccatgttatacagcgtgaacgatgagaaagacattgagccggcggctggtttgctggatgagaatcggcctgcaagatacaggaaagtgagcgtcgaagagttcagggccgggatctttggaaaattctctcgaggagagaggtacatcgactccctgaggatctgagcgatcgcttaattaattaattaagagctcatgtctgctgcggcggccttcacagtttgtttatttcctactgtttgctgcggcgattgttgttgttttctgttttataaataataaaggaggaggagatttgttttggtttgtgtttgtttccatccttgctgctccatcacactatctgtaatttgtaaacagcgacaataaataaattaataaatttggtttctcatacctatatgtgtctgtttggaggcttgtttgtttgagacatctgtctggttgtttttttgctgccagccggtagtataaattttgtttttggacgacgaaaaatagagtgaggttgatttgcggccgcacaacaaacgccggcgctctcctctcttaaggtagccggtgacacccttgggccgcatcgatcgtgaagtttctcatctaagcccccatttggacgtgaatgtagacacgtcgaaataaagatttccgaattagaataatttgtttattgctttcgcctataaatacgacggatcgtaatttgtcgttttatcaaaatgtactttcattttataataacgctgcggacatctacatttttgaattgaaaaaaaattggtaattactctttctttttctccatattgaccatcatactcattgctaattgtcttctatcattctatgtgctacccgaaaagaaaaaaaaacagacatctgttaaagagtagctgcagctttattctcttatgcatctgtcaaaccaaggagaaaaaaaatgtcaaactgaaaatttatacgtttttcctttatttcctttttaaacaattcttacattcgctattttaaacgaacatacaaatgtcttactgttttattatttcaattatcaccgttgaattgtgtcaaatttctagcaattctaggtctttaatattgactaagtaactttacccccctaaaaaaacaatagattctctctcatttccttgtgcttactaaaatcgaccctttgcaaaattatagttagaaacaccatacttgcagttgttggagtattacagtagaggctataaaaaaaaattacacagtaagaatggaagatactcgactcaaggtcaatttgacaagacaagaatacccgacctttccattaagtaacctaccaagcgtcacgactacagcaataggcataggtgaccgttgacaaaaaaaaaagagaaaaacattggtgtcctttcccatttctaattttaactattgtcttataaatagtgaaatatccctacacttcatagttttagctaatatacacagaataccacgatctcggatttggctgaactacaccgggcacactaaactcagttatcaaaagcagtatctatcattacgagaacttcctgattgcattgcaatacaacacaagaaaatactaacaacgttagaccaagtttgcataaaaattgatactttatacccaaatgtaagatctaaattgacagcaaattaatgaccctatgcacaaaacaaatgtaaacttcgtgcagcataaatagagctgttgcttcttgcacctcaaagttacttccagcataaacaaagggatgtaggaaacaacccataaatagttgtcaggaaaagtaacggggtattaccttagaa

SEQ ID NO: 11 - DNA, 1000 bp, Glycine max
  source 1..1000: mol_type = genomic DNA
aattaaggtgaattgtgactgaacggaatcttcgacatcctaacccggtatcggggaaagtacctgtaaaaatacttcgatgatcaagttagttaagtttaaaatacttatgtatcagtatatgaaatgattgaaacgtatcttgctgtgttttctttggatgtgtgtgtgtgtcctactttattagagatccattgaaaagtgagaacttcaagctattaagacggattttttcagtgtgacacttaataatttgtaaatttgttctaagatttgtattaaattttaattcttattgtattggattttttttggaaattagcagatatggtattaagctttttgaaatttcacttattatttattttttattattttctctttatttttttagtctttttatctctcttactttcacctaggttattttatacaacctgaaaaaaatcaatgaaataaaataatatttttataaaattaactttattatcattatttttttatgattttgccacccatatcattaaattataaaggatataagtgaaaaaaattaatacttcattaaaaaattaaaataataattattttagtataatttttttttcttactaacgattattattaaatgaagggaatagtagtatcatggccatttactttcttaggagagacaaagttagttgagaatcgacacaattaaaatgttagttagaggtgatctgttagttaaataaataaagagggtaggagaaaggtagattcattccatcttgtatcattctatttataagcatttactctgtgaaaggaaaacctttgtgaaggatattcattcaaaacttttgcatctatcaatttcactcctctctttccctagttcgtaaataaaaattaaaaaaaaaaatcaacacggtgactggaagccacacctacagtctacagatatgcagagtgttatttgctttaatccatcgtcccttagtctaatagtcttgta

SEQ ID NO: 12 - DNA, 1000 bp, Glycine max
  source 1..1000: mol_type = genomic DNA
aattgtcttctatcattctatgtgctacccgaaaagaaaaaaaaacagacatctgttaaagagtagctgcagctttattctcttatgcatctgtcaaaccaaggagaaaaaaaatgtcaaactgaaaatttatacgtttttcctttatttcctttttaaacaattcttacattcgctattttaaacgaacatacaaatgtcttactgttttattatttcaattatcaccgttgaattgtgtcaaatttctagcaattctaggtctttaatattgactaagtaactttacccccctaaaaaaacaatagattctctctcatttccttgtgcttactaaaatcgaccctttgcaaaattatagttagaaacaccatacttgcagttgttggagtattacagtagaggctataaaaaaaaattacacagtaagaatggaagatactcgactcaaggtcaatttgacaagacaagaatacccgacctttccattaagtaacctaccaagcgtcacgactacagcaataggcataggtgaccgttgacaaaaaaaaaagagaaaaacattggtgtcctttcccatttctaattttaactattgtcttataaatagtgaaatatccctacacttcatagttttagctaatatacacagaataccacgatctcggatttggctgaactacaccgggcacactaaactcagttatcaaaagcagtatctatcattacgagaacttcctgattgcattgcaatacaacacaagaaaatactaacaacgttagaccaagtttgcataaaaattgatactttatacccaaatgtaagatctaaattgacagcaaattaatgaccctatgcacaaaacaaatgtaaacttcgtgcagcataaatagagctgttgcttcttgcacctcaaagttacttccagcataaacaaagggatgtaggaaacaacccataaatagttgtcaggaaaagtaacggggtattaccttagaa

SEQ ID NO: 13 - DNA, 2040 bp, Glycine max
  source 1..2040: mol_type = genomic DNA
aattaaggtgaattgtgactgaacggaatcttcgacatcctaacccggtatcggggaaagtacctgtaaaaatacttcgatgatcaagttagttaagtttaaaatacttatgtatcagtatatgaaatgattgaaacgtatcttgctgtgttttctttggatgtgtgtgtgtgtcctactttattagagatccattgaaaagtgagaacttcaagctattaagacggattttttcagtgtgacacttaataatttgtaaatttgttctaagatttgtattaaattttaattcttattgtattggattttttttggaaattagcagatatggtattaagctttttgaaatttcacttattatttattttttattattttctctttatttttttagtctttttatctctcttactttcacctaggttattttatacaacctgaaaaaaatcaatgaaataaaataatatttttataaaattaactttattatcattatttttttatgattttgccacccatatcattaaattataaaggatataagtgaaaaaaattaatacttcattaaaaaattaaaataataattattttagtataatttttttttcttactaacgattattattaaatgaagggaatagtagtatcatggccatttactttcttaggagagacaaagttagttgagaatcgacacaattaaaatgttagttagaggtgatctgttagttaaataaataaagagggtaggagaaaggtagattcattccatcttgtatcattctatttataagcatttactctgtgaaaggaaaacctttgtgaaggatattcattcaaaacttttgcatctatcaatttcactcctctctttccctagttcgtaaataaaaattaaaaaaaaaaatcaacacggtgactggaagccacacctacagtctacagatatgcagagtgttatttgctttaatccatcgtcccttagtctaatagtcttgtagccggtagttataattttttcccgtggcaacttcccttctaattgtcttctatcattctatgtgctacccgaaaagaaaaaaaaacagacatctgttaaagagtagctgcagctttattctcttatgcatctgtcaaaccaaggagaaaaaaaatgtcaaactgaaaatttatacgtttttcctttatttcctttttaaacaattcttacattcgctattttaaacgaacatacaaatgtcttactgttttattatttcaattatcaccgttgaattgtgtcaaatttctagcaattctaggtctttaatattgactaagtaactttacccccctaaaaaaacaatagattctctctcatttccttgtgcttactaaaatcgaccctttgcaaaattatagttagaaacaccatacttgcagttgttggagtattacagtagaggctataaaaaaaaattacacagtaagaatggaagatactcgactcaaggtcaatttgacaagacaagaatacccgacctttccattaagtaacctaccaagcgtcacgactacagcaataggcataggtgaccgttgacaaaaaaaaaagagaaaaacattggtgtcctttcccatttctaattttaactattgtcttataaatagtgaaatatccctacacttcatagttttagctaatatacacagaataccacgatctcggatttggctgaactacaccgggcacactaaactcagttatcaaaagcagtatctatcattacgagaacttcctgattgcattgcaatacaacacaagaaaatactaacaacgttagaccaagtttgcataaaaattgatactttatacccaaatgtaagatctaaattgacagcaaattaatgaccctatgcacaaaacaaatgtaaacttcgtgcagcataaatagagctgttgcttcttgcacctcaaagttacttccagcataaacaaagggatgtaggaaacaacccataaatagttgtcaggaaaagtaacggggtattaccttagaa

SEQ ID NO: 14 - DNA, 30 bp, synthetic construct
  source 1..30: mol_type = other DNA; note = primer
gtcgttttatcaaaatgtactttcatttta

SEQ ID NO: 15 - DNA, 27 bp, synthetic construct
  source 1..27: mol_type = other DNA; note = primer
cgggtagcacatagaatgatagaagac

SEQ ID NO: 16 - DNA, 16 bp, synthetic construct
  source 1..16: mol_type = other DNA; note = probe
taacgctgcggacatc

SEQ ID NO: 17 - DNA, 20 bp, synthetic construct
  source 1..20: mol_type = other DNA; note = primer
ccctgtgatcgcagactcaa

SEQ ID NO: 18 - DNA, 20 bp, synthetic construct
  source 1..20: mol_type = other DNA; note = primer
cggagatgctgtcgtgagaa

SEQ ID NO: 19 - DNA, 16 bp, synthetic construct
  source 1..16: mol_type = other DNA; note = probe
taacgctgcggacatc

SEQ ID NO: 20 - DNA, 26 bp, synthetic construct
  source 1..26: mol_type = other DNA; note = primer
tgcagagtgttatttgctttaatcca

SEQ ID NO: 21 - DNA, 16 bp, synthetic construct
  source 1..16: mol_type = other DNA; note = probe
tggcaacttcccttct

SEQ ID NO: 22 - DNA, 285 bp, Agrobacterium tumefaciens
  source 1..285: mol_type = genomic DNA
cgaagctcggtcccgtgggtgttctgtcgtctcgttgtacaacgaaatccattcccattccgcgctcaagatggcttcccctcggcagttcatcagggctaaatcaatctagccgacttgtccggtgaaatgggctgcactccaacagaaacaatcaaacaaacatacacagcgacttattcacacgagctcaaattacaacggtatatatcctgccagtcagcatcatcacaccaaaagttaggcccgaatagtttgaaattagaaagctcgcaattgaggtct

SEQ ID NO: 23 - DNA, 541 bp, Arabidopsis thaliana
  source 1..541: mol_type = genomic DNA
tttattagagtagattagaatcttttatgccaagtattgataaattaaatcaagaagataaactatcataatcaacatgaaattaaaagaaaaatctcatatatagtattagtattctctatatatattatgattgcttattcttaatgggttgggttaaccaagacatagtcttaatggaaagaatcttttttgaactttttccttattgattaaattcttctatagaaaagaaagaaattatttgaggaaaagtatatacaaaaagaaaaatagaaaaatgtcagtgaagcagatgtaatggatgacctaatccaaccaccaccataggatgtttctacttgagtcggtcttttaaaaacgcacggtggaaaatatgacacgtatcatatgattccttcctttagtttcgtgataataatcctcaactgatatcttcctttttttgttttggctaaagatattttattctcattaatagaaaagacggttttgggcttttggtttgcgatataaagaagaccttcgtgtggaagataat

SEQ ID NO: 24 - DNA, 89 bp, Arabidopsis thaliana
  source 1..89: mol_type = genomic DNA
aattcatcctttcgtctttttctgactcttcaatctctcccaaagcctaaagcgatctctgcaaatctctcgcgactctctctttcaag

SEQ ID NO: 25 - DNA, 378 bp, Arabidopsis thaliana
  source 1..378: mol_type = genomic DNA
gtatattttctgattctttttgtttttgattcgtatctgatctccaatttttgttatgtggattattgaatcttttgtataaattgcttttgacaatattgttcgtttcgtcaatccagcttctaaattttgtcctgattactaagatatcgattcgtagtgtttacatctgtgtaatttcttgcttgattgtgaaattaggattttcaaggacgatctattcaatttttgtgttttctttgttcgattctctctgttttaggtttcttatgtttagatccgtttctctttggtgttgttttgatttctcttacggcttttgatttggtatatgttcgctgattggtttctacttgttctattgttttatttcagggt

SEQ ID NO: 26 - DNA, 204 bp, Arabidopsis thaliana
  source 1..204: mol_type = genomic DNA
atggcgacggctacgacgactgctacggcggcgtttagtggtgtagtcagtgtaggaacggagactcgaaggatttattcgttttctcatcttcaaccttctgcggcttttccggcgaagcctagttccttcaaatctctcaaattaaagcagagcgcgaggctcacacggcggcttgatcatcggccgttcgttgtccgatgt

SEQ ID NO: 27 - DNA, 1023 bp, synthetic construct
  source 1..1023: mol_type = other DNA; note = codon optimized for plant
atgctcactttcgttagaaacgcttggtacgttgctgcacttcctgaggagttgagcgagaagcctctaggaagaactatcctcgatactccactagctctctatcgtcaacctgacggagttgtcgctgccctgcttgatatttgtccgcatcgcttcgctccgttgagtgacggtattctagtcaacggacatctccagtgtccatatcacggtctggaatttgacggaggtggccagtgtgtccacaacccgcacggcaacggagcccgccctgcttctctgaacgtgcgatcattccctgtcgtggaaagagacgcattgatctggatctggcctggagatccagcactcgcagatcccggtgctatccctgactttgggtgtcgtgttgatccagcttaccgtactgtcggaggttacggtcacgtggactgcaactacaagctccttgtggataacctcatggatcttggacacgctcagtacgtgcaccgcgctaacgcccaaacagacgccttcgatagacttgagcgtgaggtgatcgttggcgacggcgagatccaggcgctcatgaagatccctggtggcacaccctcagttctcatggctaagttcttgcgtggtgctaacacaccagttgacgcctggaacgacatccggtggaataaggtgtcggctatgctgaacttcatcgcggtcgcgccggaagggacgccgaaggagcagtcaatccactcccgaggaacccatatccttactcctgagaccgaggcaagctgccattacttcttcggtagttcccgcaacttcggtatagacgatccagagatggacggtgttctcaggagctggcaagctcaagccctggtgaaggaggacaaagtggtcgttgaagctatcgaaaggcggagggcttacgtcgaagcgaacgggatcagacccgccatgttgtcctgcgacgaggcagccgtcagggtatccagggagattgagaagctcgaacaactagaagcggcgtga

SEQ ID NO: 28 - DNA, 500 bp, Medicago truncatula
  source 1..500: mol_type = genomic DNA
gagtactctccaacatggacacaccatgggattgtgtaacataaataatgtgtcgtttgtaatgaatgctcgcaacttttctagctaattaagctagagttgaacttgagctacttttatgtaccctaaagaggcacaatctttgctgttgatgtactatgatcatgttataatatgatgaaaatggagtgtgcctcattttataatttttattttcctgagtatatgtttttagggctaaacaccttataaaaaaaggtcacttagaatatgaaacatgaacttttgtaaaaaagtagagattaaaattgaaatcaaaaatttttataggatcaatattcgaagaatttttttagagggattaaaattaaatatagtttcggactgacccaaggcacaatccggctccgctcgggttcgacctgagtccaccatgcatctgtcaccttaccattgacacgccctaaaatacattagatcgcagtacaaattgagagtta

SEQ ID NO: 29 - DNA, 500 bp, synthetic construct
  source 1..500: mol_type = other DNA; note = engineered promoter
catagagcagtcttgtctcaaaaaaatcgaattaagcagttgaaagtgttcgtcttatcaagatgaaaatctgacaaaaattttgttaaaatttggtagtatttgataagatatgtaccttaaaattgatgttaataatcttcttatccaaaaaaagaaccaatccattgcagtcttaaatgaaatatttcaagaaaggattgagccaaaaaccgtgtttataaaattttcaaatccacaatttccacattcacttggatattacaagtgtggcatctcataaaaaaaaagaaaaagaaaaaccacgtggactattatatccagccacgtggctttaaaatcttatccaaaatcacctctcatccaacggataaggtaaccacacagccttatccaaccacatcacacgaatccactctatatatactccatataaccatcactcacacgtctttcatcacacaagtaaaacagcatcactaaaagaaaaaaaacaagaa

SEQ ID NO: 30 - DNA, 310 bp, synthetic construct
  source 1..310: mol_type = other DNA; note = engineered intron
tttcaggtttagctttctctccgatctcggctttcgatctgatctcgtctgatctccgatttcgatccccgtccgatctcgatctgatcccatccgatctgtgattgattcgttgagattcagatctgatcttaggatctctctgatagatcatagattcaatctctcgagatagagatgattgatgttgttcagatcggttttgatctctgatctgatcagctcgattgattcgatttgattctcgattcgatttgatcgacaattgatctgattctctaattgatgttctgtttttgttacaggtttt

SEQ ID NO: 31 - DNA, 552 bp, synthetic construct
  source 1..552: mol_type = other DNA; note = codon optimized for plant
atgtctcccgagcgacggccagtagagattagacccgcaacagccgccgacatggcggcagtatgtgacatcgtaaatcactacatcgagacatccacagtaaactttcgtaccgaaccacaaactcctcaggaatggattgatgacttggagagactacaagaccgctatccgtggctcgtggcagaggtcgaaggcgtcgtcgcgggaatcgcctacgctggaccgtggaaggctcgaaatgcttacgactggactgtggaatccactgtttacgtctcgcatcgccatcagaggctgggtctgggcagcaccctgtacacccacttactcaaatctatggaggctcagggctttaagtcagtggtggccgtcatcggactacctaacgatccttctgtgcgcctacatgaagcactaggctacacggcacgcggaacattgagggccgccgggtacaaacacggaggatggcacgacgttggattctggcaacgggatttcgaacttccagcacctccaagacccgttagaccagttacccagatttga

SEQ ID NO: 32 - DNA, 500 bp, Medicago truncatula
  source 1..500: mol_type = genomic DNA
acacatattcaagctctattactctgtttttaggttgagaatattgtgaagtgtgttgtgtgtgcgttctgttttatgtaaaagtaaattctgtgaattatattttgtaaggaataaattatgtaagaatggtttaattatctattgtctcacatcgttttttatgtctattttaagtattggtattgtattctatttatacacaagagttaataatagcataacataaattccacaaggatcaagtaaaataatatccacatgaaacatctaagaaaggaaaacaatatggctcaatttcctatctttctttatttatgttttagtcatctttctttccctatttcttgttgaaagctaataggagtatggtactttctacgccatttttttttaagtactctcttttacacaatattttatcaaattgtagtgacattttattttacgatgcagttaatgttcgttgtgttactgtcaatgattgtccaaaggtgaaa

SEQ ID NO: 33 - DNA, 829 bp, Arabidopsis thaliana
  source 1..829: mol_type = genomic DNA
gttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttctt

SEQ ID NO: 34 - DNA, 67 bp, Arabidopsis thaliana
  source 1..67: mol_type = genomic DNA
cacaattcagatttcaatttctcaaaatcttaaaaactttctctcaattctctctaccgtgatcaag

SEQ ID NO: 35 - DNA, 306 bp, Arabidopsis thaliana
  source 1..306: mol_type = genomic DNA
gtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacaggt

SEQ ID NO: 36 - DNA, 888 bp, synthetic construct
  source 1..888: mol_type = other DNA; note = codon optimized variant
atgcacgcggctttgacacctttgaccaacaagtatcggttcatcgacgttcaaccactcacaggcgtgctcggcgcagagattaccggagtggacctgagggagcccttagacgactccacttggaacgagatcctcgacgcctttcacacctaccaagttatctactttcctggacaggcgatcaccaacgagcagcacattgccttctcaaggaggttcggaccggtagatccagttccaattctcaaatccattgagggttatcccgaggtgcagatgattagacgagaagccaacgagtcctcacggtacataggcgacgattggcacgcagacagcaccttccttgacgctcctccggctgccgtggttatgcgcgcaatagaggtgccggagtacggcggcgataccggtttcctatcaatgtactctgcatgggagacgctctcaccaacgatgcaagccaccattgaaggtctaaacgtggttcactcagctactaaggtcttcggaagtctttaccaggcgacgaattggaggttcagtaacaccagtgtgaaggtgatggatgtggacgctggagacagggagacggtgcatccactcgtagttacacaccctgtaactggacgcagagccctttactgcaaccaggtttactgccagaagatccagggaatgactgatgcggagtctaagtccctgcttcaattcctctacgaacacgccacccaattcgacttcacttgtcgtgttcggtggaagaaggaccaagtgctcgtgtgggataacctttgcaccatgcaccgagcagtaccagactacgccgggaaattccgctatctcacccgcactacagtggccggagacaagcctagccgctga

SEQ ID NO: 37 - DNA, 500 bp, Medicago truncatula
  source 1..500: mol_type = genomic DNA
gcatgaataatcaagctcataaatttcatggctttgaatttgtactattttggttactagaaagtgtatttgtgtgtttatgcagtaataaatctctaagagatatatgtttgttattttttataattatccaaaaaatcgttaatgttgaaaattgattcaaaattgatattgaagttctgaaaaaatcgtggcgtgattaaaaatccaaacttttttataaaataatattgtgtctatatcttttataaatgacgataaatgggataaagtaaatgaaacaaaaccgttaatgcaatgttcatctgcacaatatatataattaaaaaacattataaaaccttgtttcttcactcatttacaatcttgaaattttagtctttaccatttgaaagtacaatcttttcatgaaagtttatagtacaaatcaagagtttggataagctgctctgctttttataatcactgggaaatgatttatgacttggaaaaacaacttg

SEQ ID NO: 38 - DNA, 458 bp, synthetic construct
  source 1..458: mol_type = other DNA; note = engineered promoter
aattaaattcaacacgtttgttatatattttttattgaaattattcttcattcgtcttttaatggataaaaaggtataatcaagtatattttatacacatctttctatttgtgtgtaccaaatgttaaaatggccaattttgaccaaaaaaccgcataattttcttaatttcttaaatatgattaattcatcaataacttggaatttcacaatacacaaaagtgggtgtagttaccgttattatatttatacacaacaactcatctcctcatagaaagaaaagaaaaataaaataagaaatcaaaaaacgacaagataaccaatctccacatcatccacgtggcgtaaggataaggtcacaaccaccactcagccacgtggcagaatcttatccaatcactctcaccacacaaaccttatccacttctatatataatctcttcttctcattatcactc

SEQ ID NO: 39 - DNA, 42 bp, synthetic construct
  source 1..42: mol_type = other DNA; note = engineered 5' UTR
accacacatccttgcaaaagtaaagagaaaaaacaaacaaga

SEQ ID NO: 40 - DNA, 300 bp, synthetic construct
  source 1..300: mol_type = other DNA; note = engineered intron
caggtaaacccagatctctttcttctcttctcttcatctcgatctctccattttcataaacccaattttttctctgatttgtttgatttggtttggatctttctgtgtttccatggttttaggaattttaggatagatttttgtttgttcatgttattcatcggatatatagatttcaaattcttttgcaatttttctctctctttagttttgctcaattttggttgttgttgtgatgagtgttctctttatgggtttatctgagcttggtgagagttttttgatattgattttgcaggt

SEQ ID NO: 41 - DNA, 1056 bp, synthetic construct
  source 1..1056: mol_type = other DNA; note = codon optimized for plant
atggctgacgagtcatggagggcgccggcgatagtgcaagagctggcggcagccggcgtcgaggagccgccgagccgatacctgctacgggagaaagaccgttctgacgtcaagctggtcgccgccgagctgccggagcctctcccagtcgttgatctcagccggctagatggtgccgaggaggccaccaagctcagggtggctctgcagaattggggcttcttcctgcttaccaaccatggagtagaagcctctctgatggacagcgtgatgaacttgtcgagagagttcttcaaccagccaatcgaacggaagcagaaattcagcaacttgatcgatggcaagaacttccagattcaagggtatggaactgaccgggtggttacccaagatcagatcctggactggtctgatcggttgcatctcagagttgaacccaaggaggagcaagatcttgccttctggcctgaccatcctgaatctttcagggatgttctgaacaagtatgcatcaggaactaaaagaattagagacgatatcattcaggctatggccaagcttcttgagcttgatgaggattacttcttggaccgactcaacgaagctcctgcattcgcaagattcaactactaccctccctgtccaaggcctgaccttgtgttcggcatcaggcctcactccgacggcaccctcttgacgattcttctcgtcgacaaagatgtcagtggcctgcaagttcagagggatggcaagtggtccaacgttgaggcaactcctcacacattgctgatcaacttaggtgacaccatggaggtaatgtgcaatggcatcttcaggagcccggtgcacagggtggtgacaaacgccgagaaggagaggatctccctggccatgttatacagcgtgaacgatgagaaagacattgagccggcggctggtttgctggatgagaatcggcctgcaagatacaggaaagtgagcgtcgaagagttcagggccgggatctttggaaaattctctcgaggagagaggtacatcgactccctgaggatctga

SEQ ID NO: 42 - DNA, 300 bp, synthetic construct
  source 1..300: mol_type = other DNA; note = engineered 3' UTR
atgtctgctgcggcggccttcacagtttgtttatttcctactgtttgctgcggcgattgttgttgttttctgttttataaataataaaggaggaggagatttgttttggtttgtgtttgtttccatccttgctgctccatcacactatctgtaatttgtaaacagcgacaataaataaattaataaatttggtttctcatacctatatgtgtctgtttggaggcttgtttgtttgagacatctgtctggttgtttttttgctgccagccggtagtataaattttgtttttggacgacgaa

SEQ ID NO: 43 - DNA, 442 bp, Agrobacterium tumefaciens
  source 1..442: mol_type = genomic DNA
ctcatctaagcccccatttggacgtgaatgtagacacgtcgaaataaagatttccgaattagaataatttgtttattgctttcgcctataaatacgacggatcgtaatttgtcgttttatcaaaatgtactttcattttataataacgctgcggacatctacatttttgaattgaaaaaaaattggtaattactctttctttttctccatattgaccatcatactcattgctgatccatgtagatttcccggacatgaagccatttacaattgaatatatcctgccgccgctgccgctttgcacccggtggagcttgcatgttggtttctacgcagaactgagccggttaggcagataatttccattgagaactgagccatgtgcaccttccccccaacacggtgagcgacggggcaacggagtgatccacatgggactttt

SEQ ID NO: 44 - DNA, 3684 bp, synthetic construct
  source 1..3684: mol_type = other DNA; note = codon optimized for plant
atgtcgaagctcgagaagttcaccaactgctactcgctgagcaagacgctgcggttcaaggcgatccccgtcgggaagacccaggagaacatcgacaacaagcggctcctggtcgaggacgagaagcgcgccgaggactacaagggcgtcaagaagctgctggaccggtactacctctccttcatcaacgacgtcctgcactcgatcaagctcaagaacctgaacaactacatctcgctgttccgcaagaagacacggaccgagaaggagaacaaggagctcgagaacctcgagatcaacctgcgcaaggagatcgcgaaggcgttcaagggcaacgaggggtacaagagcctgttcaagaaagacatcatcgagaccatcctgccggagttcctggacgacaaggacgagatcgcgctggtgaactcgttcaacgggttcaccacggccttcaccgggtttttcgacaaccgggagaacatgttcagcgaggaggccaagtcgaccagcatcgccttccggtgcatcaacgagaacctcacccgctacatcagcaacatggacatcttcgagaaggtggacgccatcttcgacaagcacgaggtccaggagatcaaggaaaagatcctgaactcggactacgacgtggaagacttctttgagggcgagttcttcaacttcgtcctcacccaggagggcatcgacgtctacaacgccatcatcggcggcttcgtgacggagagcggcgagaagatcaagggcctcaacgagtacatcaacctctacaaccagaagactaagcagaagctcccgaagttcaagccgctgtacaagcaagtcctgagcgaccgggagtccctctcgttctacggcgagggctacacgagcgacgaggaggtgctggaggtgttccgcaacacgctgaacaagaacagcgagatcttcagctcgatcaagaaactcgagaagctgttcaagaacttcgacgagtacagcagcgccggcatcttcgtcaagaacgggcccgcgatcagcaccatcagcaaggacatcttcggggagtggaacgtgatccgcgacaagtggaacgccgagtacgacgacatccacctcaagaaaaaggcggtggtcacggagaagtacgaggacgaccgccggaagtccttcaagaaaatcgggagcttcagcctcgagcagctccaggagtacgcggacgccgacctgagcgtggtggagaagctcaaggagatcatcatccagaaggtcgacgagatctacaaggtctacggctcgagcgagaagctgttcgacgcggacttcgtgctggagaagtccctcaagaagaacgacgccgtggtggccatcatgaaggatctgctcgacagcgtgaagtcgttcgagaactacatcaaggcattctttggggagggcaaggagacgaaccgggacgagtccttctacggggacttcgtgctcgcgtacgacatcctcctgaaggtcgaccacatctacgacgcgatccggaactacgtcacgcagaagccctacagcaaggacaagttcaagctctacttccagaacccgcagttcatgggcgggtgggacaaggacaaggagaccgactaccgggccacgatcctgcggtacgggtccaagtactacctcgccatcatggacaagaagtacgccaagtgcctccagaagattgacaaggacgacgtgaacgggaactacgagaagatcaactacaagctcctcccggggcccaacaagatgctgccgaaggtgttcttcagcaagaagtggatggcctactacaacccctcggaggacatccagaagatatacaagaacggcacgttcaaaaagggggacatgttcaacctgaacgactgccacaagctgatcgactttttcaaggacagcatcagccgctacccgaagtggtcgaacgcctacgacttcaacttctcggagacggagaagtacaaggacattgcgggcttctaccgggaggtggaggagcagggctacaaggtctccttcgagagcgcctccaagaaagaggtggacaagctcgtggaggagggcaagctgtacatgttccagatctacaacaaggacttctcggacaagtcgcacggcaccccgaacctccacacgatgtacttcaagctgctgttcgacgagaacaaccacgggcagatccgcctcagcggcggggcggagctgttcatgcgccgcgcgtccctcaagaaggaggagctggtcgtgcaccccgccaactccccgatcgcgaacaagaaccccgacaaccccaagaagacaaccaccctctcgtacgacgtctacaaggacaagcggttctcggaggaccagtacgagctgcacatcccgatcgccatcaacaagtgccccaagaacatcttcaagatcaacaccgaggtgcgggtgctgctcaagcacgacgacaacccctacgtcatcgggatcgaccgcggcgagcggaacctgctctacatcgtggtcgtggacgggaaggggaacatcgtggagcagtacagcctgaacgagatcatcaacaacttcaacggcatccgcatcaagacggactaccacagcctcctggacaagaaggagaaggagcggttcgaggcgcggcagaactggacctccatcgagaacatcaaggagctgaaggccggctacatcagccaggtcgtgcacaagatctgcgagctcgtggagaagtacgacgcggtgatcgcgctggaggacttgaacagcgggttcaagaactcccgggtcaaggtcgagaagcaggtctaccagaagttcgagaagatgctgatcgacaagctcaactacatggtggacaagaagtccaacccctgcgccaccggcggcgccctcaagggctaccagatcaccaacaagttcgagtccttcaagtcgatgtctacgcagaacgggttcattttctacatcccggcgtggctcaccagcaagatcgacccgagcacgggcttcgtcaacctcctgaagaccaagtacaccagcatcgcggacagcaagaagttcatctcctcgttcgaccgcatcatgtacgtccccgaggaagacctgttcgagttcgccctcgactacaagaacttctcccggacggacgccgactacatcaaaaagtggaagctctacagctacggcaaccggatccgcatcttccgcaaccccaagaagaacaatgtgttcgactgggaggaggtgtgcctgacgagcgcctacaaggagctcttcaacaagtacggcatcaactaccagcaaggggacatccgcgcgctgctctgcgagcagtccgacaaggcgttctactcgtcgttcatggccctgatgagcctcatgctccagatgcgcaacagcatcaccggccggacggacgtggacttcctgatcagcccggtcaagaacagcgacggcattttctacgacagccggaactacgaggcccaggagaacgccatcctccccaagaacgccgacgcgaacggcgcctacaacatcgcgcggaaggtgctgtgggccatcggccagtttaaaaaggcggaggacgagaagctggacaaggtcaagatcgccatcagcaacaaggagtggctcgagtacgcgcagacgagcgtgaagcac

SEQ ID NO: 45 - protein, 1228 aa, unidentified
  source 1..1228: note = Lachnospiraceae bacterium ND2006
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 46 - protein, 1228 aa, unidentified
  source 1..1228: note = Lachnospiraceae bacterium ND2006
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMRGWDKDKETDYRATILRYGSKYYLAIMDKKYAKCLQKIDKDDVNGNYEKINYKLLPGPNKMLPRVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 47 - protein, 1228 aa, unidentified
  source 1..1228: note = Lachnospiraceae bacterium ND2006
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMRGWDKDVETDRRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 48 - protein, 1300 aa, Francisella novicida
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 49 - DNA, 21 bp, synthetic construct
  source 1..21: mol_type = other DNA; note = gRNA repeat
gaatttctactaagtgtagat

SEQ ID NO: 50 - DNA, 21 bp, synthetic construct
  source 1..21: mol_type = other DNA; note = gRNA repeat
gtaatttctactgttgtagat

SEQ ID NO: 51 - DNA, 50 bp, synthetic construct
  source 1..50: mol_type = other DNA; note = gRNA
taatttctactgttgtagatgttaaataaataaagagggtaggttttttt

SEQ ID NO: 52 - DNA, 50 bp, synthetic construct
  source 1..50: mol_type = other DNA; note = gRNA
taatttctactgttgtagatgtcattggactgaacacgagtgtttttttt

SEQ ID NO: 53 - DNA, 50 bp, synthetic construct
  source 1..50: mol_type = other DNA; note = gRNA
taatttctactgttgtagatggccgtgacggccacgagcgaacttttttt

SEQ ID NO: 54 - DNA, 50 bp, synthetic construct
  source 1..50: mol_type = other DNA; note = gRNA
taatttctactgttgtagatgcgcgccaattaaattcaacacgttttttt

SEQ ID NO: 55 - DNA, 50 bp, synthetic construct
  source 1..50: mol_type = other DNA; note = gRNA
taatttctactgttgtagatgagtattacagtagaggctataattttttt

SEQ ID NO: 56 - DNA, 1368 bp, synthetic construct
  source 1..1368: mol_type = other DNA; note = codon optimized for plant
atgcttcacggtgcaagcagccgtccagcaactgctcgtaagtcctctggtctttctggaaccgtccgtattccaggtgacaagtctatctcccacaggtccttcatgtttggaggtctcgctagcggtgaaactcgtatcaccggtcttttggaaggtgaagatgttatcaacactggtaaggctatgcaagctatgggtgccagaatccgtaaggaaggtgatacttggatcattgatggtgttggtaacggtggactccttgctcctgaggctcctctcgatttcggtaacgctgcaactggttgccgtttgactatgggtcttgttggtgtttacgatttcgatagcactttcattggtgacgcttctctcactaagcgtccaatgggtcgtgtgttgaacccacttcgcgaaatgggtgtgcaggtgaagtctgaagacggtgatcgtcttccagttaccttgcgtggaccaaagactccaacgccaatcacctacagggtacctatggcttccgctcaagtgaagtccgctgttctgcttgctggtctcaacaccccaggtatcaccactgttatcgagccaatcatgactcgtgaccacactgaaaagatgcttcaaggttttggtgctaaccttaccgttgagactgatgctgacggtgtgcgtaccatccgtcttgaaggtcgtggtaagctcaccggtcaagtgattgatgttccaggtgatccatcctctactgctttcccattggttgctgccttgcttgttccaggttccgacgtcaccatccttaacgttttgatgaacccaacccgtactggtctcatcttgactctgcaggaaatgggtgccgacatcgaagtgatcaacccacgtcttgctggtggagaagacgtggctgacttgcgtgttcgttcttctactttgaagggtgttactgttccagaagaccgtgctccttctatgatcgacgagtatccaattctcgctgttgcagctgcattcgctgaaggtgctaccgttatgaacggtttggaagaactccgtgttaaggaaagcgaccgtctttctgctgtcgcaaacggtctcaagctcaacggtgttgattgcgatgaaggtgagacttctctcgtcgtgcgtggtcgtcctgacggtaagggtctcggtaacgcttctggagcagctgtcgctacccacctcgatcaccgtatcgctatgagcttcctcgttatgggtctcgtttctgaaaaccctgttactgttgatgatgctactatgatcgctactagcttcccagagttcatggatttgatggctggtcttggagctaagatcgaactctccgacactaaggctgcttga

SEQ ID NO: 57 - protein, 455 aa, Agrobacterium sp.
  source 1..455: note = Agrobacterium sp. CP4 strain
MLHGASSRPATARKSSGLSGTVRIPGDKSISHRSFMFGGLASGETRITGLLEGEDVINTGKAMQAMGARIRKEGDTWIIDGVGNGGLLAPEAPLDFGNAATGCRLTMGLVGVYDFDSTFIGDASLTKRPMGRVLNPLREMGVQVKSEDGDRLPVTLRGPKTPTPITYRVPMASAQVKSAVLLAGLNTPGITTVIEPIMTRDHTEKMLQGFGANLTVETDADGVRTIRLEGRGKLTGQVIDVPGDPSSTAFPLVAALLVPGSDVTILNVLMNPTRTGLILTLQEMGADIEVINPRLAGGEDVADLRVRSSTLKGVTVPEDRAPSMIDEYPILAVAAAFAEGATVMNGLEELRVKESDRLSAVANGLKLNGVDCDEGETSLVVRGRPDGKGLGNASGAAVATHLDHRIAMSFLVMGLVSENPVTVDDATMIATSFPEFMDLMAGLGAKIELSDTKAA

SEQ ID NO: 58 - DNA, 50 bp, Glycine max
  source 1..50: mol_type = other DNA
aattaaggtgaattgtgactgaacggaatcttcgacatcctaacccggta

SEQ ID NO: 59 - DNA, 50 bp, Glycine max
  source 1..50: mol_type = other DNA
tcggggaaagtacctgtaaaaatacttcgatgatcaagttagttaagttt

SEQ ID NO: 60 - DNA, 50 bp, Glycine max
  source 1..50: mol_type = other DNA
aaaatacttatgtatcagtatatgaaatgattgaaacgtatcttgctgtg

SEQ ID NO: 61 - DNA, 50 bp, Glycine max
  source 1..50: mol_type = other DNA
ttttctttggatgtgtgtgtgtgtcctactttattagagatccattgaaa

SEQ ID NO: 62 - DNA, 50 bp, Glycine max
  source 1..50: mol_type = other DNA
agtgagaacttcaagctattaagacggattttttcagtgtgacacttaat

SEQ ID NO: 63 - DNA, 50 bp, Glycine max
  source 1..50: mol_type = other DNA
aatttgtaaatttgttctaagatttgtattaaattttaattcttattgta

SEQ ID NO: 64 - DNA, 50 bp, Glycine max
  source 1..50: mol_type = other DNA
ttggattttttttggaaattagcagatatggtattaagctttttgaaatt

SEQ ID NO: 65 - DNA, 50 bp, Glycine max
  source 1..50: mol_type = other DNA
tcacttattatttattttttattattttctctttatttttttagtctttt

SEQ ID NO: 66 - DNA, 50 bp, Glycine max
  source 1..50: mol_type = other DNA
tatctctcttactttcacctaggttattttatacaacctgaaaaaaatca

SEQ ID NO: 67 - DNA, 50 bp, Glycine max
  source 1..50: mol_type = other DNA
atgaaataaaataatatttttataaaattaactttattatcattattttt

SEQ ID NO: 68 - DNA, 50 bp, Glycine max
  source 1..50: mol_type = other DNA
ttatgattttgccacccatatcattaaattataaaggatataagtgaaaa

SEQ ID NO: 69 - DNA, 50 bp, Glycine max
  source 1..50: mol_type = other DNA
aaattaatacttcattaaaaaattaaaataataattattttagtataatt

SEQ ID NO: 70 - DNA, 50 bp, Glycine max
  source 1..50: mol_type = other DNA
tttttttcttactaacgattattattaaatgaagggaatagtagtatcat

SEQ ID NO: 71 - DNA, 50 bp, Glycine max
  source 1..50: mol_type = other DNA
ggccatttactttcttaggagagacaaagttagttgagaatcgacacaat

SEQ ID NO: 72 - DNA, 50 bp, Glycine max
  source 1..50: mol_type = other DNA
taaaatgttagttagaggtgatctgttagttaaataaataaagagggtag

SEQ ID NO: 73 - DNA, 50 bp, Glycine max
  source 1..50: mol_type = other DNA
gagaaaggtagattcattccatcttgtatcattctatttataagcattta

SEQ ID NO: 74 - DNA, 50 bp, Glycine max
  source 1..50: mol_type = other DNA
ctctgtgaaaggaaaacctttgtgaaggatattcattcaaaacttttgca

SEQ ID NO: 75 - DNA, 50 bp, Glycine max
  source 1..50: mol_type = other DNA
tctatcaatttcactcctctctttccctagttcgtaaataaaaattaaaa

SEQ ID NO: 76 - DNA, 50 bp, Glycine max
  source 1..50: mol_type = other DNA
aaaaaaatcaacacggtgactggaagccacacctacagtctacagatatg

SEQ ID NO: 77 - DNA, 50 bp, Glycine max
  source 1..50: mol_type = other DNA
cagagtgttatttgctttaatccatcgtcccttagtctaatagtcttgta

SEQ ID NO: 78 - DNA, 50 bp, Glycine max
  source 1..50: mol_type = other DNA
aattgtcttctatcattctatgtgctacccgaaaagaaaaaaaaacagac

SEQ ID NO: 79 - DNA, 50 bp, Glycine max
  source 1..50: mol_type = other DNA
atctgttaaagagtagctgcagctttattctcttatgcatctgtcaaacc

SEQ ID NO: 80 - DNA, 50 bp, Glycine max
  source 1..50: mol_type = other DNA
aaggagaaaaaaaatgtcaaactgaaaatttatacgtttttcctttattt

SEQ ID NO: 81 - DNA, 50 bp, Glycine max
  source 1..50: mol_type = other DNA
cctttttaaacaattcttacattcgctattttaaacgaacatacaaatgt

SEQ ID NO: 82 - DNA, 50 bp, Glycine max
  source 1..50: mol_type = other DNA
cttactgttttattatttcaattatcaccgttgaattgtgtcaaatttct

SEQ ID NO: 83 - DNA, 50 bp, Glycine max
  source 1..50: mol_type = other DNA
agcaattctaggtctttaatattgactaagtaactttacccccctaaaaa

SEQ ID NO: 84 - DNA, 50 bp, Glycine max
  source 1..50: mol_type = other DNA
aacaatagattctctctcatttccttgtgcttactaaaatcgaccctttg

SEQ ID NO: 85 - DNA, 50 bp, Glycine max
  source 1..50: mol_type = other DNA
caaaattatagttagaaacaccatacttgcagttgttggagtattacagt

SEQ ID NO: 86 - DNA, 50 bp, Glycine max
  source 1..50: mol_type = other DNA
agaggctataaaaaaaaattacacagtaagaatggaagatactcgactca

SEQ ID NO: 87 - DNA, 50 bp, Glycine max
  source 1..50: mol_type = other DNA
aggtcaatttgacaagacaagaatacccgacctttccattaagtaaccta

SEQ ID NO: 88 - DNA, 50 bp, Glycine max
  source 1..50: mol_type = other DNA
ccaagcgtcacgactacagcaataggcataggtgaccgttgacaaaaaaa

SEQ ID NO: 89 - DNA, 50 bp, Glycine max
  source 1..50: mol_type = other DNA
aaagagaaaaacattggtgtcctttcccatttctaattttaactattgtc

SEQ ID NO: 90 - DNA, 50 bp, Glycine max
  source 1..50: mol_type = other DNA
ttataaatagtgaaatatccctacacttcatagttttagctaatatacac

SEQ ID NO: 91 - DNA, 50 bp, Glycine max
  source 1..50: mol_type = other DNA
agaataccacgatctcggatttggctgaactacaccgggcacactaaact

SEQ ID NO: 92 - DNA, 50 bp, Glycine max
  source 1..50: mol_type = other DNA
cagttatcaaaagcagtatctatcattacgagaacttcctgattgcattg

SEQ ID NO: 93 - DNA, 50 bp, Glycine max
  source 1..50: mol_type = other DNA
caatacaacacaagaaaatactaacaacgttagaccaagtttgcataaaa

SEQ ID NO: 94 - DNA, 50 bp, Glycine max
  source 1..50: mol_type = other DNA
attgatactttatacccaaatgtaagatctaaattgacagcaaattaatg

SEQ ID NO: 95 - DNA, 50 bp, Glycine max
  source 1..50: mol_type = other DNA
accctatgcacaaaacaaatgtaaacttcgtgcagcataaatagagctgt

SEQ ID NO: 96 - DNA, 50 bp, Glycine max
  source 1..50: mol_type = other DNA
tgcttcttgcacctcaaagttacttccagcataaacaaagggatgtagga

SEQ ID NO: 97 - DNA, 50 bp, Glycine max
  source 1..50: mol_type = other DNA
aacaacccataaatagttgtcaggaaaagtaacggggtattaccttagaa

SEQ ID NO: 98 - DNA, 5000 bp, Glycine max
  source 1..5000: mol_type = genomic DNA
ccatattatatttattatttctctcttttcttttcagttaactatattccaaaaatgcgtgtaagttgagcattttccaaaaagaacccctctatgaaaagtattattccggtcatgtcaagtttggcaatgagtcttgtcaagttaagaatgagaagagaagaagagaaagctactggggcaatcctacgttaaccatgatccatcatctgcataacaattagtgctacctgttccttttctgttttttcaccatattccacgagacaaacttttccaacaccaatctttacttttacaatatatatatatatatatatatatatatatatatatatatatatatatatatatatatatatatatatatgtatattcacaatcatagtctgtcctctgccgcccatcgggtatttttaattaaagtaatatttaaaaattaaaaaggaaaaagtaatagtacataattaaattgaataaatataagtgattatatttaagtatgaagaataaattgtaaaaatagaaattaataaaataatataaataactgagatttaagataaacaattaaatataaaataatacaactaattaaaactgtttttttttaatatatgggctttaagttcaatatattccaagtaatcacgatcccccacaatcagtaatgagaaataagtaggtatttctctcgtagctctacaatttgaactctaaataaaaaaaattatcaaagtgaaagaataacaaaaaaaatttgttgggagaaaatgtcacctatcctggacaatccggtttgggtacccttatggccttatccaaaaaaaatcacatatggtccaatgaacagagagataacgttgaataccatatttgtgaaaaatgatcggactgacaattgaataaaaaacccgtttagtaataataaaaaataaccagttttgtaataagtctgaataggttatccgttaacagttaaagagagaatggactaaaatagggtaaaaactagtggtaaagaacaacagttcaaatcgtcactttatttttactttctaaaacattagattttcttaaaaatgtatttaaaaatgaaaagaaagactaatatacgaattttttactcgaattactatcatttaatactaattatgaattttatagtttataattaaaacataaaatataattattttataatagtttattggggtactatttaataatttgtggaatgctaaaataagtttaattattagcaatttgatcacgagttaactataattaaatgaatcaatttaaaatgaatgtaaattatgtaatgttcgaagcaattaacagtttggtgttagaaatataataaggatttatttttagtcgggggttagataagtgttggggggagttgtttaacaccgacacaaaatgaatagggtctttggactttgatatcaatgtataatttgttcacaaatatatcacaaaaagaaatataaaaatataaaatttaatctttaaaagtgtaaaaaatataataaatatatctcattattttattcgttaccattaataaaataatttatgtggtatatagaaacaaatttatctctaaattacacatgacactaataactctttatccttaaaaaaaactggtggctccaaagaggacaacccgatttgtgggattggaggaccaaagcaaagcaaaggttttctgattgtttttattttcatctctctttgggtgggtggtacaggtgatggtaggcctttgtttcctcacatttgtttggttgctttctgctttgtcttatagttgatgcacatgtcataaagagtgagggagatggaatataacatgaaataaattaaatggcatatgcataacaagatggaataataaatgatgatattattgaagtagttcacggtgaaaaaaaaaataaattaatcacattctttatatgatcggttaaaattaattgaaaaaccactcattttaatgaattttactttttaatcaataaccatattttatgatttataagtatgattcgttaaaattattattttttaataaattataatgaatcataaacaaaatgtttgagagaatattgatatcattttaaaaaaataggtttatttaatcagttgtttggataatcaacatgataataagggtatctttgtagctaatataattattggatgtttccattaagtaccagctcttaataagatataactgttgaagtgcaatcttgataagcacatgctgcttctataagcactgaattcttatttatttttgtgtgtcatatgttatggtagcatatgtcagtttttctgataaaaaaattgaaaattatgatatgtaaggatgtgggaccaatttaataaagtttttagagttggataaaaaactgtgttacttaaatcataaaaagtatatatatatatatatatatatatatatatatatatatatatatatatatatatatatatatatatatataaagtgatttgtaatggtgagactcatttgaaaatcttcagaaataaaaaataagtcttttgtatggagaatgacccaaatagaaaaaaaattgtcccatattccaaatttaaaaaacaaaggtatatatacatataagatgaaaaaactggaataaaattcaaataataattgtttaaaataagtttttataaaacattttttcctgcaggataatctctttgttgttagtctttttttgtattttttattttatttatcgataaatgttaattgttagtaagatggattcaacatgcaattttttcatttttttactttttcttaatctctcaacaaacctatatgatctccatgtattttttatttgttctctttatactagagttgacaatagacttaatgaactacacttataaatatacacgtcaaatgagtttgaatccaagtataaacgggtaattacccattctaggcatttgcttctcacgtatgcacatggatgcgattaaggaccactttcctaaaatgtggatatggatatgataactctaatatcctgtccaaatcttatttattatcatctttgctttgaaagtcactacttttctattttagagggattgaccgtaaaacttgtgatgtttggagtaactaaagcaaaaattgtgacatttagaaggggaaaaaataacaaagaaacggttcttgataggaaaataaataaataaaaatttataagaggaatgaaaaaaatatttcatgagataatttattttgaaaaaagaggctaaatgaaaaagtaagaattatttaaaaaattagaaaatttaaagtaattatggtgttgccagtgttatgttaaatatttcatgagatcttctattctctctcattttttagtaggaaaaacaactaatagttatacatcattagttcattatattatcactacgccaacagtcgaagccaataaagtcaatccaacattactcgatccgtaacgttgttatgacaaccgaacaacaacatgccccttgctaaaaatctactacacaaaaaaggaaagcatggaccatggtaattggtaggtgaaacattcaatgcccaacttaacaaaatggacactaaaactcatcagagagtgagagaaggaagaggacggcacaatttgcatcatgcagcattccgaatccataatttcctcaaaattatgaacctatactcagtttaatccatatagttatacttatttgttagttaatatttcacaattttaaattatactaaatacattaacccaattttgagaacaaataggaacatagatatgatatagagatattttaatatttgagactacctaacctaatttctaatagttgagatattatatattattcctactgaattccgacgagttgtgactgagttatgatcgagtgtgatcaagttctgactgagttgtggccgaattaaggtgaattgtgactgaacggaatcttcgacatcctaacccggtatcggggaaagtacctgtaaaaatacttcgatgatcaagttagttaagtttaaaatacttatgtatcagtatatgaaatgattgaaacgtatcttgctgtgttttctttggatgtgtgtgtgtgtcctactttattagagatccattgaaaagtgagaacttcaagctattaagacggattttttcagtgtgacacttaataatttgtaaatttgttctaagatttgtattaaattttaattcttattgtattggattttttttggaaattagcagatatggtattaagctttttgaaatttcacttattatttattttttattattttctctttatttttttagtctttttatctctcttactttcacctaggttattttatacaacctgaaaaaaatcaatgaaataaaataatatttttataaaattaactttattatcattatttttttatgattttgccacccatatcattaaattataaaggatataagtgaaaaaaattaatacttcattaaaaaattaaaataataattattttagtataatttttttttcttactaacgattattattaaatgaagggaatagtagtatcatggccatttactttcttaggagagacaaagttagttgagaatcgacacaattaaaatgttagttagaggtgatctgttagttaaataaataaagagggtaggagaaaggtagattcattccatcttgtatcattctatttataagcatttactctgtgaaaggaaaacctttgtgaaggatattcattcaaaacttttgcatctatcaatttcactcctctctttccctagttcgtaaataaaaattaaaaaaaaaaatcaacacggtgactggaagccacacctacagtctacagatatgcagagtgttatttgctttaatccatcgtcccttagtctaatagtcttgta

SEQ ID NO: 99 - DNA, 5000 bp, Glycine max
  source 1..5000: mol_type = genomic DNA
aattgtcttctatcattctatgtgctacccgaaaagaaaaaaaaacagacatctgttaaagagtagctgcagctttattctcttatgcatctgtcaaaccaaggagaaaaaaaatgtcaaactgaaaatttatacgtttttcctttatttcctttttaaacaattcttacattcgctattttaaacgaacatacaaatgtcttactgttttattatttcaattatcaccgttgaattgtgtcaaatttctagcaattctaggtctttaatattgactaagtaactttacccccctaaaaaaacaatagattctctctcatttccttgtgcttactaaaatcgaccctttgcaaaattatagttagaaacaccatacttgcagttgttggagtattacagtagaggctataaaaaaaaattacacagtaagaatggaagatactcgactcaaggtcaatttgacaagacaagaatacccgacctttccattaagtaacctaccaagcgtcacgactacagcaataggcataggtgaccgttgacaaaaaaaaaagagaaaaacattggtgtcctttcccatttctaattttaactattgtcttataaatagtgaaatatccctacacttcatagttttagctaatatacacagaataccacgatctcggatttggctgaactacaccgggcacactaaactcagttatcaaaagcagtatctatcattacgagaacttcctgattgcattgcaatacaacacaagaaaatactaacaacgttagaccaagtttgcataaaaattgatactttatacccaaatgtaagatctaaattgacagcaaattaatgaccctatgcacaaaacaaatgtaaacttcgtgcagcataaatagagctgttgcttcttgcacctcaaagttacttccagcataaacaaagggatgtaggaaacaacccataaatagttgtcaggaaaagtaacggggtattaccttagaaacaggattttaatagaatcgcattgtttagaccgcatacgaataaatttaatatcctacctaccataatattattgcaatgaagtgtaatcagaggccgtgaaagtgaaattgttttttaaaaaaggcgccaaaatattagtacgaaatatactcatcccacgacacgtgagtttgtactagtttgtaaaaataaacaaaccttcaaacatataacaccttaatacattaaccacccgaaatcaatccttgggaaaatactaatatttatcaccaaactcaacattacgtaaaagtatagttgcgaaaaataaaacatgcattaacagagccatgtaagaagtcattcatcaagtggcatgagtcacactccagccatgctgttataaatcgcctgataaccagttgcaagatcacaagacaggacaatgcaagggagataaacaaagcagcgggacaattagcagaaaccataaatgaaaccatatatatataatataaccggcattataaaaattatgcacaaagaaccaacattagtaaaagatctctttcatacacatatcagaagggatttccaactttaaaaagtgacgtatttcatacaaaatatacatattaaaaaataatcaaacctctatctatatccacaatatcggtaaaagaaaaaatatcattaacaaaataggagagattatttgatagtcgatcgatcaacaggtttccatgttctgtactaagagaaaccactcaactaagatctacctgccaaatactttccgatccatgattgatttcttaaatctcaacgcagcttgcttctgcaaatcaagaactacaagaagaagggatggagattgagagctttacatttcaaaaaccgaacagcaaagtctagaacaagtagttgaagacaatatttcacatatgataagatagagtctagaaccctatggaggacgatattttttaatctagtattggaggacaagtagttgaagacattttcgtacacaatgtacaatgccttttaggaggaagagtaaaatcaagaactagaccataaaaattaaagcaagaaaagagaaagcttgagatttgaatgattaccagtaaccagctgcagctataagcgagagtttcatcctttcgtagcccaatagacaggttatgtcatgagccatccccatcacctttggcaatgcagaaaaattctgtttgtttatttctgacttggctttaccatggccaatatacccaacaatctcaaatcccaaatgtcctgttgaacacatccatagaattaggcatccgatattttgaatcatcatatcctctataaaacttgttaaatccaagtctttcatttctcaaaagctctgccacacccagaccatttccattccgcccatccaattggccatttgacaagactgcatttctagactgctgcagagataactgaggaaatgaagccagattactaacttgtgattgatttaaccttgaagaaatgccataagaatcaccaaactgagaaaaggaattcccaatttcagaaaatcgaaggtcttgctgtggtgagggagatctttgcatcaataattgaactctggcctcattttcaaagtagttaaactgttcagggcctttactaactgccaaaatagcaggatccataaattcaacgtcacctggaccaccaatatttccatttgagggtgtttgataagaatttctcagtaagaaagaagggtccagcaatgaattccctgtaacgggcataacattaaaatgaaaccaatatgcagaaagacatgtaaaagtagaaagaaacaataactaaccagagagggagtcaaaagcctgccccattctctcaactgaagagaaaccaggaggtggtgccctgttcggaacagagaatcctggcggtgctgaaatttgtgctcttgaaacagctgaaagtacatgataaaggaatgtcattacatctctaaacgaagaaaagacacttctataagctgtcatgccactttaacttggtgacttaactctcaaagtcagtgatctacaataaaccaccacaaaaattgaaaaaggtatgttatccaaacatttaacttgagtaaaacctgagaagtggacctttcatgggttttttgggcaatacaagtgtacttaagcaaatttttattctcccaaacgtgataaaaactcaatttttgttaatctacacacaagtttaatattgaaattttgtgcagagtaccattagttaagtcagctcttgactatagacttttccctagttatttctggcaaatctcaccaaaagtaagacatttttcaaaatacaggttacttaagaagaatattcaacaagaatagcacaaaataagacaagcatggctatgaaaagagcagcaaaagtaccaatgataagaaatgcttctataaaagctacaatagccagtttaagtaagctattaatactaactaacctgaaagcttattgtttgaagcaatgaaatgaccactgccaagatttccagattccttgaggttattggaggggaagccatttgcaaaacccagcttgttcaaagaaaagtctctttccacaaaatcctggatgagtgagccattctttggaaatggctgggtagcaccagaagatggaggtacattggcttggaatttggattcctcctgccatgcaaaagaaaatcttgattgattgttactttgaactttcctagaactagatttttttaacggaccattctggctgtcagtgttgtcacccaacaacttggtcaaactctcatgtgatgccagcgagtcatcccaggcatcaaactccatcgataatatatttgaaattatactactctcacccttgtccataacagcatcacttccaatattaactgcatcactaactaatcttccaatgtggtgctcattgcttgcatcatggcggaaccaataagaactggtactgatctcatcagaatatccattgcatatgttatgtgcatgtaatagtgagttatcctcaactttatcattggcatctaaaaaaccagcatttacaacattacatgggtcaccacgctgcagaagaagaggactagaatgatttgagacatgaaggaaactggctgactcgggcaaataagaaccaataacttctggatccttaagtctctgattatcaaaagttgttacatcatcttccacttcagtattagctgatactacttgagattgagaatccaatgacaaatcacactgttcctttaaattacaaacctcattatataatgcagcagctttactgtctgcatttttaatcattaaatctttcaatttgtcagtattatactgcaattcttgagattgcatagatttaaccaacacattatcaggtgagctgctaggtatggataggctgtagtgtttattttgggcatttctatcaatgtaaacagaagaaaactcgcaggacaaattctcaatctcttcacaggtagatataactgcttcctcaggaccaaagcttaatggttgtcgagtgatgtcaattgaactagttgtgttagttgcagtaaagctgcctctaccactatctccggctaatggtagagaagacaactggttgttcaatttaacagctgaaagagaaacatcagaaaccaaagttctttcaccagcactatccaggatatccatgcttctattttgttcaacaggtttcaataattcattttttatttgaggtgtcatactacgactcccatcactcaacagtggcctctttgtcacatcactatttaatgaagcctgaattgtgcatgcaacagcagaggaaaagggtaggctgctgctgattgtatcaggtttaggcctggacagtccatttggacataataaaccatcagcagctggctgacaatttgtagcccgtgtacccctagcaaacacattgattggaa

SEQ ID NO: 100 - DNA, 50 bp, Glycine max
  source 1..50: mol_type = other DNA
ccatattatatttattatttctctcttttcttttcagttaactatattcc

SEQ ID NO: 101 - DNA, 50 bp, Glycine max
  source 1..50: mol_type = other DNA
gtattattccggtcatgtcaagtttggcaatgagtcttgtcaagttaaga

SEQ ID NO: 102 - DNA, 50 bp, Glycine max
  source 1..50: mol_type = other DNA
atccatcatctgcataacaattagtgctacctgttccttttctgtttttt

SEQ ID NO: 103 - DNA, 50 bp, Glycine max
  source 1..50: mol_type = other DNA
atatatatatatatatatatatatatatatatatatatatatatatatat

SEQ ID NO: 104 - DNA, 50 bp, Glycine max
  source 1..50: mol_type = other DNA
cgcccatcgggtatttttaattaaagtaatatttaaaaattaaaaaggaa

SEQ ID NO: 105 - DNA, 50 bp, Glycine max
  source 1..50: mol_type = other DNA
gtatgaagaataaattgtaaaaatagaaattaataaaataatataaataa

SEQ ID NO: 106 - DNA, 50 bp, Glycine max
  source 1..50: mol_type = other DNA
ctgtttttttttaatatatgggctttaagttcaatatattccaagtaatc

SEQ ID NO: 107 - DNA, 50 bp, Glycine max
  source 1..50: mol_type = other DNA
ctctacaatttgaactctaaataaaaaaaattatcaaagtgaaagaataa

SEQ ID NO: 108 - DNA, 50 bp, Glycine max
  source 1..50: mol_type = other DNA
tgggtacccttatggccttatccaaaaaaaatcacatatggtccaatgaa

SEQ ID NO: 109 - DNA, 50 bp, Glycine max
  source 1..50: mol_type = other DNA
attgaataaaaaacccgtttagtaataataaaaaataaccagttttgtaa

SEQ ID NO: 110 - DNA, 50 bp, Glycine max
  source 1..50: mol_type = other DNA
tagggtaaaaactagtggtaaagaacaacagttcaaatcgtcactttatt

SEQ ID NO: 111 - DNA, 50 bp, Glycine max
  source 1..50: mol_type = other DNA
agaaagactaatatacgaattttttactcgaattactatcatttaatact

SEQ ID NO: 112 - DNA, 50 bp, Glycine max
  source 1..50: mol_type = other DNA
taatagtttattggggtactatttaataatttgtggaatgctaaaataag

SEQ ID NO: 113 - DNA, 50 bp, Glycine max
  source 1..50: mol_type = other DNA
tttaaaatgaatgtaaattatgtaatgttcgaagcaattaacagtttggt

SEQ ID NO: 114 - DNA, 50 bp, Glycine max
  source 1..50: mol_type = other DNA
ggggggagttgtttaacaccgacacaaaatgaatagggtctttggacttt

SEQ ID NO: 115 - DNA, 50 bp, Glycine max
  source 1..50: mol_type = other DNA
aatataaaatttaatctttaaaagtgtaaaaaatataataaatatatctc

SEQ ID NO: 116 - DNA, 50 bp, Glycine max
  source 1..50: mol_type = other DNA
caaatttatctctaaattacacatgacactaataactctttatccttaaa

SEQ ID NO: 117 - DNA, 50 bp, Glycine max
  source 1..50: mol_type = other DNA
caaagcaaagcaaaggttttctgattgtttttattttcatctctctttgg

SEQ ID NO: 118 - DNA, 50 bp, Glycine max
  source 1..50: mol_type = other DNA
tgctttctgctttgtcttatagttgatgcacatgtcataaagagtgaggg

SEQ ID NO: 119 - DNA, 50 bp, Glycine max
  source 1..50: mol_type = other DNA
gaataataaatgatgatattattgaagtagttcacggtgaaaaaaaaaat

SEQ ID NO: 120 - DNA, 50 bp, Glycine max
  source 1..50: mol_type = other DNA
tcattttaatgaattttactttttaatcaataaccatattttatgattta

SEQ ID NO: 121 - DNA, 50 bp, Glycine max
  source 1..50: mol_type = other DNA
taaacaaaatgtttgagagaatattgatatcattttaaaaaaataggttt

SEQ ID NO: 122 - DNA, 50 bp, Glycine max
  source 1..50: mol_type = other DNA
gctaatataattattggatgtttccattaagtaccagctcttaataagat

SEQ ID NO: 123 - DNA, 50 bp, Glycine max
  source 1..50: mol_type = other DNA
tgaattcttatttatttttgtgtgtcatatgttatggtagcatatgtcag

SEQ ID NO: 124 - DNA, 50 bp, Glycine max
  source 1..50: mol_type = other DNA
atttaataaagtttttagagttggataaaaaactgtgttacttaaatcat

SEQ ID NO: 125 - DNA, 50 bp, Glycine max
  source 1..50: mol_type = other DNA
tatatatatatatatatatatatatataaagtgatttgtaatggtgagac

SEQ ID NO: 126 - DNA, 50 bp, Glycine max
  source 1..50: mol_type = other DNA
acccaaatagaaaaaaaattgtcccatattccaaatttaaaaaacaaagg

SEQ ID NO: 127 - DNA, 50 bp, Glycine max
  source 1..50: mol_type = other DNA
gtttaaaataagtttttataaaacattttttcctgcaggataatctcttt

SEQ ID NO: 128 - DNA, 50 bp, Glycine max
  source 1..50: mol_type = other DNA
ttgttagtaagatggattcaacatgcaattttttcatttttttacttttt

SEQ ID NO: 129 - DNA, 50 bp, Glycine max
  source 1..50: mol_type = other DNA
tctttatactagagttgacaatagacttaatgaactacacttataaatat

SEQ ID NO: 130 - DNA, 50 bp, Glycine max
  source 1..50: mol_type = other DNA
aggcatttgcttctcacgtatgcacatggatgcgattaaggaccactttc

SEQ ID NO: 131 - DNA, 50 bp, Glycine max
  source 1..50: mol_type = other DNA
tttattatcatctttgctttgaaagtcactacttttctattttagaggga

SEQ ID NO: 132 - DNA, 50 bp, Glycine max
  source 1..50: mol_type = other DNA
atttagaaggggaaaaaataacaaagaaacggttcttgataggaaaataa

SEQ ID NO: 133 - DNA, 50 bp, Glycine max
  source 1..50: mol_type = other DNA
tattttgaaaaaagaggctaaatgaaaaagtaagaattatttaaaaaatt

SEQ ID NO: 134 - DNA, 50 bp, Glycine max
  source 1..50: mol_type = other DNA
tgagatcttctattctctctcattttttagtaggaaaaacaactaatagt

SEQ ID NO: 135 - DNA, 50 bp, Glycine max
  source 1..50: mol_type = other DNA
taaagtcaatccaacattactcgatccgtaacgttgttatgacaaccgaa

SEQ ID NO: 136 - DNA, 50 bp, Glycine max
  source 1..50: mol_type = other DNA
gaccatggtaattggtaggtgaaacattcaatgcccaacttaacaaaatg

SEQ ID NO: 137 - DNA, 50 bp, Glycine max
  source 1..50: mol_type = other DNA
gcatcatgcagcattccgaatccataatttcctcaaaattatgaacctat

SEQ ID NO: 138 - DNA, 50 bp, Glycine max
  source 1..50: mol_type = other DNA
attttaaattatactaaatacattaacccaattttgagaacaaataggaa

SEQ ID NO: 139 - DNA, 50 bp, Glycine max
  source 1..50: mol_type = other DNA
ttctaatagttgagatattatatattattcctactgaattccgacgagtt

SEQ ID NO: 140 - DNA, 50 bp, Glycine max
  source 1..50: mol_type = other DNA
atatcctacctaccataatattattgcaatgaagtgtaatcagaggccgt

SEQ ID NO: 141 - DNA, 50 bp, Glycine max
  source 1..50: mol_type = other DNA
tactcatcccacgacacgtgagtttgtactagtttgtaaaaataaacaaa

SEQ ID NO: 142 - DNA, 50 bp, Glycine max
  source 1..50: mol_type = other DNA
gggaaaatactaatatttatcaccaaactcaacattacgtaaaagtatag

SEQ ID NO: 143 - DNA, 50 bp, Glycine max
  source 1..50: mol_type = other DNA
caagtggcatgagtcacactccagccatgctgttataaatcgcctgataa

SEQ ID NO: 144 - DNA, 50 bp, Glycine max
  source 1..50: mol_type = other DNA
gcgggacaattagcagaaaccataaatgaaaccatatatatataatataa

SEQ ID NO: 145 - DNA, 50 bp, Glycine max
  source 1..50: mol_type = other DNA
tttcatacacatatcagaagggatttccaactttaaaaagtgacgtattt

SEQ ID NO: 146 - DNA, 50 bp, Glycine max
  source 1..50: mol_type = other DNA
aatatcggtaaaagaaaaaatatcattaacaaaataggagagattatttg

SEQ ID NO: 147 - DNA, 50 bp, Glycine max
  source 1..50: mol_type = other DNA
caactaagatctacctgccaaatactttccgatccatgattgatttctta

SEQ ID NO: 148 - DNA, 50 bp, Glycine max
  source 1..50: mol_type = other DNA
tggagattgagagctttacatttcaaaaaccgaacagcaaagtctagaac

SEQ ID NO: 149 - DNA, 50 bp, Glycine max
  source 1..50: mol_type = other DNA
tatggaggacgatattttttaatctagtattggaggacaagtagttgaag

SEQ ID NO: 150 - DNA, 50 bp, Glycine max
  source 1..50: mol_type = other DNA
agaactagaccataaaaattaaagcaagaaaagagaaagcttgagatttg

SEQ ID NO: 151 - DNA, 50 bp, Glycine max
  source 1..50: mol_type = other DNA
gtagcccaatagacaggttatgtcatgagccatccccatcacctttggca

SEQ ID NO: 152 - DNA, 50 bp, Glycine max
  source 1..50: mol_type = other DNA
atatacccaacaatctcaaatcccaaatgtcctgttgaacacatccatag

SEQ ID NO: 153 - DNA, 50 bp, Glycine max
  source 1..50: mol_type = other DNA
aatccaagtctttcatttctcaaaagctctgccacacccagaccatttcc

SEQ ID NO: 154 - DNA, 50 bp, Glycine max
  source 1..50: mol_type = other DNA
gcagagataactgaggaaatgaagccagattactaacttgtgattgattt

SEQ ID NO: 155 - DNA, 50 bp, Glycine max
  source 1..50: mol_type = other DNA
aatttcagaaaatcgaaggtcttgctgtggtgagggagatctttgcatca

SEQ ID NO: 156 - DNA, 50 bp, Glycine max
  source 1..50: mol_type = other DNA
ttactaactgccaaaatagcaggatccataaattcaacgtcacctggacc

SEQ ID NO: 157 - DNA, 50 bp, Glycine max
  source 1..50: mol_type = other DNA
aagggtccagcaatgaattccctgtaacgggcataacattaaaatgaaac

SEQ ID NO: 158 - DNA, 50 bp, Glycine max
  source 1..50: mol_type = other DNA
gagggagtcaaaagcctgccccattctctcaactgaagagaaaccaggag

SEQ ID NO: 159 - DNA, 50 bp, Glycine max
  source 1..50: mol_type = other DNA
cttgaaacagctgaaagtacatgataaaggaatgtcattacatctctaaa

SEQ ID NO: 160 - DNA, 50 bp, Glycine max
  source 1..50: mol_type = other DNA
taactctcaaagtcagtgatctacaataaaccaccacaaaaattgaaaaa

SEQ ID NO: 161 - DNA, 50 bp, Glycine max
  source 1..50: mol_type = other DNA
tcatgggttttttgggcaatacaagtgtacttaagcaaatttttattctc

SEQ ID NO: 162 - DNA, 50 bp, Glycine max
  source 1..50: mol_type = other DNA
ttgaaattttgtgcagagtaccattagttaagtcagctcttgactataga

SEQ ID NO: 163 - DNA, 50 bp, Glycine max
  source 1..50: mol_type = other DNA
aaaatacaggttacttaagaagaatattcaacaagaatagcacaaaataa

SEQ ID NO: 164 - DNA, 50 bp, Glycine max
  source 1..50: mol_type = other DNA
cttctataaaagctacaatagccagtttaagtaagctattaatactaact

SEQ ID NO: 165 - DNA, 50 bp, Glycine max
  source 1..50: mol_type = other DNA
ccagattccttgaggttattggaggggaagccatttgcaaaacccagctt

SEQ ID NO: 166 - DNA, 50 bp, Glycine max
  source 1..50: mol_type = other DNA
ttggaaatggctgggtagcaccagaagatggaggtacattggcttggaat

SEQ ID NO: 167 - DNA, 50 bp, Glycine max
  source 1..50: mol_type = other DNA
aactttcctagaactagatttttttaacggaccattctggctgtcagtgt

SEQ ID NO: 168 - DNA, 50 bp, Glycine max
  source 1..50: mol_type = other DNA
caggcatcaaactccatcgataatatatttgaaattatactactctcacc

SEQ ID NO: 169 - DNA, 50 bp, Glycine max
  source 1..50: mol_type = other DNA
ttccaatgtggtgctcattgcttgcatcatggcggaaccaataagaactg

SEQ ID NO: 170 - DNA, 50 bp, Glycine max
  source 1..50: mol_type = other DNA
tgagttatcctcaactttatcattggcatctaaaaaaccagcatttacaa

SEQ ID NO: 171 - DNA, 50 bp, Glycine max
  source 1..50: mol_type = other DNA
acatgaaggaaactggctgactcgggcaaataagaaccaataacttctgg

SEQ ID NO: 172 - DNA, 50 bp, Glycine max
  source 1..50: mol_type = other DNA
tattagctgatactacttgagattgagaatccaatgacaaatcacactgt

SEQ ID NO: 173 - DNA, 50 bp, Glycine max
  source 1..50: mol_type = other DNA
atttttaatcattaaatctttcaatttgtcagtattatactgcaattctt

SEQ ID NO: 174 - DNA, 50 bp, Glycine max
  source 1..50: mol_type = other DNA
gataggctgtagtgtttattttgggcatttctatcaatgtaaacagaaga

SEQ ID NO: 175 - DNA, 50 bp, Glycine max
  source 1..50: mol_type = other DNA
cctcaggaccaaagcttaatggttgtcgagtgatgtcaattgaactagtt

SEQ ID NO: 176 - DNA, 50 bp, Glycine max
  source 1..50: mol_type = other DNA
agaagacaactggttgttcaatttaacagctgaaagagaaacatcagaaa

SEQ ID NO: 177 - DNA, 50 bp, Glycine max
  source 1..50: mol_type = other DNA
tcaacaggtttcaataattcattttttatttgaggtgtcatactacgact

SEQ ID NO: 178 - DNA, 50 bp, Glycine max
  source 1..50: mol_type = other DNA
gaattgtgcatgcaacagcagaggaaaagggtaggctgctgctgattgta

SEQ ID NO: 179 - DNA, 50 bp, Glycine max
  source 1..50: mol_type = other DNA
tggctgacaatttgtagcccgtgtacccctagcaaacacattgattggaa

(19) United States
(12) Patent Application Publication
(10) Pub. No.: US 2023/0348927 A1
(43) Pub. Date: Nov. 2, 2023

(54) TRANSGENIC SOYBEAN EVENT GM_CSM63714 AND METHODS FOR DETECTION AND USES THEREOF

(71) Applicant: Monsanto Technology LLC, St. Louis, MO (US)

(72) Inventors: Sarah Brown, Weldon Spring, MO (US); Christine M. Ellis, Manchester, MO (US); Tracy E. Klingaman, Saint Charles, MO (US); Clayton Larue, Chesterfield, MO (US); Qungang Qi, Wildwood, MO (US); Aihua Shao, Weldon Spring, MO (US); Janice Weihe, New Baden, IL (US)

(21) Appl. No.: 18/306,581

(22) Filed: Apr. 25, 2023

Related U.S. Application Data
(60) Provisional application No. 63/335,470, filed on Apr. 27, 2022.

(51) Int. Cl. C12N 15/82; C12Q 1/6895; A01H 6/54

(52) U.S. Cl. CPC C12N 15/8275; C12Q 1/6895; A01H 6/542; C12Q 2600/13

(57) ABSTRACT

A transgenic soybean event, Gm_CSM63714, is provided. Transgenic plant cells, plant parts, plants, seeds, progeny plants, and agricultural and commodity products containing event Gm_CSM63714 are also provided. Recombinant DNA molecules unique to the event Gm_CSM63714, and methods of using and detecting Gm_CSM63714 are also provided. Soybean plants containing the event Gm_CSM63714 exhibit tolerance to benzoic acid auxins such as dicamba; phenoxy auxins such as 2,4-D; inhibitors of glutamine synthetase such as glufosinate; and β-triketone inhibitors of 4-hydroxyphenylpyruvate dioxygenase (HPPD) such as mesotrione.

### CROSS REFERENCE TO RELATED APPLICATIONS

This application claims the benefit of U.S. Provisional Application No. 63/335,470, filed Apr. 27, 2022, herein incorporated by reference in its entirety.

### INCORPORATION OF SEQUENCE LISTING

The sequence listing contained in the file named “MONS532US_ST26.xml”, which is 222 kilobytes (measured in MS-Windows), was created on Feb. 13, 2023, is filed herewith by electronic submission, and is incorporated herein by reference in its entirety.

### FIELD OF THE INVENTION

The present disclosure relates generally to the fields of agriculture, plant biotechnology and molecular biology. More specifically, the disclosure relates to compositions and methods for providing herbicide tolerance in transgenic soybean plants. More specifically, recombinant DNA molecules of soybean event Gm_CSM63714 are provided. Also provided are transgenic soybean plants, plant parts, seeds, cells, and agricultural products comprising the soybean event Gm_CSM63714, as well as methods of producing and using transgenic soybean plants, plant parts, seeds, cells, and agricultural products comprising soybean event Gm_CSM63714, methods of detecting soybean event Gm_CSM63714, and methods of controlling weeds. Transgenic soybean plants, plant parts, seeds and cells comprising soybean event Gm_CSM63714 exhibit tolerance to benzoic acid auxins such as dicamba; inhibitors of glutamine synthetase such as glufosinate; phenoxy auxins such as 2,4-D; and β-triketone herbicides (inhibitors of 4-hydroxyphenylpyruvate dioxygenase, or HPPD) such as mesotrione.

### BACKGROUND OF THE INVENTION

Increasing sustainable crop production is crucial to meet the need for food for the growing global population, feed for increased demand on animal-based diets in developing nations, and expanded use of crop products to produce biofuel, fiber, and other agricultural product-based commodities, while using limited natural resources. In agricultural systems, the effective management of weedy species in agricultural fields is essential for maintaining favorable crop growing conditions and yield. Weeds compete with crops for space, nutrients, water, and light and can contaminate harvests, and present one of the major challenges to sustainable crop production. In soybean alone, poor weed control can cause up to nearly 50% of yield reduction and up to $16 billion of annual loss in the United States (Weed Science Society of America). Selective herbicides had significantly contributed to weed management before the deployment of herbicide tolerant crops. Application of herbicides provides an important tool to reduce weed pressure, improve productivity and increase security for global crop production.

Soybean (Glycine max) is an important crop in many areas of the world. The introduction of genetically modified crops containing herbicide tolerance traits has successfully provided additional tools available to farmers to better control weeds. Transgenic herbicide tolerance enables the use of an herbicide in a crop growing environment without crop injury or with minimal crop injury (e.g., less than about 10% injury). Transgenic soybean traits have been used to impart tolerance to glyphosate and/or dicamba and are used broadly in commercial soybean production for weed management. However, weeds have evolved resistance to herbicides and weed resistance continues to present a challenge in soybean production today. Therefore, there is a need for additional herbicide tolerance trait options to manage weeds effectively and to sustain crop productivity. One of the solutions is to employ multiple herbicide modes of action.

Combinations of herbicide tolerance traits are desirable to provide weed control options that increase grower flexibility and enable the use of multiple herbicide modes of action for controlling challenging weeds. Combining multiple desired traits in the genome can be achieved by making crosses between two parents each having a desired trait, and identifying progeny plants that have combination of the desired traits, or by retransforming a transgenic plant comprising one or more desired trait(s) with one or more genes for additional desired traits, either through random integration or through targeted integration of the one or more genes for additional desired traits. Alternatively, combining multiple desired traits can be achieved by inserting multiple genes as a single DNA molecule into one location, or locus, in the genome. The combination of multiple herbicide tolerance traits at a single locus in soybean would provide a useful tool in weed control that is much simpler and less expensive to maintain during subsequent breeding into a diverse pool of elite germplasms.

The expression of transgenes in a transgenic plant, plant part, seed, cell or progeny, and thus their effectiveness, may be influenced by many factors, such as the regulatory elements used in the transgenes' expression cassettes, the combination and/or interaction of these regulatory elements, the chromosomal location of the transgene insertion site, the chromatin structure of the genome at or near the transgene insertion site, and the presence or proximity of any endogenous cis and/or trans regulatory elements or genes close to the transgene insertion site. In addition, the performance of the traits in the transgenic plant is further complicated when the transgenic insert comprises multiple expression cassettes, each having a different transgene conferring a distinct trait. These differences or factors may result in variation in the level of transgene expression or in the spatial or temporal pattern of transgene expression among different transgenic insertion events of the same expression cassettes. Furthermore, different transgenic events can also vary in terms of the molecular quality of the events. For example, a transgenic event may contain two or more copies of the transgene insertion at one or more chromosomal locations, or a transgenic insertion may be truncated relative to the intended insertion or contain vector backbone sequences, or a transgene may be inserted into an endogenous gene or in a repeated region. Such characteristics may result in undesirable outcomes, such as gene silencing, altered pattern and/or expression of the transgene, altered pattern and/or expression of the endogenous genes. There may also be undesirable phenotypic or agronomic differences among different events.

Even in the case of targeted sequence insertion, variability in the level of transgene expression between independent but genetically identical targeted sequence insertion (TSI) events was observed in a subset of transgenic events (Verkest et al., 2019). This expression variability and silencing occurred independently of the transgene sequence and could be attributed to DNA methylation that was further linked to different DNA methylation mechanisms. The fact that a considerable variation in transgene expression was observed in a subset of clean TSI events shows that even when integration events are targeted, selection remains necessary similarly to the practice for random integration events in order to identify TSI events with stable gene of interest expression over generations.

A commercially useful multi-gene transgenic event requires that each of the transgenes in the transgenic insert express in the manner necessary for that trait to be successful, and involves rigorous testing, evaluation and selection. Once one or more tolerance traits have been chosen, individual expression cassettes are designed and tested in vitro and/or in planta to select for the best expression cassettes for each trait. Such tests include testing different regulatory elements (e.g., promoters, introns, leaders, and 3′ UTRs) and combinations of different regulatory elements for desirable spatial and temporal expression of the transgenes, as well as examining whether to target the product of the transgenes (proteins) to subcellular compartments such as chloroplasts. Then the selected expression cassettes for each trait are then combined into one construct, and the construct is tested to ensure that all the expression cassettes function well together and each transgene is properly expressed. The selected combinations of expression cassettes are then used for transformation to produce transgenic plants. Since Agrobacterium-mediated transformation with a T-DNA construct comprising one or more transgene cassettes is largely variable and random in terms of where the transgene(s) can be inserted into the plant genome, each transgenic event is unique with random and unique insertion of the transgenic DNA in a different plant genomic location. Thus, the selected combinations of expression cassettes are used to produce hundreds of unique multi-gene transgenic events, each the result of a random insertion of the foreign DNA in a different plant genomic location.

For these reasons, the performance of different transformation events from the same transformation construct can vary, and the identification of transformation events conferring the most beneficial traits or characteristics without other potential off-types or concerns is needed to select a superior event for commercial use. Therefore, a large number of individual transgenic events must be produced and analyzed to select an event having superior commercial properties, which can be a significant undertaking that involves analysis and selection among many different transformation events.

To establish a multi-gene event for commercial use requires rigorous molecular characterization, greenhouse testing, and field trials over multiple years, in multiple locations and under a variety of conditions, allowing extensive agronomic, phenotypic, and molecular data to be obtained. The resulting data are then analyzed to select an event that is suitable for commercial purposes. The commercial multi-gene event, once identified as having the desired transgene expression, molecular characteristics, efficacy and field performance, can then be introgressed as a single locus having multiple herbicide tolerance traits into other soybean genetic backgrounds using plant breeding methods. The resulting soybean varieties contain the new traits combined with other desirable qualities such as native traits, disease tolerance traits, insect control traits, high-yielding germplasm or traits, and/or one or more other transgenic herbicide tolerance traits.

### SUMMARY OF THE INVENTION

Recombinant DNA molecules are provided herein. Examples of such recombinant DNA molecules include a sequence selected from the group consisting of SEQ ID NO:10, SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, a polynucleotide having a nucleotide sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO:10 or the full length of SEQ ID NO: 9, and a complete complement of any of the foregoing. In some embodiments, the recombinant DNA molecule is derived from a soybean plant, seed, plant part, plant cell, progeny plant, or commodity product comprising soybean event Gm_CSM63714 a representative sample of seed comprising the event having been deposited as ATCC Accession No. PTA-127099. In some embodiments, the recombinant DNA is comprised in a soybean plant, seed, plant part, plant cell, or progeny plant comprising soybean event Gm_CSM63714, or a commodity product produced therefrom, a representative sample of seed comprising the event having been deposited as ATCC Accession No. PTA-127099. The recombinant DNA molecule can be formed by the insertion of a heterologous nucleic acid molecule into the genomic DNA of a soybean plant or soybean cell. The recombinant DNA molecule can comprise an amplicon diagnostic for the presence of soybean event Gm_CSM63714.

DNA molecules that function as DNA probes are provided. An example of such a DNA molecule is a DNA molecule comprising a polynucleotide segment of sufficient length to function as a DNA probe that hybridizes specifically under stringent hybridization conditions with soybean event Gm_CSM63714 DNA in a sample. Detecting hybridization of the DNA molecule under the stringent hybridization conditions is diagnostic for the presence of soybean event Gm_CSM63714 in the sample.

Also provided is a DNA molecule comprising a polynucleotide segment of sufficient length to function as a DNA probe specific for detecting in a sample at least one of: a 5′ junction sequence between flanking soybean genomic DNA and the transgenic insert of soybean event Gm_CSM63714; a 3′ junction sequence between the transgenic insert of soybean event Gm_CSM63714 and flanking soybean genomic DNA; SEQ ID NO:9; and a fragment of SEQ ID NO:9 comprising a sufficient length of contiguous nucleotides of SEQ ID NO:9 to identify the sequence as a fragment of the transgenic insert of Gm_CSM63714.

The DNA probe can comprises SEQ ID NO:16. Alternatively, the DNA molecule that functions as a DNA probe can comprise a nucleotide sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, and a complement of any of the foregoing. the sample can be derived from a soybean plant, seed, plant part, plant cell, progeny plant, or commodity product.

A pair of DNA molecules is provided. The pair of DNA molecules comprises a first DNA molecule and a second DNA molecule. The first and the second DNA molecules comprise a fragment of SEQ ID NO:10 or a complement thereof and function as DNA primers when used together in an amplification reaction with DNA comprising soybean event Gm_CSM63714 to produce an amplicon diagnostic for soybean event Gm_CSM63714 in a sample. For example, the first and the second DNA molecules can comprise SEQ ID NO:14 and SEQ ID NO:15. The amplicon can comprise a nucleotide sequence selected from the group consisting of: SEQ ID NO:1; SEQ ID NO:2; SEQ ID NO:3; SEQ ID NO:4; SEQ ID NO:5; SEQ ID NO:6; SEQ ID NO:7; SEQ ID NO:8; SEQ ID NO:9; SEQ ID NO:10; and a fragment of any of SEQ ID NO:1, SEQ ID NO:2, SEQ ID NO:3, SEQ ID NO:4, SEQ ID NO:5, SEQ ID NO:6, SEQ ID NO:7, SEQ ID NO:8, wherein the fragment is at least 10 nucleotides in length and comprises nucleotides 1,000-1,001 or 11,196-11,197 of SEQ ID NO:10.

Methods for detecting the presence of soybean event Gm_CSM63714 in a sample derived from a soybean seed, plant, plant part, plant cell, progeny plant, or commodity product are provided. In a first example of such a method, the method comprises: a) contacting the sample with any of the DNA molecules that function as probes described herein; b) subjecting the sample and the DNA molecule that functions as a probe to stringent hybridization conditions; and c) detecting the hybridization of the DNA molecule that functions as a probe to a DNA molecule in the sample. The hybridization of the DNA molecule that functions as a probe to the DNA molecule in the sample is diagnostic for the presence of soybean event Gm_CSM63714 in the sample.

Another method of detecting the presence of soybean event Gm_CSM63714 in a sample derived from a soybean seed, plant, plant part or plant cell, progeny plant and commodity product is provided. The method comprises: a) contacting the sample any of the pairs of DNA molecules described herein; b) performing an amplification reaction sufficient to produce a DNA amplicon; and c) detecting the presence of the DNA amplicon; wherein the DNA amplicon comprises at least one of: a 5′ junction sequence between flanking soybean genomic DNA and the transgenic insert of soybean event Gm_CSM63714; a 3′ junction sequence between flanking soybean genomic DNA and the transgenic insert of soybean event Gm_CSM63714; SEQ ID NO: 9; and a fragment of SEQ ID NO: 9 comprising a sufficient length of contiguous nucleotides of SEQ ID NO: 9 to identify the sequence as a fragment of the transgenic insert of Gm_CSM63714. The presence of the DNA amplicon indicates the presence of soybean event Gm_CSM63714 in the sample. The DNA amplicon can be at least 10 nucleotides in length, at least 11 nucleotides in length, at least 12 nucleotides in length, at least 13 nucleotides in length, at least 14 nucleotides in length, at least 15 nucleotides in length, at least 16 nucleotides in length, at least 17 nucleotides in length, at least 18 nucleotides in length, at least 19 nucleotides in length, at least 20 nucleotides in length, at least 25 nucleotides in length, at least 30 nucleotides in length, at least 35 nucleotides in length, at least 40 nucleotides in length, at least 45 nucleotides in length, at least 50 nucleotides in length, at least 60 nucleotides in length, at least 70 nucleotides in length, at least 80 nucleotides in length, at least 90 nucleotides in length, or at least 100 nucleotides in length. The DNA amplicon can comprises a nucleotide sequence selected from the group consisting of SEQ ID NO:10; SEQ ID NO:9; SEQ ID NO:8; SEQ ID NO:7; SEQ ID NO:6; SEQ ID NO:5; SEQ ID NO:4; SEQ ID NO:3; SEQ ID NO:2; SEQ ID NO:1; and a fragment of any of SEQ ID NO:10, SEQ ID NO:8, SEQ ID NO:7, SEQ ID NO:6, SEQ ID NO:5, SEQ ID NO:4, SEQ ID NO:3, SEQ ID NO:2, and SEQ ID NO:1 that is at least 10 nucleotides in length and comprises nucleotides 1,000-1,001 or 11,196-11,197 of SEQ ID NO:10.

A further method of detecting the presence of soybean event Gm_CSM63714 in a sample of DNA derived from a soybean seed, plant, plant part, plant cell, progeny plant or commodity product is provided. The method comprises: a) contacting the sample with any of the DNA molecules that function as probes described herein; and b) performing a sequencing reaction to produce a target sequence. The target sequence comprises a nucleotide sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, a complete complement of any thereof, and a fragment of any of SEQ ID NO:1, SEQ ID NO:2, SEQ ID NO:3, SEQ ID NO:4, SEQ ID NO:5, SEQ ID NO:6, SEQ ID NO:7, SEQ ID NO:8, and SEQ ID NO:10 that is at least 10 nucleotides long and comprises nucleotides 1,000-1,001 or 11,196-11,197 of SEQ ID NO:10.

Another method of detecting the presence of soybean event Gm_CSM63714 in a sample derived from a soybean seed, plant, plant part, cell, progeny plant or commodity product is provided. The method comprises: a) contacting the sample with at least one antibody specific for at least one protein encoded by soybean event Gm_CSM63714; and b) detecting binding of the antibody to the protein in the sample. The binding of the antibody indicates the presence of soybean event Gm_CSM63714 in the sample.

DNA detection kit for detecting the presence of soybean event Gm_CSM63714 in a sample are provided. One example of such a DNA detection kit is a kit comprising any of the pairs of DNA primers described herein. Another example of a DNA detection kit is a kit comprising any of the DNA molecules that functions as a probe described herein.

Also provided are protein detection kits for detecting the presence of soybean event Gm_CSM63714 in a sample. One example of such a kit is a kit comprising at least one antibody specific for at least one protein encoded by soybean event Gm_CSM63714. Detecting binding of the at least one antibody to the at least one protein encoded by soybean event Gm_CSM63714 in a sample is diagnostic for the presence of soybean event Gm_CSM63714 in the sample.

Also provided are a soybean plants, plant seeds, plant parts, and plant cells that comprise a recombinant DNA molecule comprising a sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, a polynucleotide having a sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO:10 or the full length of SEQ ID NO: 9, and a complete complement of any of the foregoing. The soybean plant, plant seed, plant part, or plant cell can express at least one herbicide tolerance gene selected from the group consisting of dicamba monooxygenase (DMO), phosphinothricin N-acetyltransferase (PAT), alpha-ketoglutarate-dependent non-heme iron dioxygenase variant FT_Tv7, triketone dioxygenase (TDO), and any combination thereof. The soybean plant, plant seed, plant part, or plant cell can be tolerant to at least one herbicide selected from the group consisting of benzoic acid auxins, phenoxy auxins, glutamine synthetase inhibitors, β-triketone HPPD inhibitors, and combinations of any thereof. The benzoic acid auxin can comprise dicamba; the phenoxy auxin can comprise 2,4-D; the glutamine synthetase inhibitor can comprise glufosinate; and the β-triketone HPPD inhibitor can be selected from the group consisting of mesotrione, benzobicyclon (BBC), tembotrione, sulcotrione, tefuryltrione, and combinations of any thereof. For example, the β-triketone HPPD inhibitor can comprise mesotrione. The soybean plant, plant seed, plant part, or plant cell can further comprise an additional transgene for tolerance to at least one additional herbicide. For example, the at least one additional herbicide is glyphosate. Where the at least one additional herbicide is glyphosate, the additional transgene can comprise a polynucleotide sequence encoding a protein having the amino acid sequence of SEQ ID NO:57. The soybean plant, plant seed, plant part, or plant cell can comprise soybean event Gm_CSM63714, a representative sample of seed comprising the event having been deposited under ATCC Accession No. PTA-127099. The soybean plant, plant seed, plant part, or plant cell can be further defined as a progeny plant of any generation of a soybean plant comprising soybean event Gm_CSM63714, or a soybean plant part, plant seed, or plant cell derived therefrom.

Further soybean plants, plant parts, plant seeds, and plant cells are provided. The soybean plants, plant parts, plant seeds, and plant cells comprise soybean event Gm_CSM63714, a representative sample of seed comprising soybean event Gm_CSM63714 having been deposited under ATCC Accession No. PTA-127099.

Any of the soybean plant parts described herein can comprises a microspore, pollen, an anther, an ovule, an ovary, a flower, a pod, an embryo, a stem, a leaf, a root, or a callus.

Methods for controlling or preventing weed growth in an area are provided. One example of such a method comprises planting soybean comprising event Gm_CSM63714 in the area, and applying an effective amount of at least one herbicide selected from the group consisting of dicamba, glufosinate, 2,4-D, a β-triketone HPPD inhibitor, and any combination thereof, to control weeds in the area without injury to the soybean or with less than about 10% injury to the soybean. Applying the effective amount of at least one herbicide can comprise applying at least two or more herbicides selected from the group consisting of dicamba, glufosinate, 2,4-D, a β-triketone HPPD inhibitor, and any combination thereof over a growing season. The β-triketone HPPD inhibitor can be selected from the group consisting of mesotrione, benzobicyclon (BBC), tembotrione, sulcotrione, tefuryltrione, and any combination thereof. The effective amount of dicamba can be about 0.5 lb/acre to about 2 lb/acre over a growing season. The effective amount of glufosinate can be about 0.4 lb/acre to about 1.6 lb/acre over a growing season. The effective amount of 2,4-D can be about 0.5 lb/acre to about 4 lb/acre over a growing season. When the β-triketone HPPD inhibitor comprises mesotrione, the effective amount of mesotrione can be about 0.09 lb/acre to about 0.36 lb/acre over a growing season.

Methods for controlling volunteer soybean comprising soybean event Gm_CSM63714 in an area are provided. One example of such a method comprises applying an herbicidally effective amount of at least one herbicide other than dicamba, glufosinate, 2,4-D, or a β-triketone HPPD inhibitor, wherein the herbicide application prevents growth of soybean comprising soybean event Gm_CSM63714. The herbicide other than dicamba, glufosinate, 2,4-D, or a β-triketone HPPD inhibitor can be selected from the group consisting of atrazine, bronioxynil (3,5-di-bromo-4-hydroxybenzonitrile), clopyralid, pyrithiobac, isoxaflutole, topramezone, fluometuron, trifloxysulfuron, monosodium methyl arsenate (MSMA), an inhibitor of protoporphyrinogen oxidase (PPO), and combinations of any thereof. Examples of inhibitors of protoporphyrinogen oxidase (PPO) include saflufenacil, flumioxazin, sulfentrazone, and combinations of any thereof.

Methods of obtaining a seed of a soybean plant or a soybean plant that is tolerant to benzoic acid auxins, phenoxy auxins, inhibitors of glutamine synthetase, β-triketone HPPD inhibitors, or any combination thereof are provided. In one example of such a method, the method comprises: a) obtaining a population of progeny seed or plants grown therefrom, at least one of which comprises soybean event Gm_CSM63714; and b) identifying at least a first progeny seed or plant grown therefrom that comprises soybean event Gm_CSM63714. Identifying the progeny seed or plant grown therefrom that comprises soybean event Gm_CSM63714 can comprise: a) growing the progeny seed or plant to produce progeny plants; b) treating the progeny plants with an effective amount of at least one herbicide selected from the group consisting of a benzoic acid auxin, a phenoxy auxin, an inhibitor of glutamine synthetase, a β-triketone HPPD inhibitor, and any combination thereof; and c) selecting a progeny plant that is tolerant to the at least one herbicide selected from the group consisting of a benzoic acid auxin, a phenoxy auxin, an inhibitor of glutamine synthetase, a β-triketone HPPD inhibitor, and any combination thereof. The benzoic acid auxin can comprise dicamba. The phenoxy auxin can comprise 2,4-D. The glutamine synthetase inhibitor can comprise glufosinate. The β-triketone HPPD inhibitor can be selected from the group consisting of mesotrione, benzobicyclon (BBC), tembotrione, sulcotrione, tefuryltrione, and combinations of any thereof. For example, the β-triketone HPPD inhibitor can comprise mesotrione. I Alternatively or in addition, identifying the progeny seed or plant grown therefrom that comprises soybean event Gm_CSM63714 comprises detecting the presence of soybean event Gm_CSM63714 in a sample derived from the progeny seed or plant grown therefrom. Alternatively or in addition, identifying the progeny seed or plant grown therefrom that comprises soybean event Gm_CSM63714 can comprise detecting the presence of at least one protein encoded by soybean event Gm_CSM63714 in a sample derived from the progeny seed or plant grown therefrom.

Methods of determining the zygosity of a soybean plant, plant part, plant seed, or plant cell comprising soybean event Gm_CSM63714 are provided. One example of such a method comprises: a) contacting a sample comprising DNA derived from the soybean plant, plant part, plant seed, or plant cell with a primer set capable of producing a first amplicon diagnostic for the presence of soybean event Gm_CSM63714 and a second amplicon diagnostic for the wild-type soybean genomic DNA not comprising soybean event Gm_CSM63714; b) performing a nucleic acid amplification reaction; and c) detecting the first amplicon and the second amplicon. The presence of both amplicons indicates that the plant, plant part, seed or cell is heterozygous for soybean event Gm_CSM63714. The presence of only the first amplicon indicates that the plant, plant part, seed, or cell is homozygous for soybean event Gm_CSM63714. An illustrative example of a primer set that can be used is a primer set comprising SEQ ID NO:14, SEQ ID NO:15 and SEQ ID NO:20.

Another method of determining the zygosity of a soybean plant, plant part, plant seed, or plant cell comprising soybean event Gm_CSM63714 is provided. The method comprises: a) contacting a sample comprising DNA derived from the soybean plant, plant part, plant seed, or plant cell with a probe set comprising at least a first probe that specifically hybridizes to soybean event Gm_CSM63714, and at least a second probe that specifically hybridizes to soybean genomic DNA that was disrupted by insertion of the heterologous DNA of soybean event Gm_CSM63714 but does not hybridize to soybean event Gm_CSM63714; and b) hybridizing the probe set with the sample under stringent hybridization conditions. Detecting hybridization of only the first probe under the hybridization conditions is diagnostic for a soybean plant, plant part, seed or plant cell homozygous for soybean event Gm_CSM63714. Detecting hybridization of both the first probe and the second probe under the hybridization conditions is diagnostic for a soybean plant, plant part, seed, or plant cell heterozygous for soybean event Gm_CSM63714. An illustrative example of a probe set that can be used is a probe set comprising SEQ ID NO:16 and SEQ ID NO:21.

DNA constructs are provided. One example of such a DNA construct is a DNA construct comprising a first expression cassette, a second expression cassette, a third expression cassette, and a fourth expression cassette. The first expression cassette comprises in operable linkage: i) ubiquitin (UB3) promoter, leader, and intron sequences from Arabidopsis thaliana, ii) a chloroplast transit peptide coding sequence of APG6 (Albino and Pale Green 6) from Arabidopsis thaliana, iii) a codon-optimized dicamba monooxygenase coding sequence (DMO) from Stenotrophomonas maltophilia, and iv) a 3′ UTR sequence of the aluminum-induced Sali3-2 protein from Medicago truncatula. The second expression cassette comprises in operable linkage: i) a promoter and an intron sequence derived from multiple promoter and intron sequences from Arabidopsis thaliana, ii) a codon-optimized phosphinothricin N-acetyltransferase (PAT) coding sequence from Streptomyces viridochromogene, and iii) a 3′ UTR of a small heat shock protein (Hsp20) from Medicago truncatula. The third expression cassette comprises in operable linkage: i) polyubiquitin (UBQ10) promoter, leader, and intron sequences from Arabidopsis thaliana, ii) an alpha-ketoglutarate-dependent non-heme iron dioxygenase variant coding sequence (FT_Tv7) from Sphingobium herbicidovorans, and iii) a 3′ UTR sequence of a putative protein from Medicago truncatula. The fourth expression cassette comprises in operable linkage i) promoter, leader, and intron sequences derived from multiple promoter, leader and intron sequences from Arabidopsis thaliana, ii) a codon-optimized coding sequence of the triketone dioxygenase (TDO) from Oryza sativa, and iii) a 3′ UTR sequence derived from multiple 3′ UTR sequences from Zea mays. For example, the DNA construct can comprise SEQ ID NO:9. The DNA construct can further comprise at the 5′ or 3′ end of the construct: a) at least 50 contiguous nucleotides of SEQ ID NO:11 or SEQ ID NO:98; and/or b) at least 50 contiguous nucleotides of SEQ ID NO:12 or SEQ ID NO:99.

Another DNA construct is provided. The DNA construct comprises a first expression cassette, a second expression cassette, a third expression cassette, and a fourth expression cassette. The first expression cassette comprises a dicamba monooxygenase coding sequence, the second expression cassette comprises a phospinothricin N-acetyltransferase (PAT) coding sequence the third expression cassette comprises an alpha-ketoglutarate-dependent non-heme iron dioxygenase variant coding sequence (FT_Tv7) capable of degrading 2,4-D, the fourth expression cassette comprises a triketone dioxygenase (TDO) coding sequence. The DNA construct further comprises at the 5′ or 3′ end of said construct (i) at least 50 contiguous nucleotides of SEQ ID NO:11 or SEQ ID NO:98; and/or (ii) at least 50 contiguous nucleotides of SEQ ID NO:12 or SEQ ID NO:99.

A further DNA construct is provided. The construct comprises a polynucleotide having a sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO:9. The DNA construct comprises at the 5′ or 3′ end of said construct (i) at least 50 contiguous nucleotides of SEQ ID NO:11 or SEQ ID NO:98; and/or (ii) at least 50 contiguous nucleotides of SEQ ID NO:12 or SEQ ID NO:99.

Any of the DNA constructs described herein can comprise at least 50 contiguous nucleotides of SEQ ID NO:11 or SEQ ID NO:98 at the 5′ end of the construct and at least 50 contiguous nucleotides of SEQ ID NO:12 or SEQ ID NO:99 at the 3′ end of the construct. Any of the DNA constructs described herein can comprise at the 5′ end of said construct one or more nucleotide sequences selected from SEQ ID NOs:58-77 and SEQ ID NOs:100-139. Any of the DNA constructs described herein can comprise at the 3′ end of said construct one or more nucleotide sequences selected from SEQ ID NOs:78-97 and SEQ ID NOs:140-179.

Soybean plants, plant seeds, plant parts, or plant cells comprising any of the DNA constructs described herein are provided.

A method of improving tolerance to at least one herbicide selected from the group consisting of benzoic acid auxins, phenoxy auxins, inhibitors of glutamine synthetase, β-triketone HPPD inhibitors, and any combination thereof in a soybean plant is provided. The method comprises: a) inserting any of the DNA constructs described herein into the genome of a soybean cell; b) generating a soybean plant from the soybean cell; and c) selecting a soybean plant comprising the DNA construct. The selecting can comprise treating the soybean cell or plant with an effective amount of at least one herbicide selected from the group consisting of benzoic acid auxins, phenoxy auxins, inhibitors of glutamine synthetase, β-triketone HPPD inhibitors, and any combination thereof. The benzoic acid auxin can comprise dicamba, the pheoxy auxin comprise 2,4-D, the glutamine synthetase inhibitor can comprise glufosinate, and the β-triketone HPPD inhibitor can be selected from the group consisting of mesotrione, benzobicyclon (BBC), tembotrione, sulcotrione, tefuryltrione, and combinations of any thereof. For example, the 3-triketone HPPD inhibitor can comprise mesotrione.

Also provided is a soybean plant, plant seed, plant part, or plant cell tolerant to herbicides with at least three different herbicide modes of action at a single genomic location. The soybean plant, plant seed, plant part or plant cell comprises any of the DNA constructs described herein.

Also provided is a soybean plant, plant seed, plant part, or plant cell tolerant to at least one herbicide selected from the group consisting of benzoic acid auxins, phenoxy auxins, glutamine synthetase inhibitors, β-triketone HPPD inhibitors, and any combination thereof. The soybean plant, plant seed, plant part, or plant cell comprises any of the DNA constructs provided herein.

The benzoic acid auxin can comprise dicamba, the phenoxy auxin can comprise 2,4-D, the glutamine synthetase inhibitor can comprise glufosinate, and the β-triketone HPPD inhibitor can be selected from the group consisting of mesotrione, benzobicyclon (BBC), tembotrione, sulcotrione, tefuryltrione, and combinations of any thereof. For example, the β-triketone HPPD inhibitor can be mesotrione.

Any of the soybean seeds, plants, plant parts, or cells can be obtained by any of the methods of improving tolerance to at least one herbicide selected from the group consisting of benzoic acid auxins, phenoxy auxins, inhibitors of glutamine synthetase, β-triketone HPPD inhibitors, and any combination thereof in a soybean plant provided herein.

Any of the soybean plants, plant seeds, plant parts, or plant cells can be tolerant to at least an additional herbicide. For example, the at least an additional herbicide can comprise glyphosate.

A method of producing a progeny soybean plant comprising soybean event Gm_CSM63714 is provided. The method comprises: a) sexually crossing a first soybean plant that comprises soybean event Gm_CSM63714 with itself or a second soybean plant; b) collecting one or more seeds produced from the cross; c) growing one or more seeds to produce one or more progeny plants; and d) selecting at least a first progeny plant or seed comprising soybean event Gm_CSM63714. Inbred and hybrid soybean plants and seeds comprising soybean event Gm_CSM63714 that are produced by the method are also provided.

Nonliving soybean plant material and nonregenerable soybean plant material are also provided. The material can comprise any of the recombinant DNA molecules or any of the DNA constructs described herein.

Also provided is nonliving soybean plant material or nonregenerable soybean plant material comprising soybean event Gm_CSM63714, a representative sample of seed comprising the soybean event soybean event Gm_CSM63714 having been deposited under ATCC Accession No. PTA-127099.

Commodity products are also provided. An example of such a commodity product is a commodity product comprising any of the recombinant DNA molecules or any of the DNA constructs described herein. The commodity product can be produced from a transgenic soybean plant, plant part, plant seed, or plant cell comprising the soybean event Gm_CSM63714. The commodity product can comprise for example, whole or processed seeds; viable or nonviable seeds; viable plant parts (such as roots and leaves); viable plant cells; processed plant parts; processed plant tissues; dehydrated plant tissues; dehydrated plant parts; frozen plant tissues; frozen plant parts; food for human consumption such as soy oil, soy milk, soy flour, soy grits, soy protein, soy protein concentrate, hydrolyzed vegetable protein, textured soy protein, lecithin, curd, tofu, vegetable soybean (edamame), soy sprouts, soy film (yuba), roasted soybeans, miso, tempeh, soy sauce, or natto; plant parts processed for animal feed such as soy meal; soy fiber; biodiesel; bio-composite building materials such as particleboard, laminated plywood, or lumber products; soy oil-based solvents; soy oil-based industrial lubricants; soy ink; soy candles; soy crayons; soy-based hydraulic fluid; or soy-based foams.

A method of producing a commodity product is provided. The method comprises: a) obtaining a transgenic soybean plant, plant part, or plant seed comprising soybean event Gm_CSM63714; and b) producing a commodity product from the transgenic soybean plant, plant part, or plant seed.

A method of controlling, preventing, or reducing the development of herbicide-tolerant weeds is provided. The method comprises cultivating in a crop growing environment a soybean plant comprising transgenes that provide tolerance to herbicides with at least three different herbicide modes of action at a single genomic location. The at least three different herbicide modes of action can be selected from the group consisting of inhibition of glutamine synthetase, inhibition of 4-hydroxyphenylpyruvate dioxygenase (HPPD), phenoxy auxins, and benzoic acid auxins.

Also provided is a method for controlling, preventing, or reducing the development of herbicide-tolerant weeds is provided. The method comprises: a) cultivating in a crop growing environment a soybean plant comprising any of the DNA constructs described herein for providing tolerance to herbicides with at least three different herbicide modes of action at a single genomic location; and b) applying to the crop growing environment at least one herbicide selected from the group consisting of dicamba, glufosinate, 2,4-D, a β-triketone HPPD inhibitor, and any combination thereof, wherein the soybean plant is tolerant to the at least one herbicide. The soybean plant can further comprise at least one additional transgene for an additional herbicide mode of action. For example, the at least one additional transgene can be EPSPS for conferring tolerance to glyphosate. The EPSPS transgene can comprise a polynucleotide sequence encoding a protein having the amino acid sequence of SEQ ID NO:57.

A method of reducing loci for soybean breeding by inserting transgenes at a single genomic location for tolerance to at least three different classes of herbicides is provided. The transgenes can be inserted as a single molecularly linked transgenic insert. The single molecularly linked transgenic insert can provide a commercial level of tolerance to at least one herbicide for each herbicide mode of action.

Further soybean plants, plant cells, plant parts, and plant seeds are provided. The soybean plants, plant cells, plant parts, and plant seeds comprise a recombinant DNA construct integrated in chromosome 13. The recombinant DNA construct confers tolerance to at least one herbicide selected from the group consisting of benzoic acid auxins, phenoxy auxins, glutamine synthetase inhibitors, β-triketone HPPD inhibitors, and combinations of any thereof. The recombinant DNA construct is integrated in a position of said chromosome flanked by at least 50 contiguous nucleotides of SEQ ID NO:11 and 50 contiguous nucleotides of SEQ ID NO:12. The benzoic acid auxin can comprise dicamba, the phenoxy auxin can comprise 2,4-D, the glutamine synthetase inhibitor can comprise glufosinate, and the β-triketone HPPD inhibitor can be selected from the group consisting of mesotrione, benzobicyclon (BBC), tembotrione, sulcotrione, tefuryltrione, and combinations of any thereof. For example, the β-triketone HPPD inhibitor can be mesotrione. The at least 50 contiguous nucleotides of SEQ ID NO: 11 can comprise one or more nucleotide sequences selected from SEQ ID NOs:58-77. The at least 50 contiguous nucleotides of SEQ ID NO: 12 can comprise one or more nucleotide sequences selected from SEQ ID NOs:78-97.

### BRIEF DESCRIPTION OF THE DRAWINGS

FIG. 1 illustrates the sequence of the soybean event Gm_CSM63714. Horizontal lines correspond to the positions of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:11, and SEQ ID NO:12 relative to SEQ ID NO:10. The horizontal arrows (SEQ ID NO:14 and SEQ ID NO:15) represent the approximate positions of an illustrative primer pair that can be used to detect soybean event Gm_CSM63714. The horizontal line labeled SEQ ID NO:16 represents the approximate position of an illustrative DNA probe that can be used to detect soybean event Gm_CSM63714. “RB” refers to the Agrobacterium T-DNA right border; “LB” refers to the Agrobacterium T-DNA left border. “P” represents a promoter element; “L” represents a leader (5′ UTR) element; “I” represents an intron element; “CTP” represents a chloroplast transit peptide element; “T” represents a 3′ UTR. “DMO” represents a dicamba monooxygenase coding element; “PAT” represents a phosphinothricin N-acetyltransferase coding element; “FT-Tv7” represents a dioxygenase variant coding element; and “TDO” represents a triketone dioxygenase coding element. The horizontal line labeled SEQ ID NO:13 represents the relative location or position of the wild-type soybean genome where the transgenes (SEQ ID NO:9) were inserted. The dashed line represents a 40-nucleotide deletion in the soybean genome at the site of transgene (SEQ ID NO:9) insertion.

FIG. 2 illustrates the approximate timing for creation, greenhouse and field testing, molecular characterization, and selection the soybean event Gm_CSM63714.

### BRIEF DESCRIPTION OF THE SEQUENCES

SEQ ID NO:1 is a 30-nucleotide sequence representing the 5′ junction region of the soybean genomic DNA and the integrated transgene insert. SEQ ID NO:1 corresponds to nucleotide positions 986-1,015 of SEQ ID NO:10.

SEQ ID NO:2 is a 30-nucleotide sequence representing the 3′ junction region of the integrated transgene insert and the soybean genomic DNA. SEQ ID NO:2 corresponds to nucleotide positions 11,182-11,211 of SEQ ID NO:10.

SEQ ID NO:3 is a 60-nucleotide sequence representing the 5′ junction region of the soybean genomic DNA and the integrated transgene insert. SEQ ID NO:3 corresponds to nucleotide positions 971-1,030 of SEQ ID NO:10.

SEQ ID NO:4 is a 60-nucleotide sequence representing the 3′ junction region of the integrated transgene insert and the soybean genomic DNA. SEQ ID NO:4 corresponds to nucleotide positions 11,167-11,226 of SEQ ID NO:10.

SEQ ID NO:5 is a 100-nucleotide sequence representing the 5′ junction region of the soybean genomic DNA and the integrated transgene insert. SEQ ID NO:5 corresponds to nucleotide positions 951-1,050 of SEQ ID NO:10.

SEQ ID NO:6 is a 100-nucleotide sequence representing the 3′ junction region of the integrated transgene insert and the soybean genomic DNA. SEQ ID NO:6 corresponds to nucleotide positions 11,147-11,246 of SEQ ID NO:10.

SEQ ID NO:7 is a 1,050-nucleotide sequence representing the 5′ genomic flank region of the soybean genomic DNA and 50 bp of the integrated transgene insert. SEQ ID NO:7 corresponds to nucleotide positions 1-1,050 of SEQ ID NO:10.

SEQ ID NO:8 is a 1,050-nucleotide sequence representing 50 bp of the 3′ junction region of the integrated transgene insert and the 3′ genomic flank region of the soybean genomic DNA. SEQ ID NO:8 corresponds to nucleotide positions 11,147-12,196 of SEQ ID NO:10.

SEQ ID NO:9 is a 10,196-nucleotide sequence corresponding to the transgene insert of soybean event Gm_CSM63714. SEQ ID NO:9 corresponds to nucleotide positions 1,001-11,196 of SEQ ID NO:10.

SEQ ID NO:10 is a 12,196-nucleotide sequence corresponding to the contig nucleotide sequence of the 5′soybean genomic DNA sequence (SEQ ID NO:11), the transgene insert in event Gm_CSM63714 (SEQ ID NO:9), and the 3′ soybean genomic DNA sequence (SEQ ID NO:12).

SEQ ID NO:11 is a 1,000-nucleotide sequence representing the 5′ flanking soybean genomic DNA up to the transgene insert (SEQ ID NO:9). SEQ ID NO:11 corresponds to nucleotide positions 1-1,000 of SEQ ID NO:10.

SEQ ID NO:12 is a 1,000-nucleotide sequence representing the 3′ flanking soybean genomic DNA after the transgene insert (SEQ ID NO:9). SEQ ID NO:12 corresponds to nucleotide positions 11,197-12,196 of SEQ ID NO:10.

SEQ ID NO:13 is a 2,040-nucleotide sequence representing wild-type soybean genomic DNA at the location where the transgenic sequence (SEQ ID NO:9) was inserted in event Gm_CSM63714. A 40-nucleotide fragment of SEQ ID NO:13 (nucleotides 1,001-1,040) was deleted in event Gm_CSM63714 due to insertion of the T-DNA.

SEQ ID NO:14 is a 30-nucleotide sequence corresponding to a thermal amplification primer referred to as SQ21524 used in event-specific assay and zygosity assay to detect soybean event Gm_CSM63714 DNA in a sample, and is identical to the nucleotide sequence corresponding to positions 11,075-11,104 of SEQ ID NO:10.

SEQ ID NO:15 is a 27-nucleotide sequence corresponding to a thermal amplification primer referred to as SQ51589 used in event-specific assay and zygosity assay to detect soybean event Gm_CSM63714 DNA in a sample, and is identical to the reverse complement of the nucleotide sequence corresponding to positions 11,201-11,227 of SEQ ID NO:10.

SEQ ID NO:16 is a 16-nucleotide sequence corresponding to a probe referred to as PB10269 used in event-specific assay and zygosity assay to detect soybean event Gm_CSM63714 DNA in a sample, and is identical to the nucleotide sequence corresponding to positions 11,108-11,123 of SEQ ID NO:10.

SEQ ID NO:17 is a 20-nucleotide sequence corresponding to a thermal amplification primer referred to as SQ546 used as an internal control for the event assay for soybean event Gm_CSM63714 and hybridizes to a region of the soybean genome.

SEQ ID NO:18 is a 20-nucleotide sequence corresponding to a thermal amplification primer referred to as SQ549 used as an internal control for the event assay for soybean event Gm_CSM63714 and hybridizes to a region of the soybean genome.

SEQ ID NO:19 is a 16-nucleotide sequence corresponding to a probe referred to as PB50207 used as an internal control for the event assay for soybean event Gm_CSM63714 and hybridizes to a region of the soybean genome.

SEQ ID NO:20 is a 26-nucleotide sequence corresponding to a thermal amplification primer referred to as SQ52071 used in a zygosity assay for soybean event Gm_CSM63714 DNA in a sample and hybridizes to a region of the soybean genome. It corresponds to positions 949-974 of SEQ ID NO:10.

SEQ ID NO:21 is a 16-nucleotide sequence corresponding to a probe referred to as PB50681 used in a zygosity assay for soybean event Gm_CSM63714 DNA in a sample, and hybridizes to a region of the soybean genome that was deleted by T-DNA insertion.

SEQ ID NOs:22-43 are the nucleotide sequences for the genetic elements in the transgenic insert of soybean event Gm_CSM63714 and are further described in Table 1 hereinbelow.

SEQ ID NOs:44-45 are the nucleotide and amino acid sequences of Cas12a of Lachnospiraceae bacterium ND2006, respectively (LbCas12a, also known as LbCpf1).

SEQ ID NO:46 is the amino acid sequence for LbCas12a_V1 (G532R/K595R).

SEQ ID NO:47 is the amino acid sequence for LbCas12a_V2 (G532R/K538V/Y542R).

SEQ ID NO:48 is the amino acid sequence for Cas12a of Francisella_novicida (FnCas12a).

SEQ ID NO:49 is the nucleotide sequence for the gRNA repeat for LbCas12a.

SEQ ID NO:50 is the nucleotide sequence for the gRNA repeat for FnCas12a.

SEQ ID NO:51 is the nucleotide sequence for the gRNA gRNA_5F-65.

SEQ ID NO:52 is the nucleotide sequence for the gRNA gRNA_TI-605.

SEQ ID NO:53 is the nucleotide sequence for the gRNA gRNA_TI-934.

SEQ ID NO:54 is the nucleotide sequence for the gRNA gRNA_TI-946.

SEQ ID NO:55 is the nucleotide sequence for the gRNA gRNA_3F-41.

SEQ ID NOs:56-57 are the codon-optimized coding sequence and amino acid sequence of the aroA gene (also known as 5-enolpyruvylshikimate-3-phosphate synthase (EPSPS)) from Agrobacterium CP4 strain, respectively.

SEQ ID NOs:58-77 are 50-nucleotide sequences in the 5′ flank genomic sequence of event Gm_CSM63714.

SEQ ID NOs:78-97 are 50-nucleotide sequences in the 3′ flank genomic sequence of event Gm_CSM63714.

SEQ ID NO:98 is a 5,000-nucleotide sequence representing soybean genomic DNA that flanks the transgenic insert at the 5′ end of the insert. Nucleotides 4,001-5,000 of SEQ ID NO:98 are identical to nucleotides 1-1,000 of SEQ ID NO: 11. The remaining nucleotides of SEQ ID NO: 98 (nucleotides 1-4,000) are based on the genomic sequence of the Williams 82 soybean cultivar.

SEQ ID NO:99 is a 5,000-nucleotide sequence representing soybean genomic DNA that flanks the transgenic insert at the 3′ end of the insert. Nucleotides 1-1,000 of SEQ ID NO:99 are identical to nucleotides 1-1000 of SEQ NO: 12. The remaining nucleotides of SEQ ID NO:99 (nucleotides 1,001-5,000) are based on the genomic sequence of the Williams 82 soybean cultivar.

SEQ ID NOs:100-139 are additional 50-nucleotide sequences in the 5′ flank genomic sequence of event Gm_CSM63714 based on the genomic sequence of the Williams 82 soybean cultivar.

SEQ ID NOs:140-179 are additional 50-nucleotide sequences in the 3′ flank genomic sequence of event Gm_CSM63714 based on the genomic sequence of the Williams 82 soybean cultivar.

### DETAILED DESCRIPTION OF THE INVENTION

The following definitions, descriptions, and methods are provided to better define the invention and to guide those of ordinary skill in the art in the practice of the invention. Unless otherwise noted, terms are to be understood according to conventional usage by those of ordinary skill in the relevant art.

Herbicide tolerance is an important agronomic trait for effective weed control to maintain favorable crop growing conditions and crop yields, and is achieved by engineering of herbicide tolerance transgenes in crop plants using modern plant biotechnology techniques. Soybean event Gm_CSM63714 provides tolerance to five different herbicide chemistries through different modes of action for weed control and herbicide-resistant weed management.

Soybean event Gm_CSM63714 is provided. The event Gm_CSM63714 was produced by Agrobacterium-mediated transformation of soybean seed-derived embryo explants with a DNA construct harboring two T-DNAs. The first T-DNA comprises four transgene cassettes, encoding a dicamba monooxygenase (DMO), a phosphinothricin N-acetyltransferase (PAT), an alpha-ketoglutarate-dependent non-heme iron dioxygenase variant (FT_Tv7, also referred to as FT_T.1), and a triketone dioxygenase (TDO), respectively, for conferring tolerance to dicamba (3,6-dichloro-2-methoxybenzoic acid), glufosinate (2-amino-4-(hydroxymethylphosphinyl) butanoic acid), 2,4-D (2,4-dichlorophenoxyacetic acid), and β-triketone herbicides (inhibitors of HPPD) such as mesotrione (2-[4-(methylsulfonyl)-2-nitrobenzoyl]-1,3-cyclohexanedione), respectively. The second T-DNA comprises two transgene cassettes, one encoding an aminoglycoside (3″) adenylyltransferase (aadA) for selection of transformed soybean cells using spectinomycin/streptomycin as the selection, and the other encoding a sucrose phosphorylase (splA) from Agrobacterium tumefaciens (GenBank Accession AE009432) and functioning as a marker gene for identification of the presence of the linked selectable marker.

Plant transformation techniques, such as Agrobacterium-mediated or biolistic transformation, can be used to insert foreign DNA (also known as transgenic DNA) randomly into a chromosome in a plant cell to produce a genetically engineered plant cell, also referred to as a “transgenic” or “recombinant” cell. Using these transformation techniques, many individual cells can be transformed, each resulting in a unique “transgenic event” or “event” due to the random insertion of the foreign DNA into the genome. A transgenic plant can then be regenerated from each individual transgenic cell. This results in every cell of the transgenic plant containing the uniquely inserted transgenic event as a stable part of its genome. The transgenic plant can then be used to produce progeny plants, each containing the unique transgenic event. The term “transgenic” refers to a plant, plant part, plant cell, seed, progeny plant, or DNA molecule, construct, or sequence comprising a transgene—e.g., a “transgenic cell” refers to a cell comprising a transgene.

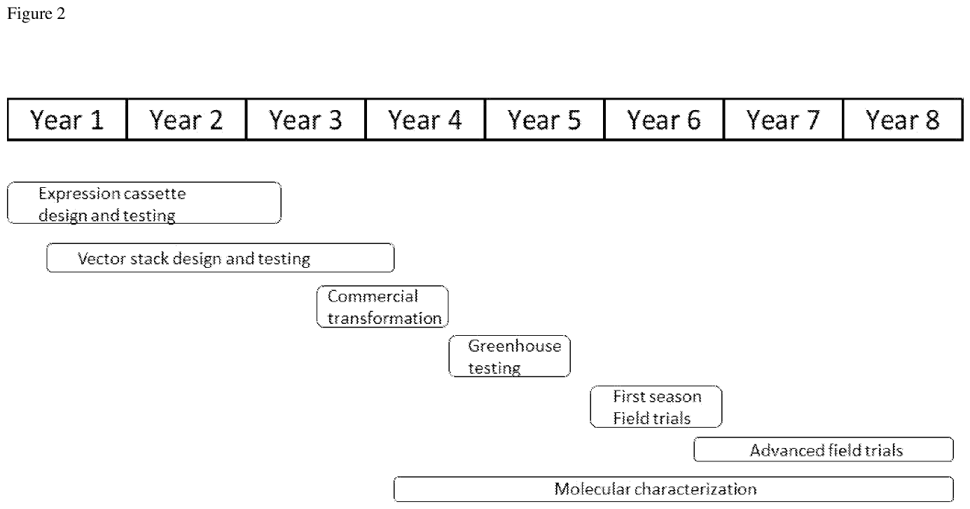

Soybean event Gm_CSM63714 was produced and identified by a complex research and development process. This process included: (i) design and selection of DNA constructs comprising four transgene cassettes based on design and testing of individual transgene cassettes with combinations of different expression elements, followed by design and testing of different combinations of individual transgene cassettes with different positions and orientations relative to each other; (ii) transformation of thousands of soybean cells with the DNA constructs that comprised the four expression cassettes; (iii) regeneration of a large population of transgenic plants each containing a unique transgenic event; and (iv) rigorous multi-year construct and event selection involving molecular characterization of the large number of transgenic events, greenhouse and field trials for herbicide tolerance efficacy and agronomic performance at different locations and in different geographies for thousands of events through tens of thousands of plants. Soybean event Gm_CSM63714 was thus produced and selected as a uniquely superior event useful for broad-scale agronomic commercial purposes. FIG. 2 illustrates the approximate timing for creation, greenhouse and field testing, molecular characterization, and selection the soybean event Gm_CSM63714.

Detailed molecular characterization was conducted on the transgenic events. Event Gm_CSM63714 was selected based on stringent molecular criteria, as well as other selection criteria such as herbicide tolerance efficacy and agronomic performance. The results from such molecular analyses confirmed that: (1) event Gm_CSM63714 contains a single T-DNA insertion with one copy of the transgenic insert comprising the four expression cassettes; (2) no additional elements from the transformation construct were present other than the four expression cassettes between the left and right borders of the first T-DNA, such as the transformation construct backbone sequence or the second T-DNA containing the aadA/sp/A cassettes; (3) the transgenic DNA was inserted in an intergenic region, far away from any endogenous genes or repeat regions; (4) the transgenic event produced the correct sized transcripts and proteins for the four transgenes by northern hybridization and western hybridization analyses, respectively. Furthermore, DNA sequence analyses were performed to: (1) determine the 5′ and 3′ transgenic insert-to-plant genome junctions; (2) confirm the organization of the elements within the insert; and (3) verify the complete nucleotide sequence of the inserted transgenic DNA (SEQ ID NO:9). In addition, primers and probes were designed, and thermal amplification assays were developed for producing specific amplicons diagnostic for the presence of event Gm_CSM63714 in a sample. As used herein, the 5′ and 3′ designations in reference to the junction, direction and side of the transgenic event insertion is relative to the 5′ to 3′ direction of the transgene, with the 5′ junction and genomic sequence being upstream of the transgene, and the 3′ junction and genomic sequence being downstream of the transgene.

As used herein, an “expression cassette” or “cassette” or “transgene cassette” is a recombinant DNA molecule or sequence comprising a combination of distinct elements for expressing an RNA and/or protein encoded by the coding sequence of a transgene in a transformed plant cell or transformed plant comprising the transgene. As provided herein, an “expression cassette” or “cassette” or “transgene cassette” includes one or more regulatory element(s) operably linked to a coding or transcribable DNA sequence. The regulatory elements can include a promoter, a leader, 5′ untranslated region (5′ UTR), intron and/or a 3′ untranslated region (3′ UTR) region. The “expression cassette” or “cassette” or “transgene cassette” is recombinant and heterologous with respect to the transformed plant cell genome. For purposes of the present disclosure, such an “expression cassette” or “cassette” or “transgene cassette” is a recombinant DNA molecule or sequence that encodes a protein for conferring tolerance to at least one class of herbicides as described herein. Table 1 provides a list of the genetic elements contained in the four transgene cassettes in the transgenic insert (SEQ ID NO:9) of soybean event Gm_CSM63714.

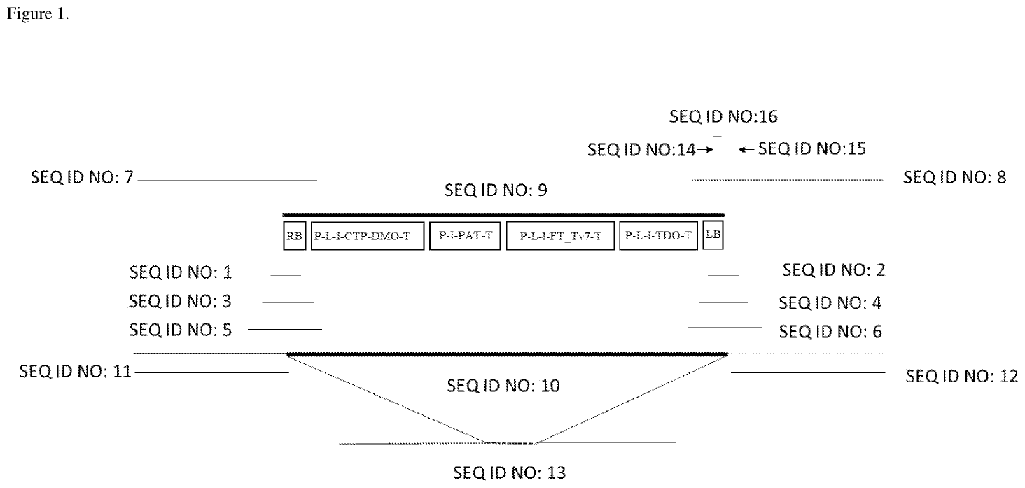

Insertion of the transgenic DNA into the genome of the soybean plant is accomplished by plant transformation methods known in the art and creates a new transgenic genomic DNA sequence, known as a “transgenic event” or an “event.” The DNA sequence of the event consists of the inserted foreign DNA (referred to as “transgenic insert”) and the genomic DNA adjacent to, or “flanking,” the transgenic insert on either side of the insertion location. As used herein, the term “flanking” in reference to a transgenic event refers to the plant genomic sequence(s) adjacent to the transgenic DNA insertion in the genome of a transformed plant, plant part, plant tissue, or plant cell comprising the transgenic event on the 5′ and/or 3′ end(s) of the transgenic event insertion. Likewise, “flanking DNA” refers to a length of genomic DNA sequence adjacent to the transgenic DNA insertion in the genome of the transformed event on the 5′ and/or 3′ end(s) of the insertion. A “5′ flank”, therefore, means the soybean genomic DNA sequence adjacent to and upstream (or on the 5′ end) of the transgenic DNA insertion. For example, a “5′ flank” can include the soybean genomic DNA sequence immediately adjacent to and upstream (on the 5′ end) of the transgenic insertion, or any soybean genomic DNA sequence upstream (on the 5′ end) of the transgenic insertion that is not immediately adjacent to the transgenic insertion but is within about 5000 nucleotides, within about 3000 nucleotides, or within about 1000 nucleotides upstream of the transgenic insertion. Likewise, a “3′ flank” means the soybean genomic DNA sequence adjacent to and downstream (or on the 3′ end) of the transgenic insert. For example, a “3′ flank” can include the soybean genomic DNA sequence immediately adjacent to and downstream (on the 3′ end) of the transgenic insertion, or any soybean genomic DNA sequence downstream (on the 3′ end) of the transgenic insertion that is not immediately adjacent to the transgenic insertion but is within about 5000 nucleotides, within about 3000 nucleotides, or within about 1000 nucleotides downstream of the transgenic insertion. The DNA sequence of an event is unique to and specific for the event and can be readily identified when compared to other DNA sequences, such as that of other events or untransformed soybean genomic DNA. Soybean event Gm_CSM63714 has the new and unique DNA sequence provided as SEQ ID NO:10, which comprises a contiguous sequence comprising the 5′ soybean genomic flanking sequence provided as SEQ ID NO:11, the transgenic insert sequence provided as SEQ ID NO:9, and the 3′ soybean genomic flanking sequence provided as SEQ ID NO:12 (FIG. 1). Soybean event Gm_CSM63714 is thus a DNA molecule that is an integral part of the chromosome of transgenic soybean cells and plants comprising the event and as such is static and may be passed on to progeny cells and plants. As is described further in the Examples hereinbelow, various gene editing tools exist that would permit modification of the transgenic insert and/or the flanking genomic DNA of soybean event Gm_CSM63714, such as by deletion, insertion, transposition, or substitution of nucleic acid sequence(s), the event is still uniquely characterized by the presence of heterologous DNA at the particular position in the genome occupied by soybean event Gm_CSM63714 relative to flanking portions of the native soybean genome.

TABLE 1
Elements and Description of Soybean Event Gm_CSM63714

| | SEQ | Position in | |
|---|---|---|---|
| | ID | SEQ ID | |
| Element | NO | NO: 10 | Description |
| 5′ Flank sequence | 11 | 1-1000 | Soybean genomic DNA sequence flanking the 5′ end of |
| | | | the transgenic insert. |
| Right border region | 22 | 1001-1071 | Right border sequence from Agrobacterium tumefaciens. |
| | | | SEQ ID NO: 22 is the full-length T-DNA right border |
| | | | sequence and is 285 nucleotides long. However, since a |
| | | | portion of this sequence was truncated at the time of the |
| | | | insertion into the soybean genomic DNA, only |
| | | | nucleotides 215-285 of SEQ ID NO: 22 are present in |
| | | | SEQ ID NO: 10. |
| Intervening sequence 1 | — | 1072-1110 | Sequence used in DNA cloning. |
| P-At.Ubq3 | 23 | 1111-1651 | Promoter sequence of a ubiquitin (UB3) protein from |
| | | | Arabidopsis thaliana. |
| L-At.Ubq3 | 24 | 1652-1740 | 5′ UTR leader sequence of a ubiquitin (UB3) protein |
| | | | from Arabidopsis thaliana. |
| I-At.Ubq3 | 25 | 1741-2118 | Intron sequence of a ubiquitin (UB3) protein from |
| | | | Arabidopsis thaliana. |
| TS-At.APG6 | 26 | 2119-2322 | APG6 (Albino and Pale Green 6) chloroplast transit |
| | | | peptide sequence from Arabidopsis thaliana. |
| CR-STEma.DMO | 27 | 2323-3345 | Codon-optimized sequence encoding a variant of |
| | | | dicamba monooxygenase (DMO) from |
| | | | Stenotrophomonas maltophilia. |
| Intervening sequence 2 | — | 3346-3364 | Sequence used in DNA cloning. |
| T-Mt.Sali3-2 | 28 | 3365-3864 | 3′ UTR sequence of aluminum-induced Sali3-2 from |
| | | | Medicago truncatula. |
| Intervening sequence 3 | — | 3865-3942 | Sequence used in DNA cloning. |
| P-At.GSP579 | 29 | 3943-4442 | Promoter sequence derived from multiple Arabidopsis |
| | | | thaliana promoter sequences. |
| I-At.GSI102 | 30 | 4443-4752 | Intron sequence derived from multiple Arabidopsis |
| | | | thaliana intron sequences. |
| Intervening sequence 4 | — | 4753-4758 | Sequence used in DNA cloning. |
| CR-STRvi.Pat | 31 | 4759-5310 | Codon-optimized coding sequence of the |
| | | | phosphinothricin N-acetyltransferase (PAT) from |
| | | | Streptomyces viridochromogenes. |
| Intervening sequence 5 | — | 5311-5318 | Sequence used in DNA cloning. |
| T-Mt.Hsp20 | 32 | 5319-5818 | 3′ UTR sequence of a small heat shock protein (Hsp20) |
| | | | from Medicago truncatula. |
| Intervening sequence 6 | — | 5819-5901 | Sequence used in DNA cloning. |
| P-At.Ubq10 | 33 | 5902-6730 | Promoter sequence of a polyubiquitin gene (UBQ10) |
| | | | from Arabidopsis thaliana. |
| L-At.Ubq10 | 34 | 6731-6797 | 5′ UTR leader sequence of a polyubiquitin gene |
| | | | (UBQ10) from Arabidopsis thaliana. |
| I-At.Ubq10 | 35 | 6798-7103 | Intron sequence of a polyubiquitin gene (UBQ10) from |
| | | | Arabidopsis thaliana. |
| Intervening sequence 7 | — | 7104-7109 | Sequence used in DNA cloning. |
| CR-SPHhe.FT_Tv7 | 36 | 7110-7997 | Codon-optimized sequence encoding an alpha- |
| | | | ketoglutarate-dependent non-heme iron dioxygenase |
| | | | variant from Sphingobium herbicidovorans. |
| Intervening sequence 8 | — | 7998-8005 | Sequence used in DNA cloning. |
| T-Mt.AC139600v16 | 37 | 8006-8505 | 3′ UTR sequence of a putative protein from Medicago |
| | | | truncatula. |
| Intervening sequence 9 | — | 8506-8643 | Sequence used in DNA cloning. |
| P-At.GSP576 | 38 | 8644-9101 | Promoter sequence derived from multiple Arabidopsis |
| | | | thaliana promoter sequences. |
| L-At.GSP576 | 39 | 9102-9143 | 5′ UTR leader sequence derived from multiple |
| | | | Arabidopsis thaliana 5′ UTR sequences. |
| I-At.GSI17 | 40 | 9144-9443 | Intron sequence derived from multiple Arabidopsis |
| | | | thaliana intron sequences. |
| Intervening sequence 10 | — | 9444-9478 | Sequence used in DNA cloning. |
| CR-Os.TDO | 41 | 9479-10534 | Codon-optimized sequence encoding triketone |
| | | | dioxygenase from Oryza sativa. |
| Intervening sequence 11 | — | 10535-10564 | Sequence used in DNA cloning. |
| T-Zm.GST7 | 42 | 10565-10864 | 3′ UTR sequence derived from multiple Zea mays 3′ |
| | | | UTR sequences. |
| Intervening sequence 12 | — | 10865-10964 | Sequence used in DNA cloning. |
| Left border region | 43 | 10965-11196 | Left border sequence from Agrobacterium tumefaciens. |
| | | | SEQ ID NO: 43 is the full-length T-DNA left border |
| | | | sequence and is 442 nucleotides long. However, since a |
| | | | portion of this sequence was truncated at the time of the |
| | | | insertion into the soybean genomic DNA, only |
| | | | nucleotides 1-232 of SEQ ID NO: 43 are present in SEQ |
| | | | ID NO: 10. |
| 3′ Flank sequence | 12 | 11197-12196 | Soybean genomic DNA sequence flanking the 3′ end of |
| | | | the transgenic insert. |

Progeny of the original transformed cell and plant that comprise soybean event Gm_CSM63714 are provided. Such progeny may be produced by selfing of a soybean plant comprising the soybean event Gm_CSM63714, or by sexual cross or outcrossing between a soybean plant comprising soybean event Gm_CSM63714 and another plant that does or does not contain the event, or by any other method known in the art including any plant cell or tissue culture method, wherein the progeny includes the soybean event Gm_CSM63714. The other plant may be a transgenic plant comprising the same and/or different event(s) or may be a non-transgenic plant, and each parental plant in a cross or outcross may be the same or different germplasm or breeding line. Soybean event Gm_CSM63714 is passed from the original parent through each generation to the progeny. A “transgenic plant” or “plant”, therefore, can be the original transformant plant regenerated from the transformed plant cell and comprising the transgenic DNA and event, or a progeny plant of the original transformant plant, which may be separated from the transformant by one or more generations, that retains the transgenic DNA and event at the same specific location and sequence context in the plant's genome. The transformant or progeny plant may be homozygous or heterozygous for event Gm_CSM63714. In addition, a “transgenic plant” may comprise a plant having the transgenes stably inserted into the genome of at least one cell of the plant (i.e., soybean event Gm_CSM63714 in at least one cell of the plant), and the plant may be chimeric or non-chimeric with respect to the transgenes and/or event. A transgenic plant is chimeric with respect to a transgene if not all cells of the plant comprise the transgenes.

The present disclosure describes introduction of event Gm_CSM63714 into soybean, and thus the term “soybean event Gm_CSM63714” is used to refer to the event herein. However, those of skill in the art will understand that event Gm_CSM63714 could be introduced into other varieties or related soybean species by crosses, such as Glycine soja and Glycine tomentella.

Soybean event Gm_CSM63714 provides to soybean cells, plants, plant parts, seeds and progeny that comprise the event tolerance to benzoic acid auxin herbicides such as dicamba which functions by increasing plant growth rate, leading to senescence and cell death; inhibitors of glutamine synthetase such as glufosinate; phenoxy auxins such as 2,4-dichlorophenoxyacetic acid (2,4-D), which mimics the action of the plant growth regulator auxin and causes uncontrolled growth and eventually death in susceptible plants; and β-triketone herbicides such as mesotrione.

Soybean event Gm_CSM63714 is characterized as a single copy insertion into one locus in the soybean genome, resulting in two new loci or junction sequences (e.g. sequences set forth in SEQ ID NO:1, SEQ ID NO:2, SEQ ID NO:3, SEQ ID NO:4, SEQ ID NO:5, SEQ ID NO:6, SEQ ID NO:7 and SEQ ID NO:8) spanning portions of the inserted DNA and the soybean genomic DNA that are not known to appear or exist naturally in the soybean genome or other transgenic soybean events, i.e., they are unique to event Gm_CSM63714. SEQ ID NO:1, SEQ ID NO:3, SEQ ID NO:5 and SEQ ID NO:7 span the 5′ junction of the soybean genomic sequence and the transgenic DNA insert, and SEQ ID NO:2, SEQ ID NO:4, SEQ ID NO:6 and SEQ ID NO:8 span the 3′ junction. These junction sequences are useful in detecting the presence of the event Gm_CSM63714 in soybean cells, seed, plants, plant parts, progeny, and plant products, such as soybean commodity products. Polynucleotide or DNA molecular probes and/or primer pairs are described herein for use in identifying the presence of these various junction sequences in biological samples containing or derived from, or suspected of containing or being derived from, soybean cells, seeds, plants, plant parts, progeny, or commodity product that contains the event Gm_CSM63714.

As used herein, the term “derived” or “derived from” in reference to a particular DNA molecule, amplicon or sequence in relation to a soybean plant, plant part, seed, progeny, cell and/or soybean plant product, such as a commodity product, means that the DNA molecule, amplicon or sequence is taken, purified, isolated, or made, directly or indirectly, from such soybean plant, plant part, seed, progeny, cell and/or soybean plant product, such as a commodity product. Alternatively, the term “derived” or “derived from” in reference to a soybean plant product, such as a commodity product, in relation to soybean plant, plant part, seed, progeny, or cell, means that the soybean plant product is taken, purified, isolated, or made, directly or indirectly, from such soybean plant, plant part, seed, progeny, or cell.

“Capable of being detected” refers to the ability of a particular DNA molecule, segment or sequence to be detected in a sample, such as by amplification and determining its presence, size or sequence such as by DNA sequence analysis, and/or binding of a probe to the target DNA molecule, segment or sequence.

A “sample” is intended to refer to any composition comprising or derived from, either directly or indirectly, a biological sample, source, or material. The sample may generally comprise soybean DNA and/or substantially or completely pure, purified, or isolated soybean DNA. A “biological sample” contains biological materials, including but not limited to DNA obtained or derived from, either directly or indirectly, the genome of a soybean cell(s), tissue(s), seed(s), plant(s), plant part(s) and/or soybean plant product(s), such as a commodity product(s). Such soybean cell(s), tissue(s), seed(s), plant(s), plant part(s) and/or soybean plant product(s), such as a commodity product(s), may comprise soybean event Gm_CSM63714, or DNA molecule(s) and/or DNA segment(s) comprising soybean event Gm_CSM63714. In some embodiments, a sample or biological sample may comprise soybean cell(s), soybean tissue(s), soybean seed(s), soybean plant(s), soybean plant part(s) and/or soybean plant product(s), whose cells or cellular membranes have been fractured (e.g., disrupted or opened) to release the contents of the soybean cell(s) including genomic DNA or proteins and/or make the contents of the soybean cell(s) including genomic DNA or proteins accessible or usable for assays or testing. “Directly” refers to directly obtaining DNA by a skilled artisan from the soybean genome by fracturing soybean cells (or by obtaining samples of soybean that contain fractured soybean cells) and exposing or using the genomic DNA or protein from soybean cells for the purposes of detection. “Indirectly” refers to obtaining by a skilled artisan a target or specific reference DNA (e.g., a novel and unique junction segment(s) described herein as being diagnostic for the presence of the event Gm_CSM63714) in a particular sample, by means other than by obtaining directly via fracturing of soybean cells or obtaining a sample of soybean that contains fractured soybean cells. Such indirect means include, but are not limited to, amplification of a DNA segment that contains a DNA sequence targeted by a particular probe(s) and/or primer set(s) designed to bind with specificity to or near the target sequence, or amplification of a DNA segment comprising all or part of a target sequence that can be measured and characterized (e.g., measured by migration or separation from other segments of DNA and/or identification in an effective matrix, such as an agarose or acrylamide gel or the like, or characterized by direct sequence analysis of the amplicon(s), or cloning of the amplicon(s) into a vector(s) and direct sequencing of the inserted amplicon(s) present within such vector(s)).

As used herein, the term “recombinant” refers to a non-naturally occurring DNA, protein, combination, or organism that would not normally be found or exist in nature, and is created by human intervention. As used herein, a “recombinant DNA molecule” is a DNA molecule comprising a combination of DNA molecules that would not naturally occur together and is the result of human intervention. Two or more elements of such combination of DNA sequences may be operably linked to one another. For example, a recombinant DNA molecule may comprise a combination of at least two DNA molecules heterologous with respect to each other, such as a DNA molecule that comprises a coding sequence operably linked to a heterologous promoter and/or other regulatory expression element(s), and/or a transgene and a heterologous plant genomic DNA adjacent to the transgene, and/or a DNA molecule that is artificially synthesized and comprises a polynucleotide sequence that deviates from any polynucleotide sequence that would normally exist in nature. A recombinant DNA molecule may comprise all or part of a junction sequence of the genome of the event and all or part of the transgenic insert of the genome of the event, and/or may comprise a recombinant or heterologous DNA fragment of soybean event Gm_CSM63714. An example of a recombinant DNA molecule is a DNA molecule comprising at least one polynucleotide sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9 and SEQ ID NO:10. As used herein, a “recombinant” in reference to a plant, plant part, seed, plant cell, or progeny is a plant, plant part, seed, plant cell or progeny that would not normally exist in nature, is the result of human intervention, and contains a transgenic DNA molecule stably integrated into the genome of the plant, plant part, seed, plant cell, or progeny. As a result of such genomic insertion, the recombinant or transgenic plant, plant part, seed, plant cell, or progeny is something new and distinctly different from any related wild-type or naturally occurring plant, plant part, seed, plant cell or progeny. An example of a recombinant plant is a soybean plant containing the soybean event Gm_CSM63714.

As used herein, the term “transgene” refers to a DNA molecule artificially incorporated into an organism's genome as a result of human intervention, such as by plant transformation methods. A transgene may be heterologous to the organism. The term “transgenic insert” as used herein refers to the foreign or heterologous DNA inserted by plant transformation techniques into the soybean genome to produce soybean event Gm_CSM63714. The sequence for the transgenic insert of soybean event Gm_CSM63714 is provided as SEQ ID NO:9.

As used herein, the term “heterologous” in reference to a combination of two or more DNA sequences or elements means that the two or more DNA sequences or elements do not normally exist together as such combination in nature without human intervention. For example, a DNA molecule may be from a first species or a recombinant DNA molecule, and inserted into the genome of a second species. The DNA molecule would thus be heterologous to the genome and the organism. As used herein, the term “heterologous” in reference to a DNA molecule, construct, sequence or protein in relation to a plant, microorganism, plant cell or plant genome means that the DNA molecule, construct, sequence or protein does not exist in nature as part of such a plant, microorganism, plant cell or plant genome, and/or does not exist in the same physical or genomic location, context or orientation as part of such a plant, microorganism, plant cell or plant genome in nature, without human intervention.

As used herein, the term “chimeric” refers to a single DNA molecule produced by fusing a first DNA molecule to a second DNA molecule, where neither first nor second DNA molecule would normally be found in that configuration fused to the other. The chimeric DNA molecule is thus a new DNA molecule not normally found in nature. An example of a chimeric DNA molecule is a DNA molecule comprising at least one sequence selected from SEQ ID NO:1-10.

As used herein, the term “isolated” in reference to a molecule means that the molecule is at least partially separated from other molecules that are normally associated with it in its native or natural state. In some embodiments, the term “isolated” refers to a DNA molecule that is at least partially separated from the nucleic acids or polynucleotide or DNA sequence(s) that normally flank and are covalently linked to the sequence of the DNA molecule in its native or natural state. An “isolated” DNA molecule may have a DNA sequence corresponding to a portion of the genome of a plant cell without other genomic DNA sequence(s) that normally flank and are covalently linked to the DNA sequence in nature. Such an “isolated” DNA molecule may comprise all or part of a transgene and/or transgenic event, which may comprise all or part of soybean event Gm_CSM63714 or the transgenes or expression cassettes described herein. Nucleic acid sequences or elements, such as a coding sequence, intron sequence, 5′ UTR, promoter sequence, 3′ UTR, and the like, that are naturally found within the DNA of the genome of an organism are not considered to be “isolated” so long as the element is within the genome of the organism and at the location within the genome in which it is naturally found. However, each of these elements, and subparts of these elements, would be “isolated” within the scope of this disclosure so long as the element or subpart is not within the genome of the organism, and at the location within the genome of the organism, in which it is naturally found. An “isolated” DNA molecule may be any recombinant DNA molecule or amplification product or amplicon, and/or may comprise any DNA sequence removed from its natural or biological state and covalently fused to another DNA molecule or sequence with which it is not associated in nature. Such an isolated DNA molecule could be created by the use of biotechnology techniques, such as by making a recombinant DNA or integrating a foreign or heterologous DNA molecule into the chromosome of a cell, plant, or seed. Thus, any DNA molecule comprising a transgenic, recombinant, chimeric or artificial nucleotide sequence, transgene or expression cassette would be considered to be an “isolated” DNA molecule since these sequences are not naturally occurring, regardless of whether the sequence, transgene or expression cassette is present within a plasmid, vector or construct used to transform plant cells, within the genome of a plant, plant part, plant tissue, plant cell or progeny, or is present in detectable amounts in tissues, progeny, biological samples or commodity products derived from a plant, plant part, plant tissue, progeny or plant cell. A recombinant DNA molecule or sequence, or any fragment derived therefrom, comprising all or part of a transgene or junction sequence of the soybean event Gm_CSM63714 would therefore also be considered to be “isolated.” An “isolated” DNA molecule may be extracted or purified from a transgenic plant(s), plant part(s), plant cell(s) and/or tissue(s), or may be present in a homogenate, extract or lysate from any such transgenic plant(s), plant part(s), plant cell(s) and/or tissue(s), or may be produced as an amplicon or amplification product from plant genomic DNA and/or extracted or purified DNA from transgenic plant(s), plant part(s), plant cell(s) and/or tissue(s), or a homogenate, extract or lysate from plant(s), plant part(s), plant cell(s) and/or tissue(s). For the purposes of this disclosure, any transgenic polynucleotide or DNA sequence, i.e., the nucleotide sequence of the DNA inserted into the genome of a plant or bacterium, or present in an extrachromosomal vector, would be considered to be an “isolated” nucleotide or DNA sequence whether it is present within the plasmid or similar structure used to transform the cells, within the genome of the plant or bacterium, or present in detectable amounts in tissues, progeny, biological samples or commodity products derived from the plant or bacterium. An “isolated” DNA molecule is a chemical or biochemical molecule, regardless of whether the molecule is referred to as a nucleic acid, a nucleic acid sequence, a polynucleotide sequence, a DNA sequence, a nucleic acid molecule, a polynucleotide molecule, a DNA molecule, or the like. An “isolated” molecule can provide industrial applicability when present in a plant cell or in a plant genome or when present outside of a plant cell, and therefore, provides and exhibits (and is intended to provide and exhibit) utility regardless of where the molecule is located. As used herein, the term “correspond” or “corresponding”, or the like, when used in the context of a nucleotide position, mutation, insertion and/or substitution in any given polynucleotide (e.g., SEQ ID NO:9) with respect to a reference polynucleotide sequence (e.g., SEQ ID NO:10) refers to the position(s) of the polynucleotide residue(s) in the given sequence that has identity to the residue(s) in the reference nucleotide sequence when the given polynucleotide is aligned to the reference polynucleotide sequence using a global or local sequence alignment algorithm.

DNA molecules, fragments, and their corresponding DNA sequences, as well as methods of detection are provided. As used herein, the terms “DNA”, “DNA molecule” and “nucleic acid molecule” refer to a deoxyribonucleic acid (DNA) molecule. A DNA molecule may be of genomic or synthetic origin and/or comprise a recombinant or heterologous DNA molecule or sequence. A DNA molecule may be described by convention from the 5′ (upstream) end to the 3′ (downstream) end. As used herein, the term “DNA sequence” refers to the polynucleotide sequence of a DNA molecule, i.e. the sequence of consecutive nucleotides in the DNA molecule. As used herein in reference to nucleotides of a polynucleotide or DNA sequence or molecule, the terms “consecutive” and “contiguous” are interchangeable and synonymous and refer to the 5′ to 3′ order of nucleotides in a polynucleotide or DNA sequence, strand or molecule without any gap or interruption between them. The nomenclature used is that required by Title 37 of the United States Code of Federal Regulations § 1.822 and set forth in the tables in WIPO Standard ST.25 (1998), Appendix 2, Tables 1 and 3. By convention, DNA sequences and fragments thereof are disclosed with reference to the 5′ to 3′ direction of only one strand of the two complementary DNA sequence strands of a DNA molecule. By implication and intent, the complementary sequences of the sequences provided here (the sequences of the complementary strand), also referred to in the art as the reverse complementary or reverse complement sequences, are within the scope of the present disclosure and are expressly intended to be within the scope of the subject matter claimed. As used herein references to SEQ ID NOs:1-10 and fragments thereof include and refer to the sequence of the complementary strand and fragments thereof.

Also provided is a nucleic acid molecule comprising a polynucleotide having a sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% identical to the full length of any one of SEQ ID NOs:1-12.

For example, a nucleic acid molecule is provided comprising a polynucleotide having a sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% identical to the full length of SEQ ID NO:10 or to the full length of SEQ ID NO: 9.

A DNA molecule, or a fragment derived therefrom, can also be extracted from plant(s), plant part(s), seed(s), progeny or plant cell(s), or a homogenate, extract or lysate from plant(s), plant part(s), plant cell(s) or seed(s) or progeny, or can be produced as an amplicon from extracted, purified or isolated DNA from plant part(s), plant cell(s) and/or tissue(s), progeny, or a homogenate, extract or lysate from plant(s), plant part(s), plant cell(s), progeny and/or seeds, which may further comprise soybean event Gm_CSM63714.

As used herein, the term “percent sequence identity” or “% sequence identity” refers to the percentage of identical nucleotides or amino acids in a linear polynucleotide or polypeptide sequence of a reference (“query”) sequence (or its complementary strand) as compared to a test (“subject”) sequence (or its complementary strand) when the two sequences are optimally aligned (with appropriate nucleotide or amino acid insertions, deletions, or gaps totaling less than 20 percent of the reference sequence over the window of comparison). Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and by computerized implementations of these algorithms such as GAP, BESTFIT, FASTA, and TFASTA available as part of the Sequence Analysis software package of the GCG® Wisconsin Package® (Accelrys Inc., San Diego, Calif.), MEGAlign (DNAStar Inc., 1228 S. Park St., Madison, Wis. 53715), and MUSCLE (version 3.6) (Edgar, “MUSCLE: multiple sequence alignment with high accuracy and high throughput” Nucleic Acids Research 32(5):1792-7 (2004)) for instance with default parameters. An “identity fraction” for aligned segments of a test sequence and a reference sequence is the number of identical components that are shared by the two aligned sequences divided by the total number of components in the portion of the reference sequence segment being aligned, that is, the entire reference sequence or a smaller defined part of the reference sequence. Percent sequence identity is represented as the identity fraction multiplied by 100. The comparison of one or more sequences may be to a full-length sequence or a portion thereof, or to a longer sequence. Soybean plants, progeny, seeds, cells, plant parts and commodity products comprising a detectable amount of a polynucleotide having a nucleotide sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO:10 or the full length of SEQ ID NO:9 are within the scope of the present disclosure.

As used herein, the term “fragment” refers to a smaller piece or sequence of a larger or whole DNA molecule or sequence. For example, a fragment of any one of SEQ ID NOs:1-12 and SEQ ID NOs:98-99 may include a sequence that is at least about 10 consecutive nucleotides, at least about 11 consecutive nucleotides, at least about 12 consecutive nucleotides, at least about 13 consecutive nucleotides, at least about 14 consecutive nucleotides, at least about 15 consecutive nucleotides, at least about 16 consecutive nucleotides, at least about 17 consecutive nucleotides, at least about 18 consecutive nucleotides, at least about 19 consecutive nucleotides, at least about 20 consecutive nucleotides, at least about 21 consecutive nucleotides, at least about 22 consecutive nucleotides, at least about 23 consecutive nucleotides, at least about 24 consecutive nucleotides, at least about 25 consecutive nucleotides, at least about 30 consecutive nucleotides, at least about 35 consecutive nucleotides, at least about 40 consecutive nucleotides, at least about 45 consecutive nucleotides, at least about 50 consecutive nucleotides, at least about 60 consecutive nucleotides, at least about 70 consecutive nucleotides, at least about 80 consecutive nucleotides, at least about 90 consecutive nucleotides, at least about 100 consecutive nucleotides, at least about 150 consecutive nucleotides, at least about 200 consecutive nucleotides, at least about 250 consecutive nucleotides, at least about 300 consecutive nucleotides, at least about 400 consecutive nucleotides, or at least about 500 consecutive nucleotides of the larger, whole or complete DNA molecule or sequence.

For example, a “fragment” of the transgenic insert sequence (SEQ ID NO: 9) of soybean event Gm_CSM63714 can comprise at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, at least about 50, at least about 60, at least about 70, at least about 80, at least about 90, at least about 100, at least about 150, at least about 200, at least about 250, at least about 300, at least about 400, or at least about 500 consecutive nucleotides of SEQ ID NO: 9. In addition, the present disclosure encompasses nucleotide sequences that are at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% identical to SEQ ID NO: 9 or any fragment thereof.

Similarly, a fragment of the 5′ flank (SEQ ID NO:11 or SEQ ID NO:98) or 3′ flank (SEQ ID NO:12 or SEQ ID NO:99) of soybean event Gm_CSM63714 can comprise at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, at least about 50, at least about 60, at least about 70, at least about 80, at least about 90, at least about 100, at least about 150, at least about 200, at least about 250, at least about 300, at least about 400, or at least about 500 consecutive nucleotides of SEQ ID NO:11 or SEQ ID NO:98; or SEQ ID NO:12 or SEQ ID NO:99. In addition, the present disclosure encompasses nucleotide sequences that are at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% identical to SEQ ID NO:11 or 12, or SEQ ID NO:98 or 99, or any fragment of either thereof.

As used herein, the term “about” indicates a value or a range of values which would be understood as an equivalent of a stated value and can be greater or lesser than the value or range of values stated. Each value or range of values preceded by the term “about” is also intended to encompass the embodiment of the stated absolute value or range of values.

The term “or” is used herein to mean “and/or” unless explicitly indicated to refer to alternatives only or the alternatives are mutually exclusive. Thus the term “and/or” as used herein in a phrase such as “X and/or Y” is intended to include “X and Y”, “X or Y”, “X” (alone), and “Y” (alone). Likewise, the term “and/or” as used in a phrase such as “X, Y, and/or Z” is intended to encompass each of the following embodiments: X (alone); Y (alone); Z (alone); X and Y; X and Z; Y and Z; X, Y, and Z; X, Y, or Z; X or Z; Y or Z; Y or Z.

When used in conjunction with the word “comprising” or other open language, the words “a” and “an” denote “one or more,” unless specifically noted otherwise. The terms “comprise,” “have,” and “include” are open-ended linking verbs. Any forms or tenses of one or more of these verbs, such as “comprises,” “comprising,” “has,” “having,” “includes,” and “including,” are also open-ended. For example, any method that “comprises,” “has,” or “includes” one or more steps is not limited to possessing only those one or more steps and also covers other unlisted steps.

Soybean event Gm_CSM63714 is characterized as a transgenic insertion into a single locus in the soybean genome, resulting in two new junctions (or joining or connection points). The DNA sequence of the region spanning the connection by phosphodiester bond linkage of one end of the transgenic insert to the flanking soybean genomic DNA is referred to herein as a “junction.” In other words, a junction is the connection point or covalent linkage of one end of the transgenic insert and the flanking genomic DNA as one contiguous molecule, and is formed by the insertion of a heterologous nucleic acid molecule into the soybean genomic DNA. One junction is found at the 5′ end of the transgenic insert and the other is found at the 3′ end of the transgenic insert, referred to herein as the 5′ and 3′ junctions, respectively. A “junction sequence” refers to a DNA sequence of any length of consecutive nucleotides that spans the 5′ or 3′ junction of a transgenic event in the plant genome. For a “junction sequence” to be specific to a junction between a transgenic event and a flanking genomic sequence, the junction sequence will generally comprise a sufficient number of consecutive nucleotides at one end of the insertion and a sufficient number of consecutive nucleotides of the flanking genomic sequence. According to some embodiments, a “junction sequence” may comprise (i) at least five (5) consecutive nucleotides, at least ten (10) consecutive nucleotides, at least fifteen (15) consecutive nucleotides, at least twenty (20) consecutive nucleotides, at least twenty five (25) consecutive nucleotides, at least thirty (30) consecutive nucleotides, at least thirty five (35) consecutive nucleotides, at least forty (40) consecutive nucleotides, at least forth five (45) consecutive nucleotides, or at least fifty (50) consecutive nucleotides at one end of the insertion and (ii) at least five (5) consecutive nucleotides, at least ten (10) consecutive nucleotides, at least fifteen (15) consecutive nucleotides, at least twenty (20) consecutive nucleotides, at least twenty five (25) consecutive nucleotides, at least thirty (30) consecutive nucleotides, at least thirty five (35) consecutive nucleotides, at least forty (40) consecutive nucleotides, at least forth five (45) consecutive nucleotides, or at least fifty (50) consecutive nucleotides of the flanking genomic DNA sequence, although it is understood that any length of consecutive nucleotides spanning a junction of a transgenic event in a plant genome may be a junction sequence. Junction sequences of soybean event Gm_CSM63714 are apparent to, and a variety of junction sequences of soybean event Gm_CSM63714 can be determined by one of skill in the art using SEQ ID NO:10. In SEQ ID NO:10, the 5′ junction is at nucleotides 1,000-1,001, and the 3′ junction is at nucleotides 11,196-11,197. Illustrative junction sequences of soybean event Gm_CSM63714 are provided as SEQ ID NOs:1-8. FIG. 1 illustrates the physical arrangement and locations of the illustrative junction sequences, arranged from 5′ to 3′ (left to right), relative to SEQ ID NO:10. The DNA sequence for the transgenic insert of soybean event Gm_CSM63714 is provided as SEQ ID NO:9. The DNA sequence of the transgenic insert and the soybean genomic DNA flanking each side of the transgenic insert is provided as SEQ ID NO:10. The 5′ junction sequences are provided as SEQ ID NO:1, SEQ ID NO:3, SEQ ID NO:5, and SEQ ID NO:7. The 3′ junction sequences are provided as SEQ ID NO:2, SEQ ID NO:4, SEQ ID NO:6, and SEQ ID NO:8. The junction sequences of soybean event Gm_CSM63714 may be present as part of the genome of a plant, seed, plant part, progeny or plant cell containing soybean event Gm_CSM63714, a DNA molecule containing all or part of event Gm_CSM63714. The identification of any one or more of the junction sequences in a DNA molecule or a sample from a plant, plant part, seed, progeny, cell or commodity product indicates that the DNA molecule or plant, plant part, seed, progeny, cell or commodity product contains or comprises event Gm_CSM63714, or was obtained from a soybean plant, plant part, seed, progeny, cell or commodity product containing or comprising event Gm_CSM63714, and is diagnostic for the presence of soybean event Gm_CSM63714.

The junction sequences described herein are diagnostic for the presence of all or part of soybean event Gm_CSM63714. Thus, the identification or detection, directly or indirectly, of one or more of SEQ ID NO:1, SEQ ID NO:2, SEQ ID NO:3, SEQ ID NO:4, SEQ ID NO:5, SEQ ID NO:6, SEQ ID NO:7, SEQ ID NO:8, and SEQ ID NO:10 in a sample or DNA molecule derived from a soybean plant, plant part, seed, progeny, cell, or a commodity product is diagnostic that the soybean plant, plant part, seed, progeny, cell, or a commodity product has or comprises all or part of soybean event Gm_CSM63714. The identification or detection, directly or indirectly, of a 5′ junction sequence and/or a 3′ junction sequence (each as provided or described herein) in a sample or DNA molecule derived from a soybean plant, plant part, seed, progeny, cell, or a commodity product is diagnostic that the soybean plant, plant part, seed, progeny, cell, or a commodity product has or comprises soybean event Gm_CSM63714. The present disclosure thus provides a DNA molecule that comprises at least one of the nucleotide sequences provided as SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, and SEQ ID NO:10. Any segment of DNA derived from transgenic soybean event Gm_CSM63714 that is sufficient to include at least one of the sequences provided as SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, and SEQ ID NO:10 is within the scope of the present disclosure. In addition, any DNA or polynucleotide molecule or sequence comprising a sequence complementary to any of the sequences described herein is also within the scope of the present disclosure.

Polynucleotide molecules are provided, which may be single or double stranded, that can be used either as primers or probes for detecting the presence of DNA comprising all or part of event Gm_CSM63714 in a sample derived from a soybean plant, plant part, seed, progeny, cell, or a commodity product. Such primers or probes are specific for a target polynucleotide sequence and, as such, are useful for the identification of soybean event Gm_CSM63714 polynucleotide by the methods described herein. A primer or probe can hybridize to a target polynucleotide sequence to allow for specific detection or amplification of a polynucleotide molecule that comprises, or is covalently linked and associated with, the target polynucleotide sequence. According to present embodiments, the primers and/or probes may be chosen to identify and distinguish detection of a particular transgenic event and not only the presence of a transgene in a plant genome. The target polynucleotide sequence may comprise all or part of soybean event Gm_CSM63714, a junction sequence and/or flanking genomic DNA. Probes and primers according to the present disclosure may have (i) complete or 100% sequence complementarity (i.e., 100% complementary) to a target polynucleotide sequence or (ii) incomplete sequence complementarity to a target polynucleotide, such as at least 60% complementary, at least 65% complementary, at least 70% complementary, at least 75% complementary, at least 80% complementary, at least 85% complementary, at least 90% complementary, at least 95% complementary, or at least 99% complementary to the target polynucleotide sequence as long as the probe or primer has sufficient complementarity to the target polynucleotide sequence to hybridize to the target polynucleotide sequence under stringent hybridization conditions that are suitable and necessary for use of the probe or primer in the relevant amplification or detection assay, reaction or method. As understood in the art, the percentage complementarity of a primer or probe may be lower if the length of the primer or probe is longer, and depends on the stringency and use. Provided are illustrative polynucleotide molecules that can be used either as primers or probes for detecting the presence of soybean event Gm_CSM63714 in a sample. Detection of the presence of soybean event Gm_CSM63714 may be done by using methods known in the art, such as thermal or isothermal amplification of nucleic acids or nucleic acid hybridization techniques (such as Northern analysis and Southern analysis).

A “probe” is a nucleic acid molecule that is complementary to a strand of a target nucleic acid and is useful in hybridization detection methods. Probes include not only deoxyribonucleic or ribonucleic acids but also polyamides and other probe materials that bind specifically to a target DNA sequence and the detection of such binding can be useful in detecting the presence or absence of the target DNA sequence. A probe may be attached to a conventional detectable label or reporter molecule, such as a radioactive isotope, ligand, chemiluminescent agent, or enzyme. Such a probe is complementary to a strand of a target nucleic acid and, in the case of the present disclosure, to a strand of DNA from event Gm_CSM63714 whether from an event Gm_CSM63714 containing plant or from a sample that includes event Gm_CSM63714 DNA. An illustrative DNA sequence useful as a probe for detecting soybean event Gm_CSM63714 is provided as SEQ ID NO:16.

A “primer” is a DNA molecule or oligonucleotide that is designed for use in specific annealing or hybridization methods that involve an in vitro amplification reaction. A pair of primers may be used with template DNA (such as a sample of soybean event Gm_CSM63714 genomic DNA) in a thermal amplification reaction (such as polymerase chain reaction (PCR)) or any other suitable amplification method known in the art to produce an amplification product or amplicon, where the amplicon produced from such reaction would have a DNA sequence corresponding to sequence of the template DNA located between the two sites where the primers hybridized to the template DNA.

DNA amplification reactions, methods and techniques are known to those skilled in art. DNA amplification can be accomplished by any of the various nucleic acid amplification methods known in the art, including thermal and isothermal amplification methods including the polymerase chain reaction or PCR. Amplification methods are known in the art and are described, inter alia, in U.S. Pat. Nos. 4,683,195 and 4,683,202 and in PCR Protocols: A Guide to Methods and Applications, ed. Innis et al., Academic Press, San Diego, 1990. PCR amplification methods have been developed to amplify up to 22 kb (kilobase) of genomic DNA and up to 42 kb of bacteriophage DNA (Cheng et al., 1994). These methods as well as other methods known in the art of DNA amplification may be used in the practice of the present disclosure. Examples of DNA amplification methods include PCR, Recombinase Polymerase Amplification (RPA) (see for example U.S. Pat. No. 7,485,428), Strand Displacement Amplification (SDA) (see for example, U.S. Pat. Nos. 5,455,166 and 5,470,723), Transcription-Mediated Amplification (TMA) (see for example, Guatelli et al., 1990), Rolling Circle Amplification (RCA) (see for example, Fire and Xu, 1995; Lui, et al., 1996; Lizardi, et al., 1998; U.S. Pat. Nos. 5,714,320 and 6,235,502), Helicase Dependent Amplification (HDA) (see for example Vincent et al., 2004; U.S. Pat. No. 7,282,328), Multiple Displacement Amplification (MDA) (see for example Dean et al., 2002) and Loop-Mediated Isothermal Amplification (LAMP) (see for example Notomi et al., 2000). A sequence of the heterologous DNA insert and/or flanking genomic DNA sequence from soybean event Gm_CSM63714 can be verified or tested by amplifying such DNA molecules from soybean seed containing event Gm_CSM63714 DNA or soybean plants grown from the soybean seed containing event Gm_CSM63714 DNA, using primers derived from the sequences provided herein, followed by standard DNA sequencing of the PCR amplicon or a cloned DNA fragment thereof.

As used herein, an “amplification product” or “amplified DNA” or “amplicon” refers to the nucleic acid or DNA molecule or segment produced by a nucleic acid amplification reaction or method as further described herein, which is directed to a target nucleic acid or DNA molecule that is part of a template nucleic acid molecule. Amplification or amplifying refers to making multiple copies of a target DNA molecule or segment from a template DNA. For example, to determine whether a soybean plant, plant part, seed, progeny or plant cell, resulting from selfing or outcross of a parent comprising soybean event Gm_CSM63714 contains soybean event Gm_CSM63714, DNA may be extracted from the soybean plant tissue sample and subjected to an amplification reaction or method using a pair of primers that are specific for a target sequence that is uniquely associated or part of soybean event Gm_CSM63714, such as, for example, a first primer derived from a genomic DNA sequence in the region flanking the heterologous inserted DNA of soybean event Gm_CSM63714 that is elongated by polymerase 5′ to 3′ in the direction of the inserted DNA, and a second primer derived from the heterologous inserted DNA molecule that is elongated by the polymerase 5′ to 3′ in the direction of the flanking genomic DNA from which the first primer is derived. The amplicon may range in length depending on the length of the intervening polynucleotide or DNA sequence between the two primer target sequences in the template DNA molecule. Alternatively, a primer pair can be derived from the genomic sequence on both sides of the inserted heterologous DNA so as to produce an amplicon that includes the entire insert polynucleotide sequence (e.g., a forward primer targeted to the genomic portion on the 5′ end of SEQ ID NO:10 (i.e. upstream of SEQ ID NO:9) and a reverse primer targeted to the genomic portion on the 3′ end of SEQ ID NO:10 (i.e. downstream of SEQ ID NO:9) that amplifies a DNA molecule comprising the inserted DNA sequence (SEQ ID NO:9) identified herein in the soybean event Gm_CSM63714 genome. The use of the term “amplicon” specifically excludes primer dimers that may be formed in a DNA amplification reaction.

The amplicon described herein may comprise a DNA sequence comprising one or more of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, or a fragment of any of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10 wherein the fragment is at least 10 nucleotides in length and comprises nucleotides 1,000-1,001 or 11,196-11,197 of SEQ ID NO:10. According to present embodiments, the sequence of an amplicon comprises at least one junction sequence or two junction sequences, such as a 5′ junction sequence and/or a 3′ junction sequences for soybean event Gm_CSM63714. Amplification and detection of such an amplicon is indicative or diagnostic for soybean event Gm_CSM63714.

For practical purposes, one should design primers which produce amplicons of a limited size range, for example, between 100 to 1000 bases. Smaller (shorter polynucleotide length) sized amplicons in general are more reliably produced in thermal amplification reactions, allow for shorter cycle times, and can be easily separated and visualized on agarose gels or adapted for use in endpoint TaqMan®-like assays. Smaller amplicons can be produced and detected by methods known in the art of DNA amplicon detection. In addition, amplicons produced using the primer pairs can be cloned into vectors, propagated, isolated, and sequenced or can be sequenced directly with methods well established in the art. Any primer pair of forward and reverse primers, which may be identical or complementary to part of SEQ ID NO:10, such as SEQ ID NOs:14 and 15, that is useful in a DNA amplification method to produce an amplicon diagnostic for soybean event Gm_CSM63714 or progeny thereof is an aspect of the disclosure. Any single isolated DNA polynucleotide primer molecule comprising at least 15 contiguous nucleotides of SEQ ID NO:10, or its complement that is useful in a DNA amplification method to produce an amplicon diagnostic for soybean event Gm_CSM63714 or progeny thereof is an aspect of the disclosure. Any single isolated DNA polynucleotide primer molecule comprising at least 15 contiguous nucleotides of SEQ ID NO:11 or SEQ ID NO:12, or its complement that is useful in a DNA amplification method to produce an amplicon diagnostic for plants comprising soybean event Gm_CSM63714 or progeny thereof is an aspect of the disclosure. Any single isolated DNA polynucleotide primer molecule comprising at least 15 contiguous nucleotides of SEQ ID NO:9, or its complement that is useful in a DNA amplification method to produce an amplicon diagnostic for soybean event Gm_CSM63714 or progeny thereof is an aspect of the disclosure.

A primer is typically designed to hybridize specifically to a complementary target DNA strand to form a hybrid between the primer and the target DNA strand. Hybridization or binding of a primer to the complementary target DNA strand is a point of recognition by a polymerase to begin extension of the primer (i.e., polymerization of additional nucleotides into a lengthening nucleotide molecule) using the target DNA strand as a template. Primer pairs refer to use of two primers binding opposite strands of a double stranded nucleotide segment for the purpose of amplifying the polynucleotide segment between the positions targeted for binding by the individual members of the primer pair, typically in a thermal amplification reaction or other conventional nucleic-acid amplification methods. Primer pairs are typically designed to hybridize to different nearby target positions of a template DNA molecule on opposing strands of the template DNA molecule such that the intervening region or sequence between the two primers can be specifically amplified for use or detection through multiple rounds of amplification.

To detect the presence or absence of soybean event Gm_CSM63714, the target positions and/or the intervening region or sequence of a template DNA molecule may comprise at least one junction sequence and/or at least a portion of the insert of soybean event Gm_CSM63714. To detect the absence of soybean event Gm_CSM63714, the target positions and/or the intervening region or sequence of a template DNA molecule may comprise soybean genomic DNA that does not include a junction sequence or any portion of the insert of soybean event Gm_CSM63714. Thus, the presence or absence of an amplicon with a primer pair may be diagnostic of the presence or absence, respectively, of soybean event Gm_CSM63714 in a DNA molecule or sample, or vice versa. This may also be possible with more than one primer pair. For example, a first primer pair may produce a first amplicon if soybean event Gm_CSM63714 is present, and a second primer pair may produce a second amplicon if soybean event Gm_CSM63714 is absent or not present. Alternatively, the size of an amplicon produced in an amplification reaction may also be diagnostic of the presence or absence of soybean event Gm_CSM63714 in a DNA molecule or sample—e.g., a primer pair may produce a first amplicon of a first size if soybean event Gm_CSM63714 is present or a second amplicon of a second size if soybean event Gm_CSM63714 is absent and not present; or a first primer pair may produce a first amplicon of a first size if soybean event Gm_CSM63714 is present, and a second primer pair may produce a second amplicon of a second size if soybean event Gm_CSM63714 is absent or not present. According to some of these embodiments, at least two primer pairs may be used wherein at least one of the primer pairs is used as an internal control and is not associated with soybean event Gm_CSM63714.

According to present embodiments, a primer pair to detect the presence or absence of all or part of soybean event Gm_CSM63714 in a DNA molecule or sample comprises a first primer and a second primer, wherein the first primer is complementary to a 5′ flanking genomic DNA sequence and the second primer is complementary to a sequence within the transgenic insert; or wherein the first primer is complementary to a 5′ flanking genomic DNA sequence and the second primer is complementary to a 3′ flanking genomic DNA sequence; or wherein the first primer is complementary to a sequence within the transgenic insert and the second primer is complementary to a 3′ flanking genomic DNA sequence. Each reference in this paragraph to a primer complementary to a 5′ flanking genomic DNA sequence, a 3′ flanking genomic DNA sequence, or a sequence within the transgenic insert of soybean event Gm_CSM63714 is also intended to potentially include a primer complementary to the reverse complement or opposing strand of the respective 5′ flanking genomic DNA sequence, 3′ flanking genomic DNA sequence, or sequence within the transgenic insert of soybean event Gm_CSM63714.

Illustrative DNA molecules useful as primers are provided as SEQ ID NO:14 and SEQ ID NO:15 and SEQ ID NO:20. The primer pair SEQ ID NO:14 and SEQ ID NO:15 can be useful as a first primer (corresponding to a sequence within the transgenic insert) and a second primer (complementary to a 3′ flanking genomic DNA sequence), wherein each primer has sufficient length of consecutive nucleotides of SEQ ID NO:10 or a sequence complementary to SEQ ID NO:10 to function as DNA primers that, when used together in an amplification reaction with template DNA derived from soybean event Gm_CSM63714, hybridize to opposite strands of the template DNA and produce an amplicon diagnostic for soybean event Gm_CSM63714 DNA in a sample. The primer pair SEQ ID NO:20 (corresponding to a 5′ flanking genomic DNA sequence) and SEQ ID NO:15 (complementary to a 3′ flanking genomic DNA sequence) are useful as a first primer and a second primer, wherein each primer has sufficient length of consecutive nucleotides of a locus within the soybean genome to function as DNA primers that, when used together in a thermal amplification reaction with template DNA, to produce an amplicon indicative or diagnostic for the wild-type DNA for the zygosity of Gm_CSM63714 event DNA in a sample. An amplicon diagnostic for event Gm_CSM63714 comprises a sequence not naturally found in the soybean genome.

A primer may further comprise an oligo tail sequence such as those used in the Kompetitive Allele-Specific PCR (KASP™) method. The allele-specific primers each harbor a unique tail sequence that corresponds with a universal FRET (fluorescence resonant energy transfer) cassette; one labelled with FAM™ dye and the other with HEX™ dye. During thermal cycling, the relevant allele-specific primer binds to the template and elongates, thus attaching the tail sequence to the newly synthesized strand. The complement of the allele-specific tail sequence is then generated during subsequent rounds of PCR, enabling the FRET cassette to bind to the DNA. The FRET cassette is no longer quenched and emits fluorescence.

Methods for designing and using primers and probes are well known in the art. DNA molecules comprising fragments of SEQ ID NOs:1-10 are useful as primers and probes for detecting soybean event Gm_CSM63714 and can readily be designed by one of skill in the art using the sequences provided herein. Such probes and primers are selected to be of sufficient length and sequence complementarity to a target sequence to hybridize specifically to a target sequence under stringency hybridization conditions. Probes and primers may have a complete sequence complementarity or identity with the target sequence, although probes and primers differing from the target sequence in terms of identity or complementarity but retaining the ability to form a stable double-stranded structure under particular hybridization conditions or reaction conditions and to hybridize to the target sequence may be designed by conventional methods.

Any conventional nucleic acid hybridization or amplification method can be used to identify or detect the presence of a target DNA from a transgenic plant, such as soybean event Gm_CSM63714, in a sample. Polynucleotide molecules or DNA molecules, also referred to as “polynucleotide segment or fragment of sufficient length” or “sufficient length of contiguous or consecutive nucleotides” therefore are capable of specifically hybridizing to a target DNA sequence under certain hybridization conditions or reaction conditions. As used herein, the term “of sufficient length” refers to any length that is sufficient to be useful in a detection method of choice. Probes and primers are generally at least about 8 nucleotides, at least about 10 nucleotides, at least about 12 nucleotides, at least about 14 nucleotides, at least about 16 nucleotides, at least about 18 nucleotides, at least about 20 nucleotides, at least about 22 nucleotides, at least about 24 nucleotides, at least about 26 nucleotides, at least about 28 nucleotides, or at least about 30 nucleotides or more in length. Such probes and primers hybridize specifically to a target DNA sequence under stringent hybridization conditions.

As used herein, two nucleic acid molecules are capable of specifically hybridizing to one another if the two molecules are capable of forming an anti-parallel, double-stranded nucleic acid structure. A nucleic acid molecule is the “complement” of another nucleic acid molecule if they exhibit complete complementarity. As used herein, two nucleic acid molecules exhibit “complete complementarity” and are “completely complementary” if every nucleotide of the first nucleic acid molecule is complementary to every nucleotide of the second nucleic acid molecule when they are aligned. Two molecules are “minimally complementary” if they can hybridize to one another with sufficient stability to permit them to remain annealed to one another under at least conventional “low-stringency” conditions. Similarly, the molecules are “complementary” if they can hybridize to one another with sufficient stability to permit them to remain annealed to one another under conventional “high-stringency” conditions. Conventional stringency conditions are described by Haymes et al., In: Nucleic Acid Hybridization, A Practical Approach, IRL Press, Washington, DC (1985), and by MR Green and J Sambrook, Molecular cloning: a laboratory manual, 4^{th} Edition, Cold Spring Harbor Laboratory Press, Cold Spring Harbor, N.Y. (2012). Departures from complete complementarity are therefore permissible, as long as such departures do not completely preclude the capacity of the molecules to form a double-stranded structure. In order for a nucleic acid molecule to serve as a primer or probe, it need only be sufficiently complementary in sequence to be able to form a stable double-stranded structure under the particular solvent and salt concentrations and other conditions employed.

As used herein, a substantially homologous or complementary sequence in relation to a reference nucleic acid sequence is a nucleic acid sequence that will specifically hybridize to the reference nucleic acid sequence or its complement to which it is being compared under high stringency conditions. As used herein, “stringent hybridization conditions” refers to conditions under which a polynucleotide will hybridize to its target sequence, typically in a complex mixture of nucleic acids, but to essentially no other sequences. “Stringent conditions” or “stringent hybridization conditions” when referring to a polynucleotide probe, refer to conditions under which a probe will hybridize to its target sequence to a detectably greater degree than to other sequences (e.g., at least 2-fold over background). Stringent conditions are sequence-dependent and will be different in different circumstances. Longer sequences hybridize specifically at higher temperatures. Generally, stringent conditions are selected to be about 5-10° C. lower than the thermal melting point (Tₘ) for the specific sequence at a defined ionic strength pH. The Tₘ is the temperature (under defined ionic strength, pH, and nucleic acid concentration) at which 50% of the probes complementary to the target hybridize to the target sequence at equilibrium (as the target sequences are present in excess, at Tₘ, 50% of the probes are occupied at equilibrium. Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g. greater than 50 nucleotides). Stringent conditions may also be achieved with the addition of destabilizing agents such as formamide. By controlling the stringency of the hybridization and/or washing conditions, target sequences that are 100% complementary to the probe can be identified (homologous probing). Alternatively, stringency conditions can be adjusted to allow some mismatching in sequences so that lower degrees of identity are detected (heterologous probing).

Appropriate stringency conditions which promote DNA hybridization, for example, 6× sodium chloride/sodium citrate (SSC) at about 45° C., followed by a wash of 2×SSC at 50° C., are known to those skilled in the art or can be found in Current Protocols in Molecular Biology, John Wiley & Sons, N.Y. (1989), 6.3.1-6.3.6. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or either the temperature or the salt concentration may be held constant while the other variable is changed. Regarding the amplification of a target polynucleotide (e.g., by PCR) using a particular amplification primer pair, “stringent conditions” or “stringent hybridization conditions” are conditions that permit the primer pair to hybridize to the target polynucleotide to which a primer having the corresponding wild-type sequence (or its complement) would bind and to produce an identifiable amplification product (the amplicon) having a soybean Gm_CSM63714 event specific region in a DNA thermal amplification reaction. The term “specific for” a target sequence indicates that a probe or primer hybridizes under stringent hybridization conditions only to the target sequence in a sample comprising the target sequence.

A polynucleotide molecule or DNA molecule of the present disclosure, such as a primer or a probe, will specifically hybridize to at least one of the nucleic acid molecule sequences selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, a polynucleotide having a nucleotide sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to SEQ ID NO:10, or a complete complement of or fragment of any of the foregoing under stringent hybridization conditions, or under moderately stringent hybridization conditions if the sequence of the polynucleotide molecule is not identical to the at least one of the nucleic acid molecules. The hybridization of a nucleic acid molecule, such as a primer or probe, to the target DNA molecule can be detected by any number of methods known to those skilled in the art, which can include, but are not limited to, fluorescent tags, radioactive tags, antibody-based tags, and chemiluminescent tags.

An illustrative DNA molecule or polynucleotide useful as a probe for detecting soybean event Gm_CSM63714 is provided as SEQ ID NO:16. In some embodiments, a DNA molecule that functions as a probe comprises a nucleotide sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, a complement of any of the foregoing or a fragment of any of the foregoing. In other embodiments, a DNA molecule comprises a polynucleotide segment of sufficient length to function as a DNA probe specific for at least one of: a) a 5′ junction sequence between flanking soybean genomic DNA and the transgenic insert of soybean event Gm_CSM63714; b) a 3′ junction sequence between the transgenic insert of soybean event Gm_CSM63714 and flanking soybean genomic DNA; c) SEQ ID NO:9; or d) a fragment of SEQ ID NO:9 comprising a sufficient length of contiguous nucleotides of SEQ ID NO:9 to identify the sequence as a fragment of the transgenic insert of Gm_CSM63714 in a sample of DNA.

A diagnostic amplicon produced by the methods described herein may be detected by a plurality of techniques known in the art, such as sequencing, restriction mapping, Southern analysis, or any other suitable polynucleotide or DNA hybridization, blotting, polymerization and/or amplification-based approach or technique. One method is Genetic Bit Analysis (Nikiforov et al., 1994) where a DNA oligonucleotide is designed that overlaps both the adjacent flanking genomic DNA sequence and the inserted DNA sequence—i.e., a junction sequence. The oligonucleotide is immobilized in wells of a microtiter plate. Following PCR of the region of interest (using, for example, one primer in the inserted sequence and one in the adjacent flanking genomic sequence), a single-stranded PCR product can be hybridized to the immobilized oligonucleotide and serve as a template for a single base extension reaction using a DNA polymerase and labeled dideoxynucleotide triphosphates (ddNTPs) specific for the expected next base. Readout may be fluorescent or ELISA-based. A signal indicates presence of the transgene/genomic junction sequence due to successful amplification, hybridization, and single base extension.

Another method is the pyrosequencing technique as described by Winge (2000). In this method, an oligonucleotide is designed that overlaps the adjacent genomic DNA and insert DNA junction. The oligonucleotide is hybridized to single-stranded PCR product from the region of interest (one primer in the inserted sequence and one in the flanking genomic sequence) and incubated in the presence of a DNA polymerase, ATP, sulfurylase, luciferase, apyrase, adenosine 5′ phosphosulfate and luciferin. DNTPs are added individually and the incorporation results in a light signal that is measured. A light signal indicates the presence of the transgene/genomic sequence due to successful amplification, hybridization, and single or multi-base extension.

Fluorescence Polarization as described by Chen et al. (1999) is a method that can be used to detect the amplicon of the present invention. Using this method an oligonucleotide is designed that overlaps the genomic flanking and inserted DNA junction. The oligonucleotide is hybridized to single-stranded PCR product from the region of interest (one primer in the inserted DNA and one in the flanking genomic DNA sequence) and incubated in the presence of a DNA polymerase and a fluorescent-labeled ddNTP. Single base extension results in incorporation of the ddNTP. Incorporation can be measured as a change in polarization using a fluorometer. A change in polarization indicates the presence of the transgene/genomic sequence due to successful amplification, hybridization, and single base extension.

Real-time polymerase chain reaction (PCR) has the ability to monitor the progress of the PCR as it occurs (i.e., in real time). Data is collected throughout the PCR process, rather than at the end of the PCR. In real-time PCR, reactions are characterized by the point in time during cycling when amplification of a target is first detected rather than the amount of target accumulated after a fixed number of cycles. In a real-time PCR assay, a positive reaction is detected by accumulation of a fluorescent signal. The higher the starting copy number of the nucleic acid target, the sooner a significant increase in fluorescence is observed. The cycle threshold (Ct value) is defined as the number of cycles required for the fluorescent signal to cross the threshold (i.e., exceeds background level). Ct levels are inversely proportional to the amount of target nucleic acid in the sample (i.e., the lower the Ct value, the greater the amount of target nucleic acid in the sample).

Taqman® (PE Applied Biosystems, Foster City, CA) is described as a method of detecting and quantifying the presence of a DNA sequence using real-time PCR and is fully understood in the instructions provided by the manufacturer. Briefly, a FRET oligonucleotide probe is designed that overlaps the genomic flanking and insert DNA junction. The FRET probe and PCR primers (one primer in the insert DNA sequence and one in the flanking genomic sequence) are cycled in the presence of a thermal stable polymerase and dNTPs. Hybridization of the FRET probe results in cleavage and release of the fluorescent moiety away from the quenching moiety on the FRET probe. A fluorescent signal indicates the presence of the transgene/genomic sequence due to successful amplification and hybridization.

Molecular beacons have been described for use in sequence detection as described in Tyangi et al. (1996). Briefly, a FRET oligonucleotide probe is designed that overlaps the flanking genomic and insert DNA junction. The unique structure of the FRET probe results in it containing secondary structure that keeps the fluorescent and quenching moieties in close proximity. The FRET probe and PCR primers (one primer in the insert DNA sequence and one in the flanking genomic sequence) are cycled in the presence of a thermostable polymerase and dNTPs. Following successful PCR amplification, hybridization of the FRET probe to the target sequence results in the removal of the probe secondary structure and spatial separation of the fluorescent and quenching moieties. A fluorescent signal results and indicates the presence of the flanking/transgene insert sequence due to successful amplification and hybridization.

Other detection methods known in the art may be used. For example, microfluidics (see, e.g., U.S. Patent Publication No. 2006/068398; U.S. Pat. No. 6,544,734) provide methods and devices that can be used to separate and amplify DNA samples or molecules. Optical dyes can be used to detect and measure specific DNA molecules (see, e.g., WO/05017181). Nanotube devices (see, e.g., WO/06024023) that comprise an electronic sensor for the detection of DNA molecules or nanobeads that bind specific DNA molecules can then be detected. Nanopore sequencing technology, such as that described in Wang et al. (2021), Tyler et al. (2018), or Pearson et al. (2019), can also be used for event detection.

The DNA molecules and corresponding nucleotide sequences provided herein are therefore useful for, among other things, identifying soybean event Gm_CSM63714, detecting the presence of DNA derived from the transgenic soybean event Gm_CSM63714 in a sample, and monitoring samples for the presence and/or absence of soybean event Gm_CSM63714 or plant parts derived from soybean plants comprising event Gm_CSM63714.

Provided are proteins that can be used to produce antibodies for detecting the presence of soybean event Gm_CSM63714 in a sample. Such antibodies are specific for one or more of the proteins that are encoded by soybean event Gm_CSM63714. Methods for preparing a polyclonal antibody or a monoclonal antibody are well-known to those skilled in the art, and can be used to make antibodies specific for one or more of the proteins encoded by soybean event Gm_CSM63714. For example, U.S. Pat. No. 7,838,729 and Wang et al. (2016) describe antibodies to DMO; U.S. Pat. No. 9,371,394 describes antibodies to the PAT enzyme. The DNA sequence encoding such proteins is provided in SEQ ID NO:10 and the start positions and stop positions of the coding sequences are indicated in Table 1. The DNA sequence encoding each protein and the protein encoded by the sequence are useful to produce antibodies for detecting the presence of soybean event Gm_CSM63714 by the methods described herein. Detection for the presence of soybean event Gm_CSM63714 may be done by using any protein detection techniques known in the art, such as western blot analysis, immuno-precipitation, enzyme-linked immunosorbent assay (ELISA), antibody attachment to a detectable label or reporter molecule (such as a radioactive isotope, ligand, chemiluminescent agent, or enzyme), or enzymatic action on a reporter molecule. One method provides for contacting a sample with an antibody that binds to the DMO, PAT, FT_Tv7 or TDO protein encoded by soybean event Gm_CSM63714 and then detecting the presence or absence of antibody binding. The binding of such antibody is diagnostic for the presence of one or more proteins encoded by soybean event Gm_CSM63714.

Nucleic acid or protein detection kits for detecting the presence of soybean event Gm_CSM63714 are provided. Variations on such kits can also be developed using the compositions and methods disclosed herein and the methods well known in the art for protein and nucleic acid detection for identification of soybean event Gm_CSM63714. Protein and nucleic acid detection kits can be applied to methods for breeding with plants comprising soybean event Gm_CSM63714. Such kits contain primers and/or probes or antibodies which are specific to soybean event Gm_CSM63714. Such DNA primers and/or probes may comprise fragments of one or more of SEQ ID NOs:1-10, or antibodies specific for a protein encoded by the soybean event Gm_CSM63714. The kits can also contain instructions for using the primers, probes, or antibodies for detecting the presence of soybean event Gm_CSM63714. Kits may optionally also comprise reagents for performing the detection or diagnostic reactions described herein.

One example of a detection kit comprises at least one DNA molecule of sufficient length of contiguous nucleotides of SEQ ID NO:10 to function as a DNA probe useful for detecting the presence or absence of soybean event Gm_CSM63714 in a sample. The DNA derived from transgenic soybean plants comprising event Gm_CSM63714 would comprise a DNA molecule having at least one sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, and SEQ ID NO:10, a complement of any of the foregoing, or a fragment of any of the foregoing. An illustrative DNA molecule sufficient for use as a probe is one comprising the sequence provided as SEQ ID NO:16. Other probes may be readily designed by one of skill in the art. The probe can include a junction sequence that spans the 5′ or 3′ junction between the soybean genomic DNA and the transgenic insert of soybean event Gm_CSM63714.

Another example of a detection kit comprises at least one primer pair that specifically hybridize to a target DNA and amplify a diagnostic amplicon under the appropriate reaction conditions useful for detecting the presence or absence of soybean event Gm_CSM63714 in a sample. A kit that contains DNA primers that are homologous or complementary to any portion of the soybean genomic region as set forth in SEQ ID NO:10 and to any portion of the inserted transgenic DNA as set forth in SEQ ID NO:9 is an object of the present disclosure. The kit may provide an agarose gel-based detection method or any number of methods of detecting the amplicon that are known in the art. Such a method may also include sequencing the amplicon or a fragment thereof. Illustrative DNA molecules sufficient for use as a primer pair are ones comprising the sequences provided as SEQ ID NO:14 and SEQ ID NO:15, and SEQ ID NO:20 and SEQ ID NO:15, respectively, wherein the primer pair SEQ ID NO:14 and SEQ ID NO:15 will produce an amplicon diagnostic for the presence of event Gm_CSM63714 in a sample; and the primer pair SEQ ID NO:20 and SEQ ID NO:15 will produce an amplicon indicative of wild-type DNA, therefore, diagnostic for the absence of event Gm_CSM63714 in a sample. Other primer pairs may be readily designed by one of skill in the art.

Another example of a detection kit comprises at least one antibody specific for at least one protein encoded by soybean event Gm_CSM63714. For example, such a kit may utilize a lateral flow strip comprising reagents activated when the tip of the strip is contacted with an aqueous solution. Illustrative proteins sufficient for use in antibody production are ones encoded by the sequence provided as SEQ ID NO:10, or any fragment thereof. Detection of binding of the at least one antibody to the at least one protein encoded by soybean event Gm_CSM63714 in a sample is diagnostic for the presence of soybean event Gm_CSM63714 in the sample.

The detection kits provided herein are useful for, among other things, identifying soybean event Gm_CSM63714, selecting plant varieties or hybrids comprising soybean event Gm_CSM63714, detecting the presence of DNA derived from the transgenic soybean plant comprising event Gm_CSM63714 in a sample, and monitoring samples for the presence and/or absence of soybean plants comprising event Gm_CSM63714, or plant parts derived from soybean plants comprising event Gm_CSM63714.

Soybean plants, progeny, seeds, cells, and plant parts comprising soybean event Gm_CSM63714 are provided, as well as commodity products produced using these. As used herein, the term “soybean” or “soy” means plant species within Glycine max and all plant varieties belonging to the genus Glycine that can be bred with Glycine max plants, including wild soybean species such as Glycine soja. The term “soybean” is intended to include soybean plants, plant parts, plant cells, plant tissue, seeds, progeny plants, and/or soybean commodity products. These soybean plants, plant parts, plant cells, plant tissues, seeds, progeny plants and commodity products contain or comprise soybean event Gm_CSM63714 or are derived from a transgenic soybean plant, plant part, plant cell, plant tissue, seed, progeny plant or commodity product containing or comprising event Gm_CSM63714. These soybean plants, plant parts, plant cells, plant tissues, seeds, progeny plants and commodity products contain a detectable amount of a polynucleotide or DNA molecule comprising at least one junction sequence and/or heterologous transgenic insert sequence of soybean event Gm_CSM63714, such as a polynucleotide or nucleic acid or DNA molecule having or comprising at least one of the sequences provided as SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, a polynucleotide comprising at least 16 consecutive nucleotides of SEQ ID NO:1, at least 23 consecutive nucleotides of SEQ ID NO:2, at least 33 consecutive nucleotides of SEQ ID NO:3, at least 31 consecutive nucleotides of SEQ ID NO:4, at least 51 consecutive nucleotides of SEQ ID NO:5, or at least 51 consecutive nucleotides of SEQ ID NO:6, a polynucleotide comprising a sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO:10 or the full length of SEQ ID NO: 9, and a complete complement of any of the foregoing. In some embodiments, the soybean plant, plant part, plant cell, plant tissue, or seed is further defined as a progeny plant of any generation of a soybean plant comprising soybean event Gm_CSM63714, or a soybean plant part, plant seed, or plant cell derived therefrom.

The soybean plants, plant parts, plant cells, plant tissues, seeds, progeny plants and commodity products express or contain at least one herbicide tolerance gene selected from the group consisting of dicamba monooxygenase (DMO), phosphinothricin N-acetyltransferase (PAT), an alpha-ketoglutarate-dependent non-heme iron dioxygenase variant (FT_Tv7), triketone dioxygenase (TDO), and any combination thereof, and are tolerant to at least one herbicide selected from the group consisting of dicamba, glufosinate, 2,4-D, a β-triketone (HPPD inhibitor) such as mesotrione, and any combination thereof.

Also provided are soybean plants, plant seeds, plant parts, and plant cells tolerant to herbicides with at least three different herbicide modes of action, wherein the genes that confer the herbicide tolerance are present at a single genomic location. For example, the soybean plants, plant seeds, plant parts, or plant cells can be tolerant to herbicides with at least four different herbicide modes of action, wherein the genes that confer the herbicide tolerance are present at a single genomic location. To make such a soybean plant, plant seed, plant part, or plant cell, three or more transgenic cassettes containing herbicide tolerance genes can be inserted at a single genomic location within the soybean genome as a contiguous polynucleotide or a single molecularly linked transgenic insert. Alternatively, three or more transgenic cassettes containing herbicide tolerance genes can be inserted at a single genomic location by inserting separate cassettes containing the herbicide tolerance genes at the same location. By “single genomic location” it is meant that the genes, together with any regulatory sequences (e.g., promoters, introns, leader sequences, 5′-UTRs, and/or 3′UTRs, etc.) and/or sequences encoding targeting peptides (e.g., chloroplast transit peptides) are present at a single location on a chromosome, and will be inherited as a single locus. While some intervening sequence may be present between each transgene cassette, the length of the intervening sequence is limited such that the transgenes cassettes are close to one another on the chromosome. For example, the intervening sequence between the transgene cassettes may be 500 nucleotides or fewer in length, 400 nucleotides or fewer in length, 300 nucleotides or fewer in length, 250 nucleotides or fewer in length, 200 nucleotides or fewer in length, or 150 nucleotides or fewer in length. For example, the soybean plant, plant seed, plant part, or plant cell can comprise any of DNA constructs described herein, and can exhibit tolerance to at least one herbicide selected from the group consisting of benzoic acid auxins such as dicamba, phenoxy auxins such as 2,4-D, glutamine synthetase inhibitors such as glufosinate, β-triketone HPPD inhibitors such as mesotrione, and any combination thereof.

The present disclosure provides soybean plants, progeny, seeds, plant cells, and plant parts such as microspores, pollen, anthers, ovules, ovaries, flowers, pods, embryos, stems, leaves, roots, and calluses derived from a transgenic soybean plant comprising soybean event Gm_CSM63714. A representative sample of seed comprising soybean event Gm_CSM63714 has been deposited according to the Budapest Treaty for the purpose of enabling the present disclosure. The ATCC repository has assigned the Accession No. PTA-127099 to the seed comprising soybean event Gm_CSM63714.

A microorganism is provided. The microorganism comprises a polynucleotide molecule having the nucleotide sequence of SEQ ID NO:9, or a nucleotide sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4% at least 99.5%, at least 99.6%, at least 99.7%, at least 99.8%, or at least 99.9% identical to the full length of SEQ ID NO:9. An example of such a microorganism is an Agrobacterium cell. Another example of such a microorganism is an E. coli cell.

A plant cell is provided comprising a polynucleotide molecule as described herein. For example, a plant cell is provided having a nucleotide sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, and a nucleic acid molecule comprising a polynucleotide having a nucleotide sequence that is at least 90%, at least 91%, at least 92%, at least 93%, at least 94%, at least 95%, at least 96%, at least 97%, at least 98%, at least 99%, at least 99.5%, at least 99.6%, at least 99.7%, at least 99.8%, or at least 99.9% identical to the full length of SEQ ID NO:10 or the full length of SEQ ID NO: 9 present in its genome.

Plant cells and microorganisms of the present disclosure are useful in many industrial applications, including but not limited to: (i) use as research tools for scientific inquiry or industrial research; (ii) use in culture for producing endogenous or recombinant carbohydrate, lipid, nucleic acid, enzymes or protein products or small molecules that may be used for subsequent scientific research or as industrial products; and (iii) for the plant cells of the present disclosure, use with modern plant tissue culture techniques to produce transgenic plants or plant tissue cultures that may then be used for agricultural research or production. The production and use of such transgenic plant cells utilize modern microbiological techniques and human intervention to produce a man-made, unique plant cell. In this process, a recombinant DNA is inserted into a plant cell's genome to create a transgenic plant cell that is separate and unique from naturally occurring plant cells. This transgenic plant cell can then be cultured much like bacteria and yeast cells using modern microbiology techniques and may exist in an undifferentiated, unicellular state. The new plant cell's genetic composition and phenotype is a technical effect created by the integration of a heterologous DNA into the genome of the cell.

Provided are method of using a plant cell, such as transgenic plant cells. These include (i) methods of producing transgenic cells by integrating a recombinant DNA into the genome of the cell and then using this cell to derive additional cells possessing the same heterologous DNA; (ii) methods of culturing cells that contain recombinant DNA using modern microbiology techniques; (iii) methods of producing and purifying endogenous or recombinant carbohydrate, lipid, nucleic acid, enzymes or protein products from cultured cells; and (iv) methods of using modern plant tissue culture techniques with transgenic plant cells to produce transgenic plants or transgenic plant tissue cultures.

Plants, progeny, seeds, cells, and plant parts may contain one or more additional desirable trait(s). Such desirable traits may be transgenic traits, native traits, or traits produced by other methods such as genome editing, base editing, prime editing or other conventional mutagenesis methods. Desirable traits may be combined with soybean event Gm_CSM63714 by, for example, crossing a soybean plant comprising soybean event Gm_CSM63714 with another soybean plant containing the additional trait(s), or transgenic events. Such traits or transgenic events include, but are not limited to, increased insect resistance, increased water use efficiency, increased yield performance, increased drought resistance, increased disease resistance, increased seed quality, improved nutritional quality, hybrid seed production, and/or increase herbicide tolerance, in which the trait is measured with respect to a soybean plant lacking such transgenic trait. For example, the Gm_CSM63714 event could be stacked by breeding or by site directed introgression with other events known in the art including, but not limited to, A2704-12 (Liberty Link® for glufosinate herbicide tolerance), A2704-21 (Liberty Link® for glufosinate herbicide tolerance), A5547-127 (Liberty Link® for glufosinate herbicide tolerance), A5547-35 (Liberty Link® for glufosinate herbicide tolerance), CV127 (Cultivance for sulfonylurea herbicide tolerance), DAS44406-6 (for glufosinate, glyphosate and 2,4-D herbicide tolerance), DAS81419 (for glufosinate tolerance and Lepidopteran resistance), DP356043 (Optimum GAT™ for glyphosate and sulfonylurea herbicide tolerance), FG72 (for glyphosate and isoxaflutole herbicide tolerance), FG72×A5547-127 (Liberty Link® GT27™ for glufosinate, glyphosate and isoxaflutole herbicide tolerance), GMB151 (for isoxaflutole herbicide tolerance), GTS 40-3-2 (Roundup Ready™ for glyphosate herbicide tolerance), GU262 (Liberty Link™ for glufosinate herbicide tolerance, and antibiotic resistance), MON87708 (Genuity® Roundup Ready™ 2 Xtend™ for glyphosate and dicamba herbicide tolerance), MON89788 (Genuity® Roundup Ready 2 Yield™ for glyphosate herbicide tolerance), SYHT0H2 (Herbicide-Tolerant Soybean Line for glufosinate and mesotrione herbicide tolerance), W62 (Liberty Link™ for glufosinate herbicide tolerance), and W98 (Liberty Link™ for glufosinate herbicide tolerance), MON87701 (for Lepidopteran insect resistance), MON87751 (for Lepidopteran insect resistance), DAS81419×DAS44406 (Conkesta Enlist E3™ for glufosinate, glyphosate and 2,4-D herbicide tolerance, and Lepidopteran insect resistance), MON87701×MON89788 (Intacta™ Roundup Ready™ 2 Pro for glyphosate herbicide tolerance and Lepidopteran insect resistance), MON87751×MON87701×MON87708×MON89788 (for glyphosate and dicamba herbicide tolerance, and Lepidopteran insect resistance) to provide herbicide tolerance and/or to control Lepidopteran pests. The Gm_CSM63714 event could also be stacked by breeding or by site directed introgression with other transgenic soybean events known in the art including, but not limited to, DP305423 (Treus™, Plenish™ for sulfonylurea herbicide tolerance, and modified oil/fatty acid), MON87705 (Vistive Gold™ for glyphosate herbicide tolerance and modified oil/fatty acid), MON87712 (for glyphosate herbicide tolerance and enhanced photosynthesis/yield), MON87769 (for glyphosate herbicide tolerance and modified oil/fatty acid), and HB4 (Verdeca HB4 Soybean for drought stress tolerance) to provide tolerance to herbicides and/or modified oils, enhanced photosynthesis/yield, or drought tolerance. The Gm_CSM63714 event could also be stacked by breeding or by site directed introgression with genome edited events known in the art including, but not limited to, high-oleic soybean trait, and high oleic low linolenic (HOLL) soybean trait.

The plants described herein can be used to produce progeny or offspring that comprise soybean event Gm_CSM63714. Such progeny may include any plant, seed, and cell and/or regenerable plant part comprising soybean event Gm_CSM63714 inherited or derived from an ancestor or parental soybean plant(s), at least one of which comprises a DNA molecule having or comprising at least one polynucleotide sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, a polynucleotide comprising at least 16 consecutive nucleotides of SEQ ID NO:1, at least 23 consecutive nucleotides of SEQ ID NO:2, at least 33 consecutive nucleotides of SEQ ID NO:3, at least 31 consecutive nucleotides of SEQ ID NO:4, at least 51 consecutive nucleotides of SEQ ID NO:5, or at least 51 consecutive nucleotides of SEQ ID NO:6, or a polynucleotide having a nucleotide sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO:10 or the full length of SEQ ID NO: 9.

Soybean plants, progeny, and seed may be homozygous or heterozygous for the event Gm_CSM63714 and the transgenes of event Gm_CSM63714. Progeny may be grown from seeds produced by a soybean plant comprising or containing event Gm_CSM63714 and/or from seeds produced by a plant fertilized with pollen from a soybean plant comprising or containing event Gm_CSM63714 (i.e., fertilized with pollen comprising or containing event Gm_CSM63714). Plants or progeny may also be obtained by tissue culture and regeneration methods from a protoplast, cell, embryo or reproductive or somatic tissue derived from a soybean plant comprising or containing soybean event Gm_CSM63714.

Progeny plants may be self-pollinated (also known as “selfing”) to generate a true breeding line of plants, i.e., plants homozygous for the soybean event Gm_CSM63714 DNA. Alternatively, progeny plants may be outcrossed, i.e., bred with another plant, to produce a varietal or a hybrid seed or plant. The other plant may be transgenic or non-transgenic. A varietal or hybrid seed or plant of the present disclosure may thus be derived by crossing a first parent that lacks the specific and unique DNA of event Gm_CSM63714 with a second parent comprising event Gm_CSM63714, resulting in a hybrid comprising the specific and unique DNA of event Gm_CSM63714. Each parent can be a hybrid or an inbred/variety, so long as the cross or breeding results in a plant or seed of the present disclosure, i.e., a seed having at least one allele comprising the specific and unique DNA of event Gm_CSM63714 and/or at least 16 consecutive nucleotides of SEQ ID NO:1, at least 23 consecutive nucleotides of SEQ ID NO:2, at least 33 consecutive nucleotides of SEQ ID NO:3, at least 31 consecutive nucleotides of SEQ ID NO:4, at least 51 consecutive nucleotides of SEQ ID NO:5, or at least 51 consecutive nucleotides of SEQ ID NO:6, or a polynucleotide having a sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO:10 or the full length of SEQ ID NO:9.

Sexually crossing one plant with another plant, i.e., cross-pollinating, may be accomplished or facilitated by human intervention, for example: by human hands collecting the pollen of one plant and contacting this pollen with the style or stigma of a second plant; by human hands and/or human actions removing, destroying, or covering the stamen or anthers of a plant (e.g., by manual intervention or by application of a chemical gametocide) so that natural self-pollination is prevented and cross-pollination would have to take place in order for fertilization to occur; by human placement of pollinating insects in a position for “directed pollination” (e.g., by placing beehives in orchards or fields or by caging plants with pollinating insects); by human opening or removing of parts of the flower to allow for placement or contact of foreign pollen on the style or stigma; by selective placement of plants (e.g., intentionally planting plants in pollinating proximity); and/or by application of chemicals to precipitate flowering or to foster receptivity (of the stigma for pollen).

Two different transgenic plants of the same or different genetic backgrounds may thus be crossed to produce inbred or hybrid offspring plants, plant parts and/or seeds that contain two independently segregating transgenes or events wherein at least one of those transgenes or events comprises or is contained within soybean event Gm_CSM63714. For example, transgenic plants comprising soybean event Gm_CSM63714 can be crossed with other transgenic soybean plants to produce a plant having the characteristics of both transgenic parents.

Back-crossing to a parental plant and out-crossing with a non-transgenic plant are also contemplated, as is vegetative propagation. Descriptions of other breeding methods that are commonly used for different traits and crops are known in the art and can be found in one of several references, e.g., Fehr, in Breeding Methods for Cultivar Development, Wilcox J. ed., American Society of Agronomy, Madison WI (1987).

A plant part is provided. As used herein, a “plant part” refers to any part of a plant that is comprised of material directly from or derived from a plant comprising soybean event Gm_CSM63714. Plant parts include but are not limited to microspores, pollen, anthers, ovules, ovaries, flowers, pods, embryos, stems, leaves, roots, and calluses, in whole or part. Plant parts may be viable or nonviable, regenerable and/or non-regenerable.

Commodity products that are produced from plants comprising soybean event Gm_CSM63714 are provided. The commodity products contain a detectable amount of DNA comprising a DNA sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, or a polynucleotide having a sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO:10 or the full length of SEQ ID NO:9. As used herein, a “commodity product” refers to any composition or product which is comprised of material from plant, seed, cell, or plant part comprising soybean event Gm_CSM63714. Commodity products may be viable or non-living plant material, that is, a material that is not living and derived from a plant, seed, cell, or plant part comprising soybean event Gm_CSM63714. Nonviable commodity products include but are not limited to nonviable seeds, whole or processed seeds, processed plant tissues or plant parts, dehydrated plant tissues or parts, frozen plant tissues or parts, food for human consumption (such as soy oil, soy milk, soy flour and grits, soy protein, soy protein concentrate, hydrolyzed vegetable protein, textured soy protein, lecithin, curd, tofu, vegetable soybean (edamame), soy sprout, soy film (yuba), roasted soybeans, miso, tempeh, soy sauce, natto), plant parts processed for animal feed such as soy meal, soy fiber, biodiesel, bio-composite building materials (such as particleboard, laminated plywood and lumber products), soy oil based solvents and industrial lubricants, soy ink, soy candle and crayon, soy-based hydraulic fluid, and soy-based foams. Viable commodity products include but are not limited to viable seeds, viable plant parts (such as root and leaf) and viable plant cells. A plant comprising event Gm_CSM63714 can thus be used to manufacture any commodity product typically acquired from a soybean plant. Any such commodity product that is derived from the plants comprising event Gm_CSM63714 may contain at least a detectable amount of the specific and unique DNA corresponding to event Gm_CSM63714, and specifically may contain a detectable amount of a polynucleotide having a nucleotide sequence of at least 16 consecutive nucleotides of SEQ ID NO:1, at least 23 consecutive nucleotides of SEQ ID NO:2, at least 33 consecutive nucleotides of SEQ ID NO:3, at least 31 consecutive nucleotides of SEQ ID NO:4, at least 51 consecutive nucleotides of SEQ ID NO:5, or at least 51 consecutive nucleotides of SEQ ID NO:6, or a polynucleotide having a sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO:10 or the full length of SEQ ID NO:9. Any standard method of detection for polynucleotide molecules may be used, including methods of detection disclosed herein.

A plant tolerant to herbicides may be produced by sexually crossing a plant comprising event Gm_CSM63714 with another plant and thereby producing seed, which is then grown into progeny plants. These progeny plants may be analyzed using diagnostic methods to select for progeny plants that comprise event Gm_CSM63714 DNA or for progeny plants tolerant to the herbicides benzoic acid auxins such as dicamba, glutamine synthetase inhibitors such as glufosinate, phenoxy auxins such as 2,4-D, β-triketones such as mesotrione, and any combination thereof. The other plant used may or may not be transgenic. The progeny plant and/or seed produced may be varietal or hybrid seed.

A plant tolerant to herbicides may be produced by selfing a plant comprising event Gm_CSM63714 comprising a polynucleotide having the nucleotide sequence of SEQ ID NOs:1-10, at least 16 consecutive nucleotides of SEQ ID NO:1, at least 23 consecutive nucleotides of SEQ ID NO:2, at least 33 consecutive nucleotides of SEQ ID NO:3, at least 31 consecutive nucleotides of SEQ ID NO:4, at least 51 consecutive nucleotides of SEQ ID NO:5, or at least 51 consecutive nucleotides of SEQ ID NO:6, and a polynucleotide having a sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO:10 or the full length of SEQ ID NO:9, and thereby producing seed, which is then grown into progeny plants. These progeny plants may then be analyzed using diagnostic methods to select for progeny plants that comprise event Gm_CSM63714 DNA, or for progeny plants tolerant to the herbicides such as dicamba, glufosinate, 2,4-D, a β-triketone HPPD inhibitor such as mesotrione, and any combination thereof.

Soybean event Gm_CSM63714 contains four expression cassettes that together provide tolerance to benzoic acid auxins such as dicamba; phenoxy auxins such as 2,4-D; inhibitors of glutamine synthetase such as glufosinate; and β-triketone HPPD inhibitors such as mesotrione.

As used herein, inhibitors of glutamine synthetase include, but are not limited to, phosphinothricin, glufosinate, glufosinate salts, glufosinate-ammonium, glufosinate-sodium, glufosinate-P, L-glufosinate-ammonium, and L-glufosinate-sodium.

As used herein, synthetic auxins include, but are not limited to, benzoic acid herbicides, phenoxy acid herbicides, arylpicolinate herbicides, and pyridinyloxy acid herbicides. Examples of a benzoic acid herbicides include, but are not limited to, dicamba (3,6-dichloro-2-methoxybenzoic acid), dicamba salts, dicamba-butotyl, dicamba-diglycolamine salt, dicamba-dimethylammonium, dicamba-diethanolammonium, dicamba-isopropylammonium, dicamba-potassium, dicamba-sodium, and dicamba-trolamine. Examples of phenoxy acid herbicides include, but are not limited to, 2,4-D (2,4-dichlorophenoxyacetic acid), 2,4-D-butotyl, 2,4-D-butyl, 2,4-D-choline, 2,4-D-dimethylammonium, 2,4-D-diolamin, 2,4-D-ethyl, 2,4-D-2-ethylhexyl, 2,4-D-isobutyl, 2,4-D-isoctyl, 2,4-D-isopropyl, 2,4-D-isopropylammonium, 2,4-D-potassium, 2,4-D-sodium, 2,4-D-triisopropanolammonium, 2,4-D-trolamine, clomeprop, dichlorprop, fenoprop, MCPA (2-methyl-4-chlorophenoxyacetic acid), MCPA-butotyl, MCPA-dimethylammonium, MCPA-2-ethylhexyl. MCPA-isopropylammonium, MCPA-potassium, MCPA-sodium, MCPA-thioethyl, 2,4-DB, MCPB (4-(4-chloro-2-methylphenoxy)butanoic acid), MCPB-methyl, MCPB-ethyl-sodium, and mecoprop. Examples of arylpicolinate herbicides include, but are not limited to, halauxifen, halauxifen-methyl, and florpyrauxifen-benzyl. Examples of pyridinyloxy acid herbicides include, but are not limited to, triclopyr, fluroxypyr, aminopyralid, and picloram.

As used herein, β-triketone HPPD inhibitors include, but are not limited to benzobicyclon (BBC), tefuryltrione, sulcotrione, mesotrione, and tembotrione.

FT_Tv7 degrades phenoxy auxins such as 2,4-D, and thus soybean containing event Gm_CSM63714 exhibits tolerance to 2,4-D. In addition, FT_Tv7 is also capable of degrading aryloxyphenoxypropionate (AOPP) inhibitors of acetyl CoA carboxylase (ACCase), including the “fop” family of herbicides. Examples of AOPP herbicide inhibitors of ACCase include, but are not limited to clodinafop, clodinafop-ethyl, clodinafop-propargyl, cyhalofop, cyhalofop-butyl, diclofop, diclofop-methyl, diclofop-P, diclofop-P-methyl, fenoxaprop, fenoxaprop-P, fenoxaprop-P-ethyl, fenthiaprop, fluazifop, fluazifop-butyl, fluazifop-P, fluazifop-P-butyl, fluroxypyr, haloxyfop, haloxyfop-etotyl, haloxyfop-methyl, haloxyfop-P, haloxyfop-P-methyl, isoxapyrifop, metamifop, propaquizafop, quizalofop, quizalafop-ethyl, quizalofop-P, quizalafop-P-ethyl, quizalafop-P-tefuryl, and trifop. Accordingly, in plants that are not naturally tolerant to such herbicides (e.g., maize), in addition to conferring tolerance to phenoxy auxin herbicides such as 2,4-D, FT_Tv7 also confers tolerance to AOPP herbicides. Additionally, in plants that are naturally tolerant to AOPP or fop herbicides, or that have been selected or engineered to be tolerant to AOPP or fop herbicides, FT_Tv7 may provide an enhanced level of tolerance to these herbicides. The ability of FT_Tv7 and related alpha-ketoglutarate-dependent non-heme iron dioxygenase variants to degrade both phenoxy auxins such as 2,4-D and AOPP herbicides is further described in U.S. Pat. No. 10,023,874, the disclosure of which is incorporated herein by reference in its entirety.

As used herein, “herbicide tolerant” or “herbicide tolerance” or “tolerance” means the ability to be wholly or partially unaffected by the presence or application of one of more herbicide(s), for example to resist the toxic effects of an herbicide when applied. A cell, seed, or plant is “herbicide tolerant” or has “improved tolerance” if it can maintain at least some normal growth or phenotype in the presence of one or more herbicide(s). A trait is an herbicide tolerance trait if its presence can confer improved tolerance to an herbicide upon a cell, plant, or seed as compared to the wild-type or control cell, plant, or seed. Crops comprising an herbicide tolerance trait can continue to grow in the presence of the herbicide and may be minimally affected by the presence of the herbicide. A protein confers “herbicide tolerance” if expression of the protein can confer improved tolerance to an herbicide upon a cell, plant, or seed as compared to the wild-type or control cell, plant, or seed. Examples of herbicide tolerance proteins are dicamba monooxygenase, phosphinothricin N-acetyltransferase, the alpha-ketoglutarate-dependent non-heme iron dioxygenase variant FT_Tv7 and triketone dioxygenase. Herbicide tolerance may be complete or partial insensitivity to a particular herbicide and may be expressed as a percent (%) tolerance or insensitivity to a particular herbicide.

As used herein “herbicide injury” or “injury” refers to injury to a plant because of the application of one or more herbicides. The “injury rate” or “percent injury” refers to the percentage of leaf area of a plant exhibiting damage such as necrosis (brown or dead tissue), chlorosis (yellow tissue or yellow spotting) and malformation (misshapen leaves or plant structures, epinasty or twisting of stem, cupping of leaves) caused by herbicide application based on visual evaluation. It is measured on a scale of 0 to 100, where “0” representing no crop injury and “100” denoting complete crop injury (death).

For soybean plants containing or comprising soybean event Gm_CSM63714, the plant will have decreased injury after application of one or more of: a synthetic auxin (such as dicamba or 2,4-D); a β-triketone HPPD inhibitor (such as mesotrione); or an inhibitor of glutamine synthetase (such as glufosinate). For example, soybean plants containing or comprising soybean event Gm_CSM63714 will have less than about 5% injury, less than about 10% injury, less than about 15% injury, or less than about 20% injury following application of a synthetic auxin such as dicamba or 2,4-D, a β-triketone HPPD inhibitor such as mesotrione, or a glutamine synthetase inhibitor such as glufosinate, as compared to otherwise identical soybean plants that do not contain soybean event Gm_CSM63714.

As used herein, a “weed” is any undesired plant. A plant may be considered generally undesirable for agriculture or horticulture purposes (for example, Amaranthus species) or may be considered undesirable in a particular situation (for example, a crop plant of one species in a field of a different species, also known as a volunteer plant). Weeds are commonly known in the art and vary by geography, season, growing environment, and time. Lists of weed species are available from agricultural and scientific societies and efforts (such as the Weed Science Society of America, the Canadian Weed Science Society, the Brazilian Weed Science Society, the International Weed Science Society, and the International Survey of Herbicide Resistant Weeds), government agencies (such as the United States Department of Agriculture and the Australia Department of the Environment and Energy), and industry and farmer associations. Major troublesome weeds in soybean production include waterhemp (Amaranthus tuberculatus), ragweed (Ambrosia spp.), common lambsquarters (Chenopodium album), morning glory species (Ipomoea spp.), horseweed/marestail (Erigeron canadensis), palmer amaranth (Amaranthus palmeri), pigweed (Amaranthus spp.), velvetleaf (Abutilon theophrasti Medik.), common cocklebur (Xanthium strumarium), foxtail (Setaria spp.), crabgrass (Digitaria spp.), barnyard grass (Echinochloa crus-galli), Johnsongrass (Sorghum halepense), and thistles (Cirsium spp.) (Heap 2021; Shoup et al, 2016)

Methods for controlling weeds in an area for soybean cultivation are provided. The methods comprise applying at least one herbicide selected from the group consisting of (i) inhibitors of glutamine synthetase such as glufosinate; (ii) benzoic auxins such as dicamba; (iii) phenoxy auxins such as 2,4-D; (iv) β-triketone HPPD inhibitors such as mesotrione; and (v) any combination thereof, where seeds or plants comprising soybean event Gm_CSM63714 are planted in the area before, at the time of, or after applying the herbicide and the herbicide application prevents or inhibits weed growth and does not injure the soybean plants comprising event Gm_CSM63714, or has about less than about 5-20% injury. The plant growth area may or may not comprise weed seeds or plants at the time of herbicide application. The herbicide(s) used in the methods described herein can be applied alone, in sequential with or in combination with one or more herbicide(s) during the growing season. The herbicide(s) used in the methods described herein can be applied in combination with one or more herbicide(s) temporally (for example, as a tank mixture or in sequential applications), spatially (for example, at different times during the growing season including before and after soybean seed planting), or both. For example, a method for controlling weeds is provided that consists of planting seed comprising soybean event Gm_CSM63714 in an area and applying an herbicidally effective amount over the growing season of one or more of dicamba, glufosinate, 2,4-D and mesotrione alone or in any combination with another herbicide, for the purpose of controlling weeds in the area with no injury or less than about 5-20% injury to the plants containing soybean event Gm_CSM63714. Such application of herbicide(s) may be pre-planting (any time prior to planting seed comprising soybean event Gm_CSM63714, including for burn-down purposes, that is application to emerging or existing weeds prior to seed plant), pre-emergence (any time after seed comprising soybean event Gm_CSM63714 is planted and before plants comprising soybean event Gm_CSM63714 emerge), or post-emergence (any time after plants comprising soybean event Gm_CSM63714 emerge). Multiple applications of one or more herbicides, or a combination of herbicides together or individually, may be used over a growing season, for example, two applications (such as a pre-planting application and a post-emergence application, or a pre-emergence application and a post-emergence application) or three or more applications (such as a pre-planting application and two post-emergence applications).

Herbicide application in practicing the methods described herein may be at the recommended commercial rate or any fraction or multiple thereof, such as twice the recommended commercial rate. Herbicide rates may be expressed as pounds acid equivalent per acre (lb ae/acre), pounds active ingredient per acre (lb ai/acre) or pounds active ingredient per hectare (lb ai/ha), depending on the herbicide and the formulation. The use of acres in the herbicide application rates as provided herein is merely instructive; herbicide application rates in the equivalent dosages to any rate provided herein may be used for areas larger or smaller than an acre. The herbicide application comprises at least one herbicide selected from the group consisting of (i) inhibitors of glutamine synthetase such as glufosinate; (ii) benzoic acid auxins such as dicamba; (iii) phenoxy auxins such as 2,4-D; and (iv) β-triketone HPPD inhibitors such as mesotrione. The plant growth area may or may not comprise weed plants at the time of herbicide application. An herbicidally effective amount of glutamine synthetase inhibitors for use in the area for controlling weeds ranges from about 0.1 lb ae/acre to as much as about 10 lb ae/acre over a growing season (for example, glufosinate could be applied at a rate of about 0.4 lb ai/acre to about 1.6 lb ai/acre). An herbicidally effective amount of a benzoic acid herbicide for use in the area for controlling weeds ranges from about 0.1 lb ae/acre to as much as about 16 lb ae/acre over a growing season (for example, dicamba could be applied at a rate of about 0.5 lb ae/acre to about 2.0 lb ae/acre). An herbicidally effective amount of a phenoxy auxin herbicide for use in the area for controlling weeds ranges from about 0.1 lb ae/acre to as much as about 16 lb ae/acre over a growing season (for example, 2,4-D could be applied at a rate of about 0.5 lb ae/acre to about 4.0 lb ae/acre). An herbicidally effective amount of β-triketone HPPD inhibitors for use in the area for controlling weeds ranges from about 0.5 lb ae/ac to about 12 lb ae/ac over a growing season (for example, mesotrione could be applied at a rate of about 0.09 lb ae/acre to about 0.36 lb ae/acre).

Methods for controlling volunteer soybean comprising soybean event Gm_CSM63714 in an area for crop cultivation are provided. The methods comprise applying one or more herbicides effective on soybean event Gm_CSM63714 in the area for crop cultivation and having a mode of action other than benzoic auxins, phenoxy auxins, inhibitors of glutamine synthetase, and P-triketone HPPD inhibitors. Illustrative examples of such herbicides are atrazine, bronioxynil (3,5-di-bromo-4-hydroxybenzonitrile), clopyralid, pyrithiobac, isoxaflutole, topramezone, fluometuron, trifloxysulfuron, monosodium methyl arsenate (MSMA), inhibitors of protoporphyrinogen oxidase (PPO) (such as saflufenacil, flumioxazin, and sulfentrazone), and combinations of any thereof, wherein the herbicide application prevents growth of volunteer soybean comprising event Gm_CSM63714. For example, to control volunteer soybean comprising event Gm_CSM63714 in a corn cultivation field, topramezone, atrazine, clopyralid or isoxaflutole can be applied preemergence and/or postemergence. To control volunteer soybean comprising event Gm_CSM63714 in a cotton cultivation field, fluometuron can be applied preemergence, and trifloxysulfuron, pyrithiobac or monosodium methyl arsenate (MSMA) can be applied postemergence.

Methods for producing plants and seeds comprising soybean event Gm_CSM63714 are provided. Plants may be bred using any method known in the art. A progeny soybean plant comprising the event Gm_CSM63714 may be produced, for example, by selfing a parent plant or line comprising the event Gm_CSM63714, wherein such parent plant or line is homozygous or hemizygous for the event Gm_CSM63714, or by crossing a first parent plant or line comprising the event Gm_CSM63714, wherein such parent plant or line is homozygous or hemizygous for the event Gm_CSM63714, with a second parent plant or line having a different genotype or germplasm than the first parent line, wherein the second parent plant or line may or may not contain or comprise the event Gm_CSM63714. As described further herein, soybean event Gm_CSM63714 comprises four independent expression cassettes or transgenes encoding a dicamba monooxygenase (DMO), a phosphinothricin N-acetyltransferase (PAT), an alpha-ketoglutarate-dependent non-heme iron dioxygenase variant (FT_Tv7), and triketone dioxygenase (TDO), respectively. According to some embodiments, the transgenic soybean plant(s) comprising the event Gm_CSM63714 is/are tolerant to benzoic auxins such as dicamba, phenoxy auxins such as 2,4-D, inhibitors of glutamine synthetase such as glufosinate, β-triketone HPPD inhibitors such as mesotrione, or any combination thereof, relative to a non-transgenic control plant. Transgenic soybean plants used in these methods may be homozygous or heterozygous for the transgenes. Progeny plants produced by these methods may be varietal or hybrid plants; may be grown from seeds produced by soybean event Gm_CSM63714 containing plant and/or from seeds produced by a plant fertilized with pollen from a soybean event Gm_CSM63714 containing plant; and may be homozygous or heterozygous for the transgenes and/or event Gm_CSM63714. Progeny plants may be subsequently self-pollinated to generate a true breeding line of plants, i.e., plants homozygous for the transgene, or alternatively may be out-crossed, e.g., bred with another unrelated plant, to produce a varietal or a hybrid seed or plant.

As used herein, the terms “line”, “breeding line”, “genotype” or “germplasm” are used interchangeably to refers to a group of plants that show little or no genetic variation between individuals for at least one trait. Such “line”, “breeding line”, “genotype” or “germplasm” can be created by self-pollination for several generations, selection, or vegetative propagation from a single parent using tissue or cell culture techniques. As used herein, the terms “cultivar” and “variety” are synonymous and refer to a line used for commercial production.

The production of double haploids may also be used to produce soybean plants and seeds homozygous for event Gm_CSM63714 DNA in a breeding program. Double haploids are produced by the doubling of a set of chromosomes (1 N) from a heterozygous plant to produce a completely homozygous individual. For example, see Wan, et al., (1989) and U.S. Pat. No. 7,135,615. This can be advantageous because the process omits the generations of selfing needed to obtain a homozygous plant from a heterozygous source. One way of producing haploid and double haploid soybean plant comprising event Gm_CSM63714 is through anther culture of flowers comprising event Gm_CSM63714 (Khan et al., 2010). Other methods such as natural polyembryony, induction with irradiated pollen, crosses with polyploid plants or wild species, unfertilized ovule and microspore culture can also be applied to produce haploid and double haploid soybean plants comprising event Gm_CSM63714.

Seed and progeny plants made by the methods described herein comprise soybean event Gm_CSM63714. Application of one or more herbicide for which soybean event Gm_CSM63714 confers tolerance may be used to select progeny that comprise soybean event Gm_CSM63714. Alternatively, progeny may be analyzed using diagnostic methods to select for plants or seeds comprising soybean event Gm_CSM63714.

Soybean transgenic events are known to one of skill in the art; for example, a list of such traits is provided by the United States Department of Agriculture's (USDA) Animal and Plant Health Inspection Service (APHIS) and can be found on their website at www.aphis.usda.gov. Two or more transgenic events may thus be combined in a progeny seed or plant by crossing two parent plants each comprising one or more transgenic event(s), collecting progeny seed, and selecting for progeny seed or plants that contain the two or more transgenic events; these steps may then be repeated until the desired combination of transgenic events in a progeny is achieved. Back-crossing to a parental plant and out-crossing with a non-transgenic plant are also contemplated, as is vegetative propagation.

Methods of detecting the presence of DNA derived from a soybean plant, plant part, plant cell, or seed comprising soybean event Gm_CSM63714 in a sample are provided. One method comprises (i) extracting a sample comprising DNA from at least one soybean plant, plant part, plant cell, or seed; (ii) contacting the sample with at least one primer that is capable of producing DNA sequence specific to event Gm_CSM63714 DNA under conditions appropriate for DNA sequencing; (iii) performing a DNA sequencing reaction; and then (iv) confirming that the nucleotide sequence comprises a nucleotide sequence specific for event Gm_CSM63714, of the transgenic insert comprised therein, such as one selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, and SEQ ID NO:10.

Another method comprises (i) extracting a sample comprising DNA from at least one soybean plant, plant part, plant cell, or seed; (ii) contacting the sample with a primer pair that is capable of producing an amplicon from event Gm_CSM63714 DNA under conditions appropriate for DNA amplification; (iii) performing a DNA amplification reaction; and then (iv) detecting the amplicon molecule and/or confirming that the nucleotide sequence of the amplicon comprises a nucleotide sequence specific for event Gm_CSM63714, such as one selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, and SEQ ID NO:10. The amplicon should be one that is specific for event Gm_CSM63714, and comprises the junction at nucleotide positions 1000-1001, and/or nucleotide positions 11,196-11,197 of SEQ ID NO:10, such as an amplicon that comprises SEQ ID NO:1, or SEQ ID NO:2, or SEQ ID NO:3, or SEQ ID NO:4, or SEQ ID NO:5, or SEQ ID NO:6, or SEQ ID NO:7, or SEQ ID NO:8, or SEQ ID NO:9, or SEQ ID NO:10. The detection of a nucleotide sequence specific for event Gm_CSM63714 in the amplicon is determinative and/or diagnostic for the presence of the soybean event Gm_CSM63714 specific DNA in the sample. An illustrative primer pair that is capable of producing an amplicon from event Gm_CSM63714 DNA under conditions appropriate for DNA amplification is provided as SEQ ID NO:14 and SEQ ID NO:15. Other primer pairs may be readily designed by one of skill in the art to produce an amplicon diagnostic for soybean event Gm_CSM63714, wherein such a primer pair comprises at least one primer within the genomic region flanking the insert and a second primer within the insert, provided that any primer pair could be designed and used that produces an amplicon comprising a junction sequence and/or all or part of the insert or transgene sequence. Detection of an amplicon could be based on any suitable method, such as sequencing, determining fragment size or migration of the amplicon in a matrix or gel, or a hybridization-based method.

Another method of detecting the presence of DNA derived from a soybean plant, plant part, plant cell, or seed comprising soybean event Gm_CSM63714 in a sample consists of (i) extracting a sample comprising DNA from at least one soybean plant, plant part, plant cell, or seed; (ii) contacting the sample with a DNA probe specific for event Gm_CSM63714 DNA; (iii) allowing the probe and the DNA in the sample to hybridize under stringent hybridization conditions; and then (iv) detecting hybridization between the probe and the target DNA in the sample. An example of the sequence of a DNA probe that is specific for event Gm_CSM63714 is provided as SEQ ID NO:16. Other probes may be readily designed by one of skill in the art. Detection of probe hybridization to the DNA in the sample is diagnostic for the presence of soybean event Gm_CSM63714 specific DNA in the sample. Absence of hybridization is alternatively diagnostic of the absence of soybean event Gm_CSM63714 specific DNA in the sample.

Methods for determining the zygosity of the event and transgene with genomic DNA derived from at least one soybean plant, plant part, plant cell or seed comprising soybean event Gm_CSM63714 in a sample are provided. One method consists of (i) extracting a sample comprising DNA from at least soybean plant, plant part, plant cell or seed; (ii) contacting the sample with a first primer pair that is capable of producing a first amplicon diagnostic for event Gm_CSM63714; (iii) contacting the sample with a second primer pair that is capable of producing a second amplicon diagnostic for wild-type genomic DNA not comprising event Gm_CSM63714; (iv) performing a DNA amplification reaction; and then (v) detecting the amplicons, wherein the presence of only the first amplicon is diagnostic of a homozygous event Gm_CSM63714 DNA in the sample, the presence of both the first amplicon and the second amplicon is diagnostic of a soybean plant heterozygous for event Gm_CSM63714, and the presence of only the second amplicon is diagnostic for the absence of event Gm_CSM63714 DNA in the sample. Illustrative sets of primer pairs are presented as SEQ ID NO:14 and SEQ ID NO:15, which produce an amplicon diagnostic for event Gm_CSM63714; and SEQ ID NO:20 and SEQ ID NO:15, which produce an amplicon diagnostic for wild-type soybean genomic DNA not comprising event Gm_CSM63714. A set of probes can also be incorporated into such an amplification method to be used in a real-time PCR format using the primer pair sets described above. An illustrative set of probes are presented as SEQ ID NO:16 (diagnostic for the amplicon for the event Gm_CSM63714) and SEQ ID NO:21 (diagnostic for the amplicon for wild-type soybean genomic DNA not comprising event Gm_CSM63714).

Another method for determining zygosity consists of (i) extracting a sample comprising DNA from at least one soybean plant, plant part, plant cell or seed; (ii) contacting the sample with a probe set which contains at least a first probe that specifically hybridizes to event Gm_CSM63714 DNA and at least a second probe that specifically hybridizes to soybean genomic DNA that was disrupted by insertion of the heterologous DNA of event Gm_CSM63714 and does not hybridize to event Gm_CSM63714 DNA; (iii) hybridizing the probe set with the sample under stringent hybridization conditions, wherein detecting hybridization of only the first probe under the hybridization conditions is diagnostic for a homozygous soybean plant, plant part, plant cell or seed for event Gm_CSM63714 DNA in the sample; wherein detecting hybridization of both the first probe and the second probe under the hybridization conditions is diagnostic for a heterozygous soybean plant, plant part, plant cell or seed for event Gm_CSM63714 in the sample; and detecting hybridization of only the second probe under the hybridization conditions is diagnostic for the absence of event Gm_CSM63714 DNA in the sample.

Yet another method for determining zygosity consists of (i) extracting a sample comprising DNA from at least one soybean plant, plant part, plant cell or seed; (ii) contacting the sample with a first primer pair that is capable of producing a first amplicon diagnostic for event Gm_CSM63714; (iii) contacting the sample with a second primer pair that is capable of producing a second amplicon of an internal standard known to be single-copy and homozygous in the soybean plant; (iv) contacting the sample with a probe set which contains at least a first probe that specifically hybridizes to the first amplicon, and at least a second probe that specifically hybridizes to the second amplicon; (v) performing a DNA amplification reaction using real-time PCR and determining the cycle thresholds (Ct values) of the first and second amplicons; (vi) calculating the difference (ΔCt) between the Ct value of the first amplicon and the second amplicon; and (vii) determining zygosity, wherein a ΔCt of about zero (0) indicates homozygosity of the event or inserted T-DNA, and a ΔCt of about one (1) indicates heterozygosity of the event or inserted T-DNA. Heterozygous and homozygous events are differentiated by a ΔCt value unit of approximately one (1). Given the normal variability observed in real-time PCR due to multiple factors such as amplification efficiency and ideal annealing temperatures, the range of “about one (1)” is defined as a ΔCt of 0.75 to 1.25, and the range of “about zero (0)” is defined as a ΔCt of −0.25 to 0.25 (or of 0.0 to 0.25 if the ΔCt is measured as an absolute value). Primer pairs and probes for the above method for determining zygosity can amplify and detect amplicons from the transgene or event DNA and the internal DNA standard.

A DNA construct is provided comprising a first expression cassette, a second expression cassette, a third expression cassette and a fourth expression cassette, wherein the first expression cassette comprises in operable linkage i) ubiquitin (UB3) promoter, leader, and intron sequences from Arabidopsis thaliana, ii) a chloroplast transit peptide coding sequence of APG6 (Albino and Pale Green 6) from Arabidopsis thaliana, iii) a codon-optimized dicamba monooxygenase coding sequence (DMO) from Stenotrophomonas maltophilia for conferring dicamba tolerance, and iv) a 3′ UTR sequence of the aluminum-induced Sali3-2 protein from Medicago truncatula; the second expression cassette comprises in operable linkage i) promoter and an intron sequences derived from multiple promoter and intron sequences from Arabidopsis thaliana, ii) a codon-optimized phosphinothricin N-acetyltransferase (PAT) coding sequence from Streptomyces viridochromogene for conferring tolerance to glutamine synthetase inhibitors, and iii) a 3′ UTR of a small heat shock protein (Hsp20) from Medicago truncatula; the third expression cassette comprises in operable linkage i) polyubiquitin (UBQ10) promoter, leader, and intron sequences from Arabidopsis thaliana, ii) a codon-optimized alpha-ketoglutarate-dependent non-heme iron dioxygenase variant coding sequence (FT_Tv7, also referred to as FT_T.1) from Sphingobium herbicidovorans for conferring tolerance to phenoxy auxins such as 2,4-D, and iii) a 3′ UTR sequence of a putative protein from Medicago truncatula, and the fourth expression cassette comprises in operable linkage i) promoter, leader, and intron sequences derived from multiple promoter, leader and intron sequences from Arabidopsis thaliana, ii) a codon-optimized coding sequence of the triketone dioxygenase (TDO) from Oryza sativa for conferring tolerance to β-triketone HPPD inhibitors such as mesotrione, and iii) a 3′ UTR sequence derived from multiple 3′ UTR sequences from Zea mays. The nucleotide sequences of the four expression cassettes were comprised in SEQ ID NO:9 and SED ID NO:10 of soybean event Gm_CSM63714. Expression of the DMO, PAT, FT_Tv7 and TDO in transgenic plants confers tolerance to herbicides with at least four different modes of action. For example, plants, plant parts, plant cells or seeds containing or comprising soybean event Gm_CSM63714 are tolerant to dicamba (a benzoic acid herbicide), glufosinate (a glutamine synthetase inhibitor), 2,4-D (a phenoxy herbicide), and mesotrione (a β-triketone HPPD inhibitor).

Any of the DNA constructs or transgenic inserts described herein can further comprise at its 5′ or 3′ end at least 50 contiguous nucleotides of SEQ ID NO:11 or SEQ ID NO:98, or at least 50 contiguous nucleotides of SEQ ID NO:12 or SEQ ID NO:99. Alternatively, any of the DNA constructs or transgenic inserts described herein can further comprise at its 5′ or 3′ end at least 100 contiguous nucleotides, at least 150 contiguous nucleotides, at least 200 contiguous nucleotides, at least 250 contiguous nucleotides, at least 300 contiguous nucleotides, at least 350 contiguous nucleotides, at least 400 contiguous nucleotides, at least 450 contiguous nucleotides, or at least 500 nucleotides of SEQ ID NO: 11 or SEQ ID NO: 98, or at least 100 contiguous nucleotides, at least 150 contiguous nucleotides, at least 200 contiguous nucleotides, at least 250 contiguous nucleotides, at least 300 contiguous nucleotides, at least 350 contiguous nucleotides, at least 400 contiguous nucleotides, at least 450 contiguous nucleotides, or at least 500 nucleotides of SEQ ID NO: 12 or SEQ ID NO: 99.

SEQ ID NOs.:11 and 12 are 1,000 nucleotide sequences representing soybean genomic DNA that flanks the transgenic insert at the 5′ and 3′ ends of the insert in soybean event Gm_CSM63714, respectively. SEQ ID NO:11 and SEQ ID NO:12 have been validated by sequencing, as further described in Example 5 hereinbelow. SEQ ID NOs:98 and 99 are 5,000 nucleotide sequences representing soybean genomic DNA that flanks the transgenic insert at the 5′ and 3′ ends of the insert, respectively. Nucleotides 4,001-5,000 of SEQ ID NO:98 are identical to nucleotides 1-1,000 of SEQ ID NO: 11. The remaining nucleotides of SEQ ID NO: 98 (nucleotides 1-4,000) are based on the genomic sequence of the Williams 82 soybean cultivar (Schmutz et al., 2010). Similarly, nucleotides 1-1000 of SEQ ID NO: 99 are identical to nucleotides 1-1,000 of SEQ NO: 12. The remaining nucleotides of SEQ ID NO: 99 (nucleotides 1,001-5,000) are based on the genomic sequence of the Williams 82 soybean cultivar.

The at least 50 contiguous nucleotides of SEQ ID NO:11 or SEQ ID NO:98 at the 5′ end of the DNA construct or transgenic insertion may be immediately adjacent to and upstream (on the 5′ end) of the transgenic insertion, or may not be immediately adjacent to, but further upstream (on the 5′ end) and within about 5000 nucleotides, within about 3000 nucleotides, or within about 1000 nucleotides of the transgenic insertion. Likewise, the at least 50 contiguous nucleotides of SEQ ID NO:12 or SEQ ID NO:99 at the 3′ end of the DNA construct or transgenic insertion may be immediately adjacent to and downstream (on the 3′ end) of the transgenic insertion, or may not be immediately adjacent to but further downstream (on the 3′ end) and within about 5000 nucleotides, within about 3000 nucleotides, or within about 1000 nucleotides of the transgenic insertion. Illustrative examples of sequences comprising 50 contiguous nucleotides of SEQ ID NO:11 are provided in SEQ ID NOs:58-77. Illustrative examples of 50 contiguous nucleotides of SEQ ID NO:12 are provided in SEQ ID NOs:78-97. Illustrative examples of 50 contiguous nucleotides of SEQ ID NO:98 are provided in SEQ ID NOs: 100-139. Illustrative examples of 50 contiguous nucleotides of SEQ ID NO:99 are provided in SEQ ID NOs: 140-179. However, any sequence comprising at least 50 contiguous nucleotides of SEQ ID NO: 11 or SEQ ID NO: 98, or at least 50 contiguous nucleotides of SEQ ID NO: 12 or SEQ ID NO: 99 is within the scope of the present disclosure.

In addition, a DNA construct comprising a first expression cassette, a second expression cassette, a third expression cassette, and a fourth expression cassette is provided. The first expression cassette comprises a dicamba monooxygenase coding sequence, the second expression cassette comprises a phospinothricin N-acetyltransferase (PAT) coding sequence, the third expression cassette comprises an alpha-ketoglutarate-dependent non-heme iron dioxygenase variant coding sequence (FT_Tv7) capable of degrading 2,4-D, and the fourth expression cassette comprises a triketone dioxygenase (TDO) coding sequence. The DNA construct further comprises at the 5′ and/or 3′ end of the construct (i) at least 50 contiguous nucleotides of SEQ ID NO: 11 or SEQ ID NO:98; and/or (ii) at least 50 contiguous nucleotides of SEQ ID NO: 12 or SEQ ID NO:99.

A further DNA construct is provided. The DNA construct comprises a polynucleotide having a sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO: 9. The DNA construct further comprises at the 5′ and/or 3′ end of the construct (i) at least 50 contiguous nucleotides of SEQ ID NO: 11 or 98; and/or (ii) at least 50 contiguous nucleotides of SEQ ID NO: 12 or 99.

For example, any of the DNA constructs can comprise at the 5′ end of said construct one or more nucleotide sequences selected from SEQ ID NOs:58-77 and SEQ ID NOs:100-139. Alternatively or in addition, any of the DNA constructs can comprise at the 3′ end of said construct one or more nucleotide sequences selected from SEQ ID NOs:78-97 and SEQ ID NOs:140-179.

Soybean plants, plant cells, plant parts, and plant seeds comprising any of the DNA constructs described herein are also provided.

Also provided are soybean plants, plant cells, plant parts, and plant seeds comprising a recombinant DNA construct integrated in chromosome 13, wherein the recombinant DNA construct confers tolerance to at least one herbicide selected from the group consisting of benzoic acid auxins, phenoxy auxins, glutamine synthetase inhibitors, β-triketone HPPD inhibitors, and combinations of any thereof. The recombinant DNA construct is integrated in a position of said chromosome flanked by at least 50 contiguous nucleotides of SEQ ID NO:11 98 and 50 contiguous nucleotides of SEQ ID NO:12 or 99. The benzoic acid auxin can comprise dicamba; the phenoxy auxin can comprise 2,4-D; the glutamine synthetase inhibitor comprises glufosinate; and the 0-triketone HPPD inhibitor can be selected from the group consisting of mesotrione, benzobicyclon (BBC), tembotrione, sulcotrione, tefuryltrione, and combinations of any thereof. The at least 50 contiguous nucleotides of SEQ ID NO: 11 or 98 can comprise one or more nucleotide sequences selected from SEQ ID NOs:58-77 or SEQ ID NOs:100-139, and the at least 50 contiguous nucleotides of SEQ ID NO: 12 or 99 can comprise one or more nucleotide sequences selected from SEQ ID NOs:78-97 or SEQ ID NOs:140-179.

Methods of improving tolerance to herbicides are provided. The methods consist of i) inserting a DNA construct comprising a first expression cassette, a second expression cassette, a third expression cassette and a fourth expression cassette, as described herein, into the genome of a plant cell, ii) generating a plant from the plant cell; and iii) selecting a regenerated transgenic plant comprising the DNA construct. Transgenic plants produced by the methods comprise a unique combination of four transgene expression cassettes in terms of orientation and positive relative to each other, each with a unique combination of expression elements for optimal expression of the transgenes. Furthermore, transgenic plants produced by the methods as described herein acquire tolerance to herbicides with four different herbicide modes of action. Selecting the regenerated plant comprising the DNA construct may be done using DNA or protein detection methods as described herein. Alternatively or additionally, selecting may comprise treating the transgenic plant or plant cell with an effective amount of at least one herbicide selected from the group consisting of benzoic acid auxins such as dicamba, phenoxy auxins such as 2,4-D, inhibitors of glutamine synthetase such as glufosinate, β-triketone HPPD inhibitors such as mesotrione, and any combination thereof.

Methods of controlling, preventing, or reducing the development of herbicide-tolerant weeds are provided. The methods comprise: a) cultivating in a crop growing environment a soybean plant comprising a DNA construct or transgenes of the present disclosure or event Gm_CSM63714 that provide tolerance to herbicides with at least three different herbicide modes of action at a single genomic location, and b) applying to the crop growing environment at least one herbicide selected from the group consisting of dicamba, glufosinate, 2,4-D, a β-triketone HPPD inhibitor such as mesotrione, and any combination thereof, wherein the soybean plant is tolerant to the at least one herbicide. The at least three different herbicide modes of action are selected from inhibition of glutamine synthetase, benzoic acid auxins, phenoxy auxins, and inhibition of HPPD. All four of these different herbicide modes of action can be provided by the DNA construct or transgenes. For example, soybean plants grown from the seeds comprising the DNA construct or transgenes of the present disclosure, or event Gm_CSM63714 are tolerant to dicamba, 2,4-D, glufosinate, mesotrione, or any combination thereof.

The soybean plants, plant seeds, plant parts, or plant cells may further comprise at least one additional transgene (such as the 5-enolpyruvylshikimate-3-phosphate synthase (EPSPS) gene from Agrobacterium CP4 strain or another EPSPS that confers tolerance to glyphosate) for additional herbicide mode(s) of action. An illustrative EPSPS coding sequence and its corresponding amino acid sequence from Agrobacterium CP4 strain are provided as SEQ ID NO:56 and SEQ ID NO:57, respectively.

The herbicide(s) used in the methods described herein can be applied alone, sequentially with or in combination with one or more herbicide(s) during the growing season. The herbicide(s) used in the methods described herein can be applied in combination with one or more herbicide(s) temporally (for example, as a tank mixture or in sequential applications), spatially (for example, at different times during the growing season including before and after soybean seed planting), or both. For example, a method for controlling the development of herbicide resistance in weeds is provided that consists of planting seed comprising soybean event Gm_CSM63714 in an area and applying an herbicidally effective amount over the growing season of one or more of benzoic acid auxins such as dicamba, phenoxy auxins such as 2,4-D, inhibitors of glutamine synthetase such as glufosinate, and β-triketone HPPD inhibitors such as mesotrione alone or in any combination with another herbicide, for the purpose of controlling the development of herbicide resistance in weeds in the area. Such application of herbicide(s) may be pre-planting (any time prior to planting seed comprising soybean event Gm_CSM63714, including for burn-down purposes, that is application to emerging or existing weeds prior to seed plant), pre-emergence (any time after seed comprising soybean event Gm_CSM63714 is planted and before plants comprising soybean event Gm_CSM63714 emerge), or post-emergence (any time after plants comprising soybean event Gm_CSM63714 emerge). Multiple applications of one or more herbicides, or a combination of herbicides together or individually, may be used over a growing season, for example, two applications (such as a pre-planting application and a post-emergence application, or a pre-emergence application and a post-emergence application) or three or more applications (such as a pre-planting application and two post-emergence applications).

Also provided are methods of reducing loci for soybean breeding by inserting multiple transgenes at a single genomic location to provide four different modes of action for herbicide tolerance. Soybean event Gm_CSM63714 contains or comprises a transgenic insert comprising four independent transgene cassettes: a first expression cassette encoding a dicamba monooxygenase coding sequence (DMO), a second expression cassette encoding a phosphinothricin N-acetyltransferase (PAT), a third expression cassette encoding an alpha-ketoglutarate-dependent non-heme iron dioxygenase variant (FT_Tv7), and a fourth expression cassette encoding a triketone dioxygenase (TDO). These four transgene cassettes were inserted at a single genomic location as a contiguous polynucleotide or DNA molecule or single molecularly linked transgenic insert, and provide a commercial level of tolerance to at least one herbicide for each herbicide mode of action in a field, such as dicamba, glufosinate, 2,4-D, mesotrione, and any combination thereof. The nucleotide sequences of the four expression cassettes are comprised in SEQ ID NO:9 and SED ID NO:10. As used herein, the term “commercial level” in reference to an herbicide refers to the recommended commercial rate (1×) for herbicide application for a specific herbicide. For example, a 1× rate of dicamba is 1 lb/acre pre-emergence and 0.5 lb/acre post-emergence; a 1× rate of 2,4-D is 1 lb/acre post-emergence; a 1× rate of glufosinate is 0.8 lb/acre; and a 1× rate of mesotrione is 0.18 lb/acre pre-emergence, and 0.09 lb/acre post-emergence. As used herein, “commercial level tolerance” refers to tolerance to one or more herbicides at the recommended commercial rates or higher as a result of transgene expression from one or more of the four expression cassettes in plants comprising event Gm_CSM63714.

Soybean event Gm_CSM63714 with the unique characteristics such as s single insertion site, stable integration and expression of the DMO, PAT, FT_Tv7 and TDO transgenes, consistent and superior combinations of efficacy, including herbicide tolerance and agronomic performance, in and across multiple environment conditions in different geographies can be bred or introgressed into elite lines or varieties as a single locus by conventional breeding methods, and maintained over subsequent generations following Mendelian inheritance of single locus. Therefore, the methods of the present disclosure allow for rapid trait integration of the multiple transgenes on segregating material, saving time and resources in a breeding program and enabling rapid development of lines, compared to cases where the individual transgenes are inserted into two or more loci, necessitating tedious and laborious multiple crosses over multiple generations to select for plants comprising the multiple genes. The newly introgressed or integrated DNA molecule or polynucleotide of event Gm_CSM63714 comprising SEQ ID NO:9 and/or SEQ ID NO:10 will maintain the expression characteristics of the transgenes, and the genomic flanking sequences and chromosomal location, where it will confer tolerance to the at least one herbicide for each herbicide mode of action in a field, such as dicamba, glufosinate, 2,4-D, or mesotrione, and any combination thereof.

### DEPOSIT INFORMATION

A deposit of a representative sample of soybean seed comprising event Gm_CSM63714 has been made on Aug. 10, 2021 according to the Budapest Treaty with the American Type Culture Collection (ATCC) Patent Repository having an address at 10801 University Boulevard, Manassas, Va., 20110, USA. The ATCC Patent Deposit Designation (accession number) for seeds comprising soybean event Gm_CSM63714 is Accession No. PTA-127099. Access to the deposits will be available during the pendency of the application to the Commissioner of Patents and Trademarks and persons determined by the Commissioner to be entitled thereto upon request. Upon issuance of the patent, all restrictions upon availability to the public will be irrevocably removed. The deposit will be maintained in the depository for a period of thirty (30) years, or five (5) years after the last request, or for the effective life of the patent, whichever is longer, and will be replaced as necessary during that period.

### EXAMPLES

The following examples are included to more fully describe the invention. Summarized are the construction and testing of 72 transformation constructs containing single gene cassettes for tolerance to dicamba, glufosinate, 2,4-D, and mesotrione, the construction and testing of six different transformation constructs containing all four of these gene cassettes, the production and testing of over 25,760 unique transformation events, and the analysis of thousands of individual plants over multiple seasons through rigorous molecular characterization, efficacy and agronomic testing both in a controlled environment and in field trials, leading to the creation, identification, and ultimate selection of the soybean event Gm_CSM63714.

The Examples illustrate certain embodiments of the present disclosure. It should be appreciated by those of skilled in the art that many modifications can be made in the specific examples which are disclosed and still obtain a similar result. Certain agents which are both chemically and physiologically related may be substituted for the agents described herein while achieving the same or similar results. All such substitutions and modifications apparent to those skilled in the art are deemed to be within the scope of the invention.

### Example 1: Expression Cassette Testing, Construct Design, Plant Testing and Construct Selection

This example describes the design of six different expression constructs for tolerance to dicamba, glufosinate, 2,4-D, and mesotrione herbicides through a vector stack, the production of 25,760 unique soybean events, and testing and molecular analysis of the resulting transgenic soybean plants for selection of the lead construct.

Transgene expression and performance in transgenic plants may be influenced by many factors. These include but are not limited to: 1) the expression elements used to drive the expression of the transgene in the expression cassette, and their interactions among themselves and with the transgene; 2) the relative position and orientation of different expression cassettes when the transgenic insert comprises multiple expression cassettes, each carrying a different transgene conferring a distinct trait; and 3) the genomic location of the transgenic insertion, also known as positional effect. A commercially useful multi-gene transgenic event requires that each of the transgenes in the transgenic insert expresses in the manner necessary for that trait to be successful, i.e., optimal transgene expression and performance across different tissues and developmental stages, in various germplasms, and under different growth conditions.

For these reasons, it is often necessary to create and screen a large number of constructs and transformation events in order to identify a construct (the lead construct), and then an event (the lead event), which demonstrates optimal expression and performance of the transgenes without phenotypic and agronomic off-types such as yield drag. Prior to such studies, it is not possible to determine whether a particular beneficial event phenotype or performance can be obtained.

In an initial proof-of-concept and early development stage study, over several years and multiple growing seasons, a total of 72 different constructs each containing single gene cassette of DMO, PAT, FT_Tv7 or TDO with different expression element combinations were designed. Genes encoding protein variants for TDO and FT_Tv7 were also tested. In addition, one DMO and PAT double stack construct, one DMO, PAT and TDO triple stack construct, forty-one DMO, PAT and FT_Tv7 triple stack constructs, and four DMO, PAT, FT_Tv7 and TDO quad stack constructs were designed. The individual gene cassettes in these constructs contained different expression element combinations and varied in relative positions and orientations of the transgene cassettes. These vectors were constructed and transformed into soybean seed-derived dry excised explants through Agrobacterium-mediated transformation using methods known in the art. Thousands of transformed plants were regenerated and tested in the greenhouse and in field trials for optimal protein expression through measurement of protein levels by ELISA, and for trait efficacy through herbicide spray treatment. Based on these results, top performing individual expression cassettes were identified, each containing individual transgenes driven by combinations of expression elements. From these individual expression cassettes, six different constructs were designed, each containing four expression cassettes (encoding DMO, PAT, FT_Tv7, and TDO, respectively). These six constructs contained different expression cassette combinations, varying by expression elements, protein coding sequence for TDO and FT, relative position and orientation of the transgene cassettes.

The six four-expression cassette constructs were cloned into plant transformation vectors, and introduced into soybean seed-derived dry excised explants through Agrobacterium-mediated transformation using methods known in the art. The transformation vectors harbored two T-DNAs, a first T-DNA molecule which was bordered by a first right border DNA sequence (RB) and a first left border DNA sequence (LB) linked to a second T-DNA molecule which is bordered by a second right border DNA sequence and a second left border DNA sequence. The two T-DNA molecules were positioned in the DNA construct to have an orientation with respect to each other consisting of LB-second T-DNA-RB-RB-first T-DNA-LB, wherein the first RB and second RB are linked. The first T-DNA contained four expression cassettes encoding DMO, PAT, FT_Tv7, and TDO, respectively for conferring herbicide tolerance. The second T-DNA contained two expression cassettes, one encoding aadA targeted to the chloroplast for selection of transgenic events resistant to spectinomycin and/or streptomycin, and one encoding sucrose phosphorylase (GenBank Accession AE009432) under the control of a seed specific promoter to function as a marker for identification of the presence of the selectable marker. A total of 25,760 unique transformation events were produced, each made by a random insertion of the transgenic insert into the soybean genome. R0 plants were subsequently regenerated from the transgenic events. Rooted plants with normal phenotype were transferred to soil for growth and further assessment.

Out of the 25,760 R0 plants generated, 22,834 were analyzed for desirable molecular characteristics. These include: 1) the transgenic insert is single copy and intact; 2) no transformation vector backbone sequence is present; and 3) the two T-DNAs are not linked (and therefore can be segregated in a later generation). Plants with the desirable molecular characteristics and sufficient seed set were advanced for herbicide tolerance efficacy testing.

As shown in Table 2, a total of 288 unique events containing a single copy of the intact transgenic insert without the presence of the transformation vector backbone, and lack of the second T-DNA sequence were advanced to R1 nursery for R1 seed production. Events with unacceptable segregation or that were unable to produce sufficient seeds were discarded. As a result, a total of 224 homozygous events were assessed in the greenhouse for herbicide tolerance. The plants were sprayed with 1.5 pound/acre (lb/acre) of 2,4-D at V3 stage, followed by a tank mix of 1.06 lb/acre of glufosinate and 1 lb/acre of dicamba at V6 stage, followed by 0.19 lb/acre of mesotrione at R1 stage, and were assessed visually for injury 7 days after each treatment, with 0% representing no injury and 100% representing complete plant death. Events with >35% injury were discarded. A few additional events were also dropped due to capacity limitations. From the greenhouse spray results, a total of 68 events were advanced to first season field trials.

TABLE 2
Summary of events from the six transformation constructs

| | | | # R1 Events | # Events |
|---|---|---|---|---|
| | | # Events | Avanced to | Advanced to |
| | # R0 | Advanced to | Greenhouse | 1st Season |
| Construct | Events | R1 Nursery | Testing | Field Trials |
| GmHT4-1 | 4886 | 50 | 41 | 17 |
| GmHT4-2 | 4247 | 47 | 41 | 18 |
| GmHT4-3 | 4685 | 54 | 47 | 15 |
| GmHT4-4 | 4751 | 43 | 34 | 18 |
| GmHT4-5 | 2678 | 37 | 28 | 0 |
| GmHT4-6 | 4513 | 57 | 33 | 0 |
| Total | 25760 | 288 | 224 | 68 |

### Example 2: Field Trials

Field trials were conducted over multiple seasons/years and across many locations in different geographies to evaluate the performance of the constructs/events, and for selecting the best performing construct/event. The field trials comprised efficacy trials for herbicide tolerance, and agronomic trials for yield performance of the events. The performance of many individual plants for each event in each field trial was analyzed as a set. Each event was thus represented by many individual plants. This allowed the performance of each event to be analyzed under many different conditions, in different locations and geographies, and for a variety of properties. Field trials were conducted on homozygous plants to assess trait efficacy for tolerance to commercial rates of dicamba, glufosinate, 2,4-D and mesotrione herbicides, and agronomic performance.

### First Season Field Trials

A total of 68 events from four constructs were tested in the first season field trials in North America (see Table 2). In the efficacy trials, the events were evaluated for tolerance to dicamba, glufosinate, 2,4-D and mesotrione. Herbicide treatments are summarized in Table 3 and consisted of dicamba at 2 lb/acre (2×) pre-emergence, 1 lb/acre (2×) at V3 and R1 stages; mesotrione at 0.36 lb/acre (2×) pre-emergence, 0.18 lb/acre (2×) at V3 and R1 stages; 2,4-D at 2 lb/acre (2×) at V3 and R1 stages; and glufosinate at 1.6 lb/acre (2×) at V3 and V6 stages. All applications were single chemistries. Plant height, flowering time, maturity and yield data were collected.

TABLE 3
Herbicide treatments in the first season field trials

Treatment Stage and Rate (pound/acre)

| Herbicide | Pre-emergence | V3 | V6 | R1 |
|---|---|---|---|---|
| Dicamba | 2 | 1 | — | 1 |
| Glufosinate | — | 1.6 | 1.6 | — |
| 2,4-D | — | 2 | — | 2 |
| Mesotrione | 0.36 | 0.18 | — | 0.18 |

In the agronomy trials, the same events were evaluated for plant height, flowering time, maturity, and yield performance (presented as bushel per acre). Fields were maintained weed-free by hand hoeing or by use of conventional herbicides.

The agronomy and efficacy trials were conducted using a Group Unbalanced Block Design (GUBD2). The efficacy trials included nine locations with three replications and five treatment blocks. These consisted of an untreated block along with blocks for herbicide treatments at 2×maximum application rates as prescribed on herbicide labels. Turbo TeeJet Induction (TTI) nozzles were used for all herbicide treatments. The agronomy trials were conducted at 18 locations with two replications. Plots were two rows of twelve feet in length, with a 30-inch row spacing and a three-foot alley. Trial maintenance was designed to optimize grain yield. Herbicide-treated event plots were compared to untreated event plots. Visual injury data were collected for the efficacy trials approximately seven days after post-emergent herbicide applications or 14 days after pre-emergent applications.

All data from both agronomy and efficacy trials were subject to analysis of variance and means separated at LSD (Least Significant Difference) at alpha 0.05. Statistical analysis was automated using ASReml software for mixed model fitting. Events were prioritized for further testing based on yield performance and crop injury. In the efficacy trials, yield of herbicide treated events was compared to that of the untreated events. In the agronomy trials, yield of events was compared to that of the untransformed (wild-type) control plants. Typically, events were not advanced for further testing if the yield was significantly negative at p value≤0.05 compared to the yield of untreated controls after herbicide treatment, or to the yield of the wild-type controls.

The results of the efficacy and agronomy trials for the individual events of the four constructs are summarized in Table 4. A black box indicates a significantly negative performance of the event for the treatment, whereas a grey box indicates no significant difference when compared to the wild-type or untreated control. Yield (bu/A or bushel/acre) refers to yield performance measured in bushel per acre compared to the wild-type control in the agronomy trials. Yield-Dicamba, Yield-Glufosinate, Yield-2,4-D, or Yield-Mesotrione refers to yield in bushel per acre after dicamba, glufosinate, 2,4-D, or mesotrione application compared to the untreated controls, respectively. Dicamba Injury, Glufosinate Injury, 2,4-D Injury or Mesotrione Injury refers to % injury after the respective herbicide application compared to the untreated controls. While none of the events showed high levels of injury following individual herbicide treatments, more than half of the events showed reduced yield (bushel/acre or lb/A) compared to the controls. Based on the first season field trial results, all events from constructs GmHT4-1 and GmHT4-2 were dropped, and seven events from construct GmHT4-3 and eight events from construct GmHT4-4 were advanced to the second season field trials.

### Second Season Field Trials

In the second season field trials conducted in South America, the events were evaluated for tolerance to dicamba, glufosinate, 2,4-D, and mesotrione in efficacy trials. Seven events from construct GmHT4-3 and eight events from construct GmHT4-4 were advanced from the first season trial. However, since the second season field trial started before the first season field trial results were obtained, all 33 events from constructs GmHT4-3 and GmHT4-4 were planted, and therefore tested in the second season trials. Herbicide treatments are summarized in Table 3 above. All applications were single chemistries. The efficacy trial was conducted across six locations using a Group Unbalanced Block Design (GUBD2), with three replications and five treatment blocks. These consisted of an untreated block along with blocks for herbicide treatments at 2×maximum application rates as prescribed in herbicide labels. Turbo TeeJet Induction (TTI) nozzles were used for all herbicide treatments. Herbicide-treated event plots were compared to untreated event plots. The agronomy trial was conducted across twelve locations and events were compared to the untransformed control.

Plots were four rows of four meters in length, with a 0.525-meter row spacing and a one-meter alley. Trial maintenance was designed to optimize grain yield. Visual injury data was collected for the efficacy trials approximately seven days after post-emergent herbicide applications or 14 days after pre-emergent applications.

All data from both agronomic and efficacy trials were subject to analysis of variance and means separated at LSD at alpha 0.05. Statistical analysis was automated using ASReml software for mixed model fitting. Events were prioritized for further testing based on yield performance and crop injury. In the efficacy trials, yield of herbicide treated events were compared to that of the untreated events. In the agronomy trials, yield of events was compared to that of the untransformed (wild-type) control plants.

The results of the second season efficacy and agronomy trials for the individual events of the two constructs are summarized in Table 5. A dark box indicates a significantly negative performance of the event for the treatment, whereas a grey box indicates no significant difference when compared to the wild-type or untreated control. A white box indicates that the event was not assessed in this trial. The results of the efficacy trial were consistent with the results of the first season trials, i.e., none of the events tested showed significant injury following individual herbicide treatments. One event for construct GmHT4-3 and two events for construct GmHT4-4 showed significant yield reduction compared to the wild-type controls in the agronomic trials. Yield (bu/A) refers to yield performance measured in bushel per acre compared to the wild-type control in the agronomy trials. Yield-Dicamba, Yield-Glufosinate, Yield-2,4-D, or Yield-Mesotrione refers to yield in bushel per acre after dicamba, glufosinate, 2,4-D, or mesotrione application compared to the untreated controls, respectively. Similarly, Dicamba Injury, Glufosinate Injury, 2,4-D Injury, or Mesotrione Injury refers to % injury after the respective herbicide application compared to the untreated controls.

Based on composite data from the first and second season field trials and the in-depth molecular characterization of the events, seven events from each of the constructs GmHT4-3 and GmHT4-4 were advanced to the third season field trials.

### Third and Fourth Season Field Trials

The third and fourth season field trials were conducted in North America and in South America, respectively. The third season field trials contained 7 events for construct GmHT4-3 and 7 events for construct GmHT4-4, and followed the same trial design, individual herbicide application method, rates and development stages, and statistical analysis method as in the first season field trials. The fourth season field trials contained 5-7 events for construct GmHT4-3 (five events in the efficacy trials, and 7 events in the agronomic trials) and 7 events for construct GmHT4-4, and followed the same trial design, individual herbicide application method, rates and development stages, and statistical analysis method as in the second season field trials. In both the third and the fourth season trials, data were collected for emergence, vigor, date of first flower, plant height, maturity, and yield. In addition, visual injury data were collected for the efficacy trials approximately seven days after post-emergent herbicide applications or fourteen days after pre-emergent applications.

In the third season efficacy trials, all events demonstrated tolerance to individual herbicide applications, most with less than 10% injury when compared to the untreated controls, although some events had >10% injury, especially at the V3 stage. One of the events for construct GmHT4-3 (GmHT4-3-3) was not tested due to lack of sufficient seeds. The results of the efficacy trials are summarized in Table 6. Mean Yield refers to cross-location yield in bushel per acre for the event, untreated, or treated with dicamba, glufosinate, 2,4-D, or mesotrione.

TABLE 6
Summary of event performance in the third season efficacy trials

| | | Untreated | Dicamba | | Gufosinate | | 2,4-D | | Mesotrione | |
|---|---|---|---|---|---|---|---|---|---|---|
| | | Mean | Mean | | Mean | | Mean | | Mean | |
| Construct | Event | Yield | Yield | p_Value | Yield | p_Value | Yield | p_Value | Yield | p_Value |
| GmHT4-3 | GmHT4-3-1 | 56.2 | 54.2 | 0.10 | 54.8 | 0.26 | 56.4 | 0.89 | 55.7 | 0.70 |
| | GmHT4-3-2 | 57.9 | 56.5 | 0.28 | 56.6 | 0.35 | 58.7 | 0.54 | 58.5 | 0.63 |
| | GmHT4-3-3 | — | — | — | — | — | — | — | — | — |
| | GmHT4-3-4 | 59.0 | 56.0 | 0.02 | 55.9 | 0.02 | 58.3 | 0.60 | 58.0 | 0.45 |
| | GmHT4-3-5 | 58.9 | 54.9 | 0.00 | 54.9 | 0.00 | 58.3 | 0.60 | 57.2 | 0.17 |
| | GmHT4-3-6 | 59.2 | 56.6 | 0.05 | 58.0 | 0.35 | 58.3 | 0.51 | 58.4 | 0.58 |
| | GmHT4-3-7 | 59.9 | 57.4 | 0.06 | 57.3 | 0.06 | 59.0 | 0.50 | 58.6 | 0.33 |
| GmHT4-4 | GmHT4-4-1 | 58.7 | 58.0 | 0.62 | 57.3 | 0.29 | 55.1 | 0.00 | 58.8 | 0.94 |
| | GmHT4-4-2 | 60.8 | 60.6 | 0.89 | 59.2 | 0.22 | 57.7 | 0.02 | 61.5 | 0.61 |
| | (Gm_CSM63714) | | | | | | | | | |
| | GmHT4-4-3 | 61.6 | 60.6 | 0.45 | 59.0 | 0.05 | 59.7 | 0.14 | 59.1 | 0.06 |
| | GmHT4-4-4 | 60.6 | 61.0 | 0.76 | 59.4 | 0.37 | 57.5 | 0.02 | 60.1 | 0.70 |
| | GmHT4-4-5 | 61.2 | 59.5 | 0.19 | 60.6 | 0.65 | 57.8 | 0.01 | 59.4 | 0.17 |
| | GmHT4-4-6 | 62.2 | 57.8 | 0.00 | 59.4 | 0.08 | 57.7 | 0.00 | 59.6 | 0.08 |
| | GmHT4-4-7 | 60.4 | 60.3 | 0.91 | 58.3 | 0.10 | 56.5 | 0.00 | 60.0 | 0.74 |

The performance for the 7 events from each of constructs GmHT4-3 and GmHT4-4 in the agronomic trials is summarized in Table 7. Mean Yield refers to cross location yield in bushel per acre for the event. The results demonstrate that the presence of the transgenic insert had no negative impact on these events, which had comparable yield compared to the wild-type control.

TABLE 7
Summary of event performance in the third season agronomic trials

| Construct | Event | Mean Yield | p_Value |
|---|---|---|---|
| GmHT4-3 | GmHT4-3-1 | 52.8 | 0.08 |
| | GmHT4-3-2 | 54.1 | 0.18 |
| | GmHT4-3-3 | 53.5 | 0.12 |
| | GmHT4-3-4 | 53.0 | 0.09 |
| | GmHT4-3-5 | 52.6 | 0.07 |
| | GmHT4-3-6 | 55.5 | 0.37 |
| | GmHT4-3-7 | 54.8 | 0.26 |
| GmHT4-4 | GmHT4-4-1 | 53.4 | 0.12 |
| | GmHT4-4-2 | 56.5 | 0.55 |
| | (Gm_CSM6371 | | |
| | GmHT4-4-3 | 56.0 | 0.46 |
| | GmHT4-4-4 | 57.4 | 0.76 |
| | GmHT4-4-5 | 56.3 | 0.51 |
| | GmHT4-4-6 | 56.0 | 0.46 |
| | GmHT4-4-7 | 54.5 | 0.22 |
| Untransformed control | | 58.4 | |

To compare the field trial data and obtain more precise estimate on the performance of different constructs and transgenic events, a statistical meta-analysis was performed using the aggregate of all plants for each of the seven events for GmHT4-3, and each of the seven events for GmHT4-4 in the multi-season, multi-location field trial data. Table 8 provides the meta-analysis results for yield for the 14 events from the two constructs in the agronomy trials. Mean Yield refers to yield in bushel per acre for the event, or for the untransformed control (wild-type). Table 9 summarizes the meta-analysis results for yield in the efficacy trials. Mean Yield refers to yield in bushel per acre for the event, untreated, or treated with dicamba, glufosinate, 2,4-D, or mesotrione. Plants comprising the soybean event GmHT4-4-2 out-performed other events in these trials. Based on the results of meta-analysis and the molecular profile from the molecular analysis of each event, GmHT4-4-2 was selected as the commercial event and was named Gm_CSM63714.

TABLE 8
Meta-analysis of yield from agronomic field trials

| | | Mean | |
|---|---|---|---|
| Construct | Event | Yield | p_Value |
| GmHT4-3 | GmHT4-3-1 | 59.7 | 0.03 |
| | GmHT4-3-2 | 61.6 | 0.44 |
| | GmHT4-3-3 | 61.2 | 0.86 |
| | GmHT4-3-4 | 60.3 | 0.24 |
| | GmHT4-3-5 | 60.6 | 0.50 |
| | GmHT4-3-6 | 61.0 | 0.96 |
| | GmHT4-3-7 | 61.1 | 0.96 |
| GmHT4-4 | GmHT4-4-1 | 61.0 | 0.94 |
| | GmHT4-4-2 | 63.4 | 0.00 |
| | (Gm_CSM63714) | | |
| | GmHT4-4-3 | 62.7 | 0.01 |
| | GmHT4-4-4 | 62.6 | 0.02 |
| | GmHT4-4-5 | 62.6 | 0.02 |
| | GmHT4-4-6 | 61.7 | 0.32 |
| | GmHT4-4-7 | 62.2 | 0.08 |
| Untransformed control | | 61.1 | |

TABLE 9
Meta-analysis of yield from efficacy field trials

| | | Untreated | Dicamba | | Gufosinate | | 2,4-D | | Mesotrione | |
|---|---|---|---|---|---|---|---|---|---|---|
| | | Mean | Mean | | Mean | | Mean | | Mean | |
| Construct | Event | Yield | Yield | p_Value | Yield | p_Value | Yield | p_Value | Yield | p_Value |
| GmHT4-3 | GmHT4-3-1 | 60.4 | 59.4 | 0.33 | 59.1 | 0.20 | 62.4 | 0.05 | 60.4 | 0.95 |
| | GmHT4-3-2 | 61.5 | 61.3 | 0.85 | 60.8 | 0.54 | 63.2 | 0.14 | 62.3 | 0.47 |
| | GmHT4-3-3 | 61.3 | 59.7 | 0.19 | 59.8 | 0.21 | 64.4 | 0.01 | 61.0 | 0.79 |
| | GmHT4-3-4 | 62.6 | 60.5 | 0.06 | 59.8 | 0.02 | 62.9 | 0.82 | 61.3 | 0.25 |
| | GmHT4-3-5 | 62.2 | 60.5 | 0.08 | 61.6 | 0.51 | 63.5 | 0.20 | 61.1 | 0.28 |
| | GmHT4-3-6 | 62.1 | 61.3 | 0.47 | 61.0 | 0.35 | 63.2 | 0.34 | 62.1 | 0.98 |
| | GmHT4-3-7 | 62.8 | 60.9 | 0.11 | 61.9 | 0.48 | 63.0 | 0.85 | 63.0 | 0.81 |
| GmHT4-4 | GmHT4-4-1 | 61.6 | 63.1 | 0.11 | 60.9 | 0.53 | 60.3 | 0.21 | 61.6 | 0.95 |
| | GmHT4-4-2 | 64.5 | 64.3 | 0.84 | 63.8 | 0.52 | 62.6 | 0.11 | 64.0 | 0.64 |
| | (Gm_CSM63714) | | | | | | | | | |
| | GmHT4-4-3 | 64.5 | 64.2 | 0.77 | 63.5 | 0.33 | 62.4 | 0.04 | 63.0 | 0.14 |
| | GmHT4-4-4 | 63.8 | 65.5 | 0.16 | 64.1 | 0.82 | 61.4 | 0.04 | 64.2 | 0.74 |
| | GmHT4-4-5 | 64.6 | 63.6 | 0.43 | 64.2 | 0.76 | 61.6 | 0.01 | 62.9 | 0.16 |
| | GmHT4-4-6 | 65.7 | 64.3 | 0.27 | 63.7 | 0.11 | 62.2 | 0.00 | 64.2 | 0.21 |
| | GmHT4-4-7 | 64.6 | 65.2 | 0.60 | 63.5 | 0.36 | 61.4 | 0.01 | 63.8 | 0.50 |

### System Trials

In addition to assessing the events for tolerance to individual herbicides at 2×commercial rates as described in the preceding sections of this Example, system trials were designed to evaluate crop tolerance to 1×commercial rates of individual herbicides and herbicide tank mix combinations similar to those potentially used by growers.

Four events from GmHT4-3 and three events from GmHT4-4 were tested. The trial was conducted at four North American locations using a Group Unbalanced Block Design (GUBD2) with two replications and twenty treatments. These consisted of an untreated block along with nineteen blocks for herbicide treatments (Table 10). Herbicide applications were at V3, V6 and R1 growth stages. Turbo TeeJet Induction (TTI) nozzles were used for all herbicide treatments. Herbicide-treated event plots were compared to untreated event plots.

Plots were two rows of twelve feet in length, with a three-foot alley with 30-inch row spacing and seeded at 235 seeds per plot. Trial maintenance was designed to optimize grain yield. Data was collected for emergence, vigor, date of first flower, plant height, maturity, and yield. In addition, visual injury data was collected for the efficacy trials approximately seven days after postemergent herbicide applications. Statistical analysis was automated using ASReml software for mixed model fitting.

Table 10 provides a summary of herbicide treatments and the corresponding plant development stages of such treatments. Treatment 1 is the untreated control. The herbicide application rate is presented as pound/acre (lb/acre) unless otherwise noted. Mean Yield (bushels/acre) of the events with different herbicide treatments in the system trials is summarized in Table 11. The soy event GmHT4-4-2 (Gm_CSM63714) performed very well with all of the herbicide treatments and showed significantly positive yield with dicamba applications (Treatment 4). Event GmHT4-3-6 showed significant reduced yield with the herbicide combination of dicamba+mesotrione+glufosinate.

TABLE 10
Herbicide treatments and plant growth stages in the system trials

| | | Herbicide | | | | |
|---|---|---|---|---|---|---|
| | | (Commercial | Application Rate | Application | | |
| Treatment | Rate | Product Name) | (pound/acre) | Stage | | |
| 1 | — | None | — | — | — | — |
| 2 | 1X | Dicamba (Xtendimax) | 0.5 | V3 | | |
| | | 2,4-D (Enlist One) | 1 | | | |
| | | Glufosinate (Liberty 280ASL) | 0.8 | | V6 | |
| | | Mesotrione (Callisto 480SC) | 0.09 | | | R1 |
| 3 | 1X | Glufosinate (Liberty 280ASL) | 0.8 | V3 | | |
| | | Mesotrione (Callisto 480SC) | 0.09 | | | |
| | | Dicamba (Xtendimax) | 0.5 | | V6 | |
| | | 2,4-D (Enlist One) | 1 | | | |
| 4 | 1X | Dicamba (Xtendimax) | 0.5 | V3 | | R1 |
| 5 | 1X | Glufosinate (Liberty 280ASL) | 0.8 | V3 | | R1 |
| 6 | 1X | 2,4-D (2,4-D Amine ) | 1 | V3 | | R1 |
| 7 | 1X | 2,4-D (Enlist One) | 1 | V3 | | R1 |
| 8 | 1X | Mesotrione (Callisto 480SC) | 0.09 | V3 | | R1 |
| 9 | 1X | Dicamba (Xtendimax) | 0.5 | V3 | | R1 |
| | | Glufosinate (Liberty 280ASL) | 0.8 | | | |
| 10 | 1X | Dicamba (Xtendimax) | 0.5 | V3 | | R1 |
| | | 2,4-D (Enlist One) | 1 | | | |
| 11 | 1X | Dicamba (Xtendimax) | 0.5 | V3 | | R1 |
| | | Mesotrione (Callisto 480SC) | 0.09 | | | |
| 12 | 1X | Glufosinate (Liberty 280ASL) | 0.8 | V3 | | R1 |
| | | 2,4-D (Enlist One) | 1 | | | |
| 13 | 1X | 2,4-D (Enlist One) | 1 | V3 | | R1 |
| | | Mesotrione (Callisto 480SC) | 0.09 | | | |
| 14 | 1X | Glufosinate (Liberty 280ASL) | 0.8 | V3 | | R1 |
| | | Mesotrione (Callisto 480SC) | 0.09 | | | |
| 15 | 1X | Dicamba (Xtendimax) | 0.5 | V3 | | R1 |
| | | Mesotrione (Callisto 480SC) | 0.09 | | | |
| | | Glufosinate (Liberty 280ASL) | 0.8 | | | |
| 16 | 1X | Dicamba (Xtendimax) | 0.5 | V3 | | R1 |
| | | Mesotrione (Callisto 480SC) | 0.09 | | | |
| | | 2,4-D (Enlist One) | 1 | | | |
| 17 | 1X | Dicamba (Xtendimax) | 0.5 | V3 | | R1 |
| | | Mesotrione (Callisto 480SC) | 0.09 | | | |
| | | Glufosinate (Liberty 280ASL) | 0.8 | | | |
| 18 | 1X | 2,4-D (Enlist One) | 1 | V3 | | R1 |
| | | Mesotrione (Callisto 480SC) | 0.09 | | | |
| | | Glufosinate (Liberty 280ASL) | 0.8 | | | |

TABLE 11
Performance of events in system trials

| | | GnHT4-3 | | | | | |
|---|---|---|---|---|---|---|---|
| | | GmHT4-3-2 | | GmHT4-3-3 | | GmHT4-3-6 | |
| Treatment | | Mean | | Mean | | Mean | |
| Number | Treatment | Yield | p_Value | Yield | p_Value | Yield | p_Value |
| 1 | Untreated | 59.3 | | 59.2 | | 60.8 | |
| 2 | Quad 1 | 59.3 | 0.98 | 60.4 | 0.56 | 61.3 | 0.79 |
| 3 | Quad 2 | 57.9 | 0.50 | 58.8 | 0.85 | 60.8 | 0.98 |
| 4 | Dicamba | 61.6 | 0.26 | 60.4 | 0.56 | 61.6 | 0.68 |
| 5 | Glufosinate | 57.5 | 0.37 | 58.1 | 0.61 | 58.2 | 0.20 |
| 6 | 2,4-D Amine | 62.8 | 0.09 | 58.0 | 0.58 | 58.6 | 0.28 |
| 7 | 2,4-D Choline | 60.9 | 0.45 | 61.8 | 0.20 | 60.2 | 0.79 |
| 8 | Mesotrione | 57.3 | 0.32 | 61.2 | 0.33 | 61.6 | 0.70 |
| 9 | Dic + Gluf | 56.6 | 0.17 | 56.7 | 0.23 | 60.1 | 0.73 |
| 10 | Dic + 2,4-D | 60.4 | 0.59 | 61.2 | 0.33 | 59.9 | 0.66 |
| 11 | Dic + Meso | 59.8 | 0.79 | 59.5 | 0.88 | 61.9 | 0.58 |
| 12 | 2,4-D + Gluf | 59.2 | 0.94 | 58.3 | 0.67 | 59.4 | 0.49 |
| 13 | 2,4-D + Meso | 61.0 | 0.42 | 59.6 | 0.82 | 60.5 | 0.90 |
| 14 | Meso + Gluf | 56.8 | 0.23 | 58.7 | 0.81 | 58.2 | 0.23 |
| 15 | Dic + Meso + Gluf | 57.7 | 0.44 | 57.7 | 0.47 | 56.5 | 0.04 |
| 16 | Dic + Meso + 2,4-D | 57.6 | 0.40 | 58.9 | 0.91 | 60.0 | 0.70 |
| 17 | Dic + 2,4-D + Gluf | 59.2 | 0.97 | 59.9 | 0.71 | 57.3 | 0.09 |
| 18 | 2,4-D + Meso + Gluf | 57.1 | 0.28 | 56.3 | 0.17 | 59.3 | 0.46 |

| | | | GmHT4-4 | | | | | |
|---|---|---|---|---|---|---|---|---|
| | GnHT4-3 | | GmHT4-4-2 | | | | | |
| | GmHT4-3-7 | | (Gm_CSM63714) | | GmHT4-4-4 | | GmHT4-4-6 | |
| Treatment | Mean | | Mean | | Mean | | Mean | |
| Number | Yield | p_Value | Yield | p_Value | Yield | p_Value | Yield | p_Value |
| 1 | 58.8 | | 60.0 | | 61.1 | | 60.4 | |
| 2 | 60.4 | 0.45 | 61.4 | 0.52 | 60.8 | 0.90 | 61.8 | 0.49 |
| 3 | 60.1 | 0.54 | 63.2 | 0.13 | 62.2 | 0.58 | 59.6 | 0.70 |
| 4 | 60.6 | 0.38 | 64.7 | 0.03 | 60.8 | 0.90 | 63.8 | 0.10 |
| 5 | 56.0 | 0.16 | 61.3 | 0.53 | 59.6 | 0.49 | 59.4 | 0.62 |
| 6 | 59.9 | 0.59 | 60.2 | 0.94 | 59.4 | 0.41 | 59.4 | 0.64 |
| 7 | 61.7 | 0.16 | 60.3 | 0.88 | 61.2 | 0.97 | 62.5 | 0.32 |
| 8 | 59.4 | 0.79 | 62.2 | 0.30 | 61.6 | 0.79 | 63.0 | 0.21 |
| 9 | 58.2 | 0.74 | 63.6 | 0.09 | 61.4 | 0.90 | 61.2 | 0.69 |
| 10 | 60.7 | 0.37 | 60.1 | 0.97 | 61.2 | 0.96 | 60.6 | 0.90 |
| 11 | 59.5 | 0.75 | 63.0 | 0.15 | 63.6 | 0.23 | 64.3 | 0.06 |
| 12 | 58.7 | 0.94 | 59.3 | 0.73 | 60.9 | 0.92 | 57.6 | 0.19 |
| 13 | 61.4 | 0.22 | 59.8 | 0.90 | 61.3 | 0.90 | 61.0 | 0.77 |
| 14 | 57.3 | 0.45 | 61.1 | 0.62 | 59.6 | 0.48 | 59.2 | 0.55 |
| 15 | 57.4 | 0.48 | 60.2 | 0.94 | 62.2 | 0.59 | 61.7 | 0.52 |
| 16 | 60.5 | 0.41 | 59.2 | 0.68 | 60.8 | 0.90 | 58.5 | 0.34 |
| 17 | 58.8 | 0.97 | 61.7 | 0.44 | 58.5 | 0.20 | 61.7 | 0.51 |
| 18 | 60.9 | 0.31 | 56.8 | 0.12 | 59.5 | 0.45 | 58.7 | 0.42 |

Meta analysis was also performed on emergence, plant height, moisture, maturity, and yield for the seven events from each of the constructs GmHT4-3 and GmHT4-4. Emergence is a visual estimation of planted seed that emerged on a scale of 1-9, where 1 indicates that 90 to 100% of the planted seeds emerged, and 9 indicates that 0 to 19% of planted seeds emerged. The results showed that the mean emergence for the transgenic events ranged from 1.1 to 1.5, with a p-value ranging from 0.01 to 0.97, which was not significantly different from the mean of 1.2 for the untransformed control.

Plant height was assessed by measuring in inches the average height of the upper most nodes of representative plants, taken prior to harvest. The mean plant height for the transgenic events ranged from 33.0 to 35.9, with a p-value ranging from 0 to 0.51, which was not significantly different from the mean of 35.5 for the untransformed control.

Moisture refers to the moisture content of harvested grain from a given plot, denoted as percent. The mean moisture content for the transgenic events ranged from 11.7 to 11.9%, with a p-value ranging from 0.02 to 0.97, which was not significantly different from the mean of 11.7 for the untransformed control.

Maturity is the number of days before or after March 1st (South America) or August 31st (North America) when the plot reaches 95% maturity; that is, when 95% of the pods in the plot are brown. The mean maturity for the transgenic events ranged from 28.5 to 30.9, with a p-value ranging from 0 to 0.97, which was not significantly different from the mean of 28.5 for the untransformed control.

Yield is the grain yield expressed in bushels per acre. While the mean yield for the transgenic events ranged from 59.7 to 63.4, the mean for the untransformed control is 61.1. Therefore, the results demonstrate that the transgenic events behaved similarly to the untransformed control. Expression of the transgenes had no significant impact on plant growth and development.

### Example 3: Molecular Analysis and Event Selection

Molecular analysis was conducted concurrently with the field trials on events that were advanced. DNA amplification and sequencing were used to confirm the composition and intactness of the insert sequence, insert copy number, absence of Agrobacterium Ti-plasmid backbone sequence and the aadA/sp/A selection cassettes carried on the second T-DNA. The insertion site in the soybean genome of each event was mapped, and it was confirmed that the transgenic insert was not inserted in or close to any endogenous gene or a repeat region. Northern analysis was done to detect and measure mRNA transcripts of the DMO, PAT, FT_Tv7, and TDO genes in transgenic plants for each event. Protein analysis of plants comprising each event was conducted using techniques known in the art. N-terminal protein sequencing of the DMO, PAT, FT_Tv7, and TDO proteins purified from transgenic plants containing each event was done to confirm the recombinant protein sequences. Western blot analysis was conducted on protein extracts to confirm the production of the DMO, PAT, FT_Tv7, and TDO proteins from each transgenic event.

Based on the comprehensive and in-depth molecular characterization for each event in combination with the trait efficacy and agronomy performance over multiple growing seasons, in multiple geographic locations and under a variety of growth conditions, the soybean event Gm_CSM63714 was selected as the commercial event. Table 12 summarizes the results of the extensive and intensive selection process, leading to the identification and selection of the soybean event Gm_CSM63714 as the best event for commercial development. #R0 Event refers to number of events generated for each of the constructs (Step 1). #Event with 1-2 Copy of the Transgenic Insert refers to the number of events for each construct that contained 1-2 copy of the transgenic insert based on molecular characterizations, and thus were advanced to further testing and/or molecular characterization (Step 2). #Event without aadA Cassette refers to the number of events that contained 1-2 copy of the transgenic insert and did not contain the aadA/splA cassettes on the second T-DNA, indicating that the second T-DNA was not linked to the first T-DNA and was segregated away, and therefore, the events did not contain the second T-DNA (Step 3). #R0 Events with Seeds represents the number of R0 events from Step 3 that were able to grow to maturity and harvested with seeds (Step 4). #Advanced to R1 Nursery represents the number of events advanced to R1 nursery for seed production and further testing (Step 5). #Meeting Molecular Criteria refers to the number of events that met all the molecular criteria, such as single copy insert, intactness of the insert sequence, absence of Agrobacterium Ti-plasmid backbone sequence and the second T-DNA, insertion of the transgene not in or close to any endogenous gene or a repeat region, detection of the DMO, PAT FT_Tv7, and TDO protein in transgenic plants for each event, and confirmation of the recombinant protein sequences (Step 6). R2 Efficacy and Nursery represents the number of events showing herbicide tolerance efficacy and were advanced to R2 nursery (Step 7). R3 Field Trials NA Y1 and R4 SA Field Trials indicate the number of R3 and R4 events that were tested in year 1 North American and South American field trials (Step 8 and 9), respectively. R5 Field NA Y2 indicates the number of R5 events that were advanced to year 2 North American field trials (Step 10). Commercial Event indicates the final event that was selected as the commercial event, which met all the molecular criteria, and showed consistent efficacy and agronomic performance in all field trials.

TABLE 12
Summary of event selection

| Event Selection | | | | | | | |
|---|---|---|---|---|---|---|---|
| Description (Step) | GmHT4-1 | GmHT4-6 | GmHT4-3 | GmHT4-4 | GmHT4-5 | GmHT4-2 | Total |
| # R0 Event (1) | 4886 | 4513 | 4,685 | 4,751 | 2678 | 4247 | 25760 |
| # Event with 1-2 Copy of | 1013 | 839 | 943 | 934 | 552 | 921 | 5202 |
| Transgenic Insert (2) | | | | | | | |
| # Event without aadA | 186 | 151 | 201 | 167 | 105 | 134 | 944 |
| Cassette (3) | | | | | | | |
| # R0 Event with Seeds (4) | 160 | 147 | 158 | 160 | 100 | 125 | 850 |
| # Advanced to R1 nursery (5) | 50 | 57 | 54 | 43 | 37 | 47 | 288 |
| # Meeting Molecular | 26 | 12 | 37 | 22 | 17 | 27 | 141 |
| Criteria (6) | | | | | | | |
| R2 Efficacy and Nursery (7) | 18 | 14 | 20 | 18 | 18 | 18 | 106 |
| R3 field trials NA Y1 (8) | 17 | — | 15 | 18 | — | 18 | 68 |
| R4 SA field trials (9) | 17 | — | 18 | 18 | — | 17 | 70 |
| R5 field NA Y2 (10) | — | — | 7 | 8 | — | — | 15 |
| Commercial Event | | | | 1 | | | 1 |

### Example 5: Molecular Characterization of Soybean Event Gm_CSM63714

As described above, soybean event Gm_CSM63714 was identified through comprehensive molecular characterization and event selection processes coupled with field performance testing including trait efficacy and yield. This example describes the extensive molecular characterization upon selection of Gm_CSM63714 as the commercial event, including confirmation of single copy of intact T-DNA at a single locus, absence of Agrobacterium Ti plasmid backbone DNA and the second T-DNA containing the aadA/sp/A selection cassettes; determination of the chromosomal location of the T-DNA insert, confirmation that the T-DNA did not interrupt any known endogenous gene and did not insert into any repeat regions; and identification of the transgene 5′ and 3′ genomic flanking sequences and the wild-type allele sequence. The transgenic insert of soy event Gm_CSM63714 contains the elements and sequences described in Table 1.

DNA sequence analysis of the soybean event Gm_CSM63714 was conducted. Southern hybridization analysis was conducted to confirm that plants of soybean event Gm_CSM63714 contained a single and intact copy of the entire transgenic insert without any transformation vector backbone and the second T-DNA sequence harboring the aadA and splA selection cassettes. The in planta transgenic insert was isolated and sequenced using methods known in the art. The results showed that the inserted in planta T-DNA sequence perfectly matched the expected T-DNA sequence from the transformation vector HT4-4.

DNA flanking the transgenic insert (5′ and 3′ flanking sequences) was also isolated and sequenced using sequence capture, enrichment, sequencing, inverse PCR, and genome walking techniques. The respective 5′ and 3′ junction sequences were subsequently determined. The sequences of the flanking DNA for soybean event Gm_CSM63714 were mapped to the known soybean genome physical assembly. The insertion site sequence information was used for bioinformatic analysis to determine the chromosomal location of the event. The insertion site integrity was determined by PCR across the wild-type allele using primers specific to the flanking regions of soybean event Gm_CSM63714. The wild-type insertion site was used to map the unique site of transgenic integration for soybean event Gm_CSM63714 to the soybean reference genome. Molecular analysis identified a 40-nucleotide deletion at the transgene insertion site, and also confirmed that the inserted T-DNA sequence perfectly matches the intended T-DNA sequence from the transformation vector, with no mutations or truncations. Whole genome sequencing (E-Southern) was performed on materials from multiple generations to confirm the identity of the progeny. Sequence information for the transgenic insert, the 5′ and 3′ flanking sequences, and the 5′ and 3′ junctions are provided herein as SEQ ID NOs:1-10.

RNA analysis of plants comprising the soybean event Gm_CSM63714 was conducted. Northern hybridization was performed on total RNA and mRNA isolated from immature seeds. The results confirmed RNA transcripts corresponding in size to the DMO, PAT, FT_Tv7, and TDO mRNA products in soy event Gm_CSM63714.

Protein analyses of plant comprising soybean event Gm_CSM63714 were also conducted. The N-terminal amino acid sequences of the expressed DMO, PAT, FT_Tv7, and TDO proteins were determined by Edman sequencing and mass spectrometry using immunopurified protein extracts from mature seeds to confirm the authentic N-terminal amino acid sequences. Western blot analysis was conducted on protein extracts from mature seed of soybean event Gm_CSM63714 to confirm the production of a single expected-sized protein for DMO, PAT, FT_Tv7, and TDO, respectively. In addition, ELISA was used to determine protein levels of DMO, PAT, FT_Tv7, and TDO in soybean event Gm_CSM63714 leaves during different developmental stages (V3, R1, and R5), under different growth conditions (greenhouse, growth chamber, and field) and over multiple generations (R1, R3, and R5). The results show that, overall, the DMO, PAT, FT_Tv7, and TDO proteins remained stable across different growth conditions and multiple generations tested.

### Example 6: Detection of Soybean Event Gm_CSM63714

This example describes methods useful in identifying or detecting the presence of soybean event Gm_CSM63714. Detection of the event in a sample can be achieved using DNA, RNA, or protein detection techniques. Illustrative detection methods and materials are provided below.

### 1) Soybean Event Gm_CSM63714 Event-Specific Endpoint Taqman™ Assays

An event-specific endpoint Applied Biosystems™ TaqMan thermal amplification method (Thermo Fisher Scientific) was developed to identify soybean event Gm_CSM63714 in a sample. The DNA primers and probe used in the endpoint assay for this example are shown in Table 13, although it will be appreciated by those of skill in the art that that other primers and probes may also be used.

TABLE 13
Primers and probe for soybean
Gm_CSM63714 event-specific assay

| SEQ ID NO. | Name | Type |
|---|---|---|
| 14 | Primer SQ21524 | Event-specific |
| 15 | Primer SQ51589 | Event-specific |
| 16 | 6FAM ™ probe PB 10269 | Event-specific |
| 17 | Primer SQ546 | Internal control |
| 18 | Primer SQ549 | Internal control |
| 19 | VIC ™ Probe PB50207 | Internal control |

6-FAM™ is a fluorescent dye product of Applied Biosystems (Foster City, Calif.) and is attached to the DNA probe. For TaqMan MGB (Minor Grove Binder) probes, the 5′ exonuclease activity of Taq DNA polymerase cleaves the probe from the 5′-end, between the fluorophore and quencher. When hybridized to the target DNA strand, quencher and fluorophore are separated enough to produce a fluorescent signal, thus releasing fluorescence. The pair of primers when used with these reaction methods and the probe produce a DNA amplicon that is diagnostic for soybean event Gm_CSM63714. The controls for this analysis should include a positive control containing soybean event Gm_CSM63714, a negative control from a non-transgenic plant, and a negative control that contains no template DNA. Additionally, a control for the PCR reaction should optimally include internal control primers and an internal control probe, specific to a single copy gene in the soybean genome. These assays are optimized for use with the Applied Biosystems GeneAmp® PCR System 9700 (Thermo Fisher Scientific) run at maximum speed, but other equipment may be used.

Examples of PCR reaction components and cycling conditions useful for the event-specific qualitative endpoint TaqMan PCR assay for soybean event Gm_CSM63714 are presented in Table 14 and Table 15. The extracted DNA template was a DNA sample to be analyzed, a negative control (non-transgenic soybean DNA), no template (water) control, or a positive control containing soybean event Gm_CSM63714 DNA.

TABLE 14
Gm_CSM63714 event-specific endpoint TaqMan ™ PCR
reaction components

| | | Stock | | Final |
|---|---|---|---|---|
| | | Concentration | Volume | Concen- |
| Step | Reagent | (μM) | (μl) | tration |
| — | Reaction Volume | | 5 | |
| 1 | 2 × Master Mix | | 2.28 | 1 X |
| 2 | Event Specific Primer | 100 | 0.05 | 0.9 μM |
| | SQ21524 | | | |
| 3 | Event Specific Primer | 100 | 0.05 | 0.9 μM |
| | SQ51589 | | | |
| 4 | Event Specific 6FAM ™ | 100 | 0.01 | 0.2 uM |
| | probe PB10269 | | | |
| 5 | Internal Control Primer | 100 | 0.05 | 0.9 μM |
| | SQ546 | | | |
| 6 | Internal Control Primer | 100 | 0.05 | 0.9 μM |
| | SQ549 | | | |
| 7 | Internal Control VIC ™ | 100 | 0.01 | 0.2 μM |
| | Probe PB50207 | | | |
| 8 | Extracted DNA template | | 2.5 | |

TABLE 15
Endpoint TaqMan ™ thermocycler conditions

| Step No. | Cycle No. | Settings |
|---|---|---|
| 1 | 1 | 95° C., 20 seconds |
| 2 | 35 | 95° C., 3 seconds |
| | | 60° C., 20 seconds |

### 2) Detection of Soybean Event Gm_CSM63714 Using AntibodiesPG-3″

Another example of detection of soybean event Gm_CSM63714 involves the use of one or more antibodies specific for at least one protein encoded by soybean event Gm_CSM63714. For example, a detection kit comprising at least such antibody can be used. Such a kit may utilize a lateral flow strip comprising reagents activated when the tip of the strip is contacted with an aqueous solution. Illustrative proteins sufficient for use in antibody production are ones encoded by the sequence provided as SEQ ID NO:10, or any fragment thereof.

A protein detection method is developed to determine whether a sample is from a plant, seed, cell, or plant part comprising soybean event Gm_CSM63714. At least one antibody specific for at least one protein encoded by soybean event Gm_CSM63714 is used to detect a protein encoded by soybean event Gm_CSM63714 in a sample. A detection kit comprising one or more antibodies specific for one or more proteins encoded by soybean event Gm_CSM63714 may utilize a lateral flow strip containing reagent activated when the tip of the strip is contacted with an aqueous solution. Samples of soybean tissues may be ground up and protein extracted for analysis using water or an aqueous buffer (for example, phosphate buffered saline containing detergent and bovine serum albumin). Following centrifugation, the aqueous supernatant is analyzed using the ELISA method in a sandwich format on a lateral flow strip containing an absorbent pad. Detection is activated by dipping the tip of the strip into the aqueous solution containing the sample to be tested.

The aqueous solution is carried up the strip by capillary action and solubilizes gold labeled antibodies on the strip. The gold-labeled antibodies are specific for at least one protein encoded by soybean event Gm_CSM63714 and will bind to an epitope on the protein in the sample to form an antibody-antigen complex. The gold labeled antibody-antigen complex is then carried up the strip to a nitrocellulose membrane. The membrane comprises a test line of immobilized antibodies that bind to a second, separate epitope on the protein encoded by soybean event Gm_CSM63714, causing a visible line to appear across the test strip if the protein encoded by soybean event Gm_CSM63714 is present in the sample.

### 3) Detection of Soybean Event Gm_CSM63714 by Southern Analysis

Another method to detect the presence of soybean event Gm_CSM63714 in a plant sample is Southern analysis as generally understood in the art. One of skill in art, based on the present disclosure and description of soybean event Gm_CSM63714, would understand how to design Southern hybridization probe(s) specific for the event and a second Southern hybridization probe specific for a plant which is null for the event (wild-type). With Southern analysis, a signal detected only from the first Southern hybridization probe will be indicative of a plant positive for soybean event Gm_CSM63714; a signal detected only from the second Southern hybridization probe will be indicative that the DNA was extracted from a plant that is null for the event (wild-type).

### Example 7: Zygosity Assays for Soybean Event Gm_CSM63714

This example describes methods useful in determining the zygosity of event Gm_CSM63714. The zygosity assay determines whether a plant comprising soybean event Gm_CSM63714 is heterozygous or homozygous for the event or the wild-type allele. Illustrative detection methods and materials are provided below.

A zygosity assay is developed to determine whether a plant comprising soybean event Gm_CSM63714 is heterozygous or homozygous for the event or the wild-type allele. An amplification reaction assay can be designed using the sequence information provided herein. For example, such a PCR assay would include design of at least three primers: primer-1, primer-2 and primer-3, where primer-1 is specific to soybean genomic DNA on the 3′ flanking DNA of soybean event Gm_CSM63714 (for example, SEQ ID NO:15); primer-2 is specific to soybean event Gm_CSM63714 transgenic insert (for example, SEQ ID NO:14); and primer-3 is specific to the wild-type allele (for example, SEQ ID NO:20). When used as a primer pair in an amplification reaction, primer-1 with primer-2 will produce a PCR amplicon specific for soybean event Gm_CSM63714. When used as a primer pair in an amplification reaction, primer-1 with primer-3 will produce a PCR amplicon specific for wild-type allele. In a PCR reaction performed on soybean event Gm_CSM63714, the respective PCR amplicons generated from primer-1+primer-2 and those generated from primer-1+primer-3 will differ in sequence and size of the amplicon. When the three primers are included in a PCR reaction with DNA extracted from a plant homozygous for soybean event Gm_CSM63714, only the primer-1+primer-2 amplicon (specific for the soybean event Gm_CSM63714) will be generated. When the three primers are included in a PCR reaction with DNA extracted from a plant heterozygous for soybean event Gm_CSM63714, both the primer-1+primer-2 amplicon (specific for the soybean event Gm_CSM63714 insert) and the primer-1+primer-3 amplicon (specific for the wild-type allele or absence of the soybean event Gm_CSM63714 insert) will be generated. When the three primers are mixed together in a PCR reaction with DNA extracted from a plant that is null for soybean event Gm_CSM63714 (that is wild-type), only the primer-1+primer-3 amplicon (specific for the wild-type allele) will be generated. The amplicons produced using the PCR reaction may be identified or distinguished using any method known in the art.

Another zygosity assay for soybean event Gm_CSM63714 is a Tagman™ thermal amplification method. Two fluorescently labeled probes are included in addition to the primers as described in the proceeding section. Probe-1, containing a fluorescent label (for example, the 6-FAM™-labeled SEQ ID NO:16), is specific for soybean event Gm_CSM63714; whereas Probe-2, containing a different fluorescent label (for example, the VIC™-labeled SEQ ID NO:21), is specific for a wild-type soybean plant that is null for soybean event Gm_CSM63714.

When the three primers (SEQ ID NO:14, SEQ ID NO:15, and SEQ ID NO:20) and two probes (SEQ ID NO:16 and SEQ ID NO:21) are mixed together in a PCR reaction with DNA extracted from a plant homozygous for soybean event Gm_CSM63714, a fluorescent signal from the 6FAM™-labeled probe PB10269 (SEQ ID NO:16) is released, which is indicative of and diagnostic for a plant homozygous for soybean event Gm_CSM63714. When the three primers and two probes are mixed together in a PCR reaction with DNA extracted from a plant heterozygous for soybean event Gm_CSM63714, two distinct fluorescent signals are generated, one from the 6FAM™-labeled probe PB10269 (SEQ ID NO:16), and one from the VIC™_labeled probe PB50681 (SEQ ID NO:21). When the three primers and the two probes are mixed together in a PCR reaction with DNA extracted from a wild-type plant which is null for soybean event Gm_CSM63714, a fluorescent signal from the VIC™-labeled probe PB50681 (SEQ ID NO:21) is generated.

Examples of PCR reaction components and cycling conditions useful for TaqMan™ PCR zygosity assay for soybean event Gm_CSM63714 are presented in Table 16 and Table 17. The extracted DNA template was a DNA sample to be analyzed, a negative control (non-transgenic soybean DNA), no template (water) control, or a positive control containing soybean event Gm_CSM63714 DNA.

TABLE 16
Gm_CSM63714 zygosity TaqMan ™ PCR reaction components

| | | Stock | | Final |
|---|---|---|---|---|
| | | Concentration | Volume | Concen- |
| Step | Reagent | (μM) | (μl) | tration |
| — | Reaction Volume | | 5 | |
| 1 | 18 megohm water | | 0.05 | |
| 2 | 2 × Master Mix | | 2.28 | 1 X |
| 3 | Event Specific Primer | 100 | 0.05 | 0.9 μM |
| | SQ21524 | | | |
| 4 | Event Specific Primer | 100 | 0.05 | 0.9 μM |
| | SQ51589 | | | |
| 5 | Event Specific 6FAM ™ | 100 | 0.01 | 0.2 μM |
| | probe PB10269 | | | |
| 6 | Wild-type allele primer | 100 | 0.05 | 0.9 μM |
| | SQ52071 | | | |
| 7 | Wild-type allele VIC ™ | 100 | 0.01 | 0.2 μM |
| | probe PB50681 | | | |
| 8 | Extracted DNA template | | 2.5 | |

TABLE 17
Zygosity TaqMan ™ thermocycler conditions

| Step No. | Cycle No. | Settings |
|---|---|---|
| 1 | 1 | 95° C., 20 seconds |
| 2 | 35 | 95° C., 3 seconds |
| | | 60° C., 20 seconds |

Another method to detect the presence and zygosity of soybean event Gm_CSM63714 in a plant sample is Southern blot analysis. One of skill in art would understand how to design a first Southern hybridization probe(s) specific for soybean event Gm_CSM63714 and a second Southern hybridization probe specific for a soybean plant which is null for the soybean event Gm_CMS63714 (wild-type). With Southern blot analysis, a signal detected only from the first Southern hybridization probe is indicative a plant homozygous for soybean event Gm_CSM63714; a signal detected from both the first and the second hybridization probes is indicative of a plant heterozygous for soybean event Gm_CSM63714; and a signal detected only from the second Southern hybridization probe indicates that the DNA was extracted from a plant that is null for soybean event Gm_CSM63714 (wild-type).

### Example 8: Modification of Soybean Event Gm_CSM63714 with Genome Editing Techniques Using a Single Guide RNA

This example describes how one may alter or excise all or a part of the transgenic insertion present in soybean event Gm_CSM63714, as well as flanking genomic DNA segments, such as by making one or more insertions, deletions, substitutions, or transpositions using genomic editing techniques. For example, such alterations can be made using Clustered Regularly Interspersed Short Palindromic Repeats (CRISPR) editing systems comprising a single guide RNA by genome editing methods. Sequences useful in excision of the event Gm_CSM63714 transgenic insertion or expression cassettes within SEQ ID NO:9 or SEQ ID NO:10 can be introduced through genome editing using a variety of methods. In one embodiment, Clustered Regularly Interspersed Short Palindromic Repeats (CRISPR) editing systems comprising a CRISPR associated protein and cognate guide RNAs may be used for targeted excision. The CRISPR-associated protein is an RNA guided nuclease and can be selected from a Type I CRISPR-associated protein, a Type II CRISPR-associated protein, a Type III CRISPR-associated protein, a Type IV CRISPR-associated protein, a Type V CRISPR-associated protein, or a Type VI CRISPR-associated protein, such as, but not limited to, Cas1, Cas1B, Cas2, Cas3, Cas4, Cas5, Cas6, Cas7, Cas8, Cas9 (also known as Csn1 and Csx12), Cas10, Cas 12a (also known as Cpf1), Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, CasX, CasY, and Mad7. The CRISPR-associated protein and one or more guide RNAs (gRNA) can be introduced into a plant cell corresponding to soybean event Gm_CSM63714 to target a specific sequence within the transgene insertion locus. In one embodiment, the CRISPR nuclease system cleaves at two identical guide RNA hybridization sites thereby permitting the excision of the intervening sequence. Following DNA cleavage, the genomic sequence can be repaired via a double strand break repair pathway, which may include, for example, non-homologous end-joining (NHEJ), microhomology-mediated end joining (MMEJ), homologous recombination, synthesis-dependent strand annealing (SDSA), single-strand annealing (SSA), or a combination of any thereof, at the genomic target site. One or more guide RNA hybridization sequences can be inserted within the event Gm_CSM63714 transgene insertion locus which can subsequently allow for the excision of the transgene insertion from event Gm_CSM63714 or specific expression cassettes within SEQ ID NO:9 or SEQ ID NO:10.

Sequences corresponding to the 5′ and 3′ flanking genomic sequences of event Gm_CSM63714 (presented as SEQ ID NOs:11 and 12), the 5′ and 3′ junction regions (presented as SEQ ID NOs:1-6) and the transgenic insertion (presented as SEQ ID NO:9) were scanned for potential originator guide RNA recognition sites (OgRRS). As used herein, the term “originator guide RNA recognition site” or “OgRRS” refers to an endogenous DNA polynucleotide comprising a protospacer adjacent motif (PAM) site operably linked to a guide RNA hybridization site (i.e., protospacer sequence). In some embodiments, an OgRRS can be located in the flanking 5′ or 3′ genomic sequence (i.e., in non-transgenic DNA of a junction polynucleotide). In some embodiments, an OgRRS can be located in the 5′ or 3′ junction region (i.e., in both transgenic DNA and non-transgenic DNA of a junction polynucleotide, or spanning transgenic and non-transgenic DNA in a DNA junction polynucleotide). In some embodiments, an OgRRS can be located in the transgenic insert. The OgRRS can be determined based upon the specific CRISPR editing system chosen. For example, Cas9 recognizes a G-rich protospacer-adjacent motif (PAM) that is 3′ to its guide RNA hybridization site whereas Cas12a systems recognize a T-rich protospacer-adjacent motif (PAM) that is 5′ to its guide RNA hybridization site.

The OgRRS sequence is then used to define a cognate guide RNA recognition site (CgRRS) which is inserted into the transgenic insertion locus of event Gm_CSM63714 using a CRISPR editing system. As used herein, the term “cognate guide RNA recognition site” or “CgRRS” refers to a DNA polynucleotide comprising a PAM site operably linked to a guide RNA hybridization site (i.e. protospacer sequence), where the CgRRS is absent from event Gm_CSM63714 comprising the original transgenic locus that is unmodified and where the CgRRS and its corresponding OgRRS can hybridize to a single gRNA. A CgRRS can be located in the flanking 5′ or 3′ genomic sequence (i.e., in non-transgenic DNA of a junction polynucleotide), in the 5′ or 3′ junction region (i.e., in both transgenic DNA and non-transgenic DNA of a junction polynucleotide, or spanning transgenic and non-transgenic DNA in a DNA junction polynucleotide), or in the transgenic insert. A CgRRS comprises the same gRNA target sequence as the corresponding OgRRS. The CgRRS is inserted in a region within the transgenic insertion locus of event Gm_CSM63714 that is on the opposite side of the transgenic insertion, relative to the OgRRS in a manner that will permit the excision of a fragment of DNA corresponding to either the entire transgenic insertion of event Gm_CSM63714, or a fragment within the transgene insert of event Gm_CSM63714 such as an expression cassette or genetic element within the transgene cassette, using a single gRNA. For example, if the OgRRS is located within the 3′ flanking genomic sequence or the 3′ junction region, then the CgRRS can be inserted within the 5′ flanking genomic sequence, or the 5′ junction region, or within the transgene insert such as between expression cassettes or genetic elements within an expression cassette. Insertion of the CgRRS on the opposite side of the transgenic insertion or within the region between expression cassettes, relative to the OgRRS allows for excision of the transgenic insertion or specific expression cassette(s) to be excised using a single gRNA. An OgRRS located between the expression cassettes of event Gm_CSM63714 can be used to design a CgRRS that can be inserted in either the 5′ or 3′-flanking genomic sequence to permit excision of one or more expression cassettes using a single gRNA.

Table 18 shows OgRRS sequences located within the 5′ and 3′-flanking genomic sequences and in the transgenic insertion of event Gm_CSM63714 that can be used in a CRISPR editing system employing Cas12a, a Type V CRISPR-associated protein. The analysis was performed for four Cas12a endonucleases. Fn (SEQ ID NO:48) refers to Francisella novicida U112 Cas12a (also known as FnCas12a or FnCpf1), and requires the PAM sequence of 5′-TTN, where N is A, C, G or T (Zetsche et al., 2015). Lb (SEQ ID NO:45) refers to the Cas12a from Lachnospiraceae bacterium ND2006 (also known as LbCas12a or LbCpf1), and requires the PAM sequence of 5′-TTTV, where V is A, C, or G. Lb_V1 and Lb_V2 refer to engineered variants of Lachnospiraceae bacterium ND2006 Cas12a (Gao et al., 2017). The Lb_V1 variant (SEQ ID NO:46) contains the G532R/K595R mutations and recognizes 5′-TYCV PAM; whereas the Lb_V2 variant (SEQ ID NO:47) contains the G532R/K538V/Y542R mutations and recognizes 5′-TATV PAM, where Y is C or T, and V is A, C or G. The PAM sequence, the coordinates of the gRNA hybridization site (also known as OgRRS) relative to SEQ ID NO:10, and the corresponding Cas12a endonuclease are shown under the headings of “PAM”, “Cas12a Nuclease”, and “Start . . . End of gRNA Hybridization Site in SED ID NO:10”, respectively. “Strand of SEQ ID NO:10” indicates whether the identified gRNA hybridization site along with its PAM sequence is on the forward strand (+) or the complementary strand (−).

TABLE 18
OgRRS sequences within event Gm_CSM63714

| Name of | | | | | |
|---|---|---|---|---|---|
| gRNA | | | Start . . . End | | |
| Hybri- | | | of gRNA | Strand | |
| dization | | | Hybridization | of | |
| Site | | Cas12a | Site in | SEQ ID | Target |
| (OgRRS) | PAM | Nuclease | SEQ ID NO: 10 | NO: 10 | Region |
| 5F-1 | TTA | Fn | 6 . . . 28 | + | 5′ Flanking Genomic DNA |
| 5F-2 | TTG | Fn | 16 . . . 38 | + | 5′ Flanking Genomic DNA |
| 5F-3 | TCCG | Lb_V1 | 1 . . . 23 | − | 5′ Flanking Genomic DNA |
| 5F-4 | TTCC | Lb_V1 | 2 . . . 24 | − | 5′ Flanking Genomic DNA |
| 5F-5 | TTC | Fn | 3 . . . 25 | − | 5′ Flanking Genomic DNA |
| 5F-6 | TTA | Fn | 18 . . . 40 | − | 5′ Flanking Genomic DNA |
| 5F-7 | TCCC | Lb_V1 | 31 . . . 53 | − | 5′ Flanking Genomic DNA |
| 5F-8 | TTCC | Lb_V1 | 32 . . . 54 | − | 5′ Flanking Genomic DNA |
| 5F-9 | TTC/ | Fn/Lb_V1/ | 33 . . . 55 | − | 5′ Flanking Genomic DNA |
| | TTTC/ | Lb_V2/Lb | | | |
| | TTTC | | | | |
| 5F-10 | TTC | Fn | 79 . . . 101 | + | 5′ Flanking Genomic DNA |
| 5F-11 | TTCG | Lb_V1 | 80 . . . 102 | + | 5′ Flanking Genomic DNA |
| 5F-12 | TTA | Fn | 71 . . . 93 | − | 5′ Flanking Genomic DNA |
| 5F-13 | TTA/ | Fn/Lb_V1/ | 77 . . . 99 | − | 5′ Flanking Genomic DNA |
| | TTTA/ | Lb_V2/Lb | | | |
| | TTTA | | | | |
| 5F-14 | TTA/ | Fn/Lb_V1/ | 102 . . . 124 | + | 5′ Flanking Genomic DNA |
| | TTTA/ | Lb_V2/Lb | | | |
| | TTTA | | | | |
| 5F-15 | TTA | Fn | 111 . . . 133 | + | 5′ Flanking Genomic DNA |
| 5F-16 | TATG | Lb_V2 | 113 . . . 135 | + | 5′ Flanking Genomic DNA |
| 5F-17 | TATC | Lb_V2 | 117 . . . 139 | + | 5′ Flanking Genomic DNA |
| 5F-18 | TATA | Lb_V2 | 123 . . . 145 | + | 5′ Flanking Genomic DNA |
| 5F-19 | TATG | Lb_V2 | 125 . . . 147 | + | 5′ Flanking Genomic DNA |
| 5F-20 | TTCA | Lb_V1 | 109 . . . 131 | − | 5′ Flanking Genomic DNA |
| 5F-21 | TTC/ | Fn/Lb_V1/ | 110 . . . 132 | − | 5′ Flanking Genomic DNA |
| | TTTC/ | Lb_V2/Lb | | | |
| | TTTC | | | | |
| 5F-22 | TTC/ | Fn/Lb_V1/ | 156 . . . 178 | + | 5′ Flanking Genomic DNA |
| | TTTC/ | Lb_V2/Lb | | | |
| | TTTC | | | | |
| 5F-23 | TTG/ | Fn/Lb_V1/ | 160 . . . 182 | + | 5′ Flanking Genomic DNA |
| | TTTG/ | Lb_V2/Lb | | | |
| | TTTG | | | | |
| 5F-24 | TTCA | Lb_V1 | 173 . . . 195 | − | 5′ Flanking Genomic DNA |
| 5F-25 | TTC/ | Fn/Lb_V1/ | 174 . . . 196 | − | 5′ Flanking Genomic DNA |
| | TTTC/ | Lb_V2/Lb | | | |
| | TTTC | | | | |
| 5F-26 | TCCG | Lb_V1 | 202 . . . 224 | − | 5′ Flanking Genomic DNA |
| 5F-27 | TTC/ | Fn/Lb_V1/ | 236 . . . 258 | + | 5′ Flanking Genomic DNA |
| | TTTC/ | Lb_V2/Lb | | | |
| | TTTC | | | | |
| 5F-28 | TTCA | Lb_V1 | 237 . . . 259 | + | 5′ Flanking Genomic DNA |
| 5F-29 | TTA | Fn | 249 . . . 271 | + | 5′ Flanking Genomic DNA |
| 5F-30 | TTA/ | Fn/Lb_V1/ | 234 . . . 256 | − | 5′ Flanking Genomic DNA |
| | TTTA/ | Lb_V2/Lb | | | |
| | TTTA | | | | |
| 5F-31 | TTG/ | Fn/Lb_V1/ | 257 . . . 279 | + | 5′ Flanking Genomic DNA |
| | TTTG/ | Lb_V2/Lb | | | |
| | TTTG | | | | |
| 5F-32 | TTA | Fn | 245 . . . 267 | − | 5′ Flanking Genomic DNA |
| 5F-33 | TTA/ | Fn/Lb_V1/ | 257 . . . 279 | − | 5′ Flanking Genomic DNA |
| | TTTA/ | Lb_V2/Lb | | | |
| | TTTA | | | | |
| 5F-34 | TTA | Fn | 264 . . . 286 | − | 5′ Flanking Genomic DNA |
| 5F-35 | TTG/ | Fn/Lb_V1/ | 315 . . . 337 | + | 5′ Flanking Genomic DNA |
| | TTTG/ | Lb_V2/Lb | | | |
| | TTTG | | | | |
| 5F-36 | TTA | Fn | 312 . . . 334 | − | 5′ Flanking Genomic DNA |
| 5F-37 | TTA/ | Fn/Lb_V1/ | 403 . . . 425 | + | 5′ Flanking Genomic DNA |
| | TTTA/ | Lb_V2/Lb | | | |
| | TTTA | | | | |
| 5F-38 | TATC | Lb_V2 | 405 . . . 427 | + | 5′ Flanking Genomic DNA |
| 5F-39 | TATA | Lb_V2 | 407 . . . 429 | − | 5′ Flanking Genomic DNA |
| 5F-40 | TTG/ | Fn/Lb_V1/ | 512 . . . 534 | + | 5′ Flanking Genomic DNA |
| | TTTG/ | Lb_V2/Lb | | | |
| | TTTG | | | | |
| 5F-41 | TTA | Fn | 527 . . . 549 | + | 5' Flanking Genomic DNA |
| 5F-42 | TTA/ | Fn/Lb_V1/ | 509 . . . 531 | − | 5' Flanking Genomic DNA |
| | TTTA/ | Lb_V2/Lb | | | |
| | TTTA | | | | |
| 5F-43 | TATC | Lb_V2 | 514 . . . 536 | − | 5' Flanking Genomic DNA |
| 5F-44 | TATA | Lb_V2 | 516 . . . 538 | − | 5' Flanking Genomic DNA |
| 5F-45 | TTA | Fn | 612 . . . 634 | + | 5' Flanking Genomic DNA |
| 5F-46 | TTA | Fn | 622 . . . 644 | + | 5' Flanking Genomic DNA |
| 5F-47 | TTA | Fn | 625 . . . 647 | + | 5' Flanking Genomic DNA |
| 5F-48 | TTA | Fn | 628 . . . 650 | + | 5' Flanking Genomic DNA |
| 5F-49 | TTCA | Lb_V1 | 607 . . . 629 | − | 5' Flanking Genomic DNA |
| 5F-50 | TTC | Fn | 608 . . . 630 | − | 5' Flanking Genomic DNA |
| 5F-51 | TCCC | Lb_V1 | 611 . . . 633 | − | 5' Flanking Genomic DNA |
| 5F-52 | TTCC | Lb_V1 | 612 . . . 634 | − | 5' Flanking Genomic DNA |
| 5F-53 | TTC | Fn | 613 . . . 635 | − | 5' Flanking Genomic DNA |
| 5F-54 | TATC | Lb_V2 | 649 . . . 671 | + | 5' Flanking Genomic DNA |
| 5F-55 | TTA/ | Fn/Lb_V1/ | 660 . . . 682 | + | 5' Flanking Genomic DNA |
| | TTTA/ | Lb_V2/Lb | | | |
| | TTTA | | | | |
| 5F-56 | TTC/ | Fn/Lb_V1/ | 665 . . . 687 | + | 5' Flanking Genomic DNA |
| | TTTC/ | Lb_V2/Lb | | | |
| | TTTC | | | | |
| 5F-57 | TTA | Fn | 668 . . . 690 | + | 5' Flanking Genomic DNA |
| 5F-58 | TTG/ | Fn/Lb_V1/ | 652 . . . 674 | − | 5' Flanking Genomic DNA |
| | TTTG/ | Lb_V2/Lb | | | |
| | TTTG | | | | |
| 5F-59 | TTC | Fn | 665 . . . 687 | − | 5' Flanking Genomic DNA |
| 5F-60 | TTG | Fn | 674 . . . 696 | − | 5' Flanking Genomic DNA |
| 5F-61 | TTA/ | Fn/Lb_V1/ | 678 . . . 700 | − | 5' Flanking Genomic DNA |
| | TTTA/ | Lb_V2/Lb | | | |
| | TTTA | | | | |
| 5F-62 | TTA | Fn | 703 . . . 725 | + | 5' Flanking Genomic DNA |
| 5F-63 | TTA | Fn | 711 . . . 733 | + | 5' Flanking Genomic DNA |
| 5F-64 | TTA | Fn | 715 . . . 737 | + | 5' Flanking Genomic DNA |
| 5F-65 | TTA | Fn | 729 . . . 751 | + | 5' Flanking Genomic DNA |
| 5F-66 | TTA/ | Fn/Lb_V1/ | 708 . . . 730 | − | 5' Flanking Genomic DNA |
| | TTTA/ | Lb_V2/Lb | | | |
| | TTTA | | | | |
| 5F-67 | TTA | Fn | 733 . . . 755 | + | 5' Flanking Genomic DNA |
| 5F-68 | TTA/ | Fn/Lb_V1/ | 712 . . . 734 | − | 5' Flanking Genomic DNA |
| | TTTA/ | Lb_V2/Lb | | | |
| | TTTA | | | | |
| 5F-69 | TTA/ | Fn/Lb_V1/ | 716 . . . 738 | − | 5' Flanking Genomic DNA |
| | TTTA/ | Lb_V2/Lb | | | |
| | TTTA | | | | |
| 5F-70 | TTC/ | Fn/Lb_V1/ | 730 . . . 752 | − | 5' Flanking Genomic DNA |
| | TTTC/ | Lb_V2/Lb | | | |
| | TTTC | | | | |
| 5F-71 | TTC | Fn | 766 . . . 788 | + | 5' Flanking Genomic DNA |
| 5F-72 | TTCA | Lb_V1 | 767 . . . 789 | + | 5' Flanking Genomic DNA |
| 5F-73 | TTC | Fn | 770 . . . 792 | + | 5' Flanking Genomic DNA |
| 5F-74 | TTCC | Lb_V1 | 771 . . . 793 | + | 5' Flanking Genomic DNA |
| 5F-75 | TCCA | Lb_V1 | 772 . . . 794 | + | 5' Flanking Genomic DNA |
| 5F-76 | TTG | Fn | 777 . . . 799 | + | 5' Flanking Genomic DNA |
| 5F-77 | TATC | Lb_V2 | 781 . . . 803 | + | 5' Flanking Genomic DNA |
| 5F-78 | TTC | Fn | 785 . . . 807 | + | 5' Flanking Genomic DNA |
| 5F-79 | TATA | Lb_V2 | 766 . . . 788 | − | 5' Flanking Genomic DNA |
| 5F-80 | TTA | Fn | 768 . . . 790 | − | 5' Flanking Genomic DNA |
| 5F-81 | TTA/ | Fn/Lb_V1/ | 791 . . . 813 | + | 5' Flanking Genomic DNA |
| | TTTA/ | Lb_V2/Lb | | | |
| | TTTA | | | | |
| 5F-82 | TATA | Lb_V2 | 793 . . . 815 | + | 5' Flanking Genomic DNA |
| 5F-83 | TTCA | Lb_V1 | 783 . . . 805 | − | 5' Flanking Genomic DNA |
| 5F-84 | TTC/ | Fn/Lb_V1/ | 784 . . . 806 | − | 5' Flanking Genomic DNA |
| | TTTC/ | Lb_V2/Lb | | | |
| | TTTC | | | | |
| 5F-85 | TTCC | Lb_V1 | 788 . . . 810 | − | 5' Flanking Genomic DNA |
| 5F-86 | TTC/ | Fn/Lb_V1/ | 789 . . . 811 | − | 5' Flanking Genomic DNA |
| | TTTC/ | Lb_V2/Lb | | | |
| | TTTC | | | | |
| 5F-87 | TTG/ | Fn/Lb_V1/ | 815 . . . 837 | − | 5' Flanking Genomic DNA |
| | TTTG/ | Lb_V2/Lb | | | |
| | TTTG | | | | |
| 5F-88 | TTG/ | Fn/Lb_V1/ | 849 . . . 871 | + | 5' Flanking Genomic DNA |
| | TTTG/ | Lb_V2/Lb | | | |
| | TTTG | | | | |
| 5F-89 | TATC | Lb_V2 | 857 . . . 879 | + | 5' Flanking Genomic DNA |
| 5F-90 | TTC/ | Fn/Lb_V1/ | 863 . . . 885 | + | 5' Flanking Genomic DNA |
| | TTTC/ | Lb_V2/Lb | | | |
| | TTTC | | | | |
| 5F-91 | TTCA | Lb_V1 | 864 . . . 886 | + | 5' Flanking Genomic DNA |
| 5F-92 | TTA/ | Fn/Lb_V1/ | 862 . . . 884 | − | 5' Flanking Genomic DNA |
| | TTTA/ | Lb_V2/Lb | | | |
| | TTTA | | | | |
| 5F-93 | TTA/ | Fn/Lb_V1/ | 866 . . . 888 | − | 5' Flanking Genomic DNA |
| | TTTA/ | Lb_V2/Lb | | | |
| | TTTA | | | | |
| 5F-94 | TATC | Lb_V2 | 922 . . . 944 | − | 5' Flanking Genomic DNA |
| 5F-95 | TATG | Lb_V2 | 951 . . . 973 | + | 5' Flanking Genomic DNA |
| 5F-96 | TTA | Fn | 961 . . . 983 | + | 5' Flanking Genomic DNA |
| 5F-97 | TTG/ | Fn/Lb_V1/ | 965 . . . 987 | + | 5' Flanking Genomic DNA |
| | TTTG/ | Lb_V2/Lb | | | |
| | TTTG | | | | |
| 5F-98 | TTA | Fn | 965 . . . 987 | − | 5' Flanking Genomic DNA |
| 5F-99 | TTA | Fn | 945 . . . 967 | − | 5' Flanking Genomic DNA |
| 5F-100 | TTA/ | Fn/Lb_V1/ | 970 . . . 992 | + | 5' Flanking Genomic DNA |
| | TTTA/ | Lb_V2/Lb | | | |
| | TTTA | | | | |
| 5F-101 | TCCA | Lb_V1 | 975 . . . 997 | + | 5' Flanking Genomic DNA |
| 5J-1 | TCCC | Lb_V1 | 982 . . . 1004 | + | 5' Junction |
| 5J-2 | TTA | Fn | 985 . . . 1007 | + | 5' Junction |
| 5J-3 | TTG | Fn | 999 . . . 1021 | + | 5' Junction |
| 5J-4 | TTG/ | Fn/Lb_V1/ | 998 . . . 1020 | − | 5' Junction |
| | TTTG/ | Lb_V2/Lb | | | |
| | TTTG | | | | |
| TI-1 | TTA | Fn | 1030 . . . 1052 | + | Transgenic Insert |
| TI-2 | TTG/ | Fn/Lb_V1/ | 1045 . . . 1067 | + | Transgenic Insert |
| | TTTG/ | Lb_V2/Lb | | | |
| | TTTG | | | | |
| TI-3 | TTC/ | Fn/Lb_V1/ | 1028 . . . 1050 | − | Transgenic Insert |
| | TTTC/ | Lb_V2/Lb | | | |
| | TTTC | | | | |
| TI-4 | TTA | Fn | 1051 . . . 1073 | + | Transgenic Insert |
| TI-5 | TTG | Fn | 1037 . . . 1059 | − | Transgenic Insert |
| TI-6 | TTG | Fn | 1066 . . . 1088 | + | Transgenic Insert |
| TI-7 | TTA | Fn | 1062 . . . 1084 | − | Transgenic Insert |
| TI-8 | TATA | Lb_V2 | 1066 . . . 1088 | − | Transgenic Insert |
| TI-9 | TTA | Fn | 1068 . . . 1090 | − | Transgenic Insert |
| TI-10 | TATA | Lb_V2 | 1093 . . . 1115 | + | Transgenic Insert |
| TI-11 | TTA | Fn | 1077 . . . 1099 | − | Transgenic Insert |
| TI-12 | TTC | Fn | 1105 . . . 1127 | − | Transgenic Insert |
| TI-13 | TTA/ | Fn/Lb_V1/ | 1138 . . . 1160 | + | Transgenic Insert |
| | TTTA/ | Lb_V2/Lb | | | |
| | TTTA | | | | |
| TI-14 | TATG | Lb_V2 | 1140 . . . 1162 | + | Transgenic Insert |
| TI-15 | TTA | Fn | 1158 . . . 1180 | + | Transgenic Insert |
| TI-16 | TTG | Fn | 1138 . . . 1160 | − | Transgenic Insert |
| TI-17 | TTC | Fn | 1141 . . . 1163 | − | Transgenic Insert |
| TI-18 | TATC | Lb_V2 | 1144 . . . 1166 | − | Transgenic Insert |
| TI-19 | TATG | Lb_V2 | 1154 . . . 1176 | − | Transgenic Insert |
| TI-20 | TTG | Fn | 1160 . . . 1182 | − | Transgenic Insert |
| TI-21 | TTCA | Lb_V1 | 1165 . . . 1187 | − | Transgenic Insert |
| TI-22 | TTC/ | Fn/Lb_V1/ | 1166 . . . 1188 | − | Transgenic Insert |
| | TTTC/ | Lb_V2/Lb | | | |
| | TTTC | | | | |
| TI-23 | TTA/ | Fn/Lb_V1/ | 1171 . . . 1193 | − | Transgenic Insert |
| | TTTA/ | Lb_V2/Lb | | | |
| | TTTA | | | | |
| TI-24 | TATA | Lb_V2 | 1214 . . . 1236 | + | Transgenic Insert |
| TI-25 | TATA | Lb_V2 | 1216 . . . 1238 | + | Transgenic Insert |
| TI-26 | TTA | Fn | 1222 . . . 1244 | + | Transgenic Insert |
| TI-27 | TTC | Fn | 1228 . . . 1250 | + | Transgenic Insert |
| TI-28 | TATA | Lb_V2 | 1207 . . . 1229 | − | Transgenic Insert |
| TI-29 | TATA | Lb_V2 | 1209 . . . 1231 | − | Transgenic Insert |
| TI-30 | TATA | Lb_V2 | 1211 . . . 1233 | − | Transgenic Insert |
| TI-31 | TATA | Lb_V2 | 1234 . . . 1256 | + | Transgenic Insert |
| TI-32 | TATA | Lb_V2 | 1236 . . . 1258 | + | Transgenic Insert |
| TI-33 | TATA | Lb_V2 | 1238 . . . 1260 | + | Transgenic Insert |
| TI-34 | TTA | Fn | 1241 . . . 1263 | + | Transgenic Insert |
| TI-35 | TATG | Lb_V2 | 1243 . . . 1265 | + | Transgenic Insert |
| TI-36 | TTG | Fn | 1247 . . . 1269 | + | Transgenic Insert |
| TI-37 | TTA | Fn | 1251 . . . 1273 | + | Transgenic Insert |
| TI-38 | TTA | Fn | 1232 . . . 1254 | − | Transgenic Insert |
| TI-39 | TTC | Fn | 1254 . . . 1276 | + | Transgenic Insert |
| TI-40 | TTA | Fn | 1257 . . . 1279 | + | Transgenic Insert |
| TI-41 | TTG | Fn | 1265 . . . 1287 | + | Transgenic Insert |
| TI-42 | TTA | Fn | 1245 . . . 1267 | - | Transgenic Insert |
| TI-43 | TTA | Fn | 1270 . . . 1292 | + | Transgenic Insert |
| TI-44 | TTG | Fn | 1249 . . . 1271 | − | Transgenic Insert |
| TI-45 | TATG | Lb_V2 | 1254 . . . 1276 | − | Transgenic Insert |
| TI-46 | TTA | Fn | 1262 . . . 1284 | − | Transgenic Insert |
| TI-47 | TCCA | Lb_V1 | 1265 . . . 1287 | − | Transgenic Insert |
| TI-48 | TTCC | Lb_V1 | 1266 . . . 1288 | − | Transgenic Insert |
| TI-49 | TTC/ | Fn/Lb_V1/ | 1267 . . . 1289 | − | Transgenic Insert |
| | TTTC/ | Lb_V2/Lb | | | |
| | TTTC | | | | |
| TI-50 | TTC | Fn | 1271 . . . 1293 | − | Transgenic Insert |
| TI-51 | TTC/ | Fn/Lb_V1/ | 1315 . . . 1337 | + | Transgenic Insert |
| | TTTC/ | Lb_V2/Lb | | | |
| | TTTC | | | | |
| TI-52 | TTCC | Lb_V1 | 1316 . . . 1338 | + | Transgenic Insert |
| TI-53 | TTA | Fn | 1319 . . . 1341 | + | Transgenic Insert |
| TI-54 | TATA | Lb_V2 | 1311 . . . 1333 | − | Transgenic Insert |
| TI-55 | TTC/ | Fn/Lb_V1/ | 1315 . . . 1337 | − | Transgenic Insert |
| | TTTC/ | Lb_V2/Lb | | | |
| | TTTC | | | | |
| TI-56 | TCCA | Lb_V1 | 1389 . . . 1411 | − | Transgenic Insert |
| TI-57 | TTA | Fn | 1398 . . . 1420 | − | Transgenic Insert |
| TI-58 | TTG | Fn | 1403 . . . 1425 | − | Transgenic Insert |
| TI-59 | TCCA | Lb_V1 | 1428 . . . 1450 | + | Transgenic Insert |
| TI-60 | TATG | Lb_V2 | 1413 . . . 1435 | − | Transgenic Insert |
| TI-61 | TTA/ | Fn/Lb_V1/ | 1443 . . . 1465 | − | Transgenic Insert |
| | TTTA/ | Lb_V2/Lb | | | |
| | TTTA | | | | |
| TI-62 | TATG | Lb_V2 | 1490 . . . 1512 | + | Transgenic Insert |
| TI-63 | TATC | Lb_V2 | 1499 . . . 1521 | + | Transgenic Insert |
| TI-64 | TATG | Lb_V2 | 1504 . . . 1526 | + | Transgenic Insert |
| TI-65 | TTC | Fn | 1508 . . . 1530 | + | Transgenic Insert |
| TI-66 | TTCC | Lb_V1 | 1509 . . . 1531 | + | Transgenic Insert |
| TI-67 | TTC | Fn | 1512 . . . 1534 | + | Transgenic Insert |
| TI-68 | TTCC | Lb_V1 | 1513 . . . 1535 | + | Transgenic Insert |
| TI-69 | TTA/ | Fn/Lb_V1/ | 1517 . . . 1539 | + | Transgenic Insert |
| | TTTA/ | Lb_V2/Lb | | | |
| | TTTA | | | | |
| TI-70 | TTC/ | Fn/Lb_V1/ | 1522 . . . 1544 | + | Transgenic Insert |
| | TTTC/ | Lb_V2/Lb | | | |
| | TTTC | | | | |
| TI-71 | TTCG | Lb_V1 | 1523 . . . 1545 | + | Transgenic Insert |
| TI-72 | TATC | Lb_V2 | 1501 . . . 1523 | − | Transgenic Insert |
| TI-73 | TTA | Fn | 1503 . . . 1525 | − | Transgenic Insert |
| TI-74 | TTA | Fn | 1506 . . . 1528 | − | Transgenic Insert |
| TI-75 | TTG | Fn | 1513 . . . 1535 | − | Transgenic Insert |
| TI-76 | TATC | Lb_V2 | 1518 . . . 1540 | − | Transgenic Insert |
| TI-77 | TTG/ | Fn/Lb_V1/ | 1614 . . . 1636 | + | Transgenic Insert |
| | TTTG/ | Lb_V2/Lb | | | |
| | TTTG | | | | |
| TI-78 | TTG/ | Fn/Lb_V1/ | 1619 . . . 1641 | + | Transgenic Insert |
| | TTTG/ | Lb_V2/Lb | | | |
| | TTTG | | | | |
| TI-79 | TATA | Lb_V2 | 1626 . . . 1648 | + | Transgenic Insert |
| TI-80 | TTC | Fn | 1638 . . . 1660 | + | Transgenic Insert |
| TI-81 | TTCG | Lb_V1 | 1639 . . . 1661 | + | Transgenic Insert |
| TI-82 | TCCA | Lb_V1 | 1618 . . . 1640 | − | Transgenic Insert |
| TI-83 | TTCC | Lb_V1 | 1619 . . . 1641 | − | Transgenic Insert |
| TI-84 | TTC | Fn | 1620 . . . 1642 | − | Transgenic Insert |
| TI-85 | TATC | Lb_V2 | 1623 . . . 1645 | − | Transgenic Insert |
| TI-86 | TTA | Fn | 1625 . . . 1647 | − | Transgenic Insert |
| TI-87 | TTA | Fn | 1628 . . . 1650 | − | Transgenic Insert |
| TI-88 | TTC/ | Fn/Lb_V1/ | 1674 . . . 1696 | + | Transgenic Insert |
| | TTTC/ | Lb_V2/Lb | | | |
| | TTTC | | | | |
| TI-89 | TTC | Fn | 1683 . . . 1705 | + | Transgenic Insert |
| TI-90 | TTCA | Lb_V1 | 1684 . . . 1706 | + | Transgenic Insert |
| TI-91 | TCCC | Lb_V1 | 1693 . . . 1715 | + | Transgenic Insert |
| TI-92 | TTA/ | Fn/Lb_V1/ | 1676 . . . 1698 | − | Transgenic Insert |
| | TTTA/ | Lb_V2/Lb | | | |
| | TTTA | | | | |
| TI-93 | TTG/ | Fn/Lb_V1/ | 1690 . . . 1712 | − | Transgenic Insert |
| | TTTG/ | Lb_V2/Lb | | | |
| | TTTG | | | | |
| TI-94 | TTG | Fn | 1714 . . . 1736 | − | Transgenic Insert |
| TI-95 | TATA | Lb_V2 | 1719 . . . 1741 | − | Transgenic Insert |
| TI-96 | TTG/ | Fn/Lb_V1/ | 1833 . . . 1855 | + | Transgenic Insert |
| | TTTG/ | Lb_V2/Lb | | | |
| | TTTG | | | | |
| TI-97 | TTG | Fn | 1842 . . . 1864 | + | Transgenic Insert |
| TI-98 | TTC | Fn | 1845 . . . 1867 | + | Transgenic Insert |
| TI-99 | TTCG | Lb_V1 | 1846 . . . 1868 | + | Transgenic Insert |
| TI-100 | TTG | Fn | 1829 . . . 1851 | − | Transgenic Insert |
| TI-101 | TTA/ | Fn/Lb_V1/ | 1841 . . . 1863 | − | Transgenic Insert |
| | TTTA/ | Lb_V2/Lb | | | |
| | TTTA | | | | |
| TI-102 | TTG/ | Fn/Lb_V1/ | 1873 . . . 1895 | + | Transgenic Insert |
| | TTTG/ | Lb_V2/Lb | | | |
| | TTTG | | | | |
| TI-103 | TTA | Fn | 1882 . . . 1904 | + | Transgenic Insert |
| TI-104 | TATC | Lb_V2 | 1892 . . . 1914 | + | Transgenic Insert |
| TI-105 | TTC | Fn | 1897 . . . 1919 | + | Transgenic Insert |
| TI-106 | TTCG | Lb_V1 | 1898 . . . 1920 | + | Transgenic Insert |
| TI-107 | TTA/ | Fn/Lb_V1/ | 1907 . . . 1929 | + | Transgenic Insert |
| | TTTA/ | Lb_V2/Lb | | | |
| | TTTA | | | | |
| TI-108 | TTA | Fn | 1892 . . . 1914 | − | Transgenic Insert |
| TI-109 | TTC/ | Fn/Lb_V1/ | 1922 . . . 1944 | + | Transgenic Insert |
| | TTTC/ | Lb_V2/Lb | | | |
| | TTTC | | | | |
| TI-110 | TTCA | Lb_V1 | 1910 . . . 1932 | − | Transgenic Insert |
| TI-111 | TTC/ | Fn/Lb_V1/ | 1911 . . . 1933 | − | Transgenic Insert |
| | TTTC/ | Lb_V2/Lb | | | |
| | TTTC | | | | |
| TI-112 | TTA/ | Fn/Lb_V1/ | 2002 . . . 2024 | + | Transgenic Insert |
| | TTTA/ | Lb_V2/Lb | | | |
| | TTTA | | | | |
| TI-113 | TTC/ | Fn/Lb_V1/ | 2008 . . . 2030 | + | Transgenic Insert |
| | TTTC/ | Lb_V2/Lb | | | |
| | TTTC | | | | |
| TI-114 | TTA | Fn | 2011 . . . 2033 | + | Transgenic Insert |
| TI-115 | TATG | Lb_V2 | 2013 . . . 2035 | + | Transgenic Insert |
| TI-116 | TTA/ | Fn/Lb_V1/ | 2017 . . . 2039 | + | Transgenic Insert |
| | TTTA/ | Lb_V2/Lb | | | |
| | TTTA | | | | |
| TI-117 | TTG/ | Fn/Lb_V1/ | 2063 . . . 2085 | + | Transgenic Insert |
| | TTTG/ | Lb_V2/Lb | | | |
| | TTTG | | | | |
| TI-118 | TTG/ | Fn/Lb_V1/ | 2068 . . . 2090 | + | Transgenic Insert |
| | TTTG/ | Lb_V2/Lb | | | |
| | TTTG | | | | |
| TI-119 | TATA | Lb_V2 | 2073 . . . 2095 | + | Transgenic Insert |
| TI-120 | TATG | Lb_V2 | 2075 . . . 2097 | + | Transgenic Insert |
| TI-121 | TTC | Fn | 2078 . . . 2100 | + | Transgenic Insert |
| TI-122 | TTCG | Lb_V1 | 2079 . . . 2101 | + | Transgenic Insert |
| TI-123 | TTA/ | Fn/Lb_V1/ | 2110 . . . 2132 | + | Transgenic Insert |
| | TTTA/ | Lb_V2/Lb | | | |
| | TTTA | | | | |
| TI-124 | TTC/ | Fn/Lb_V1/ | 2114 . . . 2136 | + | Transgenic Insert |
| | TTTC/ | Lb_V2/Lb | | | |
| | TTTC | | | | |
| TI-125 | TTCA | Lb_V1 | 2115 . . . 2137 | + | Transgenic Insert |
| TI-126 | TATG | Lb_V2 | 2122 . . . 2144 | + | Transgenic Insert |
| TI-127 | TTA/ | Fn/Lb_V1/ | 2156 . . . 2178 | + | Transgenic Insert |
| | TTTA/ | Lb_V2/Lb | | | |
| | TTTA | | | | |
| TI-128 | TTCC | Lb_V1 | 2150 . . . 2172 | − | Transgenic Insert |
| TI-129 | TTC | Fn | 2151 . . . 2173 | − | Transgenic Insert |
| TI-130 | TCCG | Lb_V1 | 2154 . . . 2176 | − | Transgenic Insert |
| TI-131 | TTCG | Lb_V1 | 2162 . . . 2184 | − | Transgenic Insert |
| TI-132 | TTC | Fn | 2163 . . . 2185 | − | Transgenic Insert |
| TI-133 | TTC/ | Fn/Lb_V1/ | 2205 . . . 2227 | + | Transgenic Insert |
| | TTTC/ | Lb_V2/Lb | | | |
| | TTTC | | | | |
| TI-134 | TTC/ | Fn/Lb_V1/ | 2231 . . . 2253 | + | Transgenic Insert |
| | TTTC/ | Lb_V2/Lb | | | |
| | TTTC | | | | |
| TI-135 | TTCC | Lb_V1 | 2232 . . . 2254 | + | Transgenic Insert |
| TI-136 | TCCG | Lb_V1 | 2233 . . . 2255 | + | Transgenic Insert |
| TI-137 | TTC | Fn | 2247 . . . 2269 | + | Transgenic Insert |
| TI-138 | TTCC | Lb_V1 | 2248 . . . 2270 | + | Transgenic Insert |
| TI-139 | TTG/ | Fn/Lb_V1/ | 2227 . . . 2249 | − | Transgenic Insert |
| | TTTG/ | Lb_V2/Lb | | | |
| | TTTG | | | | |
| TI-140 | TTC | Fn | 2251 . . . 2273 | + | Transgenic Insert |
| TI-141 | TTCA | Lb_V1 | 2252 . . . 2274 | + | Transgenic Insert |
| TI-142 | TTG/ | Fn/Lb_V1/ | 2236 . . . 2258 | − | Transgenic Insert |
| | TTTG/ | Lb_V2/Lb | | | |
| | TTTG | | | | |
| TI-143 | TTA/ | Fn/Lb_V1/ | 2241 . . . 2263 | − | Transgenic Insert |
| | TTTA/ | Lb_V2/Lb | | | |
| | TTTA | | | | |
| TI-144 | TTA | Fn | 2266 . . . 2288 | + | Transgenic Insert |
| TI-145 | TTG | Fn | 2297 . . . 2319 | + | Transgenic Insert |
| TI-146 | TTC | Fn | 2311 . . . 2333 | + | Transgenic Insert |
| TI-147 | TTCG | Lb_V1 | 2312 . . . 2334 | + | Transgenic Insert |
| TI-148 | TTG | Fn | 2315 . . . 2337 | + | Transgenic Insert |
| TI-149 | TCCG | Lb_V1 | 2319 . . . 2341 | + | Transgenic Insert |
| TI-150 | TATG | Lb_V2 | 2326 . . . 2348 | + | Transgenic Insert |
| TI-151 | TTC/ | Fn/Lb_V1/ | 2335 . . . 2357 | + | Transgenic Insert |
| | TTTC/ | Lb_V2/Lb | | | |
| | TTTC | | | | |
| TI-152 | TTCG | Lb_V1 | 2336 . . . 2358 | + | Transgenic Insert |
| TI-153 | TTC/ | Fn/Lb_V1/ | 2316 . . . 2338 | − | Transgenic Insert |
| | TTTC/ | Lb_V2/Lb | | | |
| | TTTC | | | | |
| TI-154 | TTA | Fn | 2339 . . . 2361 | + | Transgenic Insert |
| TI-155 | TTG | Fn | 2349 . . . 2371 | + | Transgenic Insert |
| TI-156 | TTG | Fn | 2357 . . . 2379 | + | Transgenic Insert |
| TI-157 | TTC | Fn | 2366 . . . 2388 | + | Transgenic Insert |
| TI-158 | TTCC | Lb_V1 | 2367 . . . 2389 | + | Transgenic Insert |
| TI-159 | TTG | Fn | 2377 . . . 2399 | + | Transgenic Insert |
| TI-160 | TTC | Fn | 2359 . . . 2381 | − | Transgenic Insert |
| TI-161 | TTCC | Lb_V1 | 2369 . . . 2391 | − | Transgenic Insert |
| TI-162 | TTC | Fn | 2370 . . . 2392 | − | Transgenic Insert |
| TI-163 | TTC | Fn | 2373 . . . 2395 | − | Transgenic Insert |
| TI-164 | TATC | Lb_V2 | 2404 . . . 2426 | + | Transgenic Insert |
| TI-165 | TATC | Lb_V2 | 2384 . . . 2406 | − | Transgenic Insert |
| TI-166 | TCCA | Lb_V1 | 2416 . . . 2438 | + | Transgenic Insert |
| TI-167 | TATC | Lb_V2 | 2429 . . . 2451 | + | Transgenic Insert |
| TI-168 | TTG | Fn | 2408 . . . 2430 | − | Transgenic Insert |
| TI-169 | TCCG | Lb_V1 | 2416 . . . 2438 | − | Transgenic Insert |
| TI-170 | TTG | Fn | 2447 . . . 2469 | + | Transgenic Insert |
| TI-171 | TATC | Lb_V2 | 2438 . . . 2460 | − | Transgenic Insert |
| TI-172 | TTG | Fn | 2462 . . . 2484 | + | Transgenic Insert |
| TI-173 | TTG/ | Fn/Lb_V1/ | 2469 . . . 2491 | + | Transgenic Insert |
| | TTTG/ | Lb_V2/Lb | | | |
| | TTTG | | | | |
| TI-174 | TCCG | Lb_V1 | 2473 . . . 2495 | + | Transgenic Insert |
| TI-175 | TTC | Fn | 2482 . . . 2504 | + | Transgenic Insert |
| TI-176 | TTCG | Lb_V1 | 2483 . . . 2505 | + | Transgenic Insert |
| TI-177 | TCCG | Lb_V1 | 2488 . . . 2510 | + | Transgenic Insert |
| TI-178 | TTG | Fn | 2491 . . . 2513 | + | Transgenic Insert |
| TI-179 | TTC | Fn | 2504 . . . 2526 | + | Transgenic Insert |
| TI-180 | TTG | Fn | 2485 . . . 2507 | − | Transgenic Insert |
| TI-181 | TCCG | Lb_V1 | 2488 . . . 2510 | − | Transgenic Insert |
| TI-182 | TCCA | Lb_V1 | 2523 . . . 2545 | + | Transgenic Insert |
| TI-183 | TATG | Lb_V2 | 2505 . . . 2527 | − | Transgenic Insert |
| TI-184 | TCCA | Lb_V1 | 2530 . . . 2552 | + | Transgenic Insert |
| TI-185 | TATC | Lb_V2 | 2534 . . . 2556 | + | Transgenic Insert |
| TI-186 | TCCA | Lb_V1 | 2517 . . . 2539 | − | Transgenic Insert |
| TI-187 | TTCC | Lb_V1 | 2518 . . . 2540 | − | Transgenic Insert |
| TI-188 | TTC | Fn | 2519 . . . 2541 | − | Transgenic Insert |
| TI-189 | TTG/ | Fn/Lb_V1/ | 2549 . . . 2571 | + | Transgenic Insert |
| | TTTG/ | Lb_V2/Lb | | | |
| | TTTG | | | | |
| TI-190 | TCCG | Lb_V1 | 2527 . . . 2549 | − | Transgenic Insert |
| TI-191 | TTG | Fn | 2548 . . . 2570 | − | Transgenic Insert |
| TI-192 | TCCA | Lb_V1 | 2571 . . . 2593 | + | Transgenic Insert |
| TI-193 | TTG | Fn | 2560 . . . 2582 | − | Transgenic Insert |
| TI-194 | TCCG | Lb_V1 | 2563 . . . 2585 | − | Transgenic Insert |
| TI-195 | TTC | Fn | 2604 . . . 2626 | + | Transgenic Insert |
| TI-196 | TTCA | Lb_V1 | 2583 . . . 2605 | − | Transgenic Insert |
| TI-197 | TTC | Fn | 2584 . . . 2606 | − | Transgenic Insert |
| TI-198 | TTC | Fn | 2623 . . . 2645 | + | Transgenic Insert |
| TI-199 | TTCC | Lb_V1 | 2624 . . . 2646 | + | Transgenic Insert |
| TI-200 | TCCC | Lb_V1 | 2625 . . . 2647 | + | Transgenic Insert |
| TI-201 | TCCA | Lb_V1 | 2607 . . . 2629 | − | Transgenic Insert |
| TI-202 | TTCC | Lb_V1 | 2608 . . . 2630 | − | Transgenic Insert |
| TI-203 | TTC/ | Fn/Lb_V1/ | 2609 . . . 2631 | − | Transgenic Insert |
| | TTTC/ | Lb_V2/Lb | | | |
| | TTTC | | | | |
| TI-204 | TTG | Fn | 2647 . . . 2669 | + | Transgenic Insert |
| TI-205 | TCCA | Lb_V1 | 2627 . . . 2649 | − | Transgenic Insert |
| TI-206 | TCCA | Lb_V1 | 2638 . . . 2660 | − | Transgenic Insert |
| TI-207 | TCCA | Lb_V1 | 2671 . . . 2693 | + | Transgenic Insert |
| TI-208 | TCCC | Lb_V1 | 2686 . . . 2708 | + | Transgenic Insert |
| TI-209 | TATC | Lb_V2 | 2695 . . . 2717 | + | Transgenic Insert |
| TI-210 | TCCC | Lb_V1 | 2697 . . . 2719 | + | Transgenic Insert |
| TI-211 | TTG/ | Fn/Lb_V1/ | 2705 . . . 2727 | + | Transgenic Insert |
| | TTTG/ | Lb_V2/Lb | | | |
| | TTTG | | | | |
| TI-212 | TTG | Fn | 2717 . . . 2739 | + | Transgenic Insert |
| TI-213 | TCCA | Lb_V1 | 2722 . . . 2744 | + | Transgenic Insert |
| TI-214 | TTA | Fn | 2727 . . . 2749 | + | Transgenic Insert |
| TI-215 | TCCG | Lb_V1 | 2713 . . . 2735 | − | Transgenic Insert |
| TI-216 | TTA | Fn | 2745 . . . 2767 | + | Transgenic Insert |
| TI-217 | TCCA | Lb_V1 | 2730 . . . 2752 | − | Transgenic Insert |
| TI-218 | TTG | Fn | 2737 . . . 2759 | − | Transgenic Insert |
| TI-219 | TTG | Fn | 2743 . . . 2765 | − | Transgenic Insert |
| TI-220 | TCCA | Lb_V1 | 2754 . . . 2776 | − | Transgenic Insert |
| TI-221 | TTG | Fn | 2777 . . . 2799 | + | Transgenic Insert |
| TI-222 | TATC | Lb_V2 | 2756 . . . 2778 | − | Transgenic Insert |
| TI-223 | TTA | Fn | 2758 . . . 2780 | − | Transgenic Insert |
| TI-224 | TCCA | Lb_V1 | 2766 . . . 2788 | − | Transgenic Insert |
| TI-225 | TCCA | Lb_V1 | 2773 . . . 2795 | − | Transgenic Insert |
| TI-226 | TTG | Fn | 2798 . . . 2820 | + | Transgenic Insert |
| TI-227 | TTA | Fn | 2800 . . . 2822 | − | Transgenic Insert |
| TI-228 | TTG/ | Fn/Lb_V1/ | 2807 . . . 2829 | − | Transgenic Insert |
| | TTTG/ | Lb_V2/Lb | | | |
| | TTTG | | | | |
| TI-229 | TATC | Lb_V2 | 2822 . . . 2844 | − | Transgenic Insert |
| TI-230 | TTC | Fn | 2845 . . . 2867 | + | Transgenic Insert |
| TI-231 | TTCG | Lb_V1 | 2846 . . . 2868 | + | Transgenic Insert |
| TI-232 | TTG | Fn | 2855 . . . 2877 | + | Transgenic Insert |
| TI-233 | TTG | Fn | 2873 . . . 2895 | + | Transgenic Insert |
| TI-234 | TCCA | Lb_V1 | 2889 . . . 2911 | + | Transgenic Insert |
| TI-235 | TTCA | Lb_V1 | 2874 . . . 2896 | − | Transgenic Insert |
| TI-236 | TTC | Fn | 2875 . . . 2897 | − | Transgenic Insert |
| TI-237 | TCCC | Lb_V1 | 2907 . . . 2929 | + | Transgenic Insert |
| TI-238 | TTC | Fn | 2927 . . . 2949 | + | Transgenic Insert |
| TI-239 | TTA | Fn | 2911 . . . 2933 | − | Transgenic Insert |
| TI-240 | TTC | Fn | 2941 . . . 2963 | + | Transgenic Insert |
| TI-241 | TTG | Fn | 2944 . . . 2966 | + | Transgenic Insert |
| TI-242 | TTA | Fn | 2929 . . . 2951 | − | Transgenic Insert |
| TI-243 | TTG | Fn | 2966 . . . 2988 | + | Transgenic Insert |
| TI-244 | TCCA | Lb_V1 | 2948 . . . 2970 | − | Transgenic Insert |
| TI-245 | TTCC | Lb_V1 | 2949 . . . 2971 | − | Transgenic Insert |
| TI-246 | TTC | Fn | 2950 . . . 2972 | − | Transgenic Insert |
| TI-247 | TCCA | Lb_V1 | 2963 . . . 2985 | − | Transgenic Insert |
| TI-248 | TCCG | Lb_V1 | 2985 . . . 3007 | + | Transgenic Insert |
| TI-249 | TTCC | Lb_V1 | 2964 . . . 2986 | − | Transgenic Insert |
| TI-250 | TTC | Fn | 2965 . . . 2987 | − | Transgenic Insert |
| TI-251 | TTA | Fn | 2968 . . . 2990 | − | Transgenic Insert |
| TI-252 | TTCA | Lb_V1 | 2985 . . . 3007 | − | Transgenic Insert |
| TI-253 | TATG | Lb_V2 | 3007 . . . 3029 | + | Transgenic Insert |
| TI-254 | TTC | Fn | 2986 . . . 3008 | − | Transgenic Insert |
| TI-255 | TTC | Fn | 3016 . . . 3038 | + | Transgenic Insert |
| TI-256 | TTCA | Lb_V1 | 3017 . . . 3039 | + | Transgenic Insert |
| TI-257 | TCCG | Lb_V1 | 3006 . . . 3028 | − | Transgenic Insert |
| TI-258 | TTCC | Lb_V1 | 3007 . . . 3029 | − | Transgenic Insert |
| TI-259 | TTC | Fn | 3008 . . . 3030 | − | Transgenic Insert |
| TI-260 | TCCC | Lb_V1 | 3011 . . . 3033 | − | Transgenic Insert |
| TI-261 | TTCG | Lb_V1 | 3018 . . . 3040 | − | Transgenic Insert |
| TI-262 | TTC | Fn | 3019 . . . 3041 | − | Transgenic Insert |
| TI-263 | TTG | Fn | 3030 . . . 3052 | − | Transgenic Insert |
| TI-264 | TCCA | Lb_V1 | 3060 . . . 3082 | + | Transgenic Insert |
| TI-265 | TCCC | Lb_V1 | 3065 . . . 3087 | + | Transgenic Insert |
| TI-266 | TTCC | Lb_V1 | 3044 . . . 3066 | − | Transgenic Insert |
| TI-267 | TTC | Fn | 3045 . . . 3067 | − | Transgenic Insert |
| TI-268 | TATG | Lb_V2 | 3050 . . . 3072 | − | Transgenic Insert |
| TI-269 | TATC | Lb_V2 | 3079 . . . 3101 | + | Transgenic Insert |
| TI-270 | TTA | Fn | 3083 . . . 3105 | + | Transgenic Insert |
| TI-271 | TTG | Fn | 3075 . . . 3097 | − | Transgenic Insert |
| TI-272 | TTA | Fn | 3111 . . . 3133 | + | Transgenic Insert |
| TI-273 | TTC | Fn | 3115 . . . 3137 | + | Transgenic Insert |
| TI-274 | TTC | Fn | 3118 . . . 3140 | + | Transgenic Insert |
| TI-275 | TTCG | Lb_V1 | 3119 . . . 3141 | + | Transgenic Insert |
| TI-276 | TTC | Fn | 3126 . . . 3148 | + | Transgenic Insert |
| TI-277 | TTCC | Lb_V1 | 3127 . . . 3149 | + | Transgenic Insert |
| TI-278 | TTG | Fn | 3106 . . . 3128 | − | Transgenic Insert |
| TI-279 | TCCC | Lb_V1 | 3128 . . . 3150 | + | Transgenic Insert |
| TI-280 | TTC | Fn | 3136 . . . 3158 | + | Transgenic Insert |
| TI-281 | TTCG | Lb_V1 | 3137 . . . 3159 | + | Transgenic Insert |
| TI-282 | TATA | Lb_V2 | 3115 . . . 3137 | − | Transgenic Insert |
| TI-283 | TATA | Lb_V2 | 3142 . . . 3164 | + | Transgenic Insert |
| TI-284 | TCCA | Lb_V1 | 3151 . . . 3173 | + | Transgenic Insert |
| TI-285 | TCCA | Lb_V1 | 3132 . . . 3154 | − | Transgenic Insert |
| TI-286 | TTC | Fn | 3167 . . . 3189 | + | Transgenic Insert |
| TI-287 | TTG | Fn | 3155 . . . 3177 | − | Transgenic Insert |
| TI-288 | TTG | Fn | 3161 . . . 3183 | − | Transgenic Insert |
| TI-289 | TTCA | Lb_V1 | 3171 . . . 3193 | − | Transgenic Insert |
| TI-290 | TTC | Fn | 3172 . . . 3194 | − | Transgenic Insert |
| TI-291 | TTG/ | Fn/Lb_V1/ | 3181 . . . 3203 | − | Transgenic Insert |
| | TTTG/ | Lb_V2/Lb | | | |
| | TTTG | | | | |
| TI-292 | TTCA | Lb_V1 | 3193 . . . 3215 | − | Transgenic Insert |
| TI-293 | TTC | Fn | 3194 . . . 3216 | − | Transgenic Insert |
| TI-294 | TTG | Fn | 3218 . . . 3240 | + | Transgenic Insert |
| TI-295 | TTCG | Lb_V1 | 3202 . . . 3224 | − | Transgenic Insert |
| TI-296 | TTC/ | Fn/Lb_V1/ | 3203 . . . 3225 | − | Transgenic Insert |
| | TTTC/ | Lb_V2/Lb | | | |
| | TTTC | | | | |
| TI-297 | TATC | Lb_V2 | 3226 . . . 3248 | + | Transgenic Insert |
| TI-298 | TCCG | Lb_V1 | 3209 . . . 3231 | − | Transgenic Insert |
| TI-299 | TTA | Fn | 3243 . . . 3265 | + | Transgenic Insert |
| TI-300 | TTCG | Lb_V1 | 3223 . . . 3245 | − | Transgenic Insert |
| TI-301 | TTC | Fn | 3224 . . . 3246 | − | Transgenic Insert |
| TI-302 | TTCG | Lb_V1 | 3228 . . . 3250 | − | Transgenic Insert |
| TI-303 | TTC | Fn | 3229 . . . 3251 | − | Transgenic Insert |
| TI-304 | TCCC | Lb_V1 | 3233 . . . 3255 | − | Transgenic Insert |
| TI-305 | TTG | Fn | 3277 . . . 3299 | + | Transgenic Insert |
| TI-306 | TATC | Lb_V2 | 3306 . . . 3328 | + | Transgenic Insert |
| TI-307 | TCCC | Lb_V1 | 3285 . . . 3307 | − | Transgenic Insert |
| TI-308 | TCCA | Lb_V1 | 3308 . . . 3330 | + | Transgenic Insert |
| TI-309 | TTC | Fn | 3295 . . . 3317 | − | Transgenic Insert |
| TI-310 | TTG | Fn | 3317 . . . 3339 | + | Transgenic Insert |
| TI-311 | TTCG | Lb_V1 | 3301 . . . 3323 | − | Transgenic Insert |
| TI-312 | TTC | Fn | 3302 . . . 3324 | − | Transgenic Insert |
| TI-313 | TTG | Fn | 3305 . . . 3327 | − | Transgenic Insert |
| TI-314 | TTC | Fn | 3311 . . . 3333 | − | Transgenic Insert |
| TI-315 | TTG | Fn | 3352 . . . 3374 | − | Transgenic Insert |
| TI-316 | TCCA | Lb_V1 | 3377 . . . 3399 | + | Transgenic Insert |
| TI-317 | TCCA | Lb_V1 | 3357 . . . 3379 | − | Transgenic Insert |
| TI-318 | TCCC | Lb_V1 | 3369 . . . 3391 | − | Transgenic Insert |
| TI-319 | TTG | Fn | 3399 . . . 3421 | + | Transgenic Insert |
| TI-320 | TTA | Fn | 3378 . . . 3400 | − | Transgenic Insert |
| TI-321 | TATG | Lb_V2 | 3381 . . . 3403 | − | Transgenic Insert |
| TI-322 | TTA/ | Fn/Lb_V1/ | 3383 . . . 3405 | − | Transgenic Insert |
| | TTTA/ | Lb_V2/Lb | | | |
| | TTTA | | | | |
| TI-323 | TTA | Fn | 3387 . . . 3409 | − | Transgenic Insert |
| TI-324 | TTA | Fn | 3401 . . . 3423 | − | Transgenic Insert |
| TI-325 | TTCA | Lb_V1 | 3404 . . . 3426 | − | Transgenic Insert |
| TI-326 | TTC | Fn | 3405 . . . 3427 | − | Transgenic Insert |
| TI-327 | TTG | Fn | 3414 . . . 3436 | − | Transgenic Insert |
| TI-328 | TTC/ | Fn/Lb_V1/ | 3446 . . . 3468 | + | Transgenic Insert |
| | TTTC/ | Lb_V2/Lb | | | |
| | TTTC | | | | |
| TI-329 | TTA | Fn | 3456 . . . 3478 | + | Transgenic Insert |
| TI-330 | TTCA | Lb_V1 | 3442 . . . 3464 | − | Transgenic Insert |
| TI-331 | TTC | Fn | 3443 . . . 3465 | − | Transgenic Insert |
| TI-332 | TTA/ | Fn/Lb_V1/ | 3484 . . . 3506 | + | Transgenic Insert |
| | TTTA/ | Lb_V2/Lb | | | |
| | TTTA | | | | |
| TI-333 | TATG | Lb_V2 | 3486 . . . 3508 | + | Transgenic Insert |
| TI-334 | TTG/ | Fn/Lb_V1/ | 3510 . . . 3532 | + | Transgenic Insert |
| | TTTG/ | Lb_V2/Lb | | | |
| | TTTG | | | | |
| TI-335 | TTG | Fn | 3516 . . . 3538 | + | Transgenic Insert |
| TI-336 | TATA | Lb_V2 | 3510 . . . 3532 | − | Transgenic Insert |
| TI-337 | TTA | Fn | 3512 . . . 3534 | − | Transgenic Insert |
| TI-338 | TTCA | Lb_V1 | 3520 . . . 3542 | − | Transgenic Insert |
| TI-339 | TTC/ | Fn/Lb_V1/ | 3521 . . . 3543 | − | Transgenic Insert |
| | TTTC/ | Lb_V2/Lb | | | |
| | TTTC | | | | |
| TI-340 | TTCA | Lb_V1 | 3620 . . . 3642 | − | Transgenic Insert |
| TI-341 | TTC | Fn | 3621 . . . 3643 | − | Transgenic Insert |
| TI-342 | TTA/ | Fn/Lb_V1/ | 3692 . . . 3714 | + | Transgenic Insert |
| | TTTA/ | Lb_V2/Lb | | | |
| | TTTA | | | | |
| TI-343 | TTA | Fn | 3729 . . . 3751 | + | Transgenic Insert |
| TI-344 | TTA | Fn | 3735 . . . 3757 | + | Transgenic Insert |
| TI-345 | TATA | Lb_V2 | 3741 . . . 3763 | + | Transgenic Insert |
| TI-346 | TTC/ | Fn/Lb_V1/ | 3746 . . . 3768 | + | Transgenic Insert |
| | TTTC/ | Lb_V2/Lb | | | |
| | TTTC | | | | |
| TI-347 | TTCG | Lb_V1 | 3747 . . . 3769 | + | Transgenic Insert |
| TI-348 | TTG | Fn | 3732 . . . 3754 | − | Transgenic Insert |
| TI-349 | TTG | Fn | 3739 . . . 3761 | − | Transgenic Insert |
| TI-350 | TCCG | Lb_V1 | 3769 . . . 3791 | + | Transgenic Insert |
| TI-351 | TCCG | Lb_V1 | 3775 . . . 3797 | + | Transgenic Insert |
| TI-352 | TTC | Fn | 3784 . . . 3806 | + | Transgenic Insert |
| TI-353 | TTCG | Lb_V1 | 3785 . . . 3807 | + | Transgenic Insert |
| TI-354 | TCCA | Lb_V1 | 3796 . . . 3818 | + | Transgenic Insert |
| TI-355 | TTA/ | Fn/Lb_V1/ | 3806 . . . 3828 | − | Transgenic Insert |
| | TTTA/ | Lb_V2/Lb | | | |
| | TTTA | | | | |
| TI-356 | TTA | Fn | 3841 . . . 3863 | + | Transgenic Insert |
| TI-357 | TTG | Fn | 3858 . . . 3880 | + | Transgenic Insert |
| TI-358 | TTA | Fn | 3865 . . . 3887 | + | Transgenic Insert |
| TI-359 | TATG | Lb_V2 | 3867 . . . 3889 | + | Transgenic Insert |
| TI-360 | TTCG | Lb_V1 | 3855 . . . 3877 | − | Transgenic Insert |
| TI-361 | TTC | Fn | 3856 . . . 3878 | − | Transgenic Insert |
| TI-362 | TTC | Fn | 3930 . . . 3952 | + | Transgenic Insert |
| TI-363 | TTC | Fn | 3933 . . . 3955 | + | Transgenic Insert |
| TI-364 | TTG | Fn | 3936 . . . 3958 | + | Transgenic Insert |
| TI-365 | TATG | Lb_V2 | 3920 . . . 3942 | − | Transgenic Insert |
| TI-366 | TTG/ | Fn/Lb_V1/ | 3938 . . . 3960 | − | Transgenic Insert |
| | TTTG/ | Lb_V2/Lb | | | |
| | TTTG | | | | |
| TI-367 | TTA | Fn | 3977 . . . 3999 | + | Transgenic Insert |
| TI-368 | TTG | Fn | 3985 . . . 4007 | + | Transgenic Insert |
| TI-369 | TTG | Fn | 3978 . . . 4000 | − | Transgenic Insert |
| TI-370 | TTCA | Lb_V1 | 3983 . . . 4005 | − | Transgenic Insert |
| TI-371 | TTC/ | Fn/Lb_V1/ | 3984 . . . 4006 | − | Transgenic Insert |
| | TTTC/ | Lb_V2/Lb | | | |
| | TTTC | | | | |
| TI-372 | TTA | Fn | 4031 . . . 4053 | + | Transgenic Insert |
| TI-373 | TTG/ | Fn/Lb_V1/ | 4038 . . . 4060 | + | Transgenic Insert |
| | TTTG/ | Lb_V2/Lb | | | |
| | TTTG | | | | |
| TI-374 | TTA/ | Fn/Lb_V1/ | 4040 . . . 4062 | − | Transgenic Insert |
| | TTTA/ | Lb_V2/Lb | | | |
| | TTTA | | | | |
| TI-375 | TTA | Fn | 4065 . . . 4087 | + | Transgenic Insert |
| TI-376 | TTG | Fn | 4071 . . . 4093 | + | Transgenic Insert |
| TI-377 | TTG/ | Fn/Lb_V1/ | 4068 . . . 4090 | − | Transgenic Insert |
| | TTTG | Lb | | | |
| TI-378 | TCCA | Lb_V1 | 4110 . . . 4132 | + | Transgenic Insert |
| TI-379 | TTG | Fn | 4113 . . . 4135 | + | Transgenic Insert |
| TI-380 | TTA/ | Fn/Lb_V1/ | 4096 . . . 4118 | − | Transgenic Insert |
| | TTTA/ | Lb_V2/Lb | | | |
| | TTTA | | | | |
| TI-381 | TTA | Fn | 4121 . . . 4143 | + | Transgenic Insert |
| TI-382 | TTCA | Lb_V1 | 4100 . . . 4122 | − | Transgenic Insert |
| TI-383 | TTC/ | Fn/Lb_V1/ | 4101 . . . 4123 | − | Transgenic Insert |
| | TTTC/ | Lb_V2/Lb | | | |
| | TTTC | | | | |
| TI-384 | TTG | Fn | 4110 . . . 4132 | − | Transgenic Insert |
| TI-385 | TTC/ | Fn/Lb_V1/ | 4113 . . . 4135 | − | Transgenic Insert |
| | TTTC/ | Lb_V2/Lb | | | |
| | TTTC | | | | |
| TI-386 | TTG/ | Fn/Lb_V1/ | 4126 . . . 4148 | − | Transgenic Insert |
| | TTTG/ | Lb_V2/Lb | | | |
| | TTTG | | | | |
| TI-387 | TTC/ | Fn/Lb_V1/ | 4174 . . . 4196 | + | Transgenic Insert |
| | TTTC/ | Lb_V2/Lb | | | |
| | TTTC | | | | |
| TI-388 | TTCA | Lb_V1 | 4175 . . . 4197 | + | Transgenic Insert |
| TI-389 | TTC/ | Fn/Lb_V1/ | 4188 . . . 4210 | + | Transgenic Insert |
| | TTTC/ | Lb_V2/Lb | | | |
| | TTTC | | | | |
| TI-390 | TTCC | Lb_V1 | 4189 . . . 4211 | + | Transgenic Insert |
| TI-391 | TCCA | Lb_V1 | 4190 . . . 4212 | + | Transgenic Insert |
| TI-392 | TTC | Fn | 4195 . . . 4217 | + | Transgenic Insert |
| TI-393 | TTCA | Lb_V1 | 4196 . . . 4218 | + | Transgenic Insert |
| TI-394 | TCCA | Lb_V1 | 4175 . . . 4197 | − | Transgenic Insert |
| TI-395 | TATC | Lb_V2 | 4177 . . . 4199 | − | Transgenic Insert |
| TI-396 | TTG | Fn | 4200 . . . 4222 | + | Transgenic Insert |
| TI-397 | TTG | Fn | 4184 . . . 4206 | − | Transgenic Insert |
| TI-398 | TATG | Lb_V2 | 4198 . . . 4220 | − | Transgenic Insert |
| TI-399 | TTA/ | Fn/Lb_V1/ | 4200 . . . 4222 | − | Transgenic Insert |
| | TTTA/ | Lb_V2/Lb | | | |
| | TTTA | | | | |
| TI-400 | TTA/ | Fn/Lb_V1/ | 4255 . . . 4277 | − | Transgenic Insert |
| | TTTA/ | Lb_V2/Lb | | | |
| | TTTA | | | | |
| TI-401 | TCCA | Lb_V1 | 4292 . . . 4314 | + | Transgenic Insert |
| TI-402 | TCCA | Lb_V1 | 4309 . . . 4331 | + | Transgenic Insert |
| TI-403 | TTA | Fn | 4296 . . . 4318 | − | Transgenic Insert |
| TI-404 | TTA | Fn | 4335 . . . 4357 | + | Transgenic Insert |
| TI-405 | TTG | Fn | 4314 . . . 4336 | − | Transgenic Insert |
| TI-406 | TATC | Lb_V2 | 4337 . . . 4359 | + | Transgenic Insert |
| TI-407 | TCCA | Lb_V1 | 4339 . . . 4361 | + | Transgenic Insert |
| TI-408 | TTCG | Lb_V1 | 4327 . . . 4349 | − | Transgenic Insert |
| TI-409 | TTC | Fn | 4328 . . . 4350 | − | Transgenic Insert |
| TI-410 | TCCA | Lb_V1 | 4358 . . . 4380 | + | Transgenic Insert |
| TI-411 | TATA | Lb_V2 | 4338 . . . 4360 | − | Transgenic Insert |
| TI-412 | TATA | Lb_V2 | 4340 . . . 4362 | − | Transgenic Insert |
| TI-413 | TATA | Lb_V2 | 4342 . . . 4364 | − | Transgenic Insert |
| TI-414 | TATA | Lb_V2 | 4365 . . . 4387 | + | Transgenic Insert |
| TI-415 | TATA | Lb_V2 | 4367 . . . 4389 | + | Transgenic Insert |
| TI-416 | TATA | Lb_V2 | 4369 . . . 4391 | + | Transgenic Insert |
| TI-417 | TATG | Lb_V2 | 4349 . . . 4371 | − | Transgenic Insert |
| TI-418 | TATA | Lb_V2 | 4351 . . . 4373 | − | Transgenic Insert |
| TI-419 | TCCA | Lb_V1 | 4374 . . . 4396 | + | Transgenic Insert |
| TI-420 | TTA | Fn | 4353 . . . 4375 | − | Transgenic Insert |
| TI-421 | TATA | Lb_V2 | 4378 . . . 4400 | + | Transgenic Insert |
| TI-422 | TTG | Fn | 4382 . . . 4404 | − | Transgenic Insert |
| TI-423 | TTA/ | Fn/Lb_V1/ | 4386 . . . 4408 | − | Transgenic Insert |
| | TTTA/ | Lb_V2/Lb | | | |
| | TTTA | | | | |
| TI-424 | TTC/ | Fn/Lb_V1/ | 4447 . . . 4469 | + | Transgenic Insert |
| | TTTC/ | Lb_V2/Lb | | | |
| | TTTC | | | | |
| TI-425 | TTCA | Lb_V1 | 4448 . . . 4470 | | Transgenic Insert |
| TI-426 | TTA/ | Fn/Lb_V1/ | 4454 . . . 4476 | + | Transgenic Insert |
| | TTTA/ | Lb_V2/Lb | | | |
| | TTTA | | | | |
| TI-427 | TTC/ | Fn/Lb_V1/ | 4460 . . . 4482 | + | Transgenic Insert |
| | TTTC/ | Lb_V2/Lb | | | |
| | TTTC | | | | |
| TI-428 | TCCG | Lb_V1 | 4466 . . . 4488 | + | Transgenic Insert |
| TI-429 | TTC/ | Fn/Lb_V1/ | 4478 . . . 4500 | + | Transgenic Insert |
| | TTTC/ | Lb_V2/Lb | | | |
| | TTTC | | | | |
| TI-430 | TTCG | Lb_V1 | 4479 . . . 4501 | + | Transgenic Insert |
| TI-431 | TCCC | Lb_V1 | 4512 . . . 4534 | + | Transgenic Insert |
| TI-432 | TCCG | Lb_V1 | 4518 . . . 4540 | + | Transgenic Insert |
| TI-433 | TCCC | Lb_V1 | 4534 . . . 4556 | + | Transgenic Insert |
| TI-434 | TCCG | Lb_V1 | 4539 . . . 4561 | + | Transgenic Insert |
| TI-435 | TTG | Fn | 4550 . . . 4572 | + | Transgenic Insert |
| TI-436 | TTC | Fn | 4554 . . . 4576 | + | Transgenic Insert |
| TI-437 | TTCG | Lb_V1 | 4555 . . . 4577 | + | Transgenic Insert |
| TI-438 | TTG | Fn | 4558 . . . 4580 | + | Transgenic Insert |
| TI-439 | TTC | Fn | 4564 . . . 4586 | + | Transgenic Insert |
| TI-440 | TTCA | Lb_V1 | 4565 . . . 4587 | + | Transgenic Insert |
| TI-441 | TTA | Fn | 4577 . . . 4599 | + | Transgenic Insert |
| TI-442 | TATC | Lb_V2 | 4564 . . . 4586 | − | Transgenic Insert |
| TI-443 | TATG | Lb_V2 | 4571 . . . 4593 | − | Transgenic Insert |
| TI-444 | TTG | Fn | 4579 . . . 4601 | − | Transgenic Insert |
| TI-445 | TTC | Fn | 4603 . . . 4625 | + | Transgenic Insert |
| TI-446 | TTCA | Lb_V1 | 4604 . . . 4626 | + | Transgenic Insert |
| TI-447 | TATC | Lb_V2 | 4590 . . . 4612 | − | Transgenic Insert |
| TI-448 | TTG/ | Fn/Lb_V1/ | 4648 . . . 4670 | + | Transgenic Insert |
| | TTTG/ | Lb_V2/Lb | | | |
| | TTTG | | | | |
| TI-449 | TTG | Fn | 4673 . . . 4695 | + | Transgenic Insert |
| TI-450 | TTC | Fn | 4677 . . . 4699 | + | Transgenic Insert |
| TI-451 | TTCG | Lb_V1 | 4678 . . . 4700 | + | Transgenic Insert |
| TI-452 | TTG/ | Fn/Lb_V1/ | 4683 . . . 4705 | + | Transgenic Insert |
| | TTTG/ | Lb_V2/Lb | | | |
| | TTTG | | | | |
| TI-453 | TTC | Fn | 4687 . . . 4709 | + | Transgenic Insert |
| TI-454 | TTC | Fn | 4694 . . . 4716 | + | Transgenic Insert |
| TI-455 | TTCG | Lb_V1 | 4695 . . . 4717 | + | Transgenic Insert |
| TI-456 | TTG/ | Fn/Lb_V1/ | 4700 . . . 4722 | + | Transgenic Insert |
| | TTTG/ | Lb_V2/Lb | | | |
| | TTTG | | | | |
| TI-457 | TTG | Fn | 4682 . . . 4704 | − | Transgenic Insert |
| TI-458 | TTG | Fn | 4711 . . . 4733 | + | Transgenic Insert |
| TI-459 | TTA | Fn | 4699 . . . 4721 | − | Transgenic Insert |
| TI-460 | TTG/ | Fn/Lb_V1/ | 4754 . . . 4776 | + | Transgenic Insert |
| | TTTG/ | Lb_V2/Lb | | | |
| | TTTG | | | | |
| TI-461 | TCCC | Lb_V1 | 4768 . . . 4790 | + | Transgenic Insert |
| TI-462 | TTA | Fn | 4790 . . . 4812 | + | Transgenic Insert |
| TI-463 | TTG | Fn | 4773 . . . 4795 | − | Transgenic Insert |
| TI-464 | TATG | Lb_V2 | 4824 . . . 4846 | + | Transgenic Insert |
| TI-465 | TTA/ | Fn/Lb_V1/ | 4809 . . . 4831 | − | Transgenic Insert |
| | TTTA/ | Lb_V2/Lb | | | |
| | TTTA | | | | |
| TI-466 | TCCA | Lb_V1 | 4856 . . . 4878 | + | Transgenic Insert |
| TI-467 | TTA/ | Fn/Lb_V1/ | 4836 . . . 4858 | − | Transgenic Insert |
| | TTTA/ | Lb_V2/Lb | | | |
| | TTTA | | | | |
| TI-468 | TTC/ | Fn/Lb_V1/ | 4868 . . . 4890 | + | Transgenic Insert |
| | TTTC/ | Lb_V2/Lb | | | |
| | TTTC | | | | |
| TI-469 | TTCG | Lb_V1 | 4869 . . . 4891 | + | Transgenic Insert |
| TI-470 | TTCG | Lb_V1 | 4849 . . . 4871 | − | Transgenic Insert |
| TI-471 | TTC | Fn | 4850 . . . 4872 | − | Transgenic Insert |
| TI-472 | TTG/ | Fn/Lb_V1/ | 4856 . . . 4878 | − | Transgenic Insert |
| | TTTG/ | Lb_V2/Lb | | | |
| | TTTG | | | | |
| TI-473 | TTCC | Lb_V1 | 4867 . . . 4889 | − | Transgenic Insert |
| TI-474 | TTC | Fn | 4868 . . . 4890 | − | Transgenic Insert |
| TI-475 | TCCA | Lb_V1 | 4871 . . . 4893 | − | Transgenic Insert |
| TI-476 | TTG | Fn | 4901 . . . 4923 | + | Transgenic Insert |
| TI-477 | TCCA | Lb_V1 | 4884 . . . 4906 | − | Transgenic Insert |
| TI-478 | TTG | Fn | 4909 . . . 4931 | + | Transgenic Insert |
| TI-479 | TTG | Fn | 4895 . . . 4917 | − | Transgenic Insert |
| TI-480 | TATC | Lb_V2 | 4931 . . . 4953 | + | Transgenic Insert |
| TI-481 | TCCG | Lb_V1 | 4933 . . . 4955 | + | Transgenic Insert |
| TI-482 | TTCG | Lb_V1 | 4927 . . . 4949 | − | Transgenic Insert |
| TI-483 | TTC | Fn | 4928 . . . 4950 | − | Transgenic Insert |
| TI-484 | TCCC | Lb_V1 | 4942 . . . 4964 | − | Transgenic Insert |
| TI-485 | TTCC | Lb_V1 | 4943 . . . 4965 | − | Transgenic Insert |
| TI-486 | TTC | Fn | 4944 . . . 4966 | − | Transgenic Insert |
| TI-487 | TCCA | Lb_V1 | 4957 . . . 4979 | − | Transgenic Insert |
| TI-488 | TCCA | Lb_V1 | 4964 . . . 4986 | − | Transgenic Insert |
| TI-489 | TTCC | Lb_V1 | 4965 . . . 4987 | − | Transgenic Insert |
| TI-490 | TTC | Fn | 4966 . . . 4988 | − | Transgenic Insert |
| TI-491 | TTCG | Lb_V1 | 4973 . . . 4995 | − | Transgenic Insert |
| TI-492 | TTC/ | Fn/Lb_V1/ | 4974 . . . 4996 | − | Transgenic Insert |
| | TTTC/ | Lb_V2/Lb | | | |
| | TTTC | | | | |
| TI-493 | TTA | Fn | 5007 . . . 5029 | + | Transgenic Insert |
| TI-494 | TCCA | Lb_V1 | 4988 . . . 5010 | − | Transgenic Insert |
| TI-495 | TCCA | Lb_V1 | 4995 . . . 5017 | − | Transgenic Insert |
| TI-496 | TTCC | Lb_V1 | 4996 . . . 5018 | − | Transgenic Insert |
| TI-497 | TTC | Fn | 4997 . . . 5019 | − | Transgenic Insert |
| TI-498 | TCCA | Lb_V1 | 5027 . . . 5049 | + | Transgenic Insert |
| TI-499 | TTA/ | Fn/Lb_V1/ | 5034 . . . 5056 | + | Transgenic Insert |
| | TTTA/ | Lb_V2/Lb | | | |
| | TTTA | | | | |
| TI-500 | TTA | Fn | 5089 . . . 5111 | + | Transgenic Insert |
| TI-501 | TTG/ | Fn/Lb_V1/ | 5068 . . . 5090 | − | Transgenic Insert |
| | TTTG/ | Lb_V2/Lb | | | |
| | TTTG | | | | |
| TI-502 | TCCA | Lb_V1 | 5076 . . . 5098 | − | Transgenic Insert |
| TI-503 | TATG | Lb_V2 | 5101 . . . 5123 | + | Transgenic Insert |
| TI-504 | TTA | Fn | 5092 . . . 5114 | − | Transgenic Insert |
| TI-505 | TTA/ | Fn/Lb_V1/ | 5117 . . . 5139 | + | Transgenic Insert |
| | TTTA/ | Lb_V2/Lb | | | |
| | TTTA | | | | |
| TI-506 | TCCG | Lb_V1 | 5113 . . . 5135 | − | Transgenic Insert |
| TI-507 | TTA | Fn | 5122 . . . 5144 | − | Transgenic Insert |
| TI-508 | TTC | Fn | 5157 . . . 5179 | + | Transgenic Insert |
| TI-509 | TTCA | Lb_V1 | 5146 . . . 5168 | − | Transgenic Insert |
| TI-510 | TTC | Fn | 5147 . . . 5169 | − | Transgenic Insert |
| TI-511 | TCCG | Lb_V1 | 5170 . . . 5192 | − | Transgenic Insert |
| TI-512 | TTCC | Lb_V1 | 5171 . . . 5193 | − | Transgenic Insert |
| TI-513 | TTC | Fn | 5172 . . . 5194 | − | Transgenic Insert |
| TI-514 | TTG | Fn | 5203 . . . 5225 | + | Transgenic Insert |
| TI-515 | TTG/ | Fn/Lb_V1/ | 5194 . . . 5216 | − | Transgenic Insert |
| | TTTG/ | Lb_V2/Lb | | | |
| | TTTG | | | | |
| TI-516 | TCCG | Lb_V1 | 5200 . . . 5222 | − | Transgenic Insert |
| TI-517 | TCCA | Lb_V1 | 5218 . . . 5240 | − | Transgenic Insert |
| TI-518 | TTG | Fn | 5243 . . . 5265 | + | Transgenic Insert |
| TI-519 | TTC | Fn | 5248 . . . 5270 | + | Transgenic Insert |
| TI-520 | TTG | Fn | 5228 . . . 5250 | − | Transgenic Insert |
| TI-521 | TCCC | Lb_V1 | 5232 . . . 5254 | − | Transgenic Insert |
| TI-522 | TTCG | Lb_V1 | 5239 . . . 5261 | − | Transgenic Insert |
| TI-523 | TTC | Fn | 5240 . . . 5262 | − | Transgenic Insert |
| TI-524 | TTC/ | Fn/Lb_V1/ | 5263 . . . 5285 | + | Transgenic Insert |
| | TTTC/ | Lb_V2/Lb | | | |
| | TTTC | | | | |
| TI-525 | TTCG | Lb_V1 | 5264 . . . 5286 | + | Transgenic Insert |
| TI-526 | TTC | Fn | 5270 . . . 5292 | + | Transgenic Insert |
| TI-527 | TTCC | Lb_V1 | 5271 . . . 5293 | + | Transgenic Insert |
| TI-528 | TCCA | Lb_V1 | 5272 . . . 5294 | + | Transgenic Insert |
| TI-529 | TTG | Fn | 5256 . . . 5278 | − | Transgenic Insert |
| TI-530 | TCCA | Lb_V1 | 5281 . . . 5303 | + | Transgenic Insert |
| TI-531 | TTA | Fn | 5291 . . . 5313 | + | Transgenic Insert |
| TI-532 | TTA | Fn | 5300 . . . 5322 | + | Transgenic Insert |
| TI-533 | TTG/ | Fn/Lb_V1/ | 5310 . . . 5332 | + | Transgenic Insert |
| | TTTG/ | Lb_V2/Lb | | | |
| | TTTG | | | | |
| TI-534 | TTA | Fn | 5289 . . . 5311 | − | Transgenic Insert |
| TI-535 | TTA | Fn | 5314 . . . 5336 | + | Transgenic Insert |
| TI-536 | TTA/ | Fn/Lb_V1/ | 5293 . . . 5315 | − | Transgenic Insert |
| | TTTA/ | Lb_V2/Lb | | | |
| | TTTA | | | | |
| TI-537 | TTA | Fn | 5318 . . . 5340 | + | Transgenic Insert |
| TI-538 | TATG | Lb_V2 | 5299 . . . 5321 | − | Transgenic Insert |
| TI-539 | TTA/ | Fn/Lb_V1/ | 5351 . . . 5373 | + | Transgenic Insert |
| | TTTA/ | Lb_V2/Lb | | | |
| | TTTA | | | | |
| TI-540 | TTG | Fn | 5356 . . . 5378 | + | Transgenic Insert |
| TI-541 | TTG | Fn | 5365 . . . 5387 | + | Transgenic Insert |
| TI-542 | TTG/ | Fn/Lb_V1/ | 5426 . . . 5448 | + | Transgenic Insert |
| | TTTG | Lb | | | |
| TI-543 | TTA | Fn | 5440 . . . 5462 | + | Transgenic Insert |
| TI-544 | TATG | Lb_V2 | 5442 . . . 5464 | + | Transgenic Insert |
| TI-545 | TTC | Fn | 5422 . . . 5444 | − | Transgenic Insert |
| TI-546 | TTA | Fn | 5430 . . . 5452 | − | Transgenic Insert |
| TI-547 | TTA/ | Fn/Lb_V1/ | 5455 . . . 5477 | + | Transgenic Insert |
| | TTTA | Lb | | | |
| TI-548 | TTTA | Lb_V2/Lb | 5455 . . . 5477 | + | Transgenic Insert |
| TI-549 | TTA/ | Fn/Lb_V1/ | 5494 . . . 5516 | + | Transgenic Insert |
| | TTTA/ | Lb_V2/Lb | | | |
| | TTTA | | | | |
| TI-550 | TTG | Fn | 5501 . . . 5523 | + | Transgenic Insert |
| TI-551 | TTG | Fn | 5507 . . . 5529 | + | Transgenic Insert |
| TI-552 | TTC | Fn | 5512 . . . 5534 | + | Transgenic Insert |
| TI-553 | TATA | Lb_V2 | 5493 . . . 5515 | − | Transgenic Insert |
| TI-554 | TTA/ | Fn/Lb_V1/ | 5518 . . . 5540 | + | Transgenic Insert |
| | TTTA/ | Lb_V2/Lb | | | |
| | TTTA | | | | |
| TI-555 | TATA | Lb_V2 | 5520 . . . 5542 | + | Transgenic Insert |
| TI-556 | TTG | Fn | 5499 . . . 5521 | − | Transgenic Insert |
| TI-557 | TTA | Fn | 5506 . . . 5528 | − | Transgenic Insert |
| TI-558 | TTA | Fn | 5509 . . . 5531 | − | Transgenic Insert |
| TI-559 | TTA | Fn | 5531 . . . 5553 | + | Transgenic Insert |
| TI-560 | TATG | Lb_V2 | 5515 . . . 5537 | − | Transgenic Insert |
| TI-561 | TTA | Fn | 5517 . . . 5539 | − | Transgenic Insert |
| TI-562 | TATG | Lb_V2 | 5520 . . . 5542 | − | Transgenic Insert |
| TI-563 | TTA/ | Fn/Lb_V1/ | 5522 . . . 5544 | − | Transgenic Insert |
| | TTTA/ | Lb_V2/Lb | | | |
| | TTTA | | | | |
| TI-564 | TTG | Fn | 5531 . . . 5553 | − | Transgenic Insert |
| TI-565 | TTG | Fn | 5538 . . . 5560 | − | Transgenic Insert |
| TI-566 | TTA/ | Fn/Lb_V1/ | 5542 . . . 5564 | − | Transgenic Insert |
| | TTTA/ | Lb_V2/Lb | | | |
| | TTTA | | | | |
| TI-567 | TATC | Lb_V2 | 5577 . . . 5599 | + | Transgenic Insert |
| TI-568 | TCCA | Lb_V1 | 5579 . . . 5601 | + | Transgenic Insert |
| TI-569 | TTA | Fn | 5567 . . . 5589 | − | Transgenic Insert |
| TI-570 | TTC/ | Fn/Lb_V1/ | 5570 . . . 5592 | − | Transgenic Insert |
| | TTTC/ | Lb_V2/Lb | | | |
| | TTTC | | | | |
| TI-571 | TTCC | Lb_V1 | 5574 . . . 5596 | − | Transgenic Insert |
| TI-572 | TTC/ | Fn/Lb_V1/ | 5575 . . . 5597 | − | Transgenic Insert |
| | TTTC/ | Lb_V2/Lb | | | |
| | TTTC | | | | |
| TI-573 | TATG | Lb_V2 | 5610 . . . 5632 | + | Transgenic Insert |
| TI-574 | TTA/ | Fn/Lb_V1/ | 5644 . . . 5666 | + | Transgenic Insert |
| | TTTA/ | Lb_V2/Lb | | | |
| | TTTA | | | | |
| TI-575 | TTC/ | Fn/Lb_V1/ | 5654 . . . 5676 | + | Transgenic Insert |
| | TTTC/ | Lb_V2/Lb | | | |
| | TTTC | | | | |
| TI-576 | TTC/ | Fn/Lb_V1/ | 5658 . . . 5680 | + | Transgenic Insert |
| | TTTC/ | Lb_V2/Lb | | | |
| | TTTC | | | | |
| TI-577 | TTCC | Lb_V1 | 5659 . . . 5681 | + | Transgenic Insert |
| TI-578 | TCCC | Lb_V1 | 5660 . . . 5682 | + | Transgenic Insert |
| TI-579 | TTC/ | Fn/Lb_V1/ | 5666 . . . 5688 | + | Transgenic Insert |
| | TTTC/ | Lb_V2/Lb | | | |
| | TTTC | | | | |
| TI-580 | TTG | Fn | 5669 . . . 5691 | + | Transgenic Insert |
| TI-581 | TTCA | Lb_V1 | 5647 . . . 5669 | − | Transgenic Insert |
| TI-582 | TTC/ | Fn/Lb_V1/ | 5648 . . . 5670 | − | Transgenic Insert |
| | TTTC | Lb | | | |
| TI-583 | TTG | Fn | 5672 . . . 5694 | + | Transgenic Insert |
| TI-584 | TTA | Fn | 5654 . . . 5676 | − | Transgenic Insert |
| TI-585 | TTA/ | Fn/Lb_V1/ | 5767 . . . 5789 | + | Transgenic Insert |
| | TTTA/ | Lb_V2/Lb | | | |
| | TTTA | | | | |
| TI-586 | TTA | Fn | 5778 . . . 5800 | + | Transgenic Insert |
| TI-587 | TTC | Fn | 5784 . . . 5806 | + | Transgenic Insert |
| TI-588 | TTCG | Lb_V1 | 5785 . . . 5807 | + | Transgenic Insert |
| TI-589 | TTG | Fn | 5788 . . . 5810 | + | Transgenic Insert |
| TI-590 | TTA | Fn | 5793 . . . 5815 | + | Transgenic Insert |
| TI-591 | TTG | Fn | 5774 . . . 5796 | − | Transgenic Insert |
| TI-592 | TTG | Fn | 5806 . . . 5828 | + | Transgenic Insert |
| TI-593 | TTG/ | Fn/Lb_V1/ | 5785 . . . 5807 | − | Transgenic Insert |
| | TTTG/ | Lb_V2/Lb | | | |
| | TTTG | | | | |
| TI-594 | TCCA | Lb_V1 | 5810 . . . 5832 | + | Transgenic Insert |
| TI-595 | TTCA | Lb_V1 | 5791 . . . 5813 | − | Transgenic Insert |
| TI-596 | TTC/ | Fn/Lb_V1/ | 5792 . . . 5814 | − | Transgenic Insert |
| | TTTC/ | Lb_V2/Lb | | | |
| | TTTC | | | | |
| TI-597 | TTCG | Lb_V1 | 5814 . . . 5836 | − | Transgenic Insert |
| TI-598 | TTC | Fn | 5815 . . . 5837 | − | Transgenic Insert |
| TI-599 | TTG/ | Fn/Lb_V1/ | 5908 . . . 5930 | + | Transgenic Insert |
| | TTTG/ | Lb_V2/Lb | | | |
| | TTTG | | | | |
| TI-600 | TATC | Lb_V2 | 5914 . . . 5936 | + | Transgenic Insert |
| TI-601 | TTA | Fn | 5910 . . . 5932 | − | Transgenic Insert |
| TI-602 | TTCA | Lb_V1 | 5913 . . . 5935 | − | Transgenic Insert |
| TI-603 | TTC/ | Fn/Lb_V1/ | 5914 . . . 5936 | − | Transgenic Insert |
| | TTTC/ | Lb_V2/Lb | | | |
| | TTTC | | | | |
| TI-604 | TTA | Fn | 5949 . . . 5971 | + | Transgenic Insert |
| TI-605 | TTG | Fn | 5952 . . . 5974 | + | Transgenic Insert |
| TI-606 | TTG | Fn | 5959 . . . 5981 | + | Transgenic Insert |
| TI-607 | TTA/ | Fn/Lb_V1/ | 5952 . . . 5974 | − | Transgenic Insert |
| | TTTA/ | Lb_V2/Lb | | | |
| | TTTA | | | | |
| TI-608 | TCCA | Lb_V1 | 5958 . . . 5980 | − | Transgenic Insert |
| TI-609 | TTG/ | Fn/Lb_V1/ | 5970 . . . 5992 | − | Transgenic Insert |
| | TTTG/ | Lb_V2/Lb | | | |
| | TTTG | | | | |
| TI-610 | TCCA | Lb_V1 | 6006 . . . 6028 | + | Transgenic Insert |
| TI-611 | TTG | Fn | 6009 . . . 6031 | + | Transgenic Insert |
| TI-612 | TTCA | Lb_V1 | 5991 . . . 6013 | − | Transgenic Insert |
| TI-613 | TTC | Fn | 5992 . . . 6014 | − | Transgenic Insert |
| TI-614 | TATG | Lb_V2 | 6016 . . . 6038 | + | Transgenic Insert |
| TI-615 | TTA/ | Fn/Lb_V1/ | 5996 . . . 6018 | − | Transgenic Insert |
| | TTTA/ | Lb_V2/Lb | | | |
| | TTTA | | | | |
| TI-616 | TTA | Fn | 6021 . . . 6043 | + | Transgenic Insert |
| TI-617 | TTA | Fn | 6000 . . . 6022 | − | Transgenic Insert |
| TI-618 | TTG | Fn | 6003 . . . 6025 | − | Transgenic Insert |
| TI-619 | TTTA | Lb_V1/Lb | 6009 . . . 6031 | − | Transgenic Insert |
| TI-620 | TTG/ | Fn/Lb_V1/ | 6022 . . . 6044 | − | Transgenic Insert |
| | TTTG/ | Lb_V2/Lb | | | |
| | TTTG | | | | |
| TI-621 | TTA/ | Fn/Lb_V1/ | 6065 . . . 6087 | + | Transgenic Insert |
| | TTTA/ | Lb_V2/Lb | | | |
| | TTTA | | | | |
| TI-622 | TATC | Lb_V2 | 6063 . . . 6085 | − | Transgenic Insert |
| TI-623 | TTG/ | Fn/Lb_V1/ | 6067 . . . 6089 | − | Transgenic Insert |
| | TTTG/ | Lb_V2/Lb | | | |
| | TTTG | | | | |
| TI-624 | TTA | Fn | 6102 . . . 6124 | + | Transgenic Insert |
| TI-625 | TATC | Lb_V2 | 6104 . . . 6126 | + | Transgenic Insert |
| TI-626 | TTA | Fn | 6094 . . . 6116 | − | Transgenic Insert |
| TI-627 | TATG | Lb_V2 | 6098 . . . 6120 | − | Transgenic Insert |
| TI-628 | TTG/ | Fn/Lb_V1/ | 6102 . . . 6124 | − | Transgenic Insert |
| | TTTG/ | Lb_V2/Lb | | | |
| | TTTG | | | | |
| TI-629 | TTA | Fn | 6154 . . . 6176 | + | Transgenic Insert |
| TI-630 | TTC/ | Fn/Lb_V1/ | 6172 . . . 6194 | + | Transgenic Insert |
| | TTTC/ | Lb_V2/Lb | | | |
| | TTTC | | | | |
| TI-631 | TTCA | Lb_V1 | 6173 . . . 6195 | + | Transgenic Insert |
| TI-632 | TATA | Lb_V2 | 6158 . . . 6180 | − | Transgenic Insert |
| TI-633 | TATG | Lb_V2 | 6180 . . . 6202 | + | Transgenic Insert |
| TI-634 | TATC | Lb_V2 | 6163 . . . 6185 | − | Transgenic Insert |
| TI-635 | TTA/ | Fn/Lb_V1/ | 6165 . . . 6187 | − | Transgenic Insert |
| | TTTA/ | Lb_V2/Lb | | | |
| | TTTA | | | | |
| TI-636 | TTC | Fn | 6169 . . . 6191 | − | Transgenic Insert |
| TI-637 | TTA/ | Fn/Lb_V1/ | 6225 . . . 6247 | + | Transgenic Insert |
| | TTTA/ | Lb_V2/Lb | | | |
| | TTTA | | | | |
| TI-638 | TATA | Lb_V2 | 6227 . . . 6249 | + | Transgenic Insert |
| TI-639 | TTA/ | Fn/Lb_V1/ | 6221 . . . 6243 | − | Transgenic Insert |
| | TTTA/ | Lb_V2/Lb | | | |
| | TTTA | | | | |
| TI-640 | TTG/ | Fn/Lb_V1/ | 6228 . . . 6250 | − | Transgenic Insert |
| | TTTG/ | Lb_V2/Lb | | | |
| | TTTG | | | | |
| TI-641 | TTC/ | Fn/Lb_V1/ | 6273 . . . 6295 | − | Transgenic Insert |
| | TTTC/ | Lb_V2/Lb | | | |
| | TTTC | | | | |
| TI-642 | TTC | Fn | 6316 . . . 6338 | + | Transgenic Insert |
| TI-643 | TTCA | Lb_V1 | 6317 . . . 6339 | + | Transgenic Insert |
| TI-644 | TCCC | Lb_V1 | 6301 . . . 6323 | − | Transgenic Insert |
| TI-645 | TTG | Fn | 6314 . . . 6336 | − | Transgenic Insert |
| TI-646 | TTG | Fn | 6324 . . . 6346 | − | Transgenic Insert |
| TI-647 | TATA | Lb_V2 | 6404 . . . 6426 | + | Transgenic Insert |
| TI-648 | TTA | Fn | 6421 . . . 6443 | + | Transgenic Insert |
| TI-649 | TTG | Fn | 6403 . . . 6425 | − | Transgenic Insert |
| TI-650 | TCCA | Lb_V1 | 6410 . . . 6432 | − | Transgenic Insert |
| TI-651 | TTA | Fn | 6442 . . . 6464 | + | Transgenic Insert |
| TI-652 | TATG | Lb_V2 | 6444 . . . 6466 | + | Transgenic Insert |
| TI-653 | TTC/ | Fn/Lb_V1/ | 6431 . . . 6453 | − | Transgenic Insert |
| | TTTC/ | Lb_V2/Lb | | | |
| | TTTC | | | | |
| TI-654 | TTA | Fn | 6454 . . . 6476 | + | Transgenic Insert |
| TI-655 | TTG | Fn | 6461 . . . 6483 | + | Transgenic Insert |
| TI-656 | TTG | Fn | 6467 . . . 6489 | + | Transgenic Insert |
| TI-657 | TTA | Fn | 6456 . . . 6478 | − | Transgenic Insert |
| TI-658 | TTA/ | Fn/Lb_V1/ | 6459 . . . 6481 | − | Transgenic Insert |
| | TTTA/ | Lb_V2/Lb | | | |
| | TTTA | | | | |
| TI-659 | TTG/ | Fn/Lb_V1/ | 6469 . . . 6491 | − | Transgenic Insert |
| | TTTG/ | Lb_V2/Lb | | | |
| | TTTG | | | | |
| TI-660 | TTG | Fn | 6503 . . . 6525 | + | Transgenic Insert |
| TI-661 | TTG | Fn | 6507 . . . 6529 | − | Transgenic Insert |
| TI-662 | TTA/ | Fn/Lb_V1/ | 6529 . . . 6551 | + | Transgenic Insert |
| | TTTA/ | Lb_V2/Lb | | | |
| | TTTA | | | | |
| TI-663 | TTG/ | Fn/Lb_V1/ | 6512 . . . 6534 | − | Transgenic Insert |
| | TTTG/ | Lb_V2/Lb | | | |
| | TTTG | | | | |
| TI-664 | TTG/ | Fn/Lb_V1/ | 6519 . . . 6541 | − | Transgenic Insert |
| | TTTG/ | Lb_V2/Lb | | | |
| | TTTG | | | | |
| TI-665 | TTG/ | Fn/Lb_V1/ | 6594 . . . 6616 | + | Transgenic Insert |
| | TTTG/ | Lb_V2/Lb | | | |
| | TTTG | | | | |
| TI-666 | TTG | Fn | 6586 . . . 6608 | − | Transgenic Insert |
| TI-667 | TTA/ | Fn/Lb_V1/ | 6628 . . . 6650 | + | Transgenic Insert |
| | TTTA/ | Lb_V2/Lb | | | |
| | TTTA | | | | |
| TI-668 | TTA | Fn | 6631 . . . 6653 | + | Transgenic Insert |
| TI-669 | TTA | Fn | 6634 . . . 6656 | + | Transgenic Insert |
| TI-670 | TTG | Fn | 6641 . . . 6663 | + | Transgenic Insert |
| TI-671 | TTC | Fn | 6645 . . . 6667 | + | Transgenic Insert |
| TI-672 | TTCA | Lb_V1 | 6646 . . . 6668 | + | Transgenic Insert |
| TI-673 | TTA | Fn | 6654 . . . 6676 | + | Transgenic Insert |
| TI-674 | TTC/ | Fn/Lb_V1/ | 6660 . . . 6682 | + | Transgenic Insert |
| | TTTC/ | Lb_V2/Lb | | | |
| | TTTC | | | | |
| TI-675 | TATA | Lb_V2 | 6703 . . . 6725 | + | Transgenic Insert |
| TI-676 | TTC | Fn | 6726 . . . 6748 | + | Transgenic Insert |
| TI-677 | TTC | Fn | 6729 . . . 6751 | + | Transgenic Insert |
| TI-678 | TTG | Fn | 6710 . . . 6732 | − | Transgenic Insert |
| TI-679 | TTC | Fn | 6732 . . . 6754 | + | Transgenic Insert |
| TI-680 | TTCA | Lb_V1 | 6733 . . . 6755 | + | Transgenic Insert |
| TI-681 | TTG | Fn | 6722 . . . 6744 | − | Transgenic Insert |
| TI-682 | TTG/ | Fn/Lb_V1/ | 6730 . . . 6752 | − | Transgenic Insert |
| | TTTG/ | Lb_V2/Lb | | | |
| | TTTG | | | | |
| TI-683 | TTC/ | Fn/Lb_V1/ | 6772 . . . 6794 | + | Transgenic Insert |
| | TTTC/ | Lb_V2/Lb | | | |
| | TTTC | | | | |
| TI-684 | TTC | Fn | 6781 . . . 6803 | + | Transgenic Insert |
| TI-685 | TTG | Fn | 6771 . . . 6793 | − | Transgenic Insert |
| TI-686 | TTA/ | Fn/Lb_V1/ | 6776 . . . 6798 | − | Transgenic Insert |
| | TTTA/ | Lb_V2/Lb | | | |
| | TTTA | | | | |
| TI-687 | TTG/ | Fn/Lb_V1/ | 6801 . . . 6823 | − | Transgenic Insert |
| | TTTG/ | Lb_V2/Lb | | | |
| | TTTG | | | | |
| TI-688 | TTC/ | Fn/Lb_V1/ | 6846 . . . 6868 | + | Transgenic Insert |
| | TTTC/ | Lb_V2/Lb | | | |
| | TTTC | | | | |
| TI-689 | TTCG | Lb_V1 | 6847 . . . 6869 | + | Transgenic Insert |
| TI-690 | TTC | Fn | 6850 . . . 6872 | + | Transgenic Insert |
| TI-691 | TTCG | Lb_V1 | 6851 . . . 6873 | + | Transgenic Insert |
| TI-692 | TCCC | Lb_V1 | 6856 . . . 6878 | + | Transgenic Insert |
| TI-693 | TTC/ | Fn/Lb_V1/ | 6862 . . . 6884 | + | Transgenic Insert |
| | TTTC/ | Lb_V2/Lb | | | |
| | TTTC | | | | |
| TI-694 | TTCG | Lb_V1 | 6863 . . . 6885 | + | Transgenic Insert |
| TI-695 | TATA | Lb_V2 | 6867 . . . 6889 | + | Transgenic Insert |
| TI-696 | TATG | Lb_V2 | 6869 . . . 6891 | + | Transgenic Insert |
| TI-697 | TTC | Fn | 6872 . . . 6894 | + | Transgenic Insert |
| TI-698 | TTG/ | Fn/Lb_V1/ | 6876 . . . 6898 | + | Transgenic Insert |
| | TTTG/ | Lb_V2/Lb | | | |
| | TTTG | | | | |
| TI-699 | TTA/ | Fn/Lb_V1/ | 6881 . . . 6903 | + | Transgenic Insert |
| | TTTA/ | Lb_V2/Lb | | | |
| | TTTA | | | | |
| TI-700 | TTC | Fn | 6886 . . . 6908 | + | Transgenic Insert |
| TI-701 | TTA | Fn | 6866 . . . 6888 | − | Transgenic Insert |
| TI-702 | TTCG | Lb_V1 | 6877 . . . 6899 | − | Transgenic Insert |
| TI-703 | TTC | Fn | 6878 . . . 6900 | − | Transgenic Insert |
| TI-704 | TTC/ | Fn/Lb_V1/ | 6914 . . . 6936 | + | Transgenic Insert |
| | TTTC/ | Lb_V2/Lb | | | |
| | TTTC | | | | |
| TI-705 | TTG/ | Fn/Lb_V1/ | 6922 . . . 6944 | + | Transgenic Insert |
| | TTTG/ | Lb_V2/Lb | | | |
| | TTTG | | | | |
| TI-706 | TTA | Fn | 6929 . . . 6951 | + | Transgenic Insert |
| TI-707 | TATC | Lb_V2 | 6935 . . . 6957 | + | Transgenic Insert |
| TI-708 | TTA | Fn | 6916 . . . 6938 | − | Transgenic Insert |
| TI-709 | TTA | Fn | 6941 . . . 6963 | + | Transgenic Insert |
| TI-710 | TTC | Fn | 6945 . . . 6967 | + | Transgenic Insert |
| TI-711 | TTA | Fn | 6952 . . . 6974 | + | Transgenic Insert |
| TI-712 | TATG | Lb_V2 | 6935 . . . 6957 | − | Transgenic Insert |
| TI-713 | TTC/ | Fn/Lb_V1/ | 6959 . . . 6981 | + | Transgenic Insert |
| | TTTC/ | Lb_V2/Lb | | | |
| | TTTC | | | | |
| TI-714 | TTCA | Lb_V1 | 6960 . . . 6982 | + | Transgenic Insert |
| TI-715 | TATC | Lb_V2 | 6939 . . . 6961 | − | Transgenic Insert |
| TI-716 | TATC | Lb_V2 | 6968 . . . 6990 | + | Transgenic Insert |
| TI-717 | TCCG | Lb_V1 | 6973 . . . 6995 | + | Transgenic Insert |
| TI-718 | TTG/ | Fn/Lb_V1/ | 6957 . . . 6979 | - | Transgenic Insert |
| | TTTG/ | Lb_V2/Lb | | | |
| | TTTG | | | | |
| TI-719 | TTA | Fn | 6961 . . . 6983 | − | Transgenic Insert |
| TI-720 | TTG/ | Fn/Lb_V1/ | 6998 . . . 7020 | + | Transgenic Insert |
| | TTTG/ | Lb_V2/Lb | | | |
| | TTTG | | | | |
| TI-721 | TTA | Fn | 7009 . . . 7031 | + | Transgenic Insert |
| TI-722 | TTC | Fn | 7015 . . . 7037 | + | Transgenic Insert |
| TI-723 | TTCG | Lb_V1 | 7016 . . . 7038 | + | Transgenic Insert |
| TI-724 | TTG/ | Fn/Lb_V1/ | 7021 . . . 7043 | + | Transgenic Insert |
| | TTTG/ | Lb_V2/Lb | | | |
| | TTTG | | | | |
| TI-725 | TTC/ | Fn/Lb_V1/ | 7028 . . . 7050 | + | Transgenic Insert |
| | TTTC/ | Lb_V2/Lb | | | |
| | TTTC | | | | |
| TI-726 | TATC | Lb_V2 | 7032 . . . 7054 | + | Transgenic Insert |
| TI-727 | TTA | Fn | 7046 . . . 7068 | + | Transgenic Insert |
| TI-728 | TTC/ | Fn/Lb_V1/ | 7051 . . . 7073 | + | Transgenic Insert |
| | TTTC/ | Lb_V2/Lb | | | |
| | TTTC | | | | |
| TI-729 | TTG/ | Fn/Lb_V1/ | 7058 . . . 7080 | + | Transgenic Insert |
| | TTTG/ | Lb_V2/Lb | | | |
| | TTTG | | | | |
| TI-730 | TTCG | Lb_V1 | 7041 . . . 7063 | − | Transgenic Insert |
| TI-731 | TTC | Fn | 7042 . . . 7064 | − | Transgenic Insert |
| TI-732 | TTA | Fn | 7054 . . . 7076 | − | Transgenic Insert |
| TI-733 | TTC/ | Fn/Lb_V1/ | 7092 . . . 7114 | + | Transgenic Insert |
| | TTTC/ | Lb_V2/Lb | | | |
| | TTTC | | | | |
| TI-734 | TTA | Fn | 7098 . . . 7120 | + | Transgenic Insert |
| TI-735 | TTG/ | Fn/Lb_V1/ | 7125 . . . 7147 | + | Transgenic Insert |
| | TTTG/ | Lb_V2/Lb | | | |
| | TTTG | | | | |
| TI-736 | TTG/ | Fn/Lb_V1/ | 7134 . . . 7156 | + | Transgenic Insert |
| | TTTG/ | Lb_V2/Lb | | | |
| | TTTG | | | | |
| TI-737 | TTG | Fn | 7113 . . . 7135 | − | Transgenic Insert |
| TI-738 | TTG | Fn | 7116 . . . 7138 | − | Transgenic Insert |
| TI-739 | TATC | Lb_V2 | 7147 . . . 7169 | + | Transgenic Insert |
| TI-740 | TTC | Fn | 7152 . . . 7174 | + | Transgenic Insert |
| TI-741 | TTCA | Lb_V1 | 7153 . . . 7175 | + | Transgenic Insert |
| TI-742 | TTG | Fn | 7138 . . . 7160 | − | Transgenic Insert |
| TI-743 | TTC | Fn | 7162 . . . 7184 | + | Transgenic Insert |
| TI-744 | TTCA | Lb_V1 | 7163 . . . 7185 | + | Transgenic Insert |
| TI-745 | TTA | Fn | 7195 . . . 7217 | + | Transgenic Insert |
| TI-746 | TCCG | Lb_V1 | 7173 . . . 7195 | − | Transgenic Insert |
| TI-747 | TCCA | Lb_V1 | 7178 . . . 7200 | − | Transgenic Insert |
| TI-748 | TCCC | Lb_V1 | 7187 . . . 7209 | − | Transgenic Insert |
| TI-749 | TTA | Fn | 7221 . . . 7243 | + | Transgenic Insert |
| TI-750 | TCCA | Lb_V1 | 7231 . . . 7253 | + | Transgenic Insert |
| TI-751 | TCCA | Lb_V1 | 7210 . . . 7232 | − | Transgenic Insert |
| TI-752 | TTCC | Lb_V1 | 7211 . . . 7233 | − | Transgenic Insert |
| TI-753 | TTC | Fn | 7212 . . . 7234 | − | Transgenic Insert |
| TI-754 | TTG | Fn | 7235 . . . 7257 | + | Transgenic Insert |
| TI-755 | TTC/ | Fn/Lb_V1/ | 7258 . . . 7280 | + | Transgenic Insert |
| | TTTC/ | Lb_V2/Lb | | | |
| | TTTC | | | | |
| TI-756 | TTCA | Lb_V1 | 7259 . . . 7281 | + | Transgenic Insert |
| TI-757 | TTG | Fn | 7243 . . . 7265 | − | Transgenic Insert |
| TI-758 | TTA | Fn | 7273 . . . 7295 | + | Transgenic Insert |
| TI-759 | TATC | Lb_V2 | 7275 . . . 7297 | + | Transgenic Insert |
| TI-760 | TCCA | Lb_V1 | 7260 . . . 7282 | − | Transgenic Insert |
| TI-761 | TTC/ | Fn/Lb_V1/ | 7282 . . . 7304 | + | Transgenic Insert |
| | TTTC/ | Lb_V2/Lb | | | |
| | TTTC | | | | |
| TI-762 | TTCC | Lb_V1 | 7283 . . . 7305 | + | Transgenic Insert |
| TI-763 | TTG | Fn | 7275 . . . 7297 | − | Transgenic Insert |
| TI-764 | TTG | Fn | 7315 . . . 7337 | + | Transgenic Insert |
| TI-765 | TTG/ | Fn/Lb_V1/ | 7298 . . . 7320 | − | Transgenic Insert |
| | TTTG/ | Lb_V2/Lb | | | |
| | TTTG | | | | |
| TI-766 | TTC | Fn | 7332 . . . 7354 | + | Transgenic Insert |
| TI-767 | TTCG | Lb_V1 | 7333 . . . 7355 | + | Transgenic Insert |
| TI-768 | TCCA | Lb_V1 | 7347 . . . 7369 | + | Transgenic Insert |
| TI-769 | TTG | Fn | 7328 . . . 7350 | − | Transgenic Insert |
| TI-770 | TTC | Fn | 7351 . . . 7373 | + | Transgenic Insert |
| TI-771 | TTCC | Lb_V1 | 7352 . . . 7374 | + | Transgenic Insert |
| TI-772 | TCCA | Lb_V1 | 7353 . . . 7375 | + | Transgenic Insert |
| TI-773 | TTC | Fn | 7357 . . . 7379 | + | Transgenic Insert |
| TI-774 | TTG/ | Fn/Lb_V1/ | 7335 . . . 7357 | − | Transgenic Insert |
| | TTTG/ | Lb_V2/Lb | | | |
| | TTTG | | | | |
| TI-775 | TCCA | Lb_V1 | 7366 . . . 7388 | + | Transgenic Insert |
| TI-776 | TTG | Fn | 7369 . . . 7391 | + | Transgenic Insert |
| TI-777 | TTA | Fn | 7376 . . . 7398 | + | Transgenic Insert |
| TI-778 | TATC | Lb_V2 | 7378 . . . 7400 | + | Transgenic Insert |
| TI-779 | TCCC | Lb_V1 | 7380 . . . 7402 | + | Transgenic Insert |
| TI-780 | TTA | Fn | 7396 . . . 7418 | + | Transgenic Insert |
| TI-781 | TTC | Fn | 7378 . . . 7400 | − | Transgenic Insert |
| TI-782 | TTG | Fn | 7383 . . . 7405 | − | Transgenic Insert |
| TI-783 | TATG | Lb_V2 | 7401 . . . 7423 | − | Transgenic Insert |
| TI-784 | TTG | Fn | 7439 . . . 7461 | + | Transgenic Insert |
| TI-785 | TTC | Fn | 7458 . . . 7480 | + | Transgenic Insert |
| TI-786 | TTCC | Lb_V1 | 7459 . . . 7481 | + | Transgenic Insert |
| TI-787 | TTG | Fn | 7462 . . . 7484 | + | Transgenic Insert |
| TI-788 | TCCG | Lb_V1 | 7473 . . . 7495 | + | Transgenic Insert |
| TI-789 | TTA | Fn | 7486 . . . 7508 | + | Transgenic Insert |
| TI-790 | TATG | Lb_V2 | 7488 . . . 7510 | + | Transgenic Insert |
| TI-791 | TTG | Fn | 7469 . . . 7491 | − | Transgenic Insert |
| TI-792 | TCCG | Lb_V1 | 7481 . . . 7503 | − | Transgenic Insert |
| TI-793 | TATC | Lb_V2 | 7495 . . . 7517 | − | Transgenic Insert |
| TI-794 | TTC/ | Fn/Lb_V1/ | 7530 . . . 7552 | + | Transgenic Insert |
| | TTTC/ | Lb_V2/Lb | | | |
| | TTTC | | | | |
| TI-795 | TTCC | Lb_V1 | 7531 . . . 7553 | + | Transgenic Insert |
| TI-796 | TTG | Fn | 7511 . . . 7533 | − | Transgenic Insert |
| TI-797 | TATC | Lb_V2 | 7535 . . . 7557 | + | Transgenic Insert |
| TI-798 | TCCC | Lb_V1 | 7526 . . . 7548 | − | Transgenic Insert |
| TI-799 | TTG | Fn | 7541 . . . 7563 | − | Transgenic Insert |
| TI-800 | TTG | Fn | 7549 . . . 7571 | − | Transgenic Insert |
| TI-801 | TTCA | Lb_V1 | 7560 . . . 7582 | − | Transgenic Insert |
| TI-802 | TTC | Fn | 7561 . . . 7583 | − | Transgenic Insert |
| TI-803 | TTG | Fn | 7585 . . . 7607 | + | Transgenic Insert |
| TI-804 | TTA/ | Fn/Lb_V1/ | 7568 . . . 7590 | − | Transgenic Insert |
| | TTTA/ | Lb_V2/Lb | | | |
| | TTTA | | | | |
| TI-805 | TTC | Fn | 7603 . . . 7625 | + | Transgenic Insert |
| TI-806 | TTCA | Lb_V1 | 7604 . . . 7626 | + | Transgenic Insert |
| TI-807 | TTA | Fn | 7590 . . . 7612 | − | Transgenic Insert |
| TI-808 | TCCG | Lb_V1 | 7599 . . . 7621 | − | Transgenic Insert |
| TI-809 | TTCC | Lb_V1 | 7600 . . . 7622 | − | Transgenic Insert |
| TI-810 | TTC | Fn | 7623 . . . 7645 | + | Transgenic Insert |
| TI-811 | TTC | Fn | 7601 . . . 7623 | − | Transgenic Insert |
| TI-812 | TTCG | Lb_V1 | 7624 . . . 7646 | + | Transgenic Insert |
| TI-813 | TTA/ | Fn/Lb_V1/ | 7634 . . . 7656 | + | Transgenic Insert |
| | TTTA/ | Lb_V2/Lb | | | |
| | TTTA | | | | |
| TI-814 | TTCG | Lb_V1 | 7619 . . . 7641 | − | Transgenic Insert |
| TI-815 | TTC | Fn | 7620 . . . 7642 | − | Transgenic Insert |
| TI-816 | TCCA | Lb_V1 | 7624 . . . 7646 | − | Transgenic Insert |
| TI-817 | TTG | Fn | 7649 . . . 7671 | + | Transgenic Insert |
| TI-818 | TTC | Fn | 7656 . . . 7678 | + | Transgenic Insert |
| TI-819 | TTA | Fn | 7635 . . . 7657 | − | Transgenic Insert |
| TI-820 | TTCA | Lb_V1 | 7657 . . . 7679 | + | Transgenic Insert |
| TI-821 | TTCA | Lb_V1 | 7646 . . . 7668 | − | Transgenic Insert |
| TI-822 | TTC | Fn | 7647 . . . 7669 | − | Transgenic Insert |
| TI-823 | TCCA | Lb_V1 | 7655 . . . 7677 | − | Transgenic Insert |
| TI-824 | TCCA | Lb_V1 | 7661 . . . 7683 | − | Transgenic Insert |
| TI-825 | TCCA | Lb_V1 | 7668 . . . 7690 | − | Transgenic Insert |
| TI-826 | TCCC | Lb_V1 | 7676 . . . 7698 | − | Transgenic Insert |
| TI-827 | TCCA | Lb_V1 | 7716 . . . 7738 | + | Transgenic Insert |
| TI-828 | TTA | Fn | 7726 . . . 7748 | + | Transgenic Insert |
| TI-829 | TTA | Fn | 7712 . . . 7734 | − | Transgenic Insert |
| TI-830 | TCCA | Lb_V1 | 7716 . . . 7738 | − | Transgenic Insert |
| TI-831 | TTA/ | Fn/Lb_V1/ | 7757 . . . 7779 | + | Transgenic Insert |
| | TTTA/ | Lb_V2/Lb | | | |
| | TTTA | | | | |
| TI-832 | TTG | Fn | 7737 . . . 7759 | − | Transgenic Insert |
| TI-833 | TTA/ | Fn/Lb_V1/ | 7772 . . . 7794 | + | Transgenic Insert |
| | TTTA/ | Lb_V2/Lb | | | |
| | TTTA | | | | |
| TI-834 | TTC | Fn | 7755 . . . 7777 | − | Transgenic Insert |
| TI-835 | TCCC | Lb_V1 | 7764 . . . 7786 | − | Transgenic Insert |
| TI-836 | TTCC | Lb_V1 | 7765 . . . 7787 | − | Transgenic Insert |
| TI-837 | TCCA | Lb_V1 | 7787 . . . 7809 | + | Transgenic Insert |
| TI-838 | TTC | Fn | 7766 . . . 7788 | − | Transgenic Insert |
| TI-839 | TCCG | Lb_V1 | 7778 . . . 7800 | − | Transgenic Insert |
| TI-840 | TTA | Fn | 7785 . . . 7807 | − | Transgenic Insert |
| TI-841 | TCCC | Lb_V1 | 7816 . . . 7838 | + | Transgenic Insert |
| TI-842 | TTG | Fn | 7798 . . . 7820 | − | Transgenic Insert |
| TI-843 | TTC | Fn | 7822 . . . 7844 | + | Transgenic Insert |
| TI-844 | TTCA | Lb_V1 | 7823 . . . 7845 | + | Transgenic Insert |
| TI-845 | TTC | Fn | 7827 . . . 7849 | + | Transgenic Insert |
| TI-846 | TTCC | Lb_V1 | 7828 . . . 7850 | + | Transgenic Insert |
| TI-847 | TTCG | Lb_V1 | 7809 . . . 7831 | − | Transgenic Insert |
| TI-848 | TTC | Fn | 7810 . . . 7832 | − | Transgenic Insert |
| TI-849 | TTG | Fn | 7822 . . . 7844 | − | Transgenic Insert |
| TI-850 | TTC | Fn | 7851 . . . 7873 | + | Transgenic Insert |
| TI-851 | TTCG | Lb_V1 | 7852 . . . 7874 | + | Transgenic Insert |
| TI-852 | TTC | Fn | 7857 . . . 7879 | + | Transgenic Insert |
| TI-853 | TTCA | Lb_V1 | 7858 . . . 7880 | + | Transgenic Insert |
| TI-854 | TTG | Fn | 7862 . . . 7884 | + | Transgenic Insert |
| TI-855 | TTC | Fn | 7870 . . . 7892 | + | Transgenic Insert |
| TI-856 | TTCG | Lb_V1 | 7871 . . . 7893 | + | Transgenic Insert |
| TI-857 | TCCA | Lb_V1 | 7849 . . . 7871 | − | Transgenic Insert |
| TI-858 | TTCC | Lb_V1 | 7850 . . . 7872 | − | Transgenic Insert |
| TI-859 | TTC | Fn | 7851 . . . 7873 | − | Transgenic Insert |
| TI-860 | TTC | Fn | 7854 . . . 7876 | − | Transgenic Insert |
| TI-861 | TTG | Fn | 7861 . . . 7883 | − | Transgenic Insert |
| TI-862 | TCCC | Lb_V1 | 7874 . . . 7896 | − | Transgenic Insert |
| TI-863 | TATC | Lb_V2 | 7876 . . . 7898 | − | Transgenic Insert |
| TI-864 | TTA | Fn | 7878 . . . 7900 | − | Transgenic Insert |
| TI-865 | TTG/ | Fn/Lb_V1/ | 7910 . . . 7932 | + | Transgenic Insert |
| | TTTG/ | Lb_V2/Lb | | | |
| | TTTG | | | | |
| TI-866 | TCCC | Lb_V1 | 7918 . . . 7940 | − | Transgenic Insert |
| TI-867 | TTCC | Lb_V1 | 7919 . . . 7941 | − | Transgenic Insert |
| TI-868 | TTC/ | Fn/Lb_V1/ | 7920 . . . 7942 | − | Transgenic Insert |
| | TTTC/ | Lb_V2/Lb | | | |
| | TTTC | | | | |
| TI-869 | TTC | Fn | 7950 . . . 7972 | + | Transgenic Insert |
| TI-870 | TTCC | Lb_V1 | 7951 . . . 7973 | + | Transgenic Insert |
| TI-871 | TCCG | Lb_V1 | 7952 . . . 7974 | + | Transgenic Insert |
| TI-872 | TATC | Lb_V2 | 7957 . . . 7979 | + | Transgenic Insert |
| TI-873 | TCCG | Lb_V1 | 7953 . . . 7975 | − | Transgenic Insert |
| TI-874 | TTG | Fn | 7959 . . . 7981 | − | Transgenic Insert |
| TI-875 | TTCA | Lb_V1 | 7986 . . . 8008 | − | Transgenic Insert |
| TI-876 | TTC | Fn | 7987 . . . 8009 | − | Transgenic Insert |
| TI-877 | TTA | Fn | 7990 . . . 8012 | − | Transgenic Insert |
| TI-878 | TTG | Fn | 7994 . . . 8016 | − | Transgenic Insert |
| TI-879 | TATG | Lb_V2 | 8000 . . . 8022 | − | Transgenic Insert |
| TI-880 | TTA/ | Fn/Lb_V1/ | 8002 . . . 8024 | − | Transgenic Insert |
| | TTTA/ | Lb_V2/Lb | | | |
| | TTTA | | | | |
| TI-881 | TTC/ | Fn/Lb_V1/ | 8033 . . . 8055 | + | Transgenic Insert |
| | TTTC/ | Lb_V2/Lb | | | |
| | TTTC | | | | |
| TI-882 | TTCA | Lb_V1 | 8017 . . . 8039 | − | Transgenic Insert |
| TI-883 | TTC | Fn | 8018 . . . 8040 | − | Transgenic Insert |
| TI-884 | TTG/ | Fn/Lb_V1/ | 8058 . . . 8080 | + | Transgenic Insert |
| | TTTG/ | Lb_V2/Lb | | | |
| | TTTG | | | | |
| TI-885 | TTA | Fn | 8062 . . . 8084 | + | Transgenic Insert |
| TI-886 | TTG/ | Fn/Lb_V1/ | 8078 . . . 8100 | + | Transgenic Insert |
| | TTTG/ | Lb_V2/Lb | | | |
| | TTTG | | | | |
| TI-887 | TTA/ | Fn/Lb_V1/ | 8086 . . . 8108 | + | Transgenic Insert |
| | TTTA/ | Lb_V2/Lb | | | |
| | TTTA | | | | |
| TI-888 | TATG | Lb_V2 | 8088 . . . 8110 | + | Transgenic Insert |
| TI-889 | TTA | Fn | 8068 . . . 8090 | − | Transgenic Insert |
| TI-890 | TTA/ | Fn/Lb_V1/ | 8071 . . . 8093 | − | Transgenic Insert |
| | TTTA/ | Lb_V2/Lb | | | |
| | TTTA | | | | |
| TI-891 | TTA | Fn | 8079 . . . 8101 | − | Transgenic Insert |
| TI-892 | TATC | Lb_V2 | 8084 . . . 8106 | − | Transgenic Insert |
| TI-893 | TATA | Lb_V2 | 8086 . . . 8108 | − | Transgenic Insert |
| TI-894 | TTCA | Lb_V1 | 8168 . . . 8190 | + | Transgenic Insert |
| TI-895 | TATA | Lb_V2 | 8229 . . . 8251 | − | Transgenic Insert |
| TI-896 | TTA/ | Fn/Lb_V1/ | 8254 . . . 8276 | + | Transgenic Insert |
| | TTTA/ | Lb_V2/Lb | | | |
| | TTTA | | | | |
| TI-897 | TATA | Lb_V2 | 8256 . . . 8278 | + | Transgenic Insert |
| TI-898 | TTA/ | Fn/Lb_V1/ | 8250 . . . 8272 | − | Transgenic Insert |
| | TTTA/ | Lb_V2/Lb | | | |
| | TTTA | | | | |
| TI-899 | TTA/ | Fn/Lb_V1/ | 8255 . . . 8277 | − | Transgenic Insert |
| | TTTA/ | Lb_V2/Lb | | | |
| | TTTA | | | | |
| TI-900 | TTCA | Lb_V1 | 8259 . . . 8281 | − | Transgenic Insert |
| TI-901 | TTC/ | Fn/Lb_V1/ | 8260 . . . 8282 | − | Transgenic Insert |
| | TTTC/ | Lb_V2/Lb | | | |
| | TTTC | | | | |
| TI-902 | TTG/ | Fn/Lb_V1/ | 8264 . . . 8286 | − | Transgenic Insert |
| | TTTG/ | Lb_V2/Lb | | | |
| | TTTG | | | | |
| TI-903 | TTA | Fn | 8298 . . . 8320 | + | Transgenic Insert |
| TI-904 | TTC | Fn | 8309 . . . 8331 | + | Transgenic Insert |
| TI-905 | TTCA | Lb_V1 | 8310 . . . 8332 | + | Transgenic Insert |
| TI-906 | TTG | Fn | 8293 . . . 8315 | − | Transgenic Insert |
| TI-907 | TATA | Lb_V2 | 8296 . . . 8318 | − | Transgenic Insert |
| TI-908 | TATA | Lb_V2 | 8298 . . . 8320 | − | Transgenic Insert |
| TI-909 | TATA | Lb_V2 | 8300 . . . 8322 | − | Transgenic Insert |
| TI-910 | TTA | Fn | 8302 . . . 8324 | − | Transgenic Insert |
| TI-911 | TTA/ | Fn/Lb_V1/ | 8306 . . . 8328 | − | Transgenic Insert |
| | TTTA/ | Lb_V2/Lb | | | |
| | TTTA | | | | |
| TI-912 | TATA | Lb_V2 | 8343 . . . 8365 | + | Transgenic Insert |
| TI-913 | TTG | Fn | 8351 . . . 8373 | + | Transgenic Insert |
| TI-914 | TTC/ | Fn/Lb_V1/ | 8355 . . . 8377 | + | Transgenic Insert |
| | TTTC/ | Lb_V2/Lb | | | |
| | TTTC | | | | |
| TI-915 | TTC | Fn | 8358 . . . 8380 | + | Transgenic Insert |
| TI-916 | TTG | Fn | 8344 . . . 8366 | − | Transgenic Insert |
| TI-917 | TTCA | Lb_V1 | 8350 . . . 8372 | − | Transgenic Insert |
| TI-918 | TTC/ | Fn/Lb_V1/ | 8351 . . . 8373 | − | Transgenic Insert |
| | TTTC/ | Lb_V2/Lb | | | |
| | TTTC | | | | |
| TI-919 | TTA/ | Fn/Lb_V1/ | 8383 . . . 8405 | + | Transgenic Insert |
| | TTTA/ | Lb_V2/Lb | | | |
| | TTTA | | | | |
| TI-920 | TTG | Fn | 8380 . . . 8402 | − | Transgenic Insert |
| TI-921 | TTC/ | Fn/Lb_V1/ | 8413 . . . 8435 | + | Transgenic Insert |
| | TTTC/ | Lb_V2/Lb | | | |
| | TTTC | | | | |
| TI-922 | TTCA | Lb_V1 | 8414 . . . 8436 | + | Transgenic Insert |
| TI-923 | TTA/ | Fn/Lb_V1/ | 8424 . . . 8446 | + | Transgenic Insert |
| | TTTA/ | Lb_V2/Lb | | | |
| | TTTA | | | | |
| TI-924 | TATA | Lb_V2 | 8426 . . . 8448 | + | Transgenic Insert |
| TI-925 | TTG | Fn | 8411 . . . 8433 | − | Transgenic Insert |
| TI-926 | TCCA | Lb_V1 | 8419 . . . 8441 | − | Transgenic Insert |
| TI-927 | TATC | Lb_V2 | 8421 . . . 8443 | − | Transgenic Insert |
| TI-928 | TTA | Fn | 8423 . . . 8445 | − | Transgenic Insert |
| TI-929 | TTA/ | Fn/Lb_V1/ | 8465 . . . 8487 | + | Transgenic Insert |
| | TTTA/ | Lb_V2/Lb | | | |
| | TTTA | | | | |
| TI-930 | TATA | Lb_V2 | 8467 . . . 8489 | + | Transgenic Insert |
| TI-931 | TCCA | Lb_V1 | 8468 . . . 8490 | − | Transgenic Insert |
| TI-932 | TTCC | Lb_V1 | 8469 . . . 8491 | − | Transgenic Insert |
| TI-933 | TTC/ | Fn/Lb_V1/ | 8470 . . . 8492 | − | Transgenic Insert |
| | TTTC/ | Lb_V2/Lb | | | |
| | TTTC | | | | |
| TI-934 | TTG | Fn | 8506 . . . 8528 | + | Transgenic Insert |
| TI-935 | TTCG | Lb_V1 | 8501 . . . 8523 | − | Transgenic Insert |
| TI-936 | TTC | Fn | 8502 . . . 8524 | − | Transgenic Insert |
| TI-937 | TTA/ | Fn/Lb_V1/ | 8544 . . . 8566 | + | Transgenic Insert |
| | TTTA/ | Lb_V2/Lb | | | |
| | TTTA | | | | |
| TI-938 | TTA | Fn | 8538 . . . 8560 | − | Transgenic Insert |
| TI-939 | TTG/ | Fn/Lb_V1/ | 8555 . . . 8577 | − | Transgenic Insert |
| | TTTG/ | Lb_V2/Lb | | | |
| | TTTG | | | | |
| TI-940 | TTA/ | Fn/Lb_V1/ | 8574 . . . 8596 | − | Transgenic Insert |
| | TTTA/ | Lb_V2/Lb | | | |
| | TTTA | | | | |
| TI-941 | TCCC | Lb_V1 | 8581 . . . 8603 | − | Transgenic Insert |
| TI-942 | TATC | Lb_V2 | 8583 . . . 8605 | − | Transgenic Insert |
| TI-943 | TTA | Fn | 8585 . . . 8607 | − | Transgenic Insert |
| TI-944 | TTC | Fn | 8631 . . . 8653 | + | Transgenic Insert |
| TI-945 | TTC | Fn | 8634 . . . 8656 | + | Transgenic Insert |
| TI-946 | TTG | Fn | 8637 . . . 8659 | + | Transgenic Insert |
| TI-947 | TTG | Fn | 8620 . . . 8642 | − | Transgenic Insert |
| TI-948 | TTA/ | Fn/Lb_V1/ | 8624 . . . 8646 | − | Transgenic Insert |
| | TTTA/ | Lb_V2/Lb | | | |
| | TTTA | | | | |
| TI-949 | TTA | Fn | 8649 . . . 8671 | + | Transgenic Insert |
| TI-950 | TTG | Fn | 8630 . . . 8652 | − | Transgenic Insert |
| TI-951 | TATA | Lb_V2 | 8642 . . . 8664 | − | Transgenic Insert |
| TI-952 | TATA | Lb_V2 | 8644 . . . 8666 | − | Transgenic Insert |
| TI-953 | TTA/ | Fn/Lb_V1/ | 8678 . . . 8700 | + | Transgenic Insert |
| | TTTA/ | Lb_V2/Lb | | | |
| | TTTA | | | | |
| TI-954 | TTA | Fn | 8680 . . . 8702 | − | Transgenic Insert |
| TI-955 | TTA/ | Fn/Lb_V1/ | 8737 . . . 8759 | + | Transgenic Insert |
| | TTTA/ | Lb_V2/Lb | | | |
| | TTTA | | | | |
| TI-956 | TATA | Lb_V2 | 8739 . . . 8761 | + | Transgenic Insert |
| TI-957 | TTC/ | Fn/Lb_V1/ | 8749 . . . 8771 | + | Transgenic Insert |
| | TTTC/ | Lb_V2/Lb | | | |
| | TTTC | | | | |
| TI-958 | TTG/ | Fn/Lb_V1/ | 8739 . . . 8761 | − | Transgenic Insert |
| | TTTG/ | Lb_V2/Lb | | | |
| | TTTG | | | | |
| TI-959 | TTA/ | Fn/Lb_V1/ | 8746 . . . 8768 | − | Transgenic Insert |
| | TTTA/ | Lb_V2/Lb | | | |
| | TTTA | | | | |
| TI-960 | TTTC | Lb_V1/Lb | 8816 . . . 8838 | + | Transgenic Insert |
| TI-961 | TTA | Fn | 8819 . . . 8841 | + | Transgenic Insert |
| TI-962 | TATG | Lb_V2 | 8825 . . . 8847 | + | Transgenic Insert |
| TI-963 | TTA | Fn | 8829 . . . 8851 | + | Transgenic Insert |
| TI-964 | TTC | Fn | 8833 . . . 8855 | + | Transgenic Insert |
| TI-965 | TTCA | Lb_V1 | 8834 . . . 8856 | + | Transgenic Insert |
| TI-966 | TTA | Fn | 8815 . . . 8837 | − | Transgenic Insert |
| TI-967 | TCCA | Lb_V1 | 8820 . . . 8842 | − | Transgenic Insert |
| TI-968 | TTCC | Lb_V1 | 8821 . . . 8843 | − | Transgenic Insert |
| TI-969 | TTC | Fn | 8822 . . . 8844 | − | Transgenic Insert |
| TI-970 | TTG | Fn | 8830 . . . 8852 | − | Transgenic Insert |
| TI-971 | TTG/ | Fn/Lb_V1/ | 8837 . . . 8859 | − | Transgenic Insert |
| | TTTG/ | Lb_V2/Lb | | | |
| | TTTG | | | | |
| TI-972 | TTA | Fn | 8878 . . . 8900 | + | Transgenic Insert |
| TI-973 | TTA | Fn | 8884 . . . 8906 | + | Transgenic Insert |
| TI-974 | TATA | Lb_V2 | 8862 . . . 8884 | − | Transgenic Insert |
| TI-975 | TTA | Fn | 8887 . . . 8909 | + | Transgenic Insert |
| TI-976 | TATA | Lb_V2 | 8889 . . . 8911 | + | Transgenic Insert |
| TI-977 | TATA | Lb_V2 | 8868 . . . 8890 | − | Transgenic Insert |
| TI-978 | TTA/ | Fn/Lb_V1/ | 8893 . . . 8915 | + | Transgenic Insert |
| | TTTA/ | Lb_V2/Lb | | | |
| | TTTA | | | | |
| TI-979 | TATA | Lb_V2 | 8895 . . . 8917 | + | Transgenic Insert |
| TI-980 | TTG | Fn | 8874 . . . 8896 | − | Transgenic Insert |
| TI-981 | TTG | Fn | 8877 . . . 8899 | − | Transgenic Insert |
| TI-982 | TATG | Lb_V2 | 8890 . . . 8912 | − | Transgenic Insert |
| TI-983 | TTC/ | Fn/Lb_V1/ | 8894 . . . 8916 | − | Transgenic Insert |
| | TTTC/ | Lb_V2/Lb | | | |
| | TTTC | | | | |
| TI-984 | TTC/ | Fn/Lb_V1/ | 8898 . . . 8920 | − | Transgenic Insert |
| | TTTC/ | Lb_V2/Lb | | | |
| | TTTC | | | | |
| TI-985 | TCCA | Lb_V1 | 8973 . . . 8995 | + | Transgenic Insert |
| TI-986 | TCCA | Lb_V1 | 8982 . . . 9004 | + | Transgenic Insert |
| TI-987 | TTA | Fn | 8966 . . . 8988 | − | Transgenic Insert |
| TI-988 | TATC | Lb_V2 | 8970 . . . 8992 | − | Transgenic Insert |
| TI-989 | TTA | Fn | 8972 . . . 8994 | − | Transgenic Insert |
| TI-990 | TTG | Fn | 8980 . . . 9002 | − | Transgenic Insert |
| TI-991 | TTC | Fn | 9004 . . . 9026 | − | Transgenic Insert |
| TI-992 | TTA | Fn | 9035 . . . 9057 | + | Transgenic Insert |
| TI-993 | TTG | Fn | 9014 . . . 9036 | − | Transgenic Insert |
| TI-994 | TATC | Lb_V2 | 9037 . . . 9059 | + | Transgenic Insert |
| TI-995 | TCCA | Lb_V1 | 9039 . . . 9061 | + | Transgenic Insert |
| TI-996 | TTG/ | Fn/Lb_V1/ | 9031 . . . 9053 | − | Transgenic Insert |
| | TTTG/ | Lb_V2/Lb | | | |
| | TTTG | | | | |
| TI-997 | TTA | Fn | 9063 . . . 9085 | + | Transgenic Insert |
| TI-998 | TATC | Lb_V2 | 9065 . . . 9087 | + | Transgenic Insert |
| TI-999 | TCCA | Lb_V1 | 9067 . . . 9089 | + | Transgenic Insert |
| TI-1000 | TATA | Lb_V2 | 9048 . . . 9070 | − | Transgenic Insert |
| TI-1001 | TTC | Fn | 9071 . . . 9093 | + | Transgenic Insert |
| TI-1002 | TATA | Lb_V2 | 9050 . . . 9072 | − | Transgenic Insert |
| TI-1003 | TATA | Lb_V2 | 9052 . . . 9074 | − | Transgenic Insert |
| TI-1004 | TATA | Lb_V2 | 9075 . . . 9097 | + | Transgenic Insert |
| TI-1005 | TTA | Fn | 9054 . . . 9076 | − | Transgenic Insert |
| TI-1006 | TATA | Lb_V2 | 9077 . . . 9099 | + | Transgenic Insert |
| TI-1007 | TATA | Lb_V2 | 9079 . . . 9101 | + | Transgenic Insert |
| TI-1008 | TTC | Fn | 9087 . . . 9109 | + | Transgenic Insert |
| TI-1009 | TTC | Fn | 9090 . . . 9112 | + | Transgenic Insert |
| TI-1010 | TTA | Fn | 9096 . . . 9118 | + | Transgenic Insert |
| TI-1011 | TTG/ | Fn/Lb_V1/ | 9093 . . . 9115 | − | Transgenic Insert |
| | TTTG/ | Lb_V2/Lb | | | |
| | TTTG | | | | |
| TI-1012 | TTC/ | Fn/Lb_V1/ | 9166 . . . 9188 | + | Transgenic Insert |
| | TTTC/ | Lb_V2/Lb | | | |
| | TTTC | | | | |
| TI-1013 | TTC | Fn | 9169 . . . 9191 | + | Transgenic Insert |
| TI-1014 | TTC | Fn | 9174 . . . 9196 | + | Transgenic Insert |
| TI-1015 | TTC/ | Fn/Lb_V1/ | 9216 . . . 9238 | + | Transgenic Insert |
| | TTTC | Lb | | | |
| TI-1016 | TTG/ | Fn/Lb_V1/ | 9225 . . . 9247 | + | Transgenic Insert |
| | TTTG/ | Lb_V2/Lb | | | |
| | TTTG | | | | |
| TI-1017 | TTG/ | Fn/Lb_V1/ | 9229 . . . 9251 | + | Transgenic Insert |
| | TTTG/ | Lb_V2/Lb | | | |
| | TTTG | | | | |
| TI-1018 | TTG/ | Fn/Lb_V1/ | 9234 . . . 9256 | + | Transgenic Insert |
| | TTTG/ | Lb_V2/Lb | | | |
| | TTTG | | | | |
| TI-1019 | TCCA | Lb_V1 | 9214 . . . 9236 | − | Transgenic Insert |
| TI-1020 | TTG/ | Fn/Lb_V1/ | 9239 . . . 9261 | + | Transgenic Insert |
| | TTTG/ | Lb_V2/Lb | | | |
| | TTTG | | | | |
| TI-1021 | TTG/ | Fn/Lb_V1/ | 9287 . . . 9309 | + | Transgenic Insert |
| | TTTG/ | Lb_V2/Lb | | | |
| | TTTG | | | | |
| TI-1022 | TTG/ | Fn/Lb_V1/ | 9291 . . . 9313 | + | Transgenic Insert |
| | TTTG/ | Lb_V2/Lb | | | |
| | TTTG | | | | |
| TI-1023 | TTC | Fn | 9294 . . . 9316 | + | Transgenic Insert |
| TI-1024 | TTCA | Lb_V1 | 9295 . . . 9317 | + | Transgenic Insert |
| TI-1025 | TTA | Fn | 9300 . . . 9322 | + | Transgenic Insert |
| TI-1026 | TTC | Fn | 9303 . . . 9325 | + | Transgenic Insert |
| TI-1027 | TTCA | Lb_V1 | 9304 . . . 9326 | + | Transgenic Insert |
| TI-1028 | TATC | Lb_V2 | 9284 . . . 9306 | − | Transgenic Insert |
| TI-1029 | TATA | Lb_V2 | 9286 . . . 9308 | − | Transgenic Insert |
| TI-1030 | TATA | Lb_V2 | 9288 . . . 9310 | − | Transgenic Insert |
| TI-1031 | TTG/ | Fn/Lb_V1/ | 9297 . . . 9319 | − | Transgenic Insert |
| | TTTG/ | Lb_V2/Lb | | | |
| | TTTG | | | | |
| TI-1032 | TTG/ | Fn/Lb_V1/ | 9367 . . . 9389 | + | Transgenic Insert |
| | TTTG/ | Lb_V2/Lb | | | |
| | TTTG | | | | |
| TI-1033 | TTG | Fn | 9371 . . . 9393 | + | Transgenic Insert |
| TI-1034 | TTG | Fn | 9374 . . . 9396 | + | Transgenic Insert |
| TI-1035 | TTG | Fn | 9377 . . . 9399 | + | Transgenic Insert |
| TI-1036 | TTC | Fn | 9389 . . . 9411 | + | Transgenic Insert |
| TI-1037 | TTA/ | Fn/Lb_V1/ | 9395 . . . 9417 | + | Transgenic Insert |
| | TTTA/ | Lb_V2/Lb | | | |
| | TTTA | | | | |
| TI-1038 | TATG | Lb_V2 | 9397 . . . 9419 | + | Transgenic Insert |
| TI-1039 | TTG/ | Fn/Lb_V1/ | 9439 . . . 9461 | + | Transgenic Insert |
| | TTTG/ | Lb_V2/Lb | | | |
| | TTTG | | | | |
| TI-1040 | TTG | Fn | 9469 . . . 9491 | + | Transgenic Insert |
| TI-1041 | TCCA | Lb_V1 | 9471 . . . 9493 | − | Transgenic Insert |
| TI-1042 | TATC | Lb_V2 | 9485 . . . 9507 | − | Transgenic Insert |
| TI-1043 | TTG | Fn | 9492 . . . 9514 | − | Transgenic Insert |
| TI-1044 | TATC | Lb_V2 | 9532 . . . 9554 | − | Transgenic Insert |
| TI-1045 | TCCC | Lb_V1 | 9544 . . . 9566 | − | Transgenic Insert |
| TI-1046 | TTC/ | Fn/Lb_V1/ | 9548 . . . 9570 | − | Transgenic Insert |
| | TTTC/ | Lb_V2/Lb | | | |
| | TTTC | | | | |
| TI-1047 | TTC | Fn | 9583 . . . 9605 | + | Transgenic Insert |
| TI-1048 | TTG | Fn | 9566 . . . 9588 | − | Transgenic Insert |
| TI-1049 | TCCG | Lb_V1 | 9589 . . . 9611 | − | Transgenic Insert |
| TI-1050 | TCCC | Lb_V1 | 9625 . . . 9647 | + | Transgenic Insert |
| TI-1051 | TTG | Fn | 9633 . . . 9655 | + | Transgenic Insert |
| TI-1052 | TTG | Fn | 9644 . . . 9666 | − | Transgenic Insert |
| TI-1053 | TTC | Fn | 9665 . . . 9687 | − | Transgenic Insert |
| TI-1054 | TTG | Fn | 9694 . . . 9716 | + | Transgenic Insert |
| TI-1055 | TTC | Fn | 9701 . . . 9723 | + | Transgenic Insert |
| TI-1056 | TTC | Fn | 9704 . . . 9726 | + | Transgenic Insert |
| TI-1057 | TTCC | Lb_V1 | 9705 . . . 9727 | + | Transgenic Insert |
| TI-1058 | TTA | Fn | 9711 . . . 9733 | + | Transgenic Insert |
| TI-1059 | TCCA | Lb_V1 | 9695 . . . 9717 | − | Transgenic Insert |
| TI-1060 | TTC | Fn | 9702 . . . 9724 | − | Transgenic Insert |
| TI-1061 | TCCA | Lb_V1 | 9715 . . . 9737 | − | Transgenic Insert |
| TI-1062 | TTCA | Lb_V1 | 9727 . . . 9749 | − | Transgenic Insert |
| TI-1063 | TTC | Fn | 9728 . . . 9750 | − | Transgenic Insert |
| TI-1064 | TTG | Fn | 9758 . . . 9780 | + | Transgenic Insert |
| TI-1065 | TTC | Fn | 9770 . . . 9792 | + | Transgenic Insert |
| TI-1066 | TTG | Fn | 9749 . . . 9771 | − | Transgenic Insert |
| TI-1067 | TTC | Fn | 9773 . . . 9795 | + | Transgenic Insert |
| TI-1068 | TTCA | Lb_V1 | 9774 . . . 9796 | + | Transgenic Insert |
| TI-1069 | TTG | Fn | 9757 . . . 9779 | − | Transgenic Insert |
| TI-1070 | TTCG | Lb_V1 | 9761 . . . 9783 | − | Transgenic Insert |
| TI-1071 | TTC | Fn | 9762 . . . 9784 | − | Transgenic Insert |
| TI-1072 | TCCG | Lb_V1 | 9765 . . . 9787 | − | Transgenic Insert |
| TI-1073 | TTCC | Lb_V1 | 9766 . . . 9788 | − | Transgenic Insert |
| TI-1074 | TTC | Fn | 9767 . . . 9789 | − | Transgenic Insert |
| TI-1075 | TTC/ | Fn/Lb_V1/ | 9773 . . . 9795 | − | Transgenic Insert |
| | TTTC/ | Lb_V2/Lb | | | |
| | TTTC | | | | |
| TI-1076 | TTC | Fn | 9803 . . . 9825 | + | Transgenic Insert |
| TI-1077 | TTG | Fn | 9782 . . . 9804 | − | Transgenic Insert |
| TI-1078 | TTCA | Lb_V1 | 9804 . . . 9826 | + | Transgenic Insert |
| TI-1079 | TTG | Fn | 9812 . . . 9834 | + | Transgenic Insert |
| TI-1080 | TTG | Fn | 9797 . . . 9819 | − | Transgenic Insert |
| TI-1081 | TTC | Fn | 9800 . . . 9822 | − | Transgenic Insert |
| TI-1082 | TTC | Fn | 9830 . . . 9852 | + | Transgenic Insert |
| TI-1083 | TTCC | Lb_V1 | 9831 . . . 9853 | + | Transgenic Insert |
| TI-1084 | TCCA | Lb_V1 | 9832 . . . 9854 | + | Transgenic Insert |
| TI-1085 | TTG | Fn | 9813 . . . 9835 | − | Transgenic Insert |
| TI-1086 | TTC | Fn | 9837 . . . 9859 | + | Transgenic Insert |
| TI-1087 | TTCA | Lb_V1 | 9838 . . . 9860 | + | Transgenic Insert |
| TI-1088 | TCCA | Lb_V1 | 9821 . . . 9843 | − | Transgenic Insert |
| TI-1089 | TTCC | Lb_V1 | 9822 . . . 9844 | − | Transgenic Insert |
| TI-1090 | TTC | Fn | 9823 . . . 9845 | − | Transgenic Insert |
| TI-1091 | TATG | Lb_V2 | 9846 . . . 9868 | + | Transgenic Insert |
| TI-1092 | TTA | Fn | 9864 . . . 9886 | + | Transgenic Insert |
| TI-1093 | TTG | Fn | 9843 . . . 9865 | − | Transgenic Insert |
| TI-1094 | TCCA | Lb_V1 | 9856 . . . 9878 | − | Transgenic Insert |
| TI-1095 | TTG | Fn | 9899 . . . 9921 | + | Transgenic Insert |
| TI-1096 | TTCA | Lb_V1 | 9887 . . . 9909 | − | Transgenic Insert |
| TI-1097 | TTC | Fn | 9888 . . . 9910 | − | Transgenic Insert |
| TI-1098 | TTG | Fn | 9912 . . . 9934 | + | Transgenic Insert |
| TI-1099 | TTG | Fn | 9893 . . . 9915 | − | Transgenic Insert |
| TI-1100 | TTG | Fn | 9903 . . . 9925 | − | Transgenic Insert |
| TI-1101 | TTG | Fn | 9936 . . . 9958 | + | Transgenic Insert |
| TI-1102 | TTC | Fn | 9941 . . . 9963 | + | Transgenic Insert |
| TI-1103 | TTCA | Lb_V1 | 9932 . . . 9954 | − | Transgenic Insert |
| TI-1104 | TTC | Fn | 9933 . . . 9955 | − | Transgenic Insert |
| TI-1105 | TCCC | Lb V1 | 9943 . . . 9965 | − | Transgenic Insert |
| TI-1106 | TTC/ | Fn/Lb_V1/ | 9965 . . . 9987 | + | Transgenic Insert |
| | TTTC/ | Lb_V2/Lb | | | |
| | TTTC | | | | |
| TI-1107 | TTCA | Lb_V1 | 9966 . . . 9988 | + | Transgenic Insert |
| TI-1108 | TTCA | Lb_V1 | 9952 . . . 9974 | − | Transgenic Insert |
| TI-1109 | TTC | Fn | 9975 . . . 9997 | + | Transgenic Insert |
| TI-1110 | TTC | Fn | 9953 . . . 9975 | − | Transgenic Insert |
| TI-1111 | TTG | Fn | 9956 . . . 9978 | − | Transgenic Insert |
| TI-1112 | TTCC | Lb_V1 | 9969 . . . 9991 | − | Transgenic Insert |
| TI-1113 | TTC | Fn | 9970 . . . 9992 | − | Transgenic Insert |
| TI-1114 | TTA/ | Fn/Lb_V1/ | 9974 . . . 9996 | − | Transgenic Insert |
| | TTTA/ | Lb_V2/Lb | | | |
| | TTTA | | | | |
| TI-1115 | TTA | Fn | 10008 . . . 10030 | + | Transgenic Insert |
| TI-1116 | TATC | Lb_V2 | 10019 . . . 10041 | + | Transgenic Insert |
| TI-1117 | TTC | Fn | 10023 . . . 10045 | + | Transgenic Insert |
| TI-1118 | TTCA | Lb_V1 | 10024 . . . 10046 | + | Transgenic Insert |
| TI-1119 | TATG | Lb_V2 | 10031 . . . 10053 | + | Transgenic Insert |
| TI-1120 | TTG | Fn | 10010 . . . 10032 | − | Transgenic Insert |
| TI-1121 | TTC | Fn | 10041 . . . 10063 | + | Transgenic Insert |
| TI-1122 | TTG | Fn | 10044 . . . 10066 | + | Transgenic Insert |
| TI-1123 | TTG | Fn | 10050 . . . 10072 | + | Transgenic Insert |
| TI-1124 | TTA | Fn | 10060 . . . 10082 | + | Transgenic Insert |
| TI-1125 | TTC | Fn | 10064 . . . 10086 | + | Transgenic Insert |
| TI-1126 | TCCA | Lb_V1 | 10042 . . . 10064 | − | Transgenic Insert |
| TI-1127 | TTG | Fn | 10067 . . . 10089 | + | Transgenic Insert |
| TI-1128 | TTG | Fn | 10052 . . . 10074 | − | Transgenic Insert |
| TI-1129 | TTCG | Lb_V1 | 10055 . . . 10077 | − | Transgenic Insert |
| TI-1130 | TTC | Fn | 10056 . . . 10078 | − | Transgenic Insert |
| TI-1131 | TTG | Fn | 10072 . . . 10094 | − | Transgenic Insert |
| TI-1132 | TTC | Fn | 10094 . . . 10116 | + | Transgenic Insert |
| TI-1133 | TTCG | Lb_V1 | 10095 . . . 10117 | + | Transgenic Insert |
| TI-1134 | TTG | Fn | 10079 . . . 10101 | − | Transgenic Insert |
| TI-1135 | TTC | Fn | 10103 . . . 10125 | + | Transgenic Insert |
| TI-1136 | TTCA | Lb_V1 | 10104 . . . 10126 | + | Transgenic Insert |
| TI-1137 | TCCC | Lb_V1 | 10118 . . . 10140 | + | Transgenic Insert |
| TI-1138 | TTG | Fn | 10099 . . . 10121 | − | Transgenic Insert |
| TI-1139 | TCCA | Lb_V1 | 10124 . . . 10146 | + | Transgenic Insert |
| TI-1140 | TTG | Fn | 10137 . . . 10159 | + | Transgenic Insert |
| TI-1141 | TTC | Fn | 10142 . . . 10164 | + | Transgenic Insert |
| TI-1142 | TTCG | Lb_V1 | 10143 . . . 10165 | + | Transgenic Insert |
| TI-1143 | TCCG | Lb_V1 | 10161 . . . 10183 | + | Transgenic Insert |
| TI-1144 | TTG | Fn | 10175 . . . 10197 | + | Transgenic Insert |
| TI-1145 | TTC | Fn | 10182 . . . 10204 | + | Transgenic Insert |
| TI-1146 | TTC | Fn | 10185 . . . 10207 | + | Transgenic Insert |
| TI-1147 | TTG/ | Fn/Lb_V1/ | 10169 . . . 10191 | − | Transgenic Insert |
| | TTTG/ | Lb_V2/Lb | | | |
| | TTTG | | | | |
| TI-1148 | TTG | Fn | 10188 . . . 10210 | − | Transgenic Insert |
| TI-1149 | TTC | Fn | 10218 . . . 10240 | + | Transgenic Insert |
| TI-1150 | TTCA | Lb_V1 | 10219 . . . 10241 | + | Transgenic Insert |
| TI-1151 | TCCC | Lb_V1 | 10198 . . . 10220 | − | Transgenic Insert |
| TI-1152 | TTG | Fn | 10205 . . . 10227 | − | Transgenic Insert |
| TI-1153 | TTG | Fn | 10214 . . . 10236 | − | Transgenic Insert |
| TI-1154 | TCCA | Lb_V1 | 10239 . . . 10261 | + | Transgenic Insert |
| TI-1155 | TTG | Fn | 10245 . . . 10267 | + | Transgenic Insert |
| TI-1156 | TTG | Fn | 10225 . . . 10247 | − | Transgenic Insert |
| TI-1157 | TTG | Fn | 10265 . . . 10287 | + | Transgenic Insert |
| TI-1158 | TTG | Fn | 10247 . . . 10269 | − | Transgenic Insert |
| TI-1159 | TTA | Fn | 10277 . . . 10299 | + | Transgenic Insert |
| TI-1160 | TCCA | Lb_V1 | 10264 . . . 10286 | − | Transgenic Insert |
| TI-1161 | TTA | Fn | 10270 . . . 10292 | − | Transgenic Insert |
| TI-1162 | TTG | Fn | 10277 . . . 10299 | − | Transgenic Insert |
| TI-1163 | TTC | Fn | 10313 . . . 10335 | + | Transgenic Insert |
| TI-1164 | TTCA | Lb_V1 | 10314 . . . 10336 | + | Transgenic Insert |
| TI-1165 | TTG/ | Fn/Lb_V1/ | 10315 . . . 10337 | − | Transgenic Insert |
| | TTTG/ | Lb_V2/Lb | | | |
| | TTTG | | | | |
| TI-1166 | TTC | Fn | 10325 . . . 10347 | − | Transgenic Insert |
| TI-1167 | TCCC | Lb_V1 | 10365 . . . 10387 | + | Transgenic Insert |
| TI-1168 | TATA | Lb_V2 | 10351 . . . 10373 | − | Transgenic Insert |
| TI-1169 | TTA | Fn | 10376 . . . 10398 | + | Transgenic Insert |
| TI-1170 | TATA | Lb_V2 | 10378 . . . 10400 | + | Transgenic Insert |
| TI-1171 | TTCA | Lb_V1 | 10360 . . . 10382 | − | Transgenic Insert |
| TI-1172 | TTC | Fn | 10361 . . . 10383 | − | Transgenic Insert |
| TI-1173 | TTC/ | Fn/Lb_V1/ | 10370 . . . 10392 | − | Transgenic Insert |
| | TTTC/ | Lb_V2/Lb | | | |
| | TTTC | | | | |
| TI-1174 | TTG | Fn | 10404 . . . 10426 | + | Transgenic Insert |
| TI-1175 | TCCA | Lb_V1 | 10399 . . . 10421 | − | Transgenic Insert |
| TI-1176 | TTG/ | Fn/Lb_V1/ | 10421 . . . 10443 | + | Transgenic Insert |
| | TTTG/ | Lb_V2/Lb | | | |
| | TTTG | | | | |
| TI-1177 | TTC | Fn | 10406 . . . 10428 | − | Transgenic Insert |
| TI-1178 | TTG | Fn | 10417 . . . 10439 | − | Transgenic Insert |
| TI-1179 | TATC | Lb_V2 | 10420 . . . 10442 | − | Transgenic Insert |
| TI-1180 | TTCC | Lb_V1 | 10426 . . . 10448 | − | Transgenic Insert |
| TI-1181 | TTC/ | Fn/Lb_V1/ | 10427 . . . 10449 | − | Transgenic Insert |
| | TTTC/ | Lb_V2/Lb | | | |
| | TTTC | | | | |
| TI-1182 | TTCG | Lb_V1 | 10439 . . . 10461 | − | Transgenic Insert |
| TI-1183 | TTC | Fn | 10440 . . . 10462 | − | Transgenic Insert |
| TI-1184 | TCCC | Lb_V1 | 10455 . . . 10477 | − | Transgenic Insert |
| TI-1185 | TCCA | Lb_V1 | 10463 . . . 10485 | − | Transgenic Insert |
| TI-1186 | TTCC | Lb_V1 | 10464 . . . 10486 | − | Transgenic Insert |
| TI-1187 | TTC/ | Fn/Lb_V1/ | 10465 . . . 10487 | − | Transgenic Insert |
| | TTTC/ | Lb_V2/Lb | | | |
| | TTTC | | | | |
| TI-1188 | TTC | Fn | 10496 . . . 10518 | + | Transgenic Insert |
| TI-1189 | TCCC | Lb_V1 | 10524 . . . 10546 | + | Transgenic Insert |
| TI-1190 | TTA | Fn | 10521 . . . 10543 | − | Transgenic Insert |
| TI-1191 | TTA | Fn | 10546 . . . 10568 | + | Transgenic Insert |
| TI-1192 | TTA | Fn | 10525 . . . 10547 | − | Transgenic Insert |
| TI-1193 | TTA | Fn | 10550 . . . 10572 | + | Transgenic Insert |
| TI-1194 | TTA | Fn | 10529 . . . 10551 | − | Transgenic Insert |
| TI-1195 | TTA | Fn | 10554 . . . 10576 | + | Transgenic Insert |
| TI-1196 | TTA | Fn | 10533 . . . 10555 | − | Transgenic Insert |
| TI-1197 | TTA | Fn | 10558 . . . 10580 | + | Transgenic Insert |
| TI-1198 | TTC | Fn | 10586 . . . 10608 | + | Transgenic Insert |
| TI-1199 | TTCA | Lb_V1 | 10587 . . . 10609 | + | Transgenic Insert |
| TI-1200 | TTG/ | Fn/Lb_V1/ | 10594 . . . 10616 | + | Transgenic Insert |
| | TTTG/ | Lb_V2/Lb | | | |
| | TTTG | | | | |
| TI-1201 | TTA/ | Fn/Lb_V1/ | 10598 . . . 10620 | + | Transgenic Insert |
| | TTTA/ | Lb_V2/Lb | | | |
| | TTTA | | | | |
| TI-1202 | TTC/ | Fn/Lb_V1/ | 10602 . . . 10624 | + | Transgenic Insert |
| | TTTC/ | Lb_V2/Lb | | | |
| | TTTC | | | | |
| TI-1203 | TTCC | Lb_V1 | 10603 . . . 10625 | + | Transgenic Insert |
| TI-1204 | TTA/ | Fn/Lb_V1/ | 10642 . . . 10664 | + | Transgenic Insert |
| | TTTA/ | Lb_V2/Lb | | | |
| | TTTA | | | | |
| TI-1205 | TATA | Lb_V2 | 10644 . . . 10666 | + | Transgenic Insert |
| TI-1206 | TTG/ | Fn/Lb_V1/ | 10673 . . . 10695 | + | Transgenic Insert |
| | TTTG/ | Lb_V2/Lb | | | |
| | TTTG | | | | |
| TI-1207 | TTG/ | Fn/Lb_V1/ | 10678 . . . 10700 | + | Transgenic Insert |
| | TTTG/ | Lb_V2/Lb | | | |
| | TTTG | | | | |
| TI-1208 | TTG/ | Fn/Lb_V1/ | 10684 . . . 10706 | + | Transgenic Insert |
| | TTTG/ | Lb_V2/Lb | | | |
| | TTTG | | | | |
| TI-1209 | TTC/ | Fn/Lb_V1/ | 10688 . . . 10710 | + | Transgenic Insert |
| | TTTC/ | Lb_V2/Lb | | | |
| | TTTC | | | | |
| TI-1210 | TTCC | Lb_V1 | 10689 . . . 10711 | + | Transgenic Insert |
| TI-1211 | TCCA | Lb_V1 | 10690 . . . 10712 | + | Transgenic Insert |
| TI-1212 | TTG | Fn | 10696 . . . 10718 | + | Transgenic Insert |
| TI-1213 | TCCA | Lb_V1 | 10704 . . . 10726 | + | Transgenic Insert |
| TI-1214 | TATC | Lb_V2 | 10714 . . . 10736 | + | Transgenic Insert |
| TI-1215 | TTA | Fn | 10693 . . . 10715 | − | Transgenic Insert |
| TI-1216 | TTA/ | Fn/Lb_V1/ | 10700 . . . 10722 | − | Transgenic Insert |
| | TTTA/ | Lb_V2/Lb | | | |
| | TTTA | | | | |
| TI-1217 | TTG/ | Fn/Lb_V1/ | 10723 . . . 10745 | + | Transgenic Insert |
| | TTTG/ | Lb_V2/Lb | | | |
| | TTTG | | | | |
| TI-1218 | TTG | Fn | 10710 . . . 10732 | − | Transgenic Insert |
| TI-1219 | TTA/ | Fn/Lb_V1/ | 10713 . . . 10735 | − | Transgenic Insert |
| | TTTA/ | Lb_V2/Lb | | | |
| | TTTA | | | | |
| TI-1220 | TTA/ | Fn/Lb_V1/ | 10717 . . . 10739 | − | Transgenic Insert |
| | TTTA/ | Lb_V2/Lb | | | |
| | TTTA | | | | |
| TI-1221 | TTA | Fn | 10722 . . . 10744 | − | Transgenic Insert |
| TI-1222 | TTA/ | Fn/Lb_V1/ | 10725 . . . 10747 | − | Transgenic Insert |
| | TTTA/ | Lb_V2/Lb | | | |
| | TTTA | | | | |
| TI-1223 | TTA | Fn | 10747 . . . 10769 | + | Transgenic Insert |
| TI-1224 | TTG/ | Fn/Lb_V1/ | 10756 . . . 10778 | + | Transgenic Insert |
| | TTTG/ | Lb_V2/Lb | | | |
| | TTTG | | | | |
| TI-1225 | TTC/ | Fn/Lb_V1/ | 10761 . . . 10783 | + | Transgenic Insert |
| | TTTC/ | Lb_V2/Lb | | | |
| | TTTC | | | | |
| TI-1226 | TATA | Lb_V2 | 10745 . . . 10767 | − | Transgenic Insert |
| TI-1227 | TATA | Lb_V2 | 10772 . . . 10794 | + | Transgenic Insert |
| TI-1228 | TATG | Lb_V2 | 10774 . . . 10796 | + | Transgenic Insert |
| TI-1229 | TCCA | Lb_V1 | 10759 . . . 10781 | − | Transgenic Insert |
| TI-1230 | TTG/ | Fn/Lb_V1/ | 10784 . . . 10806 | + | Transgenic Insert |
| | TTTG/ | Lb_V2/Lb | | | |
| | TTTG | | | | |
| TI-1231 | TTG | Fn | 10792 . . . 10814 | + | Transgenic Insert |
| TI-1232 | TTG/ | Fn/Lb_V1/ | 10796 . . . 10818 | + | Transgenic Insert |
| | TTTG/ | Lb_V2/Lb | | | |
| | TTTG | | | | |
| TI-1233 | TTG/ | Fn/Lb_V1/ | 10825 . . . 10847 | + | Transgenic Insert |
| | TTTG/ | Lb_V2/Lb | | | |
| | TTTG | | | | |
| TI-1234 | TTG | Fn | 10881 . . . 10903 | + | Transgenic Insert |
| TI-1235 | TTG/ | Fn/Lb_V1/ | 10886 . . . 10908 | + | Transgenic Insert |
| | TTTG/ | Lb_V2/Lb | | | |
| | TTTG | | | | |
| TI-1236 | TTG | Fn | 10871 . . . 10893 | − | Transgenic Insert |
| TI-1237 | TTG/ | Fn/Lb_V1/ | 10874 . . . 10896 | − | Transgenic Insert |
| | TTTG/ | Lb_V2/Lb | | | |
| | TTTG | | | | |
| TI-1238 | TTA | Fn | 10897 . . . 10919 | − | Transgenic Insert |
| TI-1239 | TTA | Fn | 10922 . . . 10944 | + | Transgenic Insert |
| TI-1240 | TTG | Fn | 10943 . . . 10965 | + | Transgenic Insert |
| TI-1241 | TTCA | Lb_V1 | 10934 . . . 10956 | − | Transgenic Insert |
| TI-1242 | TTC | Fn | 10935 . . . 10957 | − | Transgenic Insert |
| TI-1243 | TTA | Fn | 10948 . . . 10970 | − | Transgenic Insert |
| TI-1244 | TTG/ | Fn/Lb_V1/ | 10985 . . . 11007 | + | Transgenic Insert |
| | TTTG/ | Lb_V2/Lb | | | |
| | TTTG | | | | |
| TI-1245 | TTCG | Lb_V1 | 10981 . . . 11003 | − | Transgenic Insert |
| TI-1246 | TTC/ | Fn/Lb_V1/ | 10982 . . . 11004 | − | Transgenic Insert |
| | TTTC/ | Lb_V2/Lb | | | |
| | TTTC | | | | |
| TI-1247 | TTA/ | Fn/Lb_V1/ | 10986 . . . 11008 | − | Transgenic Insert |
| | TTTA/ | Lb_V2/Lb | | | |
| | TTTA | | | | |
| TI-1248 | TTCG | Lb_V1 | 10996 . . . 11018 | − | Transgenic Insert |
| TI-1249 | TTC | Fn | 10997 . . . 11019 | − | Transgenic Insert |
| TI-1250 | TTC/ | Fn/Lb_V1/ | 11019 . . . 11041 | + | Transgenic Insert |
| | TTTC/ | Lb_V2/Lb | | | |
| | TTTC | | | | |
| TI-1251 | TTCC | Lb_V1 | 11020 . . . 11042 | + | Transgenic Insert |
| TI-1252 | TCCG | Lb_V1 | 11021 . . . 11043 | + | Transgenic Insert |
| TI-1253 | TTC | Fn | 11003 . . . 11025 | − | Transgenic Insert |
| TI-1254 | TTA | Fn | 11026 . . . 11048 | + | Transgenic Insert |
| TI-1255 | TTA | Fn | 11006 . . . 11028 | − | Transgenic Insert |
| TI-1256 | TTG/ | Fn/Lb_V1/ | 11036 . . . 11058 | + | Transgenic Insert |
| | TTTG/ | Lb_V2/Lb | | | |
| | TTTG | | | | |
| TI-1257 | TTA/ | Fn/Lb_V1/ | 11040 . . . 11062 | + | Transgenic Insert |
| | TTTA/ | Lb_V2/Lb | | | |
| | TTTA | | | | |
| TI-1258 | TTG | Fn | 11043 . . . 11065 | + | Transgenic Insert |
| TI-1259 | TTC/ | Fn/Lb_V1/ | 11048 . . . 11070 | + | Transgenic Insert |
| | TTTC/ | Lb_V2/Lb | | | |
| | TTTC | | | | |
| TI-1260 | TTCG | Lb_V1 | 11049 . . . 11071 | + | Transgenic Insert |
| TI-1261 | TATA | Lb_V2 | 11028 . . . 11050 | − | Transgenic Insert |
| TI-1262 | TTA/ | Fn/Lb_V1/ | 11030 . . . 11052 | − | Transgenic Insert |
| | TTTA/ | Lb_V2/Lb | | | |
| | TTTA | | | | |
| TI-1263 | TATA | Lb_V2 | 11055 . . . 11077 | + | Transgenic Insert |
| TI-1264 | TCCG | Lb_V1 | 11039 . . . 11061 | − | Transgenic Insert |
| TI-1265 | TTA | Fn | 11046 . . . 11068 | − | Transgenic Insert |
| TI-1266 | TTA/ | Fn/Lb_V1/ | 11105 . . . 11127 | + | Transgenic Insert |
| | TTTA/ | Lb_V2/Lb | | | |
| | TTTA | | | | |
| TI-1267 | TATA | Lb_V2 | 11107 . . . 11129 | + | Transgenic Insert |
| TI-1268 | TTC/ | Fn/Lb_V1/ | 11170 . . . 11192 | + | Transgenic Insert |
| | TTTC/ | Lb_V2/Lb | | | |
| | TTTC | | | | |
| TI-1269 | TCCA | Lb_V1 | 11174 . . . 11196 | + | Transgenic Insert |
| TI-1270 | TTA | Fn | 11173 . . . 11195 | − | Transgenic Insert |
| 3J-1 | TTG | Fn | 11179 . . . 11201 | + | 3′ Flanking Genomic DNA |
| 3J-2 | TTG | Fn | 11195 . . . 11217 | + | 3′ Flanking Genomic DNA |
| 3F-1 | TTG | Fn | 11202 . . . 11224 | + | 3′ Flanking Genomic DNA |
| 3F-2 | TTC | Fn | 11207 . . . 11229 | + | 3′ Flanking Genomic DNA |
| 3F-3 | TTCG | Lb_V1 | 11203 . . . 11225 | − | 3′ Flanking Genomic DNA |
| 3F-4 | TTC/ | Fn/Lb_V1/ | 11204 . . . 11226 | − | 3′ Flanking Genomic DNA |
| | TTTC/ | Lb_V2/Lb | | | |
| | TTTC | | | | |
| 3F-5 | TTA | Fn | 11255 . . . 11277 | + | 3′ Flanking Genomic DNA |
| 3F-6 | TTA/ | Fn/Lb_V1/ | 11274 . . . 11296 | + | 3′ Flanking Genomic DNA |
| | TTTA/ | Lb_V2/Lb | | | |
| | TTTA | | | | |
| 3F-7 | TTC | Fn | 11277 . . . 11299 | + | 3′ Flanking Genomic DNA |
| 3F-8 | TTA | Fn | 11282 . . . 11304 | + | 3′ Flanking Genomic DNA |
| 3F-9 | TTG/ | Fn/Lb_V1/ | 11268 . . . 11290 | − | 3′ Flanking Genomic DNA |
| | TTTG/ | Lb_V2/Lb | | | |
| | TTTG | | | | |
| 3F-10 | TTG | Fn | 11273 . . . 11295 | − | 3′ Flanking Genomic DNA |
| 3F-11 | TTC/ | Fn/Lb_V1/ | 11279 . . . 11301 | − | 3′ Flanking Genomic DNA |
| | TTTC/ | Lb_V2/Lb | | | |
| | TTTC | | | | |
| 3F-12 | TTA/ | Fn/Lb_V1/ | 11355 . . . 11377 | + | 3′ Flanking Genomic DNA |
| | TTTA/ | Lb_V2/Lb | | | |
| | TTTA | | | | |
| 3F-13 | TTA/ | Fn/Lb_V1/ | 11355 . . . 11377 | − | 3′ Flanking Genomic DNA |
| | TTTA/ | Lb_V2/Lb | | | |
| | TTTA | | | | |
| 3F-14 | TTA/ | Fn/Lb_V1/ | 11380 . . . 11402 | + | 3′ Flanking Genomic DNA |
| | TTTA/ | Lb_V2/Lb | | | |
| | TTTA | | | | |
| 3F-15 | TTA/ | Fn/Lb_V1/ | 11409 . . . 11431 | + | 3′ Flanking Genomic DNA |
| | TTTA/ | Lb_V2/Lb | | | |
| | TTTA | | | | |
| 3F-16 | TTA | Fn | 11412 . . . 11434 | + | 3′ Flanking Genomic DNA |
| 3F-17 | TTC/ | Fn/Lb_V1/ | 11416 . . . 11438 | + | 3′ Flanking Genomic DNA |
| | TTTC/ | Lb_V2/Lb | | | |
| | TTTC | | | | |
| 3F-18 | TTCA | Lb_V1 | 11417 . . . 11439 | + | 3′ Flanking Genomic DNA |
| 3F-19 | TTA | Fn | 11421 . . . 11443 | + | 3′ Flanking Genomic DNA |
| 3F-20 | TATC | Lb_V2 | 11423 . . . 11445 | + | 3′ Flanking Genomic DNA |
| 3F-21 | TTCA | Lb_V1 | 11405 . . . 11427 | − | 3′ Flanking Genomic DNA |
| 3F-22 | TTC | Fn | 11406 . . . 11428 | − | 3′ Flanking Genomic DNA |
| 3F-23 | TTG | Fn | 11430 . . . 11452 | + | 3′ Flanking Genomic DNA |
| 3F-24 | TTG | Fn | 11435 . . . 11457 | + | 3′ Flanking Genomic DNA |
| 3F-25 | TTG/ | Fn/Lb_V1/ | 11415 . . . 11437 | − | 3′ Flanking Genomic DNA |
| | TTTG/ | Lb_V2/Lb | | | |
| | TTTG | | | | |
| 3F-26 | TTC/ | Fn/Lb_V1/ | 11446 . . . 11468 | + | 3′ Flanking Genomic DNA |
| | TTTC/ | Lb_V2/Lb | | | |
| | TTTC | | | | |
| 3F-27 | TTG | Fn | 11426 . . . 11448 | − | 3′ Flanking Genomic DNA |
| 3F-28 | TTC | Fn | 11455 . . . 11477 | + | 3′ Flanking Genomic DNA |
| 3F-29 | TTA | Fn | 11440 . . . 11462 | − | 3′ Flanking Genomic DNA |
| 3F-30 | TTA/ | Fn/Lb_V1/ | 11465 . . . 11487 | + | 3′ Flanking Genomic DNA |
| | TTTA/ | Lb_V2/Lb | | | |
| | TTTA | | | | |
| 3F-31 | TTA | Fn | 11450 . . . 11472 | − | 3′ Flanking Genomic DNA |
| 3F-32 | TTA | Fn | 11454 . . . 11476 | − | 3′ Flanking Genomic DNA |
| 3F-33 | TTC | Fn | 11509 . . . 11531 | | 3′ Flanking Genomic DNA |
| 3F-34 | TTA/ | Fn/Lb_V1/ | 11508 . . . 11530 | − | 3′ Flanking Genomic DNA |
| | TTTA/ | Lb_V2/Lb | | | |
| | TTTA | | | | |
| 3F-35 | TTA | Fn | 11555 . . . 11577 | + | 3′ Flanking Genomic DNA |
| 3F-36 | TATA | Lb_V2 | 11557 . . . 11579 | + | 3′ Flanking Genomic DNA |
| 3F-37 | TTA | Fn | 11561 . . . 11583 | + | 3′ Flanking Genomic DNA |
| 3F-38 | TTG | Fn | 11576 . . . 11598 | + | 3′ Flanking Genomic DNA |
| 3F-39 | TTG | Fn | 11582 . . . 11604 | + | 3′ Flanking Genomic DNA |
| 3F-40 | TCCA | Lb_V1 | 11560 . . . 11582 | − | 3′ Flanking Genomic DNA |
| 3F-41 | TTG | Fn | 11585 . . . 11607 | + | 3′ Flanking Genomic DNA |
| 3F-42 | TATA | Lb_V2 | 11580 . . . 11602 | − | 3′ Flanking Genomic DNA |
| 3F-43 | TTA/ | Fn/Lb_V1/ | 11582 . . . 11604 | − | 3′ Flanking Genomic DNA |
| | TTTA/ | Lb_V2/Lb | | | |
| | TTTA | | | | |
| 3F-44 | TTA | Fn | 11618 . . . 11640 | + | 3′ Flanking Genomic DNA |
| 3F-45 | TTG | Fn | 11622 . . . 11644 | − | 3′ Flanking Genomic DNA |
| 3F-46 | TTG | Fn | 11628 . . . 11650 | − | 3′ Flanking Genomic DNA |
| 3F-47 | TTG | Fn | 11636 . . . 11658 | − | 3′ Flanking Genomic DNA |
| 3F-48 | TTG/ | Fn/Lb_V1/ | 11658 . . . 11680 | + | 3′ Flanking Genomic DNA |
| | TTTG/ | Lb_V2/Lb | | | |
| | TTTG | | | | |
| 3F-49 | TTG | Fn | 11641 . . . 11663 | − | 3′ Flanking Genomic DNA |
| 3F-50 | TTC | Fn | 11644 . . . 11666 | − | 3′ Flanking Genomic DNA |
| 3F-51 | TTC/ | Fn/Lb_V1/ | 11683 . . . 11705 | + | 3′ Flanking Genomic DNA |
| | TTTC/ | Lb_V2/Lb | | | |
| | TTTC | | | | |
| 3F-52 | TTCC | Lb_V1 | 11684 . . . 11706 | + | 3′ Flanking Genomic DNA |
| 3F-53 | TTA | Fn | 11663 . . . 11685 | − | 3′ Flanking Genomic DNA |
| 3F-54 | TCCA | Lb_V1 | 11685 . . . 11707 | + | 3′ Flanking Genomic DNA |
| 3F-55 | TTA | Fn | 11688 . . . 11710 | + | 3′ Flanking Genomic DNA |
| 3F-56 | TTA | Fn | 11667 . . . 11689 | − | 3′ Flanking Genomic DNA |
| 3F-57 | TTG | Fn | 11675 . . . 11697 | − | 3′ Flanking Genomic DNA |
| 3F-58 | TTG | Fn | 11693 . . . 11715 | − | 3′ Flanking Genomic DNA |
| 3F-59 | TATG | Lb_V2 | 11700 . . . 11722 | − | 3′ Flanking Genomic DNA |
| 3F-60 | TTG/ | Fn/Lb_V1/ | 11716 . . . 11738 | − | 3′ Flanking Genomic DNA |
| | TTTG/ | Lb_V2/Lb | | | |
| | TTTG | | | | |
| 3F-61 | TTG | Fn | 11763 . . . 11785 | + | 3′ Flanking Genomic DNA |
| 3F-62 | TTA | Fn | 11757 . . . 11779 | − | 3′ Flanking Genomic DNA |
| 3F-63 | TTA | Fn | 11763 . . . 11785 | − | 3′ Flanking Genomic DNA |
| 3F-64 | TTA/ | Fn/Lb_V1/ | 11788 . . . 11810 | + | 3′ Flanking Genomic DNA |
| | TTTA/ | Lb_V2/Lb | | | |
| | TTTA | | | | |
| 3F-65 | TTG | Fn | 11795 . . . 11817 | + | 3′ Flanking Genomic DNA |
| 3F-66 | TTA | Fn | 11800 . . . 11822 | + | 3′ Flanking Genomic DNA |
| 3F-67 | TATA | Lb_V2 | 11802 . . . 11824 | + | 3′ Flanking Genomic DNA |
| 3F-68 | TTCA | Lb_V1 | 11784 . . . 11806 | − | 3′ Flanking Genomic DNA |
| 3F-69 | TTC/ | Fn/Lb_V1/ | 11785 . . . 11807 | − | 3′ Flanking Genomic DNA |
| | TTTC/ | Lb_V2/Lb | | | |
| | TTTC | | | | |
| 3F-70 | TATG | Lb_V2 | 11802 . . . 11824 | − | 3′ Flanking Genomic DNA |
| 3F-71 | TTA/ | Fn/Lb_V1/ | 11835 . . . 11857 | + | 3′ Flanking Genomic DNA |
| | TTTA/ | Lb_V2/Lb | | | |
| | TTTA | | | | |
| 3F-72 | TATA | Lb_V2 | 11844 . . . 11866 | + | 3′ Flanking Genomic DNA |
| 3F-73 | TCCG | Lb_V1 | 11839 . . . 11861 | − | 3′ Flanking Genomic DNA |
| 3F-74 | TTG/ | Fn/Lb_V1/ | 11870 . . . 11892 | + | 3′ Flanking Genomic DNA |
| | TTTG/ | Lb_V2/Lb | | | |
| | TTTG | | | | |
| 3F-75 | TTCA | Lb_V1 | 11849 . . . 11871 | − | 3′ Flanking Genomic DNA |
| 3F-76 | TTC | Fn | 11850 . . . 11872 | − | 3′ Flanking Genomic DNA |
| 3F-77 | TTA/ | Fn/Lb_V1/ | 11868 . . . 11890 | − | 3′ Flanking Genomic DNA |
| | TTTA/ | Lb_V2/Lb | | | |
| | TTTA | | | | |
| 3F-78 | TTG/ | Fn/Lb_V1/ | 11881 . . . 11903 | − | 3′ Flanking Genomic DNA |
| | TTTG/ | Lb_V2/Lb | | | |
| | TTTG | | | | |
| 3F-79 | TATC | Lb_V2 | 11917 . . . 11939 | + | 3′ Flanking Genomic DNA |
| 3F-80 | TATC | Lb_V2 | 11921 . . . 11943 | + | 3′ Flanking Genomic DNA |
| 3F-81 | TTA | Fn | 11925 . . . 11947 | + | 3′ Flanking Genomic DNA |
| 3F-82 | TTC | Fn | 11935 . . . 11957 | + | 3′ Flanking Genomic DNA |
| 3F-83 | TTCC | Lb_V1 | 11936 . . . 11958 | + | 3′ Flanking Genomic DNA |
| 3F-84 | TTG | Fn | 11924 . . . 11946 | − | 3′ Flanking Genomic DNA |
| 3F-85 | TTG | Fn | 11929 . . . 11951 | − | 3′ Flanking Genomic DNA |
| 3F-86 | TTG | Fn | 11934 . . . 11956 | − | 3′ Flanking Genomic DNA |
| 3F-87 | TTC/ | Fn/Lb_V1/ | 11937 . . . 11959 | − | 3′ Flanking Genomic DNA |
| | TTTC/ | Lb_V2/Lb | | | |
| | TTTC | | | | |
| 3F-88 | TATG | Lb V2 | 11967 . . . 11989 | − | 3′ Flanking Genomic DNA |
| 3F-89 | TTA/ | Fn/Lb_V1/ | 11969 . . . 11991 | − | 3′ Flanking Genomic DNA |
| | TTTA/ | Lb_V2/Lb | | | |
| | TTTA | | | | |
| 3F-90 | TTG | Fn | 12001 . . . 12023 | + | 3′ Flanking Genomic DNA |
| 3F-91 | TTA/ | Fn/Lb_V1/ | 12009 . . . 12031 | + | 3′ Flanking Genomic DNA |
| | TTTA/ | Lb_V2/Lb | | | |
| | TTTA | | | | |
| 3F-92 | TATA | Lb_V2 | 12011 . . . 12033 | + | 3′ Flanking Genomic DNA |
| 3F-93 | TTA | Fn | 11996 . . . 12018 | − | 3′ Flanking Genomic DNA |
| 3F-94 | TTA/ | Fn/Lb_V1/ | 12003 . . . 12025 | − | 3′ Flanking Genomic DNA |
| | TTTA/ | Lb_V2/Lb | | | |
| | TTTA | | | | |
| 3F-95 | TTG | Fn | 12033 . . . 12055 | + | 3′ Flanking Genomic DNA |
| 3F-96 | TTG/ | Fn/Lb_V1/ | 12014 . . . 12036 | − | 3′ Flanking Genomic DNA |
| | TTTG/ | Lb_V2/Lb | | | |
| | TTTG | | | | |
| 3F-97 | TTA | Fn | 12019 . . . 12041 | − | 3′ Flanking Genomic DNA |
| 3F-98 | TTG/ | Fn/Lb_V1/ | 12034 . . . 12056 | − | 3′ Flanking Genomic DNA |
| | TTTG/ | Lb_V2/Lb | | | |
| | TTTG | | | | |
| 3F-99 | TTC | Fn | 12076 . . . 12098 | + | 3′ Flanking Genomic DNA |
| 3F-100 | TTCG | Lb_V1 | 12077 . . . 12099 | + | 3′ Flanking Genomic DNA |
| 3F-101 | TATG | Lb_V2 | 12059 . . . 12081 | − | 3′ Flanking Genomic DNA |
| 3F-102 | TTA/ | Fn/Lb_V1/ | 12061 . . . 12083 | − | 3′ Flanking Genomic DNA |
| | TTTA/ | Lb_V2/Lb | | | |
| | TTTA | | | | |
| 3F-103 | TTC | Fn | 12103 . . . 12125 | + | 3′ Flanking Genomic DNA |
| 3F-104 | TTG | Fn | 12106 . . . 12128 | + | 3′ Flanking Genomic DNA |
| 3F-105 | TTG/ | Fn/Lb_V1/ | 12088 . . . 12110 | − | 3′ Flanking Genomic DNA |
| | TTTG/ | Lb_V2/Lb | | | |
| | TTTG | | | | |
| 3F-106 | TTA | Fn | 12119 . . . 12141 | + | 3′ Flanking Genomic DNA |
| 3F-107 | TTC | Fn | 12123 . . . 12145 | + | 3′ Flanking Genomic DNA |
| 3F-108 | TTCC | Lb_V1 | 12124 . . . 12146 | + | 3′ Flanking Genomic DNA |
| 3F-109 | TCCA | Lb V1 | 12125 . . . 12147 | + | 3′ Flanking Genomic DNA |
| 3F-110 | TATG | Lb_V2 | 12103 . . . 12125 | − | 3′ Flanking Genomic DNA |
| 3F-111 | TTA/ | Fn/Lb_V1/ | 12105 . . . 12127 | − | 3′ Flanking Genomic DNA |
| | TTTA/ | Lb_V2/Lb | | | |
| | TTTA | | | | |
| 3F-112 | TTG/ | Fn/Lb_V1/ | 12109 . . . 12131 | − | 3′ Flanking Genomic DNA |
| | TTTG/ | Lb_V2/Lb | | | |
| | TTTG | | | | |
| 3F-113 | TCCC | Lb_V1 | 12113 . . . 12135 | − | 3′ Flanking Genomic DNA |
| 3F-114 | TTCC | Lb V1 | 12121 . . . 12143 | − | 3′ Flanking Genomic DNA |
| 3F-115 | TTC/ | Fn/Lb_V1/ | 12122 . . . 12144 | − | 3′ Flanking Genomic DNA |
| | TTTC/ | Lb_V2/Lb | | | |
| | TTTC | | | | |
| 3F-116 | TTG | Fn | 12126 . . . 12148 | − | 3′ Flanking Genomic DNA |
| 3F-117 | TATG | Lb_V2 | 12131 . . . 12153 | − | 3′ Flanking Genomic DNA |
| 3F-118 | TTA/ | Fn/Lb_V1/ | 12133 . . . 12155 | − | 3′ Flanking Genomic DNA |
| | TTTA/ | Lb_V2/Lb | | | |
| | TTTA | | | | |
| 3F-119 | TTCC | Lb_V1 | 12146 . . . 12168 | − | 3′ Flanking Genomic DNA |
| 3F-120 | TTC/ | Fn/Lb_V1/ | 12147 . . . 12169 | − | 3′ Flanking Genomic DNA |
| | TTTC/ | Lb_V2/Lb | | | |
| | TTTC | | | | |

gRNAs which include a gRNA repeat of GAATTTCTACTAAGTGTAGAT (SEQ ID NO:49) for LbCas12a, or GTAATTTCTACTGTTGTAGAT (SEQ ID NO:50) for FnCas12a+an OgRRS sequence (as shown in the fourth column of Table 18)+TTTTTTT (poly-T transcript termination region) can be used to target the Cas12a nuclease to cut within both the OgRRS and CgRRS sequences. Illustrative examples of such gRNAs for FnCas12a are shown in Table 19, wherein the gRNA repeat of GTAATTTCTACTGTTGTAGAT is underlined, and the poly-T transcript termination sequence of TTTTTTT is shown in italic font.

TABLE 19
Illustrative examples of gRNAs useful in
targeting FnCas12a nuclease

| gRNA | Sequence |
|---|---|
| gRNA_5F-65 | GTAATTTCTACTGTTGTAGATGTTAAATAAA |
| (SEQ ID | TAAAGAGGGTAGGTTTTTTT |
| NO: 51) | |
| gRNA_TI-605 | GTAATTTCTACTGTTGTAGATGTCATTGGAC |
| (SEQ ID | TGAACACGAGTGTTTTTTTT |
| NO: 52) | |
| gRNA_TI-934 | GTAATTTCTACTGTTGTAGATGGCCGTGACG |
| (SEQ ID | GCCACGAGCGAACTTTTTTT |
| NO: 53) | |
| gRNA_TI-946 | GTAATTTCTACTGTTGTAGATGCGCGCCAAT |
| (SEQ ID | TAAATTCAACACGTTTTTTT |
| NO: 54) | |
| gRNA_3F-41 | GTAATTTCTACTGTTGTAGATGAGTATTACA |
| (SEQ ID | GTAGAGGCTATAATTTTTTT |
| NO: 55) | |

Any of the OgRRS sequences presented in Table 18 can be used alternatively as a site to insert a CgRRS that is designed using a different OgRRS. For example, a CgRRS can be inserted into a flanking sequence to allow for the excision of the entire transgenic insertion of event Gm_CSM63714. To illustrate this approach, OgRRS 3F-41 is selected as the OgRRS that can be used to design a corresponding CgRRS 3F-41 comprising DNA fragment, and OgRRS 5F-65 is selected as the target site into which the CgRRS 3F-41 comprising DNA fragment is inserted. Using a Cas12a editing system such as with the Fn Cas12a endonuclease, the OgRRS 5F-65 site is targeted using the gRNA, gRNA_5F-65 presented in Table 19 to cut within the OgRRS 5F-65 site. The CgRRS 3F-41 comprising DNA fragment that comprises the OgRRS 3F-41 target site is then inserted within the cut site that was introduced into the OgRRS 5F-65 sequence. After selection of a transgenic event comprising the introduced CgRRS 3F-41 site, the event can be bred into another germplasm. When desired, the transgenic insert of Gm_CSM63714 can be excised from the plant using a Cas12a editing system and the gRNA, gRNA_3F-41 as presented in Table 19.

Any of the OgRRS sequences presented in Table 18 that is within the 5′ or 3′-flanking genomic sequences of event Gm_CSM63714 can be used as a site to insert a CgRRS comprising DNA fragment, comprising an OgRRS sequence that is present between the first (DMO) and the second (PAT) expression cassettes, or between the third (FT_Tv7) and the fourth (TDO) expression cassettes of the transgenic insert, to permit the excision or removal of the first (DMO) or the fourth (TDO) expression cassette, which are next to the 5′ or 3′ flanking genomic sequence, respectively, using a single gRNA. Similar methods could also be used to remove expression cassettes that are in between the first and the fourth expression cassettes (i.e., the second or the third expression cassettes, or the PAT or the FT_Tv7 cassettes). To illustrate this approach, OgRRS TI-946 is selected as the OgRRS that will be used to design a corresponding CgRRS TI-946 comprising DNA fragment, and OgRRS 3F-41 is selected as the target site into which the CgRRS TI-946 comprising DNA fragment is inserted. Using a Cas12a editing system such as the FnCas12a endonuclease, the OgRRS 3F-41 site is targeted using the gRNA, gRNA_3F-41 presented in Table 19 to cut within the OgRRS 3F-41 site. The CgRRS TI-946 comprising DNA fragment that comprises the OgRRS TI-946 target site is then inserted within the cut site that is induced into the OgRRS 3F-41 sequence. After selection of a transgenic event comprising the introduced CgRRS TI-946 site, the event can be bred into another germplasm. When desired, the TDO expression cassette which expresses the triketone dioxygenase protein can be excised from the plant using a Cas12a editing system and the gRNA, gRNA_TI-946 as presented in Table 19.

The CgRRS can be introduced into the transgenic insertion locus through multiple methods using a CRISPR system. For example, a CRISPR system can be utilized for targeting 5′ insertion of a blunt-end double-stranded DNA fragment into a genomic target site of interest such as an OgRRS that is not the OgRRS that has been selected for the design of the CgRRS. The CRISPR-mediated endonuclease activity can introduce a double stand break (DSB) in the selected genomic target site and DNA repair, such as microhomology-driven nonhomologous end-joining DNA repair, results in insertion of the blunt-end double-stranded DNA fragment into the DSB. Blunt-end double-stranded DNA fragments can be designed with 1-10 bp of microhomology, on both the 5′ and 3′ ends of the DNA fragment, that correspond to the 5′ and 3′-flanking sequence at the cut site of the protospacer in the genomic target site.

The CRISPR system can be introduced into event Gm_CSM63714 by several methods. One or more expression cassettes encoding the gRNA and/or CRISPR associated protein components of a Type I, Type II, Type III, Type IV, Type V, or Type VI CRISPR-Cas system is transiently introduced into a cell. The introduced one or more expression cassettes encoding the gRNA and/or CRISPR associated protein, along with a DNA fragment comprising the CgRRS is provided in sufficient quantity to modify the cell but does not persist after a contemplated period of time has passed or after one or more cell divisions. In such embodiments, no further steps are needed to remove or segregate the one or more expression cassettes encoding the gRNA and/or CRISPR associated protein from the modified cell. Double-stranded DNA fragments can also be transiently introduced into a cell along with one or more expression cassettes encoding the gRNA and/or CRISPR associated protein. The introduced double-stranded DNA fragments are provided in sufficient quantity to modify the cell but do not persist after a contemplated period of time has passed or after one or more cell divisions.

Alternatively, an expression construct comprising one or more expression cassettes for the expression of one or more gRNAs, and an expression construct encoding a Type I, Type II, Type III, Type IV, Type V, or Type VI CRISPR associated protein is stably transformed into event Gm_CSM63714 to modify the plant cell in the targeted region of the transgene insertion locus, to introduce the CgRRS within the desired target locus.

### Example 9, Modification of Soybean Event Gm_CSM63714 with Genome Editing Techniques Using Two Guide RNAs

This example describes how one may excise the transgenic insertion present in soybean event Gm_CSM63714 using CRISPR editing systems comprising two guide RNAs by genome editing methods. Excision of the event Gm_CSM63714 transgenic insertion or expression cassettes within SEQ ID NO:9 or SEQ ID NO:10 can be performed through genome editing using a variety of methods. In one embodiment, Clustered Regularly Interspersed Short Palindromic Repeats (CRISPR) editing systems comprising a CRISPR associated protein and two cognate guide RNAs may be used for targeted excision. The CRISPR-associated protein is an RNA guided nuclease and can be selected from a Type I CRISPR-associated protein, a Type II CRISPR-associated protein, a Type III CRISPR-associated protein, a Type IV CRISPR-associated protein, Type V CRISPR-associated protein, or a Type VI CRISPR-associated protein, such as but not limited to, Cas1, Cas1B, Cas2, Cas3, Cas4, Cas5, Cas6, Cas7, Cas8, Cas9 (also known as Csn1 and Csx12), Cas10, Cas 12a (also known as Cpf1), Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, CasX, CasY, and Mad7. The CRISPR-associated protein and two guide RNAs (gRNA) can be introduced into a plant cell comprising the soybean event Gm_CSM63714 to target a specific sequence within the transgene insertion locus. In one embodiment, the CRISPR nuclease system cleaves at two distinct guide RNA hybridization sites thereby permitting the excision of the intervening sequence. Following DNA cleavage, the genomic sequence can be repaired via a double strand break repair pathway, which may include, for example, non-homologous end-joining (NHEJ), microhomology-mediated end joining (MMEJ), homologous recombination, synthesis-dependent strand annealing (SDSA), single-strand annealing (SSA), or a combination thereof, at the genomic target site.

Sequences corresponding to the 5′ and 3′ flanking genomic sequences, and the transgenic insert of event Gm_CSM63714 (presented as SEQ ID NOs:11, 12, and 9, respectively) and the 5′ and 3′ junction regions (presented as SEQ ID NOs:1-6) were scanned for potential guide RNA recognition sites which comprise a protospacer adjacent motif (PAM) site that will be recognized by a Cas12a endonuclease, operably linked to a guide RNA hybridization site, and the results are shown in Table 18. The identified gRNA recognition sites are located within the 5′ or 3′ flanking genomic sequence, within the 5′ or 3′ junction regions, or within the transgenic insertion.

Two functional guide RNAs (gRNAs) for an RNA guided nuclease system are created to target the event Gm_CSM63714 transgenic insertion locus in a manner that will permit the excision of a fragment of DNA corresponding to either the entire transgenic insertion of event Gm_CSM63714, or a fragment within the transgenic insertion of event Gm_CSM63714 such as an expression cassette or genetic element within the transgene cassette. Illustrative examples are described below using FnCas12a editing system (see Table 19), in which the gRNAs include a gRNA repeat of GTAATTTCTACTGTTGTAGAT (SEQ ID NO:50, underlined)+an OgRRS sequence (as shown in the fourth column of Table 18)+TTTTTTT (poly-T transcript termination region, italic) to target the FnCas12a nuclease to the gRNA recognition sites. Similar methods can be used to excise either the entire transgene insertion or a fragment within the transgene insertion such as an expression cassette by selecting gRNAs targeted to the specific regions. Alternatively, the LbCas12a editing systems can be used, in which the gRNAs include a gRNA repeat of GAATTTCTACTAAGTGTAGAT (SEQ ID NO:49)+an OgRRS sequence (as shown in the third column of Table 18)+TTTTTTT (poly-T transcript termination region).

To excise the entire transgenic insertion of event Gm_CSM63714, the first gRNA targets an area in the 5′ flanking genomic sequence such as 5F-65 (Table 18), and the second gRNA targets a region in the 3′ flanking genomic sequence such as 3F-41 (Table 18). A transfer DNA (T-DNA) construct suitable for use in Agrobacterium-mediated transformation is used. The T-DNA construct comprises several expression cassettes between a left border (LB) sequence and a right border (RB) sequence. The first expression cassette comprises a promoter that is operable in a plant cell operably linked to a polynucleotide encoding a Cas12a RNA guided nuclease. A second expression cassette comprises a promoter that is operable in a plant cell operably linked to a selection marker gene, such as aadA for conferring resistance to spectinomycin and/or streptomycin. The construct also comprises expression cassettes comprising Polymerase III promoters operable in a plant cell operably linked to polynucleotides encoding the two gRNAs gRNA_5F-65 and gRNA_3F-41 (Table 19).

To facilitate excision of a fragment within the transgenic insertion of event Gm_CSM63714 such as an expression cassette near the 3′ flanking genomic sequence (the TDO cassette), the first gRNA targets an area in the 3′ flanking genomic sequence such as 3F-41 (Table 18), and the second gRNA targets a region between the FT_Tv7 expression cassette and the TDO expression cassette such as TI-946 (Table 18). A transfer DNA (T-DNA) construct suitable for use in Agrobacterium-mediated transformation is used. The T-DNA construct comprises several expression cassettes between a left border (LB) sequence and a right border (RB) sequence. The first expression cassette comprises a promoter that is operable in a plant cell operably linked to a polynucleotide encoding a Cas12a RNA guided nuclease. A second expression cassette comprises a promoter that is operable in a plant cell operably linked to a selection marker gene, such as aadA for conferring resistance to spectinomycin and/or streptomycin. The construct also comprises expression cassettes comprising Polymerase III promoters operable in a plant cell operably linked to polynucleotides encoding the two gRNAs gRNA_3F-41 and gRNA_TI-946 (Table 19).

To facilitate excision of a fragment within the transgenic insertion of event Gm_CSM63714 such as FT_Tv7 expression cassette, the first gRNA targets a region between the PAT expression cassette and the FT_Tv7 expression cassette in the transgenic insert such as TI-605 (Table 18), and the second gRNA targets a region between the FT_Tv7 expression cassette and the TDO expression cassette such as TI-934 (Table 18). A transfer DNA (T-DNA) construct suitable for use in Agrobacterium-mediated transformation is used. The T-DNA construct comprises several expression cassettes between a left border (LB) sequence and a right border (RB) sequence. The first expression cassette comprises a promoter that is operable in a plant cell operably linked to a polynucleotide encoding a Cas12a RNA guided nuclease. A second expression cassette comprises a promoter that is operable in a plant cell operably linked to a selection marker gene, such as aadA for conferring resistance to spectinomycin and/or streptomycin. The construct also comprises expression cassettes comprising Polymerase III promoters operable in a plant cell operably linked to polynucleotides encoding the two gRNAs gRNA_TI-605 and gRNA_TI-934 (Table 19).

Following Agrobacterium-mediated transformation of soybean comprising event Gm_CSM63714, and upon expression of the integrated polynucleotides, the gRNAs guide the nuclease to each of the two target sites at the transgenic insertion locus, where the nuclease creates a double-stranded break at each target site, resulting in deletion of the region between the target sites, and non-homologous end-joining repair mechanisms joins the flanking regions. Suitable methods known in the art (e.g., PCR, DNA hybridization (Southern) blots, sequencing) are used to identify plants comprising a complete deletion.

### REFERENCES

X. Chen, L. Levine and P.-Y. Kwok (1999). Fluorescence polarization in homogeneous nucleic acid analysis. Genome Res. 9:492-498.
S. Cheng, C. Fockler, W. M. Barnes and R. Higuchi (1994). Effective amplification of long targets from cloned int Proc. Natl. Acad. Sci. USA 91:5695-5699.
F. B. Dean, S. Hosono, L. Fang, X. Wu, A. F. Faruqi, P. Bray-Ward, Z. Sun, Q. Zong, Y. Du, J. Du, M. Driscoll, W. Song, S. F. Kingsmore, M. Egholm and R. S. Lasken (2002). Proc. Natl. Acad Sci. USA 99:5261-5266.
A. Fire and S. Q. Xu (1995). Rolling replication of short DNA circles. Proc. Natl. Acad Sci. USA 92:4641-4645.
L. Gao, D. B. T. Cox, W. X. Yan, J. C. Manteiga, M. W. Schneider, T. Yamano, H. Nishimasu, O. Nureki, N. Crosetto and F. Zhang (2017). Engineered Cpf1 variants with altered PAM specificities. Nature Biotechnology 35(8):789-792.
J. C. Guatelli, K. M. Whitefield, D. Y. Kwon, K. J. Barringer, D. D. Richman and T. R. Gingeras (1990). Isothermal, in vitro amplification of nucleic acids by a multienzyme reaction modeled after retroviral replication. Proc. Natl. Acad. Sci. USA 87:1874-1878.
I. M. Heap (2021). International Survey of Herbicide Resistant Weeds. http://www.weedscience.org/Home.aspx.
S. Khan, A. Latif, S. Q, Ahmad, F. Ahmad and M. Ghaffar (2010). Double haploid technique: in soybean and other species. International Journal of Applied Agricultural Research 5:649-655.
D. Liu, S. L. Daubendiek, M. Zillman, K. Ryan and E. T. Kool (1996). Rolling circle DNA synthesis: small circular oligonucleotides as efficient templates for DNA polymerases. J. Am. Chem. Soc. 118:1587-1594.
P. M. Lizardi, X. Huang, Z. Zhu, P. Bray-Ward, D. C. Thomas and D. C. Ward (1998). Mutation detection and single-molecule counting using isothermal rolling-circle amplification. Nature Genetics 19:225-232.
T. T. Nikiforov, R. B. Rendie, P. Goelet, Y.-H. Rogers, M. L. Kotewicz, S. Anderson, G. L. Trainor and M. R. Knapp (1994). Genetic bit analysis: a solid phase method for typing single nucleotide polymorphisms. Nucleic Acids Res. 22:4167-4175.
T. Notomi, H. Okayama, H. Masubuchi, T. Yonekawa, K. Watanabe, N. Amino, T. Hase (2000). Loop-mediated isothermal amplification of DNA”. Nucleic Acids Res. 28 (12): 63e-63.
M. D. Pearson, L. Nguyen, Y. Zhao, W. L. McKenna, T. J. Morin and W. B. Dunbar (2019). Past and accurate quantification of insertion-site specific transgene levels from raw seed samples using solid-state nanopore technology. PloS One 14(12):e0226719.
D. Shoup, S. Duncan and IA. Ciampitti (2016). Soybean Production Handbook. Kansas State University Agricultural Experiment Station and Cooperative Extension Service.
J. Schmutz, S. B. Cannon, J. Schlueter, J. Ma, T. Mitros, W. Nelson, D. L. Hyten, Q. Song, J. J. Thelen, J. Cheng, D. Xu, U. Hellsten, G. D. May, Y. Yu, T. Sakurai, T. Umezawa, M. K. Bhattacharyya, D. Sandhu, B. Valliyodan, E. Lindquist, M. Peto, D. Grant, S. Shu, D. Goodstein, K. Barry, M. Futrell-Griggs, B. Abernathy, J. Du, Z. Tian, L. Zhu, N. Gill, T. Joshi, M. Libault, A. Sethuraman, X.-C. Zhang, K. Shinozaki, H. T. Nguyen, R. A. Wing, P. Cregan, J. Specht, J. Grimwood, D. Rokhsar, G. Stacey, R. D. Shoemaker and S. A. Jackson (2010). Genome sequence of the palaeopolyploid soybean. Nature 463:178-183.
A. D. Taylor, L. Mataseje, C. J. Urfano, L. Schmidt, K. S. Antonation, M. R. Mulvey and C. R. Corbett (2018). Ecaluation of Oxford Nanopore's MinION Sequencing Device for Microbial Whole Genome Sequencing Applications. Scientific Reports 8:10931.
S. Tyagi and F. R. Kramer (1996). Molecular beacons: probes that fluoresce upon hybridization. Nature Biotechnology 14:303-308.
A. Verkest, S. Bourout, J. Debaveye, K. Reynaert, B. Saey, I. Van den Brande and K. D'Halluin (2019). Impact of differential DNA methylation on transgene expression in cotton (Gossypium hirsutum L.) events generated by targeted sequence insertion. Plant Biotechnology J. 17:1236-1247.
M. Vincent, Y. Xu and H. Kong (2004). Helicase-dependent isothermal DNA amplification. EMBO Reports 5(8):795-800.
Y. Wan, J. F. Petolino and J. M. Widholm (1989). Efficient Production of Doubled Haploid Plants Through Colchicine Treatment of Anther-Derived Maize Callus. Theor. Appl. Genet. 77:889-892.
C. Wang, K. C. Glenn, C. Kessenich, E. Bell, L. A. Burzio, M. S. Koch, B. Li and A. Silvanovich (2016). Safety assessment of dicamba mono-oxygenases that confer dicamba tolerance to various crops. Regulatory Toxicology and Pharmacology 81:171-182.
Y. Wang, Y. Zhao, A. Bollas, Y. Wang and K. F. Au (2021). Nanopore sequencing technology, bioinformatics and applications. Nature Biotechnology 39:1348-1365.
M. Winge (2000). Pyrosequencing-a new approach to DNA analysis. Innovation in Pharma. Tech. 00:18-24.
B. Zetsche, J. S. Gootenberg, O. O. Abudayyeh, I. M. Slaymaker, K. S. Makarova, P. Essletzbichler, S. E. Volz, J. Joung, J. v.d. Oost, A. Regev, E. V. Koonin and F. Zhang (2015). Cpf1 is a single RNA-guided endonuclease of a class 2 CRISPR-Cas system. Cell. 163(3):759-771.

1. A recombinant DNA molecule comprising a nucleotide sequence selected from the group consisting of SEQ ID NO:10, SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, a polynucleotide having a nucleotide sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO:10 or the full length of SEQ ID NO: 9, and a complete complement of any of the foregoing.

2. The recombinant DNA molecule of claim 1, wherein the recombinant DNA molecule:
a) is derived from a soybean plant, seed, plant part, plant cell, progeny plant, or commodity product comprising soybean event Gm_CSM63714, a representative sample of seed comprising the event having been deposited as ATCC Accession No. PTA-127099;
b) is comprised in a soybean plant, seed, plant part, plant cell, or progeny plant comprising soybean event Gm_CSM63714, or a commodity product produced therefrom, a representative sample of seed comprising the event having been deposited as ATCC Accession No. PTA-127099;
c) is formed by the insertion of a heterologous nucleic acid molecule into the genomic DNA of a soybean plant or soybean cell; or
d) comprises an amplicon diagnostic for the presence of soybean event Gm_CSM63714.

3.-5. (canceled)

6. A DNA molecule comprising a polynucleotide segment of sufficient length to function as a DNA probe:
a) that hybridizes specifically under stringent hybridization conditions with soybean event Gm_CSM63714 DNA in a sample, wherein detecting hybridization of the DNA molecule under the stringent hybridization conditions is diagnostic for the presence of soybean event Gm_CSM63714 in the sample; or
b) specific for detecting in a sample at least one of:
i) a 5′ junction sequence between flanking soybean genomic DNA and the transgenic insert of soybean event Gm_CSM63714;
ii) a 3′ junction sequence between the transgenic insert of soybean event Gm_CSM63714 and flanking soybean genomic DNA;
iii) SEQ ID NO:9; or
iv) a fragment of SEQ ID NO:9 comprising a sufficient length of contiguous nucleotides of SEQ ID NO:9 to identify the sequence as a fragment of the transgenic insert of Gm_CSM63714.

7. (canceled)

8. The DNA molecule of claim 6, wherein;
a) the DNA probe comprises SEQ ID NO:16;
b) the DNA molecule comprises a nucleotide sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, and a complement of any of the foregoing; or
c) the sample is derived from a soybean plant, seed, plant part, plant cell, progeny plant, or commodity product.

9.-10. (canceled)

11. A pair of DNA molecules comprising a first DNA molecule and a second DNA molecule, wherein the first and the second DNA molecules comprise a fragment of SEQ ID NO:10 or a complement thereof and function as DNA primers when used together in an amplification reaction with DNA comprising soybean event Gm_CSM63714 to produce an amplicon diagnostic for soybean event Gm_CSM63714 in a sample.

12. The pair of DNA molecules of claim 11, wherein;
a) the first and the second DNA molecules comprise SEQ ID NO:14 and SEQ ID NO:15; or
b) the amplicon comprises a nucleotide sequence selected from the group consisting of:
SEQ ID NO:1;
SEQ ID NO:2;
SEQ ID NO:3;
SEQ ID NO:4;
SEQ ID NO:5;
SEQ ID NO:6;
SEQ ID NO:7;
SEQ ID NO:8;
SEQ ID NO:9;
SEQ ID NO:10; and
a fragment of any of SEQ ID NO:1, SEQ ID NO:2, SEQ ID NO:3, SEQ ID NO:4, SEQ ID NO:5, SEQ ID NO:6, SEQ ID NO:7, SEQ ID NO:8, wherein the fragment is at least 10 nucleotides in length and comprises nucleotides 1,000-1,001 or 11,196-11,197 of SEQ ID NO:10.

13. (canceled)

14. A method of detecting the presence of soybean event Gm_CSM63714 in a sample derived from a soybean seed, plant, plant part, plant cell, progeny plant, or commodity product, the method comprising:
a) contacting the sample with the DNA molecule that functions as a DNA probe of claim 6;
subjecting the sample and the DNA molecule that functions as a probe to stringent hybridization conditions; and
detecting the hybridization of the DNA molecule that functions as a probe to a DNA molecule in the sample,
wherein the hybridization of the DNA molecule that functions as a probe to the DNA molecule in the sample is diagnostic for the presence of soybean event Gm_CSM63714 in the sample;
b) contacting the sample with the pair of DNA molecules of claim 11;
performing an amplification reaction sufficient to produce a DNA amplicon; and
detecting the presence of the DNA amplicon;
wherein the DNA amplicon comprises at least one of:
a 5′ junction sequence between flanking soybean genomic DNA and the transgenic insert of soybean event Gm_CSM63714,
a 3′ junction sequence between flanking soybean genomic DNA and the transgenic insert of soybean event Gm_CSM63714,
SEQ ID NO: 9, and
a fragment of SEQ ID NO: 9 comprising a sufficient length of contiguous nucleotides of SEQ ID NO: 9 to identify the sequence as a fragment of the transgenic insert of Gm_CSM63714; and
wherein the presence of the DNA amplicon indicates the presence of soybean event Gm_CSM63714 in the sample;
c) contacting the sample with the DNA molecule of claim 6; and
performing a sequencing reaction to produce a target sequence,
wherein the target sequence comprises a nucleotide sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, a complete complement of any thereof, and a fragment of any of SEQ ID NO:1, SEQ ID NO:2, SEQ ID NO:3, SEQ ID NO:4, SEQ ID NO:5, SEQ ID NO:6, SEQ ID NO:7, SEQ ID NO:8, and SEQ ID NO:10 that is at least 10 nucleotides long and comprises nucleotides 1,000-1,001 or 11,196-11,197 of SEQ ID NO:10; or
d) contacting the sample with at least one antibody specific for at least one protein encoded by soybean event Gm_CSM63714; and
detecting binding of the antibody to the protein in the sample,
wherein the binding of the antibody indicates the presence of soybean event Gm_CSM63714 in the sample.

15. (canceled)

16. The method of claim 14, wherein:
a) the DNA amplicon is at least 10 nucleotides in length, at least 11 nucleotides in length, at least 12 nucleotides in length, at least 13 nucleotides in length, at least 14 nucleotides in length, at least 15 nucleotides in length, at least 16 nucleotides in length, at least 17 nucleotides in length, at least 18 nucleotides in length, at least 19 nucleotides in length, at least 20 nucleotides in length, at least 25 nucleotides in length, at least 30 nucleotides in length, at least 35 nucleotides in length, at least 40 nucleotides in length, at least 45 nucleotides in length, at least 50 nucleotides in length, at least 60 nucleotides in length, at least 70 nucleotides in length, at least 80 nucleotides in length, at least 90 nucleotides in length, or at least 100 nucleotides in length; or
b) the DNA amplicon comprises a nucleotide sequence selected from the group consisting of SEQ ID NO:10; SEQ ID NO:9; SEQ ID NO:8; SEQ ID NO:7; SEQ ID NO:6; SEQ ID NO:5; SEQ ID NO:4; SEQ ID NO:3; SEQ ID NO:2; SEQ ID NO:1; and a fragment of any of SEQ ID NO:10, SEQ ID NO:8, SEQ ID NO:7, SEQ ID NO:6, SEQ ID NO:5, SEQ ID NO:4, SEQ ID NO:3, SEQ ID NO:2, and SEQ ID NO:1 that is at least 10 nucleotides in length and comprises nucleotides 1,000-1,001 or 11,196-11,197 of SEQ ID NO:10.

17.-19. (canceled)

20. A detection kit for detecting the presence of soybean event Gm_CSM63714 in a sample, wherein the kit comprises:
a) the pair of DNA primers of claim 11;
b) the DNA molecule that functions as a probe of claim 6; or
c) at least one antibody specific for at least one protein encoded by soybean event Gm_CSM63714; wherein detecting binding of the at least one antibody to the at least one protein encoded by soybean event Gm_CSM63714 in a sample is diagnostic for the presence of soybean event Gm_CSM63714 in the sample.

21. (canceled)

22. A soybean plant, plant seed, plant part, or plant cell comprising;
a) the recombinant DNA molecule of claim 1; or
b) soybean event Gm_CSM63714, a representative sample of seed comprising soybean event Gm_CSM63714 having been deposited under ATCC Accession No. PTA-127099.

23. The soybean plant, plant seed, plant part, or plant cell of claim 22, wherein:
a) the plant, plant seed, plant part, or plant cell expresses at least one herbicide tolerance gene selected from the group consisting of dicamba monooxygenase (DMO), phosphinothricin N-acetyltransferase (PAT), alpha-ketoglutarate-dependent non-heme iron dioxygenase variant FT_Tv7, triketone dioxygenase (TDO), and any combination thereof;
b) the plant, plant seed, plant part, or plant cell is tolerant to at least one herbicide selected from the group consisting of benzoic acid auxins, phenoxy auxins, glutamine synthetase inhibitors, β-triketone HPPD inhibitors, and combinations of any thereof;
c) the plant, plant seed, plant part, or plant cell further comprises an additional transgene for tolerance to at least one additional herbicide;
d) the plant, plant seed, plant part, or plant cell is further defined as a progeny plant of any generation of a soybean plant comprising soybean event Gm_CSM63714, or a soybean plant part, plant seed, or plant cell derived therefrom; and/or
e) the plant part comprises a microspore, pollen, an anther, an ovule, an ovary, a flower, a pod, an embryo, a stem, a leaf, a root, or a callus.

24.-27. (canceled)

28. The soybean plant, plant seed, plant part, or plant cell of claim 22, wherein the at least one additional herbicide is glyphosate and/or wherein the additional transgene comprises a polynucleotide sequence encoding a protein having the amino acid sequence of SEQ ID NO:57.

29.-33. (canceled)

34. A method for controlling or preventing weed growth in an area, the method comprising planting soybean comprising event Gm_CSM63714 in the area and applying an effective amount of at least one herbicide selected from the group consisting of dicamba, glufosinate, 2,4-D, a β-triketone HPPD inhibitor, and any combination thereof, to control weeds in the area without injury to the soybean or with less than about 10% injury to the soybean.

35. The method of claim 34, wherein;
a) applying the effective amount of at least one herbicide comprises applying at least two or more herbicides selected from the group consisting of dicamba, glufosinate, 2,4-D, a β-triketone HPPD inhibitor, and any combination thereof over a growing season;
b) the β-triketone HPPD inhibitor is selected from the group consisting of mesotrione, benzobicyclon (BBC), tembotrione, sulcotrione, tefuryltrione, and any combination thereof; or
c) the effective amount of dicamba is about 0.5 lb/acre to about 2 lb/acre over a growing season; the effective amount of glufosinate is about 0.4 lb/acre to about 1.6 lb/acre over a growing season; the effective amount of 2,4-D is about 0.5 lb/acre to about 4 lb/acre over a growing season; and/or the β-triketone HPPD inhibitor comprises mesotrione and the effective amount of mesotrione is about 0.09 lb/acre to about 0.36 lb/acre over a growing season.

36.-37. (canceled)

38. A method for controlling volunteer soybean comprising soybean event Gm_CSM63714 in an area, the method comprising applying an herbicidally effective amount of at least one herbicide other than dicamba, glufosinate, 2,4-D, or a β-triketone HPPD inhibitor, wherein the herbicide application prevents growth of soybean comprising soybean event Gm_CSM63714.

39.-40. (canceled)

41. A method of obtaining a seed of a soybean plant or a soybean plant that is tolerant to benzoic acid auxins, phenoxy auxins, inhibitors of glutamine synthetase, β-triketone HPPD inhibitors, or any combination thereof, the method comprising:
a) obtaining a population of progeny seed or plants grown therefrom, at least one of which comprises soybean event Gm_CSM63714; and
b) identifying at least a first progeny seed or plant grown therefrom that comprises soybean event Gm_CSM63714.

42.-45. (canceled)

46. A method of determining the zygosity of a soybean plant, plant part, plant seed, or plant cell comprising soybean event Gm_CSM63714, the method comprising:
a) contacting a sample comprising DNA derived from the soybean plant, plant part, plant seed, or plant cell with a primer set capable of producing a first amplicon diagnostic for the presence of soybean event Gm_CSM63714 and a second amplicon diagnostic for the wild-type soybean genomic DNA not comprising soybean event Gm_CSM63714;
performing a nucleic acid amplification reaction; and
detecting the first amplicon and the second amplicon, wherein the presence of both amplicons indicates that the plant, plant part, seed or cell is heterozygous for soybean event Gm_CSM63714, and the presence of only the first amplicon indicates that the plant, plant part, seed, or cell is homozygous for soybean event Gm_CSM63714; or
b) contacting a sample comprising DNA derived from the soybean plant, plant part, plant seed, or plant cell with a probe set comprising at least a first probe that specifically hybridizes to soybean event Gm_CSM63714, and at least a second probe that specifically hybridizes to soybean genomic DNA that was disrupted by insertion of the heterologous DNA of soybean event Gm_CSM63714 but does not hybridize to soybean event Gm_CSM63714; and
hybridizing the probe set with the sample under stringent hybridization conditions, wherein detecting hybridization of only the first probe under the hybridization conditions is diagnostic for a soybean plant, plant part, seed or plant cell homozygous for soybean event Gm_CSM63714, and wherein detecting hybridization of both the first probe and the second probe under the hybridization conditions is diagnostic for a soybean plant, plant part, seed, or plant cell heterozygous for soybean event Gm_CSM63714.

47.-49. (canceled)

50. A DNA construct comprising:
a) a first expression cassette, a second expression cassette, a third expression cassette and a fourth expression cassette, wherein:
the first expression cassette comprises in operable linkage i) ubiquitin (UB3) promoter, leader, and intron sequences from Arabidopsis thaliana, ii) a chloroplast transit peptide coding sequence of APG6 (Albino and Pale Green 6) from Arabidopsis thaliana, iii) a codon-optimized dicamba monooxygenase coding sequence (DMO) from Stenotrophomonas maltophilia, and iv) a 3′ UTR sequence of the aluminum-induced Sali3-2 protein from Medicago truncatula;
the second expression cassette comprises in operable linkage i) a promoter and an intron sequence derived from multiple promoter and intron sequences from Arabidopsis thaliana, ii) a codon-optimized phosphinothricin N-acetyltransferase (PAT) coding sequence from Streptomyces viridochromogene, and iii) a 3′ UTR of a small heat shock protein (Hsp20) from Medicago truncatula;
the third expression cassette comprises in operable linkage i) polyubiquitin (UBQ10) promoter, leader, and intron sequences from Arabidopsis thaliana, ii) an alpha-ketoglutarate-dependent non-heme iron dioxygenase variant coding sequence (FT_Tv7) from Sphingobium herbicidovorans, and iii) a 3′ UTR sequence of a putative protein from Medicago truncatula, and
the fourth expression cassette comprises in operable linkage i) promoter, leader, and intron sequences derived from multiple promoter, leader and intron sequences from Arabidopsis thaliana, ii) a codon-optimized coding sequence of the triketone dioxygenase (TDO) from Oryza sativa, and iii) a 3′ UTR sequence derived from multiple 3′ UTR sequences from Zea mays;
b) a first expression cassette, a second expression cassette, a third expression cassette and a fourth expression cassette, wherein: the first expression cassette comprises a dicamba monooxygenase coding sequence;
the second expression cassette comprises a phospinothricin N-acetyltransferase (PAT) coding sequence;
the third expression cassette comprises an alpha-ketoglutarate-dependent non-heme iron dioxygenase variant coding sequence (FT_Tv7) capable of degrading 2,4-D; and the fourth expression cassette comprises a triketone dioxygenase (TDO) coding sequence; and wherein the DNA construct further comprises at the 5′ or 3′ end of said construct (i) at least 50 contiguous nucleotides of SEQ ID NO: 11 or SEQ ID NO:98; or (ii) at least 50 contiguous nucleotides of SEQ ID NO: 12 or SEQ ID NO:99; or
c) a polynucleotide having a sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO: 9; and wherein the DNA construct comprises at the 5′ or 3′ end of said construct (i) at least 50 contiguous nucleotides of SEQ ID NO: 11 or SEQ ID NO:98; or (ii) at least 50 contiguous nucleotides of SEQ ID NO: 12 or SEQ ID NO:99.

51. The DNA construct of claim 50: wherein
a) the DNA construct comprises SEQ ID NO:9;
b) the DNA construct further comprises at the 5′ or 3′ end of said construct: i) at least 50 contiguous nucleotides of SEQ ID NO: 11 or SEQ ID NO:98; or ii) at least 50 contiguous nucleotides of SEQ ID NO: 12 or SEQ ID NO:99;
c) the DNA construct comprises at least 50 contiguous nucleotides of SEQ ID NO: 11 or SEQ ID NO:98 at the 5′ end of the construct and at least 50 contiguous nucleotides of SEQ ID NO: 12 or SEQ ID NO:99 at the 3′ end of the construct;
d) the construct comprises at the 5′ end of said construct one or more nucleotide sequences selected from SEQ ID NOs:58-77 and SEQ ID NOs:100-139; or
e) the construct comprises at the 3′ end of said construct one or more nucleotide sequences selected from SEQ ID NOs:78-97 and SEQ ID NOs:140-179.

52. A method of improving tolerance to at least one herbicide selected from the group consisting of benzoic acid auxins, phenoxy auxins, inhibitors of glutamine synthetase, β-triketone HPPD inhibitors, and any combination thereof in a soybean plant comprising:
a) inserting the DNA construct of claim 50 or 51 into the genome of a soybean cell;
b) generating a soybean plant from the soybean cell; and
c) selecting a soybean plant comprising the DNA construct.

53.-54. (canceled)

55. A soybean plant, plant seed, plant part, or plant cell:
a) tolerant to herbicides with at least three different herbicide modes of action at a single genomic location; or
b) tolerant to at least one herbicide selected from the group consisting of benzoic acid auxins, phenoxy auxins, glutamine synthetase inhibitors, β-triketone HPPD inhibitors, and any combination thereof, wherein the soybean plant, plant seed, plant part, or plant cell comprises the DNA construct of claim 50.

56.-65. (canceled)

66. A commodity product comprising;
a) the recombinant DNA molecule of claim 1; or
b) the DNA construct of claim 50.

67.-69. (canceled)

70. A method of controlling, preventing, or reducing the development of herbicide-tolerant weeds comprising:
a) cultivating in a crop growing environment a soybean plant comprising transgenes that provide tolerance to herbicides with at least three different herbicide modes of action at a single genomic location; or
b) cultivating in a crop growing environment a soybean plant comprising the DNA construct of claim 50 or 51 for providing tolerance to herbicides with at least three different herbicide modes of action at a single genomic location; and applying to the crop growing environment at least one herbicide selected from the group consisting of dicamba, glufosinate, 2,4-D, a β-triketone HPPD inhibitor, and any combination thereof, wherein the soybean plant is tolerant to the at least one herbicide.

71.-75. (canceled)

76. A method of reducing loci for soybean breeding by inserting transgenes at a single genomic location for tolerance to at least three different classes of herbicides.

77.-85. (canceled)

86. A soybean plant, plant cell, plant part, or plant seed comprising a recombinant DNA construct integrated in chromosome 13, wherein the recombinant DNA construct confers tolerance to at least one herbicide selected from the group consisting of benzoic acidauxins, phenoxy auxins, glutamine synthetase inhibitors, β-triketone HPPD inhibitors, and combinations of any thereof, and wherein the recombinant DNA construct is integrated in a position of said chromosome flanked by at least 50 contiguous nucleotides of SEQ ID NO:11 or SEQ ID NO:98 and 50 contiguous nucleotides of SEQ ID NO:12 or SEQ ID NO:99.

87.-89. (canceled)
